SEQUENCE LISTING (207 sequences)

SEQ ID NO: 1 - RNA, 333 nt, synthetic construct
  source 1..333: mol_type = other RNA
tgataataggctggagcctcggtggcctagcttcttgccccttgggcctccccccagcccctcctccccttcctgcacccgtacccccgtggtctttgaataaagtctgagtgggcggccaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 2 - RNA, 333 nt, synthetic construct
  source 1..333: mol_type = other RNA
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggaggctggagcctcggtggcctagcttcttgccccttgggcctccccccagcccctcctccccttcctgcacccgtacccccgtggtctttgaataaagtctgagtgggcggc

SEQ ID NO: 3 - RNA, 223 nt, synthetic construct
  source 1..223: mol_type = other RNA
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 4 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - skipped

SEQ ID NO: 6 - RNA, 57 nt, synthetic construct
  source 1..57: mol_type = other RNA
gggaaataagagagaaaagaagagtaagaagaaatataagaccccggcgccgccacc

SEQ ID NO: 7 - RNA, 121 nt, synthetic construct
  source 1..121: mol_type = other RNA
ggatccgtgatcggaaacgtgagatccacctcagatccgctaggacacccgcagatcgagaagaaggcgaattaagagagaaaagaagagtaagaagaaatataagacaccggtcgccacc

SEQ ID NO: 8 - RNA, 236 nt, synthetic construct
  source 1..236: mol_type = other RNA
gggaaacggcaagcagcagctgccgctgttttgtcttaaccggttgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggagccccggcgccgccacc

SEQ ID NO: 9 - RNA, 73 nt, synthetic construct
  source 1..73: mol_type = other RNA
gggaaataagagagaaaaagagagagggagaggagaagagtaagaagaaatataagaccccggcgccgccacc

SEQ ID NO: 10 - RNA, 73 nt, synthetic construct
  source 1..73: mol_type = other RNA
gggaaataagagagaaaagaagagtaagaaagagagagggagaggagaaatataagaccccggcgccgccacc

SEQ ID NO: 11 - RNA, 73 nt, synthetic construct
  source 1..73: mol_type = other RNA
gggaaataagagagaaaagaagagtaagaagaaatataagacagagagagggagaggacccggcgccgccacc

SEQ ID NO: 12 - RNA, 105 nt, synthetic construct
  source 1..105: mol_type = other RNA
gggaaatttttttttgatattataagagtttttttttgatattaagaaaatttttttttgatattagaagagtaagaagaaatataagaccccggcgccgccacc

SEQ ID NO: 13 - RNA, 105 nt, synthetic construct
  source 1..105: mol_type = other RNA
gggaaataagagtttttttttgatattaagaaaatttttttttgatattagaagagtttttttttgatattataagaagaaatataagaccccggcgccgccacc

SEQ ID NO: 14 - RNA, 105 nt, synthetic construct
  source 1..105: mol_type = other RNA
gggaaataagagagaaaatttttttttgatattagaagagtttttttttgatattataagaatttttttttgatattagaaatataagaccccggcgccgccacc

SEQ ID NO: 15 - RNA, 105 nt, synthetic construct
  source 1..105: mol_type = other RNA
gggaaataagagagaaaagaagagtttttttttgatattataagaatttttttttgatattagaaatatttttttttgatattataagaccccggcgccgccacc

SEQ ID NO: 16 - RNA, 105 nt, synthetic construct
  source 1..105: mol_type = other RNA
gggaaataagagagaaaagaagagtaagaatttttttttgatattagaaatatttttttttgatattataagactttttttttgatattacccggcgccgccacc

SEQ ID NO: 17 - RNA, 105 nt, synthetic construct
  source 1..105: mol_type = other RNA
gggaaataagagagaaaagaagagtaagaagaaatatttttttttgatattataagactttttttttgatattacccggctttttttttgatattagccgccacc

SEQ ID NO: 18 - RNA, 19 nt, synthetic construct
  source 1..19: mol_type = other RNA
gggccctgaagaagggccc

SEQ ID NO: 19 - RNA, 21 nt, synthetic construct
  source 1..21: mol_type = other RNA
aatccattgcactccggattt

SEQ ID NO: 20 - RNA, 244 nt, synthetic construct
  source 1..244: mol_type = other RNA
ggatgtgtggccagttggtgctgtctgtcttcttggtgaccaggagtaatttggaccatctgggaaaccagacctcttttcacccaggctgtttcgtgtgcatcacttctgaggctgtgtgctcagtcaagaccttgccagagagttgggggattagccttaggtcgagaatattctgagcagtagtaagaaatttaaaaatcatccataatttcatcactcttattttaagagattagtttat

SEQ ID NO: 21 - protein, 117 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 22 - DNA, 351 bp, synthetic construct
  source 1..351: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctac

SEQ ID NO: 23 - protein, 198 aa, synthetic construct
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCKGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 24 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 25 - protein, 1099 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 26 - DNA, 3297 bp, synthetic construct
  source 1..3297: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 27 - protein, 1720 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSNKAPQSTGPPPAPSPGLPQPAFPPGQTAPVVFSTPQATQMNTPSQPRQHFYPSRAQPPSSAASRVQSAAPARPGPAAHVYPAGSQVMMIPSQISYPASQGAYYIPGQGRSTYVVPTQQYPVQPGAPGFYPGASPTEFGTYAGAYYPAQGVQQFPTGVAPTPVLMNQPPQIAPKRERKTIRIRDPNQGGKDITEEIMSGARTASTPTPPQTGGGLEPQANGETPQVAVIVRPDDRSQGAIIADRPGLPGPEHSPSESQPSSPSPTPSPSPVLEPGSEPNLAVLSIPGDTMTTIQMSVEESTPISRETGEPYRLSPEPTPLAEPILEVEVTLSKPVPESEFSSSPLQAPTPLASHTVEIHEPNGMVPSEDLEPEVESSPELAPPPACPSESPVPIAPTAQPEELLNGAPSPPAVDLSPVSEPEEQAKEVTASMAPPTIPSATPATAPSATSPAQEEEMEEEEEEEEGEAGEAGEAESEKGGEELLPPESTPIPANLSQNLEAAAATQVAVSVPKRRRKIKELNKKEAVGDLLDAFKEANPAVPEVENQPPAGSNPGPESEGSGVPPRPEEADETWDSKEDKIHNAENIQPGEQKYEYKSDQWKPLNLEEKKRYDREFLLGFQFIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 28 - DNA, 5160 bp, synthetic construct
  source 1..5160: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaacaaggcccctcagagcaccggccctcctcctgcccctagccctggcctgcctcagcctgccttccctcctggccagaccgcccctgtggtgttcagcacccctcaggccacccagatgaacacacctagccagcctagacagcacttctaccctagcagagcccagcctcctagcagcgccgccagcagagtgcagtccgctgcacctgccagacctggccctgccgcccacgtgtaccctgccggcagccaggtgatgatgatcccgagtcaaatcagctaccctgcaagccagggcgcctactacatcccgggccagggcagaagcacctacgtggtgcctacccagcagtaccctgtgcagcctggcgcccctggtttctatcctggcgcaagccctaccgagttcggaacttacgccggcgcttactatccagctcagggcgtgcagcagttccctaccggcgtggcccctacccctgtgctgatgaatcagccacctcagatcgcccctaagcgcgagcgcaagaccatcagaatccgcgatcctaaccagggcggcaaggacatcaccgaggagatcatgagcggcgccagaacagcaagtactccaaccccgccacaaaccggcggcggcctggagcctcaagccaacggcgagacgccacaagtggccgtgatcgtacgccctgacgaccggagccaaggtgcaatcatcgccgataggcctggcctcccaggtccggagcacagccctagcgagtcccagccgtcttcaccatcaccaacccctagtccatcccctgttctcgaaccaggcagcgagcctaacctggccgtgctgagcataccaggtgacaccatgaccaccatccagatgagcgtggaggagagcaccccaatcagcagagaaactggagagccttacagactgtccccagagccgaccccactggccgagccaatactggaggtggaggtgaccctgagcaagcctgtgcctgagagcgagttcagctcctctccactgcaggccccaactcctctcgcaagccacaccgtggagatccacgaacctaacggcatggtaccaagcgaagatcttgagccagaggtcgaatcaagcccagaactggcccctccacctgcctgcccgtctgaatctccggtccctatcgctcctacggcacagcctgaggagctgctgaacggtgccccgagccctccagcagtggacttatccccagtatcagagcctgaagaacaggccaaggaggtaactgcctctatggcgccacctaccataccttcggcaacaccggctacagcaccatctgcgactagtccggctcaggaggaggagatggaggaagaagaagaggaagaggagggcgaggccggagaggccggtgaagccgagtccgagaagggcggcgaagaacttctccctccagagtcaactcctatccctgccaaccttagtcagaatctggaggccgccgccgctactcaggttgcagtgagcgtgccaaagagacgtcgcaagatcaaggagctgaacaagaaggaggccgtgggcgacctgctggacgccttcaaggaggcaaacccggcggtgcctgaagtggagaatcagcctccggccggatcaaaccctggtcctgagagtgaaggcagcggcgtcccaccaagacctgaagaggctgacgaaacttgggacagcaaggaggacaagatccacaacgccgagaacatccagccaggcgagcagaagtacgagtacaagagcgaccagtggaagcctcttaaccttgaagagaagaagagatacgacagagagttcctgctgggcttccagttcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 29 - protein, 1099 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSIANKATPQMAQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 30 - DNA, 3297 bp, synthetic construct
  source 1..3297: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcgcgaacaaggcgacccctcagatggcgcagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 31 - protein, 1069 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 32 - DNA, 3207 bp, synthetic construct
  source 1..3207: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 33 - protein, 921 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 34 - DNA, 2763 bp, synthetic construct
  source 1..2763: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 35 - protein, 528 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSID

SEQ ID NO: 36 - DNA, 1584 bp, synthetic construct
  source 1..1584: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgac

SEQ ID NO: 37 - protein, 600 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 38 - DNA, 1800 bp, synthetic construct
  source 1..1800: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 39 - protein, 541 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 40 - DNA, 1623 bp, synthetic construct
  source 1..1623: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 41 - protein, 642 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 42 - DNA, 1926 bp, synthetic construct
  source 1..1926: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 43 - protein, 494 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 44 - DNA, 1482 bp, synthetic construct
  source 1..1482: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 45 - protein, 719 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSENSSAASASSEAGSSRSQEIEELERFIDSYVLEYQVQGLLADKTEGDGESERTQSHISQWTADCSEPLDSSCSFSRGRAPPQQNGSKDNSLDMLGTDIWAANTFDSFSGATWDLQPEKLDFTQFHRKVRHTPKQPLPHIDREGCGKGKLEDGDGINLNDIEKVLPAWQGYHPMPHEVEIAHTKKLFRRRRNDRRRQQRPPGGNKPQQHGDHQPGSAKHNRDHQKSYQGGSAPHPSGRPTHHGYSQNRRWHHGNMKHPPGDKGEAGAHRNAKETMTIENPKLEDTAGDTGHSSLEAPRSPDTLAPVASERLPPQQSGGPEVETKRKDSILPERIGERPKITLLQSSKDRLRRRLKEKDEVAVETTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 46 - DNA, 2157 bp, synthetic construct
  source 1..2157: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgagaacagtagcgccgctagcgcgagcagcgaagccgggagcagccggtctcaggagatcgaggagctggagcggttcatcgacagctacgtgctggagtaccaggtgcagggcctgctggccgacaagaccgagggcgacggcgagagcgagcggacccagagccacatcagccagtggaccgccgactgcagcgagcccctggactctagctgctcattcagtcgtggacgggcccctccacagcagaacggcagcaaggacaacagcctggacatgctgggcaccgacatctgggccgccaacaccttcgacagcttcagcggcgccacctgggatctgcagcccgagaagctggactttacccagttccaccggaaggtgcggcacactcccaagcagcccctgccccacatcgatcgggagggctgcggcaagggcaagctggaagacggcgacggcatcaacctgaacgacatcgagaaggtgctgcctgcctggcagggctaccaccccatgccccacgaggtggagatcgcccacaccaagaagctgttccggcgacgacgcaacgaccggcgtaggcagcaacggccgcctggagggaacaagccccagcagcacggagaccaccagcccggtagcgccaagcacaaccgggaccaccagaagagctaccagggcggaagcgcaccacacccctcgggcagacccacccaccacggctacagccagaaccggcggtggcatcacggtaacatgaagcacccacccggcgacaaaggagaggccggcgctcaccgtaacgccaaggagaccatgaccatcgagaaccccaagctggaggataccgccggcgatacgggtcacagcagcctggaggcaccgcggtctcccgacaccctggcacccgtggccagcgaacggctgccaccccaacagagcggcggccctgaggttgagaccaagcggaaggacagcatcctgcccgaacggatcggtgagcggcccaagatcaccttactgcagagtagcaaggaccggctgagacggcggctgaaggagaaggacgaggtggccgtggagacaaccactccccagcagaacaagatggacaagctgatcgagatcctgaacagcatgcggaacaacagcagcgacgtggacaccaagctgaccaccttcatggaggaggcccagaacagcaccaacagcgaggagatgctgggcgagatcgtgcggaccatctaccagaaggccgtgagcgaccggagcttcgccttcaccgccgccaagctgtgcgacaagatggccctgttcatggtggagggcaccaagttccggagcttactgctgaatatgctgcagaaggacttcaccgtgcgggaggagctgcagcagcaggacgtggagcggtggctgggcttcatcaccttcctgtgcgaggtgttcggcaccatgcggagcagcaccggcgaacccttccgggtgctggtgtgccccatctacacctgcctgcgggagttgctgcagagccaggacgtgaaggaggacgccgtgctgtgctgcagcatggaactgcagagcactggccggctgctggaggagcagctgcccgagatgatgaccgagctgctcgctagcgcccgggacaagatgctgtgccccagcgagagcatgctgacccggagcctgcttctggaggtgatcgagctgcacgccaacagctggaatcccctgacccctcccatcacccagtactacaaccggaccatccagaagctgaccgcc

SEQ ID NO: 47 - protein, 323 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSLNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWN

SEQ ID NO: 48 - DNA, 969 bp, synthetic construct
  source 1..969: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagcgatggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgactagatccctactgttggaggtgatcgagctgcacgccaacagctggaac

SEQ ID NO: 49 - protein, 356 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 50 - DNA, 1068 bp, synthetic construct
  source 1..1068: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaccacccctcagcagaacaagatggacaagctgatcgagatcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagaggtggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgacgcgcagcctcctgctggaggtgatcgagctgcacgccaacagctggaaccctctgacccctcctatcacccagtactacaacagaaccatccagaagctgaccgcc

SEQ ID NO: 51 - protein, 364 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPR

SEQ ID NO: 52 - DNA, 1092 bp, synthetic construct
  source 1..1092: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgccgacggcagcaagacccaggacctgttcagaagagtgagaagcatcctgaacaagctgacccctcagatgttccagcagctgatgaagcaggtgacccagctggccatcgacaccgaggagagactgaagggcgtgatcgacctgatcttcgagaaggccatcagcgagcctaacttcagcgtggcctacgccaacatgtgccggtgcctgatggccctgaaggtgcctaccaccgagaagcctaccgtgaccgtgaacttcagaaagctgctgctgaaccggtgccagaaggagttcgagaaggacaaggacgacgacgaggtgttcgagaagaagcagaaggagatggacgaggccgccaccgccgaggagagaggcagactgaaggaggagctggaggaggccagagacatcgccagaagaagaagcctgggcaacatcaagttcatcggcgagctgttcaagctgaagatgctgaccgaggccatcatgcacgactgcgtggtgaagctgctgaagaaccacgacgaggagagcctggagtgcctgtgcagactgctgaccaccatcggcaaggacctggatttcgagaaggcgaagcctagaatggaccagtacttcaaccagatggagaagatcatcaaggagaagaagaccagcagcagaatcagattcatgctgcaggacgtgctggacctgagaggcagcaactgggtgcctaga

SEQ ID NO: 53 - protein, 1168 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSFTLTNKNVIFVAGLGGIGLDTSKELLKRDPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAELQLSAGRYHYQLVWCQK

SEQ ID NO: 54 - DNA, 3504 bp, synthetic construct
  source 1..3504: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagctcgttcacgctaacgaacaagaacgtcatcttcgtagcgggacttggcggtatcggcctagacacgtcgaaggaactactaaagcgtgacccggtagtcctccaacgtcgcgattgggagaacccgggcgtaacgcaactaaaccgtcttgcggcgcacccgccgtttgcgtcgtggcgtaactcggaggaggcgcgaacggatcgtccgtcgcaacaactacgttcgctcaacggggagtggcgcttcgcgtggttcccggcgccggaggcggtaccggagtcgtggctcgagtgcgatctaccggaggcggacacggtcgtcgtaccgtcgaactggcaaatgcacggttacgacgcgccgatatacacgaacgtcacgtacccgataacggtaaacccgccgttcgtcccgacggagaacccgacggggtgctactcgctaacgttcaacgttgacgagtcgtggttgcaagagggtcaaacgcgtatcatattcgacggtgtaaactcggcgttccacctgtggtgcaacgggcgctgggtagggtacggccaagactcgcgtctaccgtcggagttcgacctatcggcgttcctacgagcgggtgagaaccggctagcggtcatggtcctacgttggtcggacggttcgtacctcgaggaccaagacatgtggcgaatgtcgggtatcttccgcgacgtatcgctcctacacaagccgacgacgcaaatctcggacttccacgtcgcgacgcgtttcaacgacgatttctcgcgggcagtcctagaggcggaggtccaaatgtgcggggagctacgtgactacctccgtgtcacggtatcgctctggcaaggtgagacgcaagtagcgtcgggtacggcgccgttcggcggtgagatcatcgacgagcgtggtgggtacgcggaccgtgtaacgctacgtctaaacgtcgagaacccgaagctctggtcggcggagatcccgaacctataccgtgcggtcgtcgagctacatacggcggacgggacgctaatagaggcggaagcgtgcgacgtcgggtttcgagaggttcgtatagagaacgggctgctacttctaaacgggaagccgttgctcatacgtggtgtcaaccgtcacgagcaccacccgctacacggtcaagtaatggacgagcaaacgatggtacaagacatcctactaatgaagcagaacaacttcaacgcggtacgctgttcgcattacccgaaccatccgttgtggtacacgctttgcgaccgatacggtctatacgtcgtagacgaggcgaacatagagacgcacgggatggtaccgatgaatcgcctaacggacgacccgcgttggctaccggcgatgtcggagcgagtcacgcgtatggtccaacgggaccgtaaccacccgtcggtaataatctggtcgctaggcaacgaatcggggcacggggcgaaccacgacgcgctataccgttggatcaagtcggtagacccgtcgcgtccggtacaatacgaaggtggcggtgcggacacgacggcgacggacatcatctgcccgatgtacgcgcgcgtcgacgaagaccaaccgttcccggcggtaccgaagtggtcgatcaagaagtggctctcgttgccgggtgaaacgcgtccgttgatactttgcgagtacgcgcacgcgatgggcaactcgttgggtgggttcgcgaagtactggcaggcgttccgtcaatacccgcgtctacagggtgggttcgtctgggactgggtagaccaatcgctaatcaagtacgacgagaacggcaacccgtggtcggcgtacggtggggacttcggggacacgccgaacgaccgccaattctgtatgaacggcctagtcttcgcggaccgaacgccgcacccggcgttgacggaggcgaagcatcaacaacaattcttccaattccgtctatcggggcaaacgatcgaggtaacgtcggagtacttgttccggcactcggacaacgagctactacactggatggtagcactagacggcaagccgctagcgtcgggagaagtccctttggacgtcgcgccgcaaggtaagcaactaatcgagctaccggagctaccgcaaccggagtcggcgggtcaactgtggttgacggtccgtgtcgttcaaccgaacgcgacggcgtggtcggaggcgggtcacatctcggcgtggcagcagtggcgtctagcggagaacctctcggtcacgctaccggcggcgtcgcacgcgataccgcatctaacgacgtcggagatggacttctgcatcgagttggggaacaagaggtggcagttcaaccgtcaatcgggattcctatcgcaaatgtggataggtgacaagaagcaactactaacgccgctacgtgatcagttcacgcgtgctccgctagacaacgacataggtgtttcggaggcgacgcgtatagacccgaacgcgtgggtggagcggtggaaggcggcggggcactaccaagcggaggcggcgctactacagtgcacggcggacacgctagcggacgcggtattgatcacgacggcgcacgcgtggcaacaccaggggaagacgctattcatctcgcgtaagacgtaccgtatcgacggttcgggccaaatggcgatcacggtcgacgtagaggtagcgtcggacacgccgcatccggcgcgcatcggtctaaactgccaactagcgcaagtagcggagcgtgtaaactggctagggctagggccgcaagagaactatccggaccgcctaacggcggcgtgcttcgaccgttgggacctaccgctttcggacatgtataccccgtacgtcttcccgtcggagaacgggttgaggtgcgggacgcgcgagctaaactacgggccgcaccagtggcgaggggacttccaattcaacatatcgcgttactcgcaacaacaactaatggagacgtcgcaccgtcacctactacacgcggaggaggggacgtggctaaacatcgacgggttccacatgggcataggtggggacgactcgtggtcgccgtcggtctcggcggagctccaactctcggcgggtcgttaccattaccaactagtttggtgccagaag

SEQ ID NO: 55 - protein, 119 aa, synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 56 - DNA, 378 bp, synthetic construct
  source 1..378: mol_type = other DNA
atgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagagatctcatatgcatctcgag

SEQ ID NO: 57 - protein, 264 aa, synthetic construct
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGGGGSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 58 - DNA, 792 bp, synthetic construct
  source 1..792: mol_type = other DNA
atggccagcaattttacccagttcgtgctggtcgacaacggcgggaccggtgacgttaccgtggcccctagcaatttcgctaacggcgtggctgagtggatctccagcaacagccggagccaagcttacaaggtgacctgtagcgtgcggcagagcagcgcccagaaccggaagtacaccatcaaggtagaagtgcctaaggtggccacacagactgtgggcggtgtcgagctgcccgtggcagcttggaggagctatctgaatatggaactgaccatccccatcttcgccactaacagcgattgtgagctgattgttaaggccatgcaaggcctgctaaaggacgggaaccccattcccagcgccatcgctgccaacagcggcatctacggaggaggcggaagcgctagcaacttcacccagtttgtgctggtggacaacggtggcaccggcgacgtgaccgtggctcccagcaactttgccaacggtgtggccgagtggatcagcagcaattctcggagccaggcctacaaggtcacgtgcagcgtgcgccaaagcagcgctcagaatcggaagtatactatcaaggtggaggtgcccaaagtggccacccagaccgtgggtggcgtggagctgcctgtggccgcttggcgatcatacctgaacatggagctcaccatccctatcttcgcaaccaacagcgactgcgagctgatcgtgaaagccatgcagggcctgctgaaggacggcaatcccatccccagcgctatcgccgccaatagcggaatctac

SEQ ID NO: 59 - protein, 600 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKAIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 60 - DNA, 1800 bp, synthetic construct
  source 1..1800: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaaggcgataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 61 - protein, 128 aa, synthetic construct
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 62 - DNA, 384 bp, synthetic construct
  source 1..384: mol_type = other DNA
atgagcaagaccatcgtgctgagcgtgggcgaggccaccagaaccctgaccgagatccagagcaccgccgacagacagatcttcgaggagaaggtgggccctctggtgggcagactgagactgaccgccagcctgagacagaacggcgccaagaccgcctacagagtgaacctgaagctggaccaggccgacgtggtggactgcagcaccagcgtgtgcggcgagctgcctaaggtgagatacacccaggtgtggagccacgacgtgaccatcgtggccaacagcaccgaggccagcagaaagagcctgtacgacctgaccaagagcctggtggtgcaggccaccagcgaggacctggtggtgaacctggtgcctctgggcaga

SEQ ID NO: 63 - protein, 667 aa, synthetic construct
MGSSHHHHHHSQDLEVLFQGPHMGRSRLLEDFRNNRYPNLQLREIAGHIMEFSQDQHGSRFIQLKLERATPAERQLVFNEILQAAYQLMVDVFGSYVIEKFFEFGSLEQKLALAERIRGHVLSLALQMYGSRVIEKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYASYVVEKMIDVAEPGQRKIVMHKIRPHIMEFSQDQHGSRFIELKLERATPAERQLVFNEILQAAYQLMVDVFGCYVIQKFFEFGSLEQKLALAERIRGHVLSLALQMYGSRVIEKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYACYVVQKMIDVAEPGQRKIVMHKIRPHIATLRKYTYGKHILAKLEKYYMKNGVDLG

SEQ ID NO: 64 - DNA, 2001 bp, synthetic construct
  source 1..2001: mol_type = other DNA
atgggcagcagccaccatcaccaccaccacagccaggacctggaggtgctgttccagggcccacacatgggccgtagccggctgctggaggacttccggaacaaccggtaccccaacctgcagctgcgggagatcgccggccacatcatggagttcagccaggaccagcacggcagccgcttcatccagctgaagttggagcgggctacacccgctgagcggcagctggtgttcaacgagatcctgcaggccgcctatcaactgatggtagacgtgttcggcagctacgtgatcgagaagttctttgagttcggcagtctggagcagaagctcgccctggccgaacggatacggggccacgtgcttagcctggccctacagatgtacggcagcagggtgatcgagaaagccctggagttcatccccagcgaccaacagaacgaaatggtgcgggagctcgacggccacgtcctgaagtgcgtgaaggaccagaacggttgccacgtggtgcagaagtgcatcgagtgcgtccagccccagtctctgcagttcattatcgacgccttcaagggccaagtgttcgccctgagtacccacccctacggaagccgggtgatcgaacggattctggagcattgcctgcccgatcagaccctgcccatcctggaggagctgcatcaacacaccgaacagcttgtgcaagaccagtacggctgctacgtgatccagcacgtgttggagcacggacggcccgaggacaagagcaagattgtggccgagatcaggggtaacgtgctggtgctgtctcagcacaaattcgccagcaacgtggtagagaagtgcgtgacccacgccagccggactgaacgggccgtgctgatcgacgaggtgtgcactatgaacgacgggccccactctgccctgtacacaatgatgaaagaccaatacgccagctacgtggtggagaagatgatcgacgtggccgagccaggccaacggaagatcgtgatgcataagatccggcctcatatcatggagtttagccaggatcaacacggcagccggttcatcgagctgaagctggaacgggccacccctgccgaacggcagctggtctttaacgagatactgcaagccgcctaccagctgatggtggacgtgtttggttgctacgttatccagaagttcttcgagttcggaagcctggagcagaaactggccctggccgagcgtatccggggccacgttctgagcttagccctgcagatgtacgggagccgggttatcgagaaggccctggaattcatccctagcgatcagcagaacgagatggtcagagagctggacggccacgtgctcaagtgcgtgaaagaccagaacggctgccacgtggtacagaagtgtatcgagtgtgtgcagccccagagcctccagtttatcattgacgcctttaagggccaggtgtttgccctgagcacacacccctacggcagccgggtaatcgagcggatcctggagcactgcctgcccgaccagaccttgcccatcctcgaggaactgcaccagcacaccgagcagctggtgcaggaccaatacggctgttacgtcatccagcacgttctggagcacggccggcctgaggacaagtctaagatcgtggccgaaatccggggcaacgtgttggtgctgagccagcacaagttcgcctcaaacgtcgtggagaagtgtgtgactcacgcctctcggaccgagcgggcagtgctgattgacgaggtgtgtaccatgaacgacggcccacacagcgccctgtacaccatgatgaaggaccagtacgcctgctacgttgtgcagaagatgattgacgtggccgaacccggtcagcgcaagattgtgatgcacaagatccggccccacatcgccaccctgcggaagtacacctacggcaagcacatcctggccaagctggagaagtactacatgaagaacggcgtggacctgggc

SEQ ID NO: 65 - protein, 667 aa, synthetic construct
MGSSHHHHHHSQDLEVLFQGPHMGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGNRVIQKALEFIPSDQQNEMVRELDGHVLKCVKDQNGCHVVQKCIECVQPQSLQFIIDAFKGQVFALSTHPYGNRVIQRILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIMEFSQDQHGNRFIQLKLERATPAERQLVFNEILQAAYQLMVDVFGNYVIQKFFEFGSLEQKLALAERIRGHVLSLALQMYGNRVIQKALEFIPSDQQNEMVRELDGHVLKCVKDQNGNHVVQKCIECVQPQSLQFIIDAFKGQVFALSTHPYGNRVIQRILEHCLPDQTLPILEELHQHTEQLVQDQYGNYVIQHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 66 - DNA, 2001 bp, synthetic construct
  source 1..2001: mol_type = other DNA
atgggcagctcccatcaccaccaccatcattcgcaggaccttgaagtcctgttccaaggtcctcatatgggtagatcaaggcttttggaagattttcgaaataaccggtatcctaacctgcagctcagggagatagcaggacacatcatggagttcagccaggaccaacacggaagcaggtttatccaactgaaactcgagagagcaacgcccgcagagcgtcagctggtgtttaacgagatcctgcaggcagcctaccagctgatggttgacgtgttcggtaactacgtaattcagaagttctttgagttcggttctctggaacagaaactcgctctggctgagcgaattcggggtcacgtgctctcactggcgctccagatgtacggcaatagagtgattcagaaggctctggaatttattccatcggaccagcagaacgaaatggtacgagaactggacggtcacgtccttaagtgtgtcaaagatcagaacggctgtcacgtagtacagaagtgcatcgagtgtgtgcagccgcagagcctccagtttatcatcgacgcctttaaagggcaggtcttcgccttgtccacccatccgtacggaaaccgagtcattcagcgcatcctggaacattgcctcccggatcagaccctgccgattctggaggagcttcaccagcacaccgagcaactagtgcaggatcagtacggctgttacgtgatacagcacgtcctggagcacggtagaccagaggacaaatcgaagattgtggccgaaatcagaggaaacgtgctggtcctttcccagcacaagttcgcttccaacgtggtggagaagtgcgtgacacacgcaagccgcaccgagagggcagtcttaatcgacgaagtgtgcacaatgaacgacggtccacactctgccttatacacaatgatgaaggaccagtacgcaaactacgtggtgcagaagatgattgacgtagctgagcctggccagcgaaagattgtcatgcataagatcaggcctcatattatggagttttctcaggatcaacacggcaatcggttcatccaattaaagctggagagggccacacctgcggagaggcagttggtctttaacgaaattctacaagccgcttaccaattaatggtggacgtctttggcaactacgtgatccagaaattcttcgagtttggaagtcttgaacagaagctggcgctggctgaaagaatcagaggtcacgtcctcagcctggcattgcaaatgtacggaaatcgagtcatccagaaagcccttgagtttatccccagcgatcaacagaacgagatggttcgcgaattggacgggcacgtgctgaagtgtgttaaggaccagaacgggaaccacgtcgtgcagaagtgcatagagtgcgtccagccacagtccctgcagtttattattgacgcatttaagggacaagtgttcgccttatctacccacccttacggcaatagggtcattcagaggatcttggaacactgtcttcccgaccagacactgccaatattggaagaactgcatcaacataccgaacagctcgtacaagatcagtacggtaattacgtcattcaacacgttctcgaacacggaaggcccgaagacaagtctaagatcgtcgcagagatacgtgggaacgtcctcgtcttaagtcagcataagttcgcaaacaacgttgtgcagaagtgtgtaacacacgcgagcagaacagagagagccgtgctgatcgacgaggtgtgtaccatgaacgacgggccacacagcgccctctacaccatgatgaaagaccaatacgctaactacgtggttcagaagatgatcgacgtcgccgaaccaggtcaacggaagatcgtgatgcacaagatcaggccacacatagccaccctgagaaagtatacgtacggtaaacatatcctggcaaagttagagaagtactatatgaagaacggcgtggacttagga

SEQ ID NO: 67 - protein, 23 aa, synthetic construct
MGNARTRRRERRAEKQAQWKAAN

SEQ ID NO: 68 - DNA, 69 bp, synthetic construct
  source 1..69: mol_type = other DNA
atgggcaacgcccggaccaggagaagagagcggcgggccgagaagcaggcccagtggaaggccgccaac

SEQ ID NO: 69 - protein, 101 aa, synthetic construct
MAVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTF

SEQ ID NO: 70 - DNA, 303 bp, synthetic construct
  source 1..303: mol_type = other DNA
atggccgtgcctgagaccagacctaaccacaccatctacatcaacaacctgaacgagaagatcaagaaggacgagctgaagaagagcctgtacgccatcttcagccagttcggccagatcctggacatcctggtgagcagaagcctgaagatgagaggccaggccttcgtgatcttcaaggaggtgagcagcgccaccaacgccctgagaagcatgcagggcttccctttctacgacaagcctatgagaatccagtacgccaagaccgacagcgacatcatcgccaagatgaagggcaccttc

SEQ ID NO: 71 - protein, 128 aa, synthetic construct
MTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 72 - DNA, 384 bp, synthetic construct
  source 1..384: mol_type = other DNA
atgaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 73 - protein, 582 aa, synthetic construct
METESGNQEKVMEEESTEKKKEVEKKKRSRVKQVLADIAKQVDFWFGDANLHKDRFLREQIEKSRDGYVDISLLVSFNKMKKLTTDGKLIARALRSSAVVELDLEGTRIRRKKPLGERPKDEDERTVYVELLPKNVNHSWIERVFGKCGNVVYISIPHYKSTGDPKGFAFVEFETKEQAAKAIEFLNNPPEEAPRKPGIFPKTVKNKPIPALRVVEEKKKKKKKKGRMKKEDNIQAKEENMDTSNTSISKMKRSRPTSEGSDIESTEPQKQCSKKKKKRDRVEASSLPEVRTGKRKRSSSEDAESLAPRSKVKKIIQKDIIKEASEASKENRDIEISTEEEKDTGDLKDSSLLKTKRKHKKKHKERHKMGEEVIPLRVLSKSEWMDLKKEYLALQKASMASLKKTISQIKSESEMETDSGVPQNTGMKNEKTANREECRTQEKVNATGPQFVSGVIVKIISTEPLPGRKQVRDTLAAISEVLYVDLLEGDTECHARFKTPEDAQAVINAYTEINKKHCWKLEILSGDHEQRYWQKILVDRQAKLNQPREKKRGTEKLITKAEKIRLAKTQQASKHIRFSEYD

SEQ ID NO: 74 - DNA, 1746 bp, synthetic construct
  source 1..1746: mol_type = other DNA
atggagaccgagagcggcaaccaggagaaggtgatggaggaggagagcaccgagaagaagaaggaggtggagaagaagaagagaagcagagtgaagcaggtgctggccgacatcgccaagcaggtggacttctggttcggcgacgccaacctgcacaaggacagattcctgagagagcagatcgagaagagcagagacggctacgtggacatcagcctgctggtgagcttcaacaagatgaagaagctgaccaccgacggcaagctgatcgccagagccctgagaagcagcgccgtggtggagctggacctggagggcaccagaatcagaagaaagaagcctctgggcgagagacctaaggacgaggacgagagaaccgtgtacgtggagctgctgcctaagaacgtgaaccacagctggatcgagagagtgttcggcaagtgcggcaacgtggtgtacatcagcatccctcactacaagagcaccggcgaccctaagggcttcgccttcgtggagttcgagaccaaggagcaggccgccaaggccatcgagttcctgaacaaccctcctgaggaggcccctagaaagcctggcatcttccctaagaccgtgaagaacaagcctatccctgccctgagagtggtggaggagaagaagaagaagaagaagaagaagggcagaatgaagaaggaggacaacatccaggccaaggaggagaacatggacaccagcaacaccagcatcagcaagatgaagagaagcagacctaccagcgagggcagcgacatcgagagcaccgagcctcagaagcagtgcagcaagaagaagaagaagagagacagagtggaggccagcagcctgcctgaggtgagaaccggcaagagaaagagaagcagcagcgaggacgccgagagcctggcccctagaagcaaggtgaagaagatcatccagaaggacatcatcaaggaggccagcgaggccagcaaggagaacagagacatcgagatcagcaccgaggaggagaaggacaccggcgacctgaaggacagcagcctgctgaagaccaagagaaagcacaagaagaagcacaaggagagacacaagatgggcgaggaggtgatccctctgagagtgctgagcaagagcgagtggatggacctgaagaaggagtacctggccctgcagaaggccagcatggccagcctgaagaagaccatcagccagatcaagagcgagagcgagatggagaccgacagcggcgtgcctcagaacaccggcatgaagaacgagaagaccgccaacagagaggagtgcagaacccaggagaaggtgaacgccaccggccctcagttcgtgagcggcgtgatcgtgaagatcatcagcaccgagcctctgcctggcagaaagcaggtgagagacaccctggccgccatcagcgaggtgctgtacgtggacctgctggagggcgacaccgagtgccacgccagattcaagacccctgaggacgcccaggccgtgatcaacgcctacaccgagatcaacaagaagcactgctggaagctggagatcctgagcggcgaccacgagcagagatactggcagaagatcctggtggacagacaggccaagctgaaccagcctagagagaagaagagaggcaccgagaagctgatcaccaaggccgagaagatcagactggccaagacccagcaggccagcaagcacatcagattcagcgagtacgac

SEQ ID NO: 75 - RNA, 22 nt, synthetic construct
  source 1..22: mol_type = other RNA
cgcattattactcacggtacga

SEQ ID NO: 76 - RNA, 22 nt, synthetic construct
  source 1..22: mol_type = other RNA
atgcccttttaacattgcactg

SEQ ID NO: 77 - RNA, 23 nt, synthetic construct
  source 1..23: mol_type = other RNA
cacaaattcggatctacagggta

SEQ ID NO: 78 - RNA, 22 nt, synthetic construct
  source 1..22: mol_type = other RNA
taaccgatttcagatggtgcta

SEQ ID NO: 79 - RNA, 24 nt, synthetic construct
  source 1..24: mol_type = other RNA
tcacaggttaaagggtctcaggga

SEQ ID NO: 80 - RNA, 22 nt, synthetic construct
  source 1..22: mol_type = other RNA
agctcccaagagcctaacccgt

SEQ ID NO: 81 - RNA, 133 nt, synthetic construct
  source 1..133: mol_type = other RNA
gctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 82 - RNA, 141 nt, synthetic construct
  source 1..141: mol_type = other RNA
tgataataggctggagcctcggtggccatgcttcttgccccttgggcctccccccagcccctcctccccttcctgcacccgtaccccccgcattattactcacggtacgagtggtctttgaataaagtctgagtgggcggc

SEQ ID NO: 83 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 84 - RNA, 142 nt, synthetic construct
  source 1..142: mol_type = other RNA
tgataataggctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 85 - RNA, 164 nt, synthetic construct
  source 1..164: mol_type = other RNA
tgataatagtccataaagtaggaaacactacagctggagcctcggtggccatgcttcttgccccttgggcctccccccagcccctcctccccttcctgcacccgtaccccccgcattattactcacggtacgagtggtctttgaataaagtctgagtgggcggc

SEQ ID NO: 86 - RNA, 188 nt, synthetic construct
  source 1..188: mol_type = other RNA
tgataatagtccataaagtaggaaacactacagctggagcctcggtggccatgcttcttgccccttgggcctccataaagtaggaaacactacatccccccagcccctcctccccttcctgcacccgtaccccctccataaagtaggaaacactacagtggtctttgaataaagtctgagtgggcggc

SEQ ID NO: 87 - RNA, 140 nt, synthetic construct
  source 1..140: mol_type = other RNA
tgataataggctggagcctcggtggccatgcttcttgccccttgggcctccccccagcccctcctccccttcctgcacccgtacccccagtagtgctttctactttatggtggtctttgaataaagtctgagtgggcggc

SEQ ID NO: 88 - RNA, 182 nt, synthetic construct
  source 1..182: mol_type = other RNA
tgataatagagtagtgctttctactttatggctggagcctcggtggccatgcttcttgccccttgggccagtagtgctttctactttatgtccccccagcccctcctccccttcctgcacccgtacccccagtagtgctttctactttatggtggtctttgaataaagtctgagtgggcggc

SEQ ID NO: 89 - RNA, 184 nt, synthetic construct
  source 1..184: mol_type = other RNA
tgataatagagtagtgctttctactttatggctggagcctcggtggccatgcttcttgccccttgggcctccataaagtaggaaacactacatccccccagcccctcctccccttcctgcacccgtacccccagtagtgctttctactttatggtggtctttgaataaagtctgagtgggcggc

SEQ ID NO: 90 - RNA, 142 nt, synthetic construct
  source 1..142: mol_type = other RNA
tgataataggctggagcctcggtggccatgcttcttgccccttgggcctccccccagcccctcctccccttcctgcacccgtacccccacccctatcacaattagcattaagtggtctttgaataaagtctgagtgggcggc

SEQ ID NO: 91 - RNA, 188 nt, synthetic construct
  source 1..188: mol_type = other RNA
tgataatagacccctatcacaattagcattaagctggagcctcggtggccatgcttcttgccccttgggccacccctatcacaattagcattaatccccccagcccctcctccccttcctgcacccgtacccccacccctatcacaattagcattaagtggtctttgaataaagtctgagtgggcggc

SEQ ID NO: 92 - RNA, 188 nt, synthetic construct
  source 1..188: mol_type = other RNA
tgataatagacccctatcacaattagcattaagctggagcctcggtggccatgcttcttgccccttgggcctccataaagtaggaaacactacatccccccagcccctcctccccttcctgcacccgtacccccacccctatcacaattagcattaagtggtctttgaataaagtctgagtgggcggc

SEQ ID NO: 93 - RNA, 142 nt, synthetic construct
  source 1..142: mol_type = other RNA
tgataatagtccataaagtaggaaacactacagctggagcctcggtggccatgcttcttgccccttgggcctccccccagcccctcctccccttcctgcacccgtacccccgtggtctttgaataaagtctgagtgggcggc

SEQ ID NO: 94 - RNA, 142 nt, synthetic construct
  source 1..142: mol_type = other RNA
tgataataggctggagcctcggtggctccataaagtaggaaacactacacatgcttcttgccccttgggcctccccccagcccctcctccccttcctgcacccgtacccccgtggtctttgaataaagtctgagtgggcggc

SEQ ID NO: 95 - RNA, 142 nt, synthetic construct
  source 1..142: mol_type = other RNA
tgataataggctggagcctcggtggccatgcttcttgccccttgggcctccataaagtaggaaacactacatccccccagcccctcctccccttcctgcacccgtacccccgtggtctttgaataaagtctgagtgggcggc

SEQ ID NO: 96 - RNA, 181 nt, synthetic construct
  source 1..181: mol_type = other RNA
tgataatagagtagtgctttctactttatggctggagcctcggtggccatgcttcttgccccttgggccagtagtgctttctactttatgtccccccagcccctctccccttcctgcacccgtacccccagtagtgctttctactttatggtggtctttgaataaagtctgagtgggcggc

SEQ ID NO: 97 - RNA, 142 nt, synthetic construct
  source 1..142: mol_type = other RNA
tgataataggctggagcctcggtggccatgcttcttgccccttccataaagtaggaaacactacatgggcctccccccagcccctcctccccttcctgcacccgtacccccgtggtctttgaataaagtctgagtgggcggc

SEQ ID NO: 98 - RNA, 142 nt, synthetic construct
  source 1..142: mol_type = other RNA
tgataataggctggagcctcggtggccatgcttcttgccccttgggcctccccccagtccataaagtaggaaacactacacccctcctccccttcctgcacccgtacccccgtggtctttgaataaagtctgagtgggcggc

SEQ ID NO: 99 - RNA, 142 nt, synthetic construct
  source 1..142: mol_type = other RNA
tgataataggctggagcctcggtggccatgcttcttgccccttgggcctccccccagcccctcctccccttctccataaagtaggaaacactacactgcacccgtacccccgtggtctttgaataaagtctgagtgggcggc

SEQ ID NO: 100 - RNA, 142 nt, synthetic construct
  source 1..142: mol_type = other RNA
tgataataggctggagcctcggtggccatgcttcttgccccttgggcctccccccagcccctcctccccttcctgcacccgtacccccgtggtctttgaataaagttccataaagtaggaaacactacactgagtgggcggc

SEQ ID NO: 101 - RNA, 164 nt, synthetic construct
  source 1..164: mol_type = other RNA
tgataatagtccataaagtaggaaacactacagctggagcctcggtggcctagcttcttgccccttgggcctccccccagcccctcctccccttcctgcacccgtaccccccgcattattactcacggtacgagtggtctttgaataaagtctgagtgggcggc

SEQ ID NO: 102 - skipped

SEQ ID NO: 103 - RNA, 142 nt, synthetic construct
  source 1..142: mol_type = other RNA
tgataataggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 104 - RNA, 141 nt, synthetic construct
  source 1..141: mol_type = other RNA
tgataataggctggagcctcggtggcctagcttcttgccccttgggcctccccccagcccctcctccccttcctgcacccgtaccccccgcattattactcacggtacgagtggtctttgaataaagtctgagtgggcggc

SEQ ID NO: 105 - RNA, 188 nt, synthetic construct
  source 1..188: mol_type = other RNA
tgataatagtccataaagtaggaaacactacagctggagcctcggtggcctagcttcttgccccttgggcctccataaagtaggaaacactacatccccccagcccctcctccccttcctgcacccgtaccccctccataaagtaggaaacactacagtggtctttgaataaagtctgagtgggcggc

SEQ ID NO: 106 - RNA, 142 nt, synthetic construct
  source 1..142: mol_type = other RNA
tgataatagtccataaagtaggaaacactacagctggagcctcggtggcctagcttcttgccccttgggcctccccccagcccctcctccccttcctgcacccgtacccccgtggtctttgaataaagtctgagtgggcggc

SEQ ID NO: 107 - RNA, 142 nt, synthetic construct
  source 1..142: mol_type = other RNA
tgataataggctggagcctcggtggctccataaagtaggaaacactacactagcttcttgccccttgggcctccccccagcccctcctccccttcctgcacccgtacccccgtggtctttgaataaagtctgagtgggcggc

SEQ ID NO: 108 - RNA, 142 nt, synthetic construct
  source 1..142: mol_type = other RNA
tgataataggctggagcctcggtggcctagcttcttgccccttgggcctccataaagtaggaaacactacatccccccagcccctcctccccttcctgcacccgtacccccgtggtctttgaataaagtctgagtgggcggc

SEQ ID NO: 109 - RNA, 142 nt, synthetic construct
  source 1..142: mol_type = other RNA
tgataataggctggagcctcggtggcctagcttcttgccccttgggcctccccccagcccctcctccccttcctgcacccgtacccccacccctatcacaattagcattaagtggtctttgaataaagtctgagtgggcggc

SEQ ID NO: 110 - RNA, 188 nt, synthetic construct
  source 1..188: mol_type = other RNA
tgataatagacccctatcacaattagcattaagctggagcctcggtggcctagcttcttgccccttgggccacccctatcacaattagcattaatccccccagcccctcctccccttcctgcacccgtacccccacccctatcacaattagcattaagtggtctttgaataaagtctgagtgggcggc

SEQ ID NO: 111 - RNA, 188 nt, synthetic construct
  source 1..188: mol_type = other RNA
tgataatagacccctatcacaattagcattaagctggagcctcggtggcctagcttcttgccccttgggcctccataaagtaggaaacactacatccccccagcccctcctccccttcctgcacccgtacccccacccctatcacaattagcattaagtggtctttgaataaagtctgagtgggcggc

SEQ ID NO: 112 - RNA, 141 nt, synthetic construct
  source 1..141: mol_type = other RNA
tgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 113 - RNA, 141 nt, synthetic construct
  source 1..141: mol_type = other RNA
tgataataggctggagcctcggtggcctagcttcttgccccttgggcctccccccagcccctcctccccttcctgcacccgtaccccccaaacaccattgtcacactccagtggtctttgaataaagtctgagtgggcggc

SEQ ID NO: 114 - RNA, 141 nt, synthetic construct; identical to SEQ ID NO: 104
  source 1..141: mol_type = other RNA

SEQ ID NO: 115 - RNA, 47 nt, synthetic construct
  source 1..47: mol_type = other RNA
gggaaataagagagaaaagaagagtaagaagaaatataagagccacc

SEQ ID NO: 116 - RNA, 70 nt, synthetic construct
  source 1..70: mol_type = other RNA
gggaaataagagtccataaagtaggaaacactacaagaaaagaagagtaagaagaaatataagagccacc

SEQ ID NO: 117 - RNA, 70 nt, synthetic construct
  source 1..70: mol_type = other RNA
gggaaataagagagaaaagaagagtaatccataaagtaggaaacactacagaagaaatataagagccacc

SEQ ID NO: 118 - RNA, 70 nt, synthetic construct
  source 1..70: mol_type = other RNA
gggaaataagagagaaaagaagagtaagaagaaatataatccataaagtaggaaacactacagagccacc

SEQ ID NO: 119 - RNA, 47 nt, synthetic construct
  source 1..47: mol_type = other RNA
aggaaataagagagaaaagaagagtaagaagaaatataagagccacc

SEQ ID NO: 120 - RNA, 57 nt, synthetic construct; identical to SEQ ID NO: 6
  source 1..57: mol_type = other RNA

SEQ ID NO: 121 - RNA, 57 nt, synthetic construct
  source 1..57: mol_type = other RNA
aggaaataagagagaaaagaagagtaagaagaaatataagaccccggcgccgccacc

SEQ ID NO: 122 - RNA, 47 nt, synthetic construct
  source 1..47: mol_type = other RNA
gggagatcagagagaaaagaagagtaagaagaaatataagagccacc

SEQ ID NO: 123 - RNA, 42 nt, synthetic construct
  source 1..42: mol_type = other RNA
gggagacaagcttggcattccggtactgttggtaaagccacc

SEQ ID NO: 124 - RNA, 47 nt, synthetic construct
  source 1..47: mol_type = other RNA
gggaattaacagagaaaagaagagtaagaagaaatataagagccacc

SEQ ID NO: 125 - RNA, 47 nt, synthetic construct
  source 1..47: mol_type = other RNA
gggaaattagacagaaaagaagagtaagaagaaatataagagccacc

SEQ ID NO: 126 - RNA, 47 nt, synthetic construct
  source 1..47: mol_type = other RNA
gggaaataagagagtaaagaacagtaagaagaaatataagagccacc

SEQ ID NO: 127 - RNA, 47 nt, synthetic construct
  source 1..47: mol_type = other RNA
gggaaaaaagagagaaaagaagactaagaagaaatataagagccacc

SEQ ID NO: 128 - RNA, 47 nt, synthetic construct
  source 1..47: mol_type = other RNA
gggaaataagagagaaaagaagagtaagaagatatataagagccacc

SEQ ID NO: 129 - RNA, 47 nt, synthetic construct
  source 1..47: mol_type = other RNA
gggaaataagagacaaaacaagagtaagaagaaatataagagccacc

SEQ ID NO: 130 - RNA, 47 nt, synthetic construct
  source 1..47: mol_type = other RNA
gggaaattagagagtaaagaacagtaagtagaattaaaagagccacc

SEQ ID NO: 131 - RNA, 47 nt, synthetic construct
  source 1..47: mol_type = other RNA
gggaaataagagagaatagaagagtaagaagaaatataagagccacc

SEQ ID NO: 132 - RNA, 47 nt, synthetic construct
  source 1..47: mol_type = other RNA
gggaaataagagagaaaagaagagtaagaagaaaattaagagccacc

SEQ ID NO: 133 - RNA, 47 nt, synthetic construct
  source 1..47: mol_type = other RNA
gggaaataagagagaaaagaagagtaagaagaaatttaagagccacc

SEQ ID NO: 134 - RNA, 92 nt, synthetic construct
  source 1..92: mol_type = other RNA
tcaagcttttggaccctcgtacagaagctaatacgactcactatagggaaataagagagaaaagaagagtaagaagaaatataagagccacc

SEQ ID NO: 135 - RNA, 236 nt, synthetic construct; identical to SEQ ID NO: 8
  source 1..236: mol_type = other RNA

SEQ ID NO: 136 - skipped

SEQ ID NO: 137 - RNA, 76 nt, synthetic construct
  source 1..76: mol_type = other RNA
gggaaatcgcaaaatttgctcttcgcgttagatttcttttagttttctcgcaactagcaagctttttgttctcgcc

SEQ ID NO: 138 - RNA, 142 nt, synthetic construct
  source 1..142: mol_type = other RNA
ggatccgtgatcggaaacgtgagatccacctcagatccgctaggacacccgcagatcgagaagaaggcgaattcgcaaaatttgctcttcgcgttagatttcttttagttttctcgcaactagcaagctttttgttctcgcc

SEQ ID NO: 139 - RNA, 126 nt, synthetic construct
  source 1..126: mol_type = other RNA
gggaaataagagagtccataaagtaggaaacactacaaaaagaagagtaatccataaagtaggaaacactacagaagaaatataatccataaagtaggaaacactacagaccccggcgccgccacc

SEQ ID NO: 140 - RNA, 123 nt, synthetic construct
  source 1..123: mol_type = other RNA
gggaaataagagagcgcattattactcacggtacgaaaaagaagagtaacgcattattactcacggtacgagaagaaatataacgcattattactcacggtacgagaccccggcgccgccacc

SEQ ID NO: 141 - RNA, 123 nt, synthetic construct
  source 1..123: mol_type = other RNA
gggaaataagagagcaaacaccattgtcacactccaaaaagaagagtaacaaacaccattgtcacactccagaagaaatataacaaacaccattgtcacactccagaccccggcgccgccacc

SEQ ID NO: 142 - skipped

SEQ ID NO: 143 - RNA, 185 nt, synthetic construct
  source 1..185: mol_type = other RNA
tgataataggctggagcctcggtgcgcattattactcacggtacgagcctagcttcttgccccttgggcccgcattattactcacggtacgatccccccagcccctcctccccttcctgcacccgtaccccccgcattattactcacggtacgagtggtctttgaataaagtctgagtgggcggc

SEQ ID NO: 144 - RNA, 185 nt, synthetic construct
  source 1..185: mol_type = other RNA
tgataatagcgcattattactcacggtacgagctggagcctcggtggcctagcttcttgccccttgggcccgcattattactcacggtacgatccccccagcccctcctccccttcctgcacccgtaccccccgcattattactcacggtacgagtggtctttgaataaagtctgagtgggcggc

SEQ ID NO: 145 - RNA, 185 nt, synthetic construct
  source 1..185: mol_type = other RNA
tgataatagcaaacaccattgtcacactccagctggagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 146 - RNA, 185 nt, synthetic construct
  source 1..185: mol_type = other RNA
tgataatagcactggtacaagggttgggagagctggagcctcggtggcctagcttcttgccccttgggcccactggtacaagggttgggagatccccccagcccctcctccccttcctgcacccgtaccccccactggtacaagggttgggagagtggtctttgaataaagtctgagtgggcggc

SEQ ID NO: 147 - RNA, 185 nt, synthetic construct
  source 1..185: mol_type = other RNA
tgataataggctggagcctcggtgatgcccttttaacattgcactggcctagcttcttgccccttgggccatgcccttttaacattgcactgtccccccagcccctcctccccttcctgcacccgtacccccatgcccttttaacattgcactggtggtctttgaataaagtctgagtgggcggc

SEQ ID NO: 148 - RNA, 188 nt, synthetic construct
  source 1..188: mol_type = other RNA
tgataataggctggagcctcggtgcacaaattcggatctacagggtagcctagcttcttgccccttgggcccacaaattcggatctacagggtatccccccagcccctcctccccttcctgcacccgtaccccccacaaattcggatctacagggtagtggtctttgaataaagtctgagtgggcggc

SEQ ID NO: 149 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 150 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 151 - RNA, 185 nt, synthetic construct
  source 1..185: mol_type = other RNA
tgataataggctggagcctcattaacactggtacaagggttgggagatataaagtaaaattcaccattttaattacactggtacaagggttgggagataataaaaataaagtatataattcactggtacaagggttgggagacaatgtacccccgtggtctttaaataaagtctaagtgggcggc

SEQ ID NO: 152 - RNA, 185 nt, synthetic construct
  source 1..185: mol_type = other RNA
tgataataggctggagcctcattaactacagtacaagggttgggagatataaagtaaaattcaccattttaattactacagtacaagggttgggagataataaaaataaagtatataattctacagtacaagggttgggagacaatgtacccccgtggtctttaaataaagtctaagtgggcggc

SEQ ID NO: 153 - RNA, 251 nt, synthetic construct
  source 1..251: mol_type = other RNA
tgataataggctggagcctcattactacagtacaagggttgggagaatataaagtaaactacagtacaagggttgggagaattcaccattttctacagtacaagggttgggagaaattataataaaaataaagtatataatctacagtacaagggttgggagatcaatgtaccccctacagtacaagggttgggagacgtggtctttaactacagtacaagggttgggagaataaagtctaagtgggcggc

SEQ ID NO: 154 - RNA, 257 nt, synthetic construct
  source 1..257: mol_type = other RNA
tgataataggctggagcctcattaatccataaagtaggaaacactacatataaagtaaaatttccataaagtaggaaacactacacaccattttaattatccataaagtaggaaacactacataataaaaataaagtccataaagtaggaaacactacatatataattcatagtccataaagtaggaaacactacatacccccgtggtcttccataaagtaggaaacactacattaaataaagtctaagtgggcggc

SEQ ID NO: 155 - RNA, 257 nt, synthetic construct
  source 1..257: mol_type = other RNA
tgataataggctggagcctcattaattacaaaagtaggaaacactacatataaagtaaaattttacaaaagtaggaaacactacacaccattttaattattacaaaagtaggaaacactacataataaaaataaagttacaaaagtaggaaacactacatatataattcatagttacaaaagtaggaaacactacatacccccgtggtctttacaaaagtaggaaacactacattaaataaagtctaagtgggcggc

SEQ ID NO: 156 - RNA, 251 nt, synthetic construct
  source 1..251: mol_type = other RNA
tgataataggctggagcctcattaactacatattactcacggtacgatataaagtaaaattctacatattactcacggtacgacaccattttaattactacatattactcacggtacgataataaaaataaagctacatattactcacggtacgatatataattcatagctacatattactcacggtacgatacccccgtggtctctacatattactcacggtacgattaaataaagtctaagtgggcggc

SEQ ID NO: 157 - RNA, 207 nt, synthetic construct
  source 1..207: mol_type = other RNA
tgataataggctggagcctcggtgcgcattattactcacggtacgagcctagcttcttgccccttgggcccgcattattactcacggtacgatccccccagcccctcctcccatgcccttttaacattgcactgcttcctgcacccgtacccccatgcccttttaacattgcactggtggtctttgaataaagtctgagtgggcggc

SEQ ID NO: 158 - RNA, 207 nt, synthetic construct
  source 1..207: mol_type = other RNA
tgataataggctggagcctcattaactacatattactcacggtacgatataaagtaaaattcaccattttctacatattactcacggtacgaaattataataaaaataaagtatatatacacttttaacattgcactgaattcatagtacccccgtggtctatacacttttaacattgcactgttaaataaagtctaagtgggcggc

SEQ ID NO: 159 - RNA, 251 nt, synthetic construct
  source 1..251: mol_type = other RNA
tgataataggctggagcctcattaaatacacttttaacattgcactgtataaagtaaaattatacacttttaacattgcactgcaccattttaattaatacacttttaacattgcactgtaataaaaataaagatacacttttaacattgcactgtatataattcatagatacacttttaacattgcactgtacccccgtggtctatacacttttaacattgcactgttaaataaagtctaagtgggcggc

SEQ ID NO: 160 - RNA, 185 nt, synthetic construct
  source 1..185: mol_type = other RNA
tgataataggctggagcctcattaacttttaccattgtcacactccatataaagtaaaattcaccattttaattacttttaccattgtcacactccataataaaaataaagtatataattcttttaccattgtcacactccacaatgtacccccgtggtctttaaataaagtctaagtgggcggc

SEQ ID NO: 161 - RNA, 251 nt, synthetic construct
  source 1..251: mol_type = other RNA
tgataataggctggagcctcattaactacaaccattgtcacactccatataaagtaaaattctacaaccattgtcacactccacaccattttaattactacaaccattgtcacactccataataaaaataaagctacaaccattgtcacactccatatataattcatagctacaaccattgtcacactccatacccccgtggtctctacaaccattgtcacactccattaaataaagtctaagtgggcggc

SEQ ID NO: 162 - RNA, 185 nt, synthetic construct
  source 1..185: mol_type = other RNA
tgataatagcactggtacaagggttgggagagctggagcctcggtggcctagcttcttgccccttgcactggtacaagggttgggagaattgtataacgagcccctcctccccttcctgcacccgtaataaccactggtacaagggttgggagagtggtctttgaataaagtctgagtgggcggc

SEQ ID NO: 163 - RNA, 188 nt, synthetic construct
  source 1..188: mol_type = other RNA
taaagctccccggggtccataaagtaggaaacactacagcctcggtggcctagcttcttgccccttgggcctccataaagtaggaaacactacatccccccagcccctcctccccttcctgcacccgtaccccctccataaagtaggaaacactacagtggtctttgaataaagtctgagtgggcggc

SEQ ID NO: 164 - RNA, 185 nt, synthetic construct
  source 1..185: mol_type = other RNA
taaagctccccggggcgcattattactcacggtacgagcctcggtggcctagcttcttgccccttgggcccgcattattactcacggtacgatccccccagcccctcctccccttcctgcacccgtaccccccgcattattactcacggtacgagtggtctttgaataaagtctgagtgggcggc

SEQ ID NO: 165 - RNA, 185 nt, synthetic construct
  source 1..185: mol_type = other RNA
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 166 - RNA, 185 nt, synthetic construct
  source 1..185: mol_type = other RNA
taaagctccccggggcactggtacaagggttgggagagcctcggtggcctagcttcttgccccttgggcccactggtacaagggttgggagatccccccagcccctcctccccttcctgcacccgtaccccccactggtacaagggttgggagagtggtctttgaataaagtctgagtgggcggc

SEQ ID NO: 167 - RNA, 185 nt, synthetic construct
  source 1..185: mol_type = other RNA
taaagctccccggggatgcccttttaacattgcactggcctcggtggcctagcttcttgccccttgggccatgcccttttaacattgcactgtccccccagcccctcctccccttcctgcacccgtacccccatgcccttttaacattgcactggtggtctttgaataaagtctgagtgggcggc

SEQ ID NO: 168 - RNA, 188 nt, synthetic construct
  source 1..188: mol_type = other RNA
taaagctccccggggcacaaattcggatctacagggtagcctcggtggcctagcttcttgccccttgggcccacaaattcggatctacagggtatccccccagcccctcctccccttcctgcacccgtaccccccacaaattcggatctacagggtagtggtctttgaataaagtctgagtgggcggc

SEQ ID NO: 169 - RNA, 185 nt, synthetic construct
  source 1..185: mol_type = other RNA
taaagctccccggggtggggtatttgacaaactgacagcctcggtggcctagcttcttgccccttgggcctggggtatttgacaaactgacatccccccagcccctcctccccttcctgcacccgtaccccctggggtatttgacaaactgacagtggtctttgaataaagtctgagtgggcggc

SEQ ID NO: 170 - RNA, 263 nt, synthetic construct
  source 1..263: mol_type = other RNA
taaagctccccgggggctggagcctcattaattacaaaagtaggaaacactacatataaagtaaaattttacaaaagtaggaaacactacacaccattttaattattacaaaagtaggaaacactacataataaaaataaagttacaaaagtaggaaacactacatatataattcatagttacaaaagtaggaaacactacatacccccgtggtctttacaaaagtaggaaacactacattaaataaagtctaagtgggcggc

SEQ ID NO: 171 - RNA, 257 nt, synthetic construct
  source 1..257: mol_type = other RNA
taaagctccccgggggcctcattaattacaaaagtaggaaacactacatataaagtaaaattttacaaaagtaggaaacactacacaccattttaattattacaaaagtaggaaacactacataataaaaataaagttacaaaagtaggaaacactacatatataattcatagttacaaaagtaggaaacactacatacccccgtggtctttacaaaagtaggaaacactacattaaataaagtctaagtgggcggc

SEQ ID NO: 172 - RNA, 194 nt, synthetic construct
  source 1..194: mol_type = other RNA
taaagctccccgggggctggagcctcattaattacaaaagtaggaaacactacatataaagtaaaattcaccattttaattattacaaaagtaggaaacactacataataaaaataaagtatataattttacaaaagtaggaaacactacacatagtacccccgtggtctttaaataaagtctaagtgggcggc

SEQ ID NO: 173 - RNA, 185 nt, synthetic construct
  source 1..185: mol_type = other RNA
taaagctccccgggggcctcattaacactggtacaagggttgggagatataaagtaaaattcaccattttaattacactggtacaagggttgggagataataaaaataaagtatataattcactggtacaagggttgggagacaatgtacccccgtggtctttaaataaagtctaagtgggcggc

SEQ ID NO: 174 - RNA, 185 nt, synthetic construct
  source 1..185: mol_type = other RNA
taaagctccccgggggcctcattaactacagtacaagggttgggagatataaagtaaaattcaccattttaattactacagtacaagggttgggagataataaaaataaagtatataattctacagtacaagggttgggagacaatgtacccccgtggtctttaaataaagtctaagtgggcggc

SEQ ID NO: 175 - RNA, 252 nt, synthetic construct
  source 1..252: mol_type = other RNA
taaagctccccgggggcctcattaactacagtacaagggttgggagaatataaagtaaactacagtacaagggttgggagaattcaccattttctacagtacaagggttgggagaaattataataaaaataaagtatataatctacagtacaagggttgggagatcaatgtaccccctacagtacaagggttgggagacgtggtctttaactacagtacaagggttgggagaataaagtctaagtgggcggc

SEQ ID NO: 176 - RNA, 251 nt, synthetic construct
  source 1..251: mol_type = other RNA
taaagctccccgggggcctcattaacgcattattactcacggtacgatataaagtaaaattcgcattattactcacggtacgacaccattttaattacgcattattactcacggtacgataataaaaataaagcgcattattactcacggtacgatatataattcatagcgcattattactcacggtacgatacccccgtggtctcgcattattactcacggtacgattaaataaagtctaagtgggcggc

SEQ ID NO: 177 - RNA, 251 nt, synthetic construct
  source 1..251: mol_type = other RNA
taaagctccccgggggcctcattaactacatattactcacggtacgatataaagtaaaattctacatattactcacggtacgacaccattttaattactacatattactcacggtacgataataaaaataaagctacatattactcacggtacgatatataattcatagctacatattactcacggtacgatacccccgtggtctctacatattactcacggtacgattaaataaagtctaagtgggcggc

SEQ ID NO: 178 - RNA, 208 nt, synthetic construct
  source 1..208: mol_type = other RNA
taaagctccccgggggcctcattaacgcattattactcacggtacgagcctagcttcttgccccttgggcccgcattattactcacggtacgatccccccagcccctcctcccatgcccttttaacattgcactgcttcctgcacccgtacccccatgcccttttaacattgcactggtggtctttgaataaagtctgagtgggcggc

SEQ ID NO: 179 - RNA, 207 nt, synthetic construct
  source 1..207: mol_type = other RNA
taaagctccccgggggcctcattaactacatattactcacggtacgatataaagtaaaattcaccattttctacatattactcacggtacgaaattataataaaaataaagtatatatacacttttaacattgcactgaattcatagtacccccgtggtctatacacttttaacattgcactgttaaataaagtctaagtgggcggc

SEQ ID NO: 180 - RNA, 251 nt, synthetic construct
  source 1..251: mol_type = other RNA
taaagctccccgggggcctcattaaatacacttttaacattgcactgtataaagtaaaattatacacttttaacattgcactgcaccattttaattaatacacttttaacattgcactgtaataaaaataaagatacacttttaacattgcactgtatataattcatagatacacttttaacattgcactgtacccccgtggtctatacacttttaacattgcactgttaaataaagtctaagtgggcggc

SEQ ID NO: 181 - RNA, 185 nt, synthetic construct
  source 1..185: mol_type = other RNA
taaagctccccgggggcctcattaactacaaccattgtcacactccatataaagtaaaattcaccattttaattactacaaccattgtcacactccataataaaaataaagtatataattctacaaccattgtcacactccacaatgtacccccgtggtctttaaataaagtctaagtgggcggc

SEQ ID NO: 182 - RNA, 251 nt, synthetic construct
  source 1..251: mol_type = other RNA
taaagctccccgggggcctcattaactacaaccattgtcacactccatataaagtaaaattctacaaccattgtcacactccacaccattttaattactacaaccattgtcacactccataataaaaataaagctacaaccattgtcacactccatatataattcatagctacaaccattgtcacactccatacccccgtggtctctacaaccattgtcacactccattaaataaagtctaagtgggcggc

SEQ ID NO: 183 - RNA, 201 nt, synthetic construct
  source 1..201: mol_type = other RNA
taaagctccccgggggcctcattaacactggtacaagggttgggagagctggagcctcggtggcctagcttcttgccccttgcactggtacaagggttgggagaattgtataacgagcccctcctccccttcctgcacccgtaataaccactggtacaagggttgggagagtggtctttgaataaagtctgagtgggcggc

SEQ ID NO: 184 - RNA, 191 nt, synthetic construct
  source 1..191: mol_type = other RNA
taaagctccccgggggctggagcctcattaattacatatttgacaaactgacatataaagtaaaattcaccattttaattattacatatttgacaaactgacataataaaaataaagtatataattttacatatttgacaaactgacacatagtacccccgtggtctttaaataaagtctaagtgggcggc

SEQ ID NO: 185 - RNA, 251 nt, synthetic construct
  source 1..251: mol_type = other RNA
taaagctccccgggggcctcattaattacatatttgacaaactgacatataaagtaaaattttacatatttgacaaactgacacaccattttaattattacatatttgacaaactgacataataaaaataaagttacatatttgacaaactgacatatataattcatagttacatatttgacaaactgacatacccccgtggtctttacatatttgacaaactgacattaaataaagtctaagtgggcggc

SEQ ID NO: 186 - RNA, 260 nt, synthetic construct
  source 1..260: mol_type = other RNA
taaagctccccgggggctggagcctcattaattacaaaagtaggaaacactacatataaagtaaaattttacaaaagtaggaaacactacacaccattttaattattacaaaagtaggaaacactacataataaaaataaagttacatatttgacaaactgacatatataattcatagttacatatttgacaaactgacatacccccgtggtctttacatatttgacaaactgacattaaataaagtctaagtgggcggc

SEQ ID NO: 187 - protein, 126 aa, synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKRSHMHLE

SEQ ID NO: 188 - protein, 128 aa, synthetic construct; identical to SEQ ID NO: 71

SEQ ID NO: 189 - RNA, 22 nt, synthetic construct
  source 1..22: mol_type = other RNA
cccaacaacaggaaactaccta

SEQ ID NO: 190 - RNA, 22 nt, synthetic construct
  source 1..22: mol_type = other RNA
cactggtacaagggttgggaga

SEQ ID NO: 191 - RNA, 23 nt, synthetic construct
  source 1..23: mol_type = other RNA
tccataaagtaggaaacactaca

SEQ ID NO: 192 - RNA, 22 nt, synthetic construct
  source 1..22: mol_type = other RNA
caaacaccattgtcacactcca

SEQ ID NO: 193 - RNA, 22 nt, synthetic construct
  source 1..22: mol_type = other RNA
tggggtatttgacaaactgaca

SEQ ID NO: 194 - RNA, 22 nt, synthetic construct
  source 1..22: mol_type = other RNA
ctacatattactcacggtacga

SEQ ID NO: 195 - RNA, 22 nt, synthetic construct
  source 1..22: mol_type = other RNA
atacacttttaacattgcactg

SEQ ID NO: 196 - RNA, 22 nt, synthetic construct
  source 1..22: mol_type = other RNA
ctacagtacaagggttgggaga

SEQ ID NO: 197 - RNA, 23 nt, synthetic construct
  source 1..23: mol_type = other RNA
ttacaaaagtaggaaacactaca

SEQ ID NO: 198 - RNA, 22 nt, synthetic construct
  source 1..22: mol_type = other RNA
ctacaaccattgtcacactcca

SEQ ID NO: 199 - RNA, 22 nt, synthetic construct
  source 1..22: mol_type = other RNA
ttacatatttgacaaactgaca

SEQ ID NO: 200 - protein, 699 aa, synthetic construct
MGKPIPNPLLGLDSTQGQEQKLISEEDLAGKPIPNPLLGLDSTQGQEQKLISEEDLAGKPIPNPLLGLDSTQGQEQKLISEEDLAGKPIPNPLLGLDSTQGQEQKLISEEDLAGKPIPNPLLGLDSTQGQEQKLISEEDLAPAAKRVKLDEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 201 - RNA, 33 nt, synthetic construct
  source 1..33: mol_type = other RNA
tatttatttatttatttgtttgtttgttttatt

SEQ ID NO: 202 - RNA, 119 nt, synthetic construct
  source 1..119: mol_type = other RNA
taatagtaagctggagcctcattaatataaagtaaaattcaccattttaattataataaaaataaagtatataattcaatgtacccccgtggtctttaaataaagtctaagtgggcggc

SEQ ID NO: 203 - RNA, 126 nt, synthetic construct
  source 1..126: mol_type = other RNA
aggaaataagagagtccataaagtaggaaacactacaaaaagaagagtaatccataaagtaggaaacactacagaagaaatataatccataaagtaggaaacactacagaccccggcgccgccacc

SEQ ID NO: 204 - RNA, 5013 nt, synthetic construct
  source 1..5013: mol_type = other RNA
atgcaaatagagctctccacctgcttctttctgtgccttttgcgattctgctttagtgccaccagaagatactacctgggtgcagtggaactgtcctgggactatatgcaaagtgatctcggtgagctgcctgtggacgcaagatttcctcctagagtgcccaagtcttttccattcaacacctcagtcgtgtacaagaagactctgtttgtagaattcacggatcaccttttcaacatcgctaagccaaggccaccctggatgggtctgctaggtcctaccatccaggctgaggtttacgatacagtggtcattacacttaagaacatggcttcccatcctgtcagtcttcacgctgttggtgtatcctactggaaagcttctgagggagctgaatacgacgatcagaccagtcaaagggagaaagaagacgataaagtcttccctggtggaagccatacatacgtctggcaggtcctgaaagagaacggtccaatggcctctgacccactgtgccttacctactcatatctttctcacgtggacctggtcaaggacttgaattcaggcctcattggagccctactagtgtgtagagaagggagtctggccaaggagaagacacagaccttgcacaaatttatactacttttcgctgtatttgacgaagggaagagttggcactcagaaacaaagaactccttgatgcaggatagggacgctgcatctgctcgggcctggcctaagatgcacacagtcaacggttacgtaaacaggtccctgccaggtctgattggctgccacaggaaatcagtctattggcacgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgtcttgtcatatctcttcccaccaacacgacggcatggaagcttacgtcaaagtagacagctgtccagaggaaccccaactacgaatgaagaataacgaagaagcggaagactacgacgacgatcttactgattctgaaatggacgtggtcaggtttgacgacgacaactctccttcctttatccaaattcgctcagttgccaagaagcatcctaagacttgggtacattacattgctgctgaagaggaggactgggactacgctcccttagtcctcgcccctgacgacagaagttataagagtcaatatttgaacaacggccctcagcggattggtaggaagtacaagaaagtccgatttatggcatacacagacgaaacctttaagactcgtgaagctattcagcacgaatcaggaatcttgggacctttactttacggggaagttggagacacactgttgattatatttaagaatcaagcaagcagaccatataacatctaccctcacggaatcactgacgtccgtcctttgtattcaaggagattacccaagggtgtcaagcatttgaaggattttccaattctgccaggagaaatattcaaatataagtggacagtgactgtagaagacgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaacgtcatcctgttcagcgtcttcgacgagaacaggagctggtacctcacagagaatatacaacgctttctccccaatccagctggagtgcagcttgaggatccagagttccaagcctccaacatcatgcacagcatcaacggctacgtgttcgatagtttgcagttgtcagtttgtttgcacgaggtggcatactggtacattctaagcattggagcacagactgacttcctgagcgtgttcttcagcggctacaccttcaaacacaagatggtctacgaagacacactcaccctattcccattctcaggagaaactgtcttcatgtcgatggagaacccaggtctgtggattctggggtgccacaactcagactttcggaacagaggcatgaccgccttactgaaggtttctagttgtgacaagaacactggtgattattacgaggacagttacgaagatatttcagcatacttgctgagtaagaacaacgccattgaaccaagaagcttctcccagaattcaagacaccctagcactaggcagaagcaatttaacgccaccacaattccagagaacgacatagagaagactgacccttggtttgcacacagaacacctatgcctaagatacagaacgtctcctctagtgatttgttgatgctcttgcgacagagtcctactccacacgggctatccttatctgatctccaagaagccaaatacgagacattctctgacgatccatcacctggagcaatagacagtaataacagcctgtctgaaatgacacacttcaggccacagctccatcacagtggggacatggtatttacccctgagtcaggcctccaattaagattaaacgagaaactggggacaactgcagcaacagagttgaagaaacttgatttcaaagtttctagtacatcaaataacctgatctccaccatccccagcgacaatttggcagcaggtactgataatacaagttccttaggaccaccaagtatgccagttcattacgatagtcaattagataccactctatttggcaagaagtcatctccccttactgagtctggtggacctctgagcttgagtgaagagaacaacgactccaaactgctggaatccggcttaatgaatagccaagaaagtagctggggcaagaacgtatcgtcacaacgggaaataactcgtactactcttcagtcagatcaagaggaaattgactacgacgataccatatcagttgaaatgaagaaggaagattttgacatttacgacgaggacgagaatcagtcaccccgcagctttcagaagaagacacgacactatttcatcgccgccgtggagaggctgtgggattacgggatgagtagctccccacacgttctaagaaacagggctcagagtggcagtgtccctcagttcaagaaagttgttttccaggaatttactgacggctcctttactcagcccttataccgtggagaactaaacgaacatttgggactcctggggccatatataagagcagaagttgaagataatatcatggtaactttcagaaatcaggcctctcgtccctattccttctattctagcctgatcagctacgaggaggaccagcgccagggagcagaacctagaaagaactttgtcaagcctaacgaaaccaagacttacttttggaaagtgcaacatcatatggcacccactaaagacgagtttgactgcaaagcctgggcttatttctctgacgttgacctggagaaagacgtgcactcaggcctgattggaccccttctggtctgccacactaacacactgaaccctgctcacgggagacaagtgacagtacaggaatttgctctgttcttcaccatctttgacgaaaccaagtcctggtacttcaccgagaatatggaaagaaactgcagggctccctgcaatatccagatggaagatcccacttttaaagagaattatcgcttccacgcaatcaacggctacataatggatacactacctggcttagtaatggctcaggatcaaaggattaggtggtatctgctcagcatgggcagcaacgagaacatccattctattcatttcagtggacacgtgttcactgtacggaagaaagaggagtataagatggcactgtacaatctctatccaggtgtgttcgagacagtggaaatgttaccatccaaagctggaatttggcgggtggagtgccttattggcgagcatctacacgctgggatgagcacactgttcctggtgtacagcaataagtgtcagactcccctgggaatggcttctggacacattagagattttcagattacagcttcaggacaatacggacagtgggccccaaagctggccagacttcattattccggatcaatcaacgcctggtccaccaaggagcccttcagctggatcaaggtggatctgttggcaccaatgattattcacggcatcaagacccagggtgcccgtcagaagttctccagcctctacatctctcagtttatcatcatgtatagtcttgacgggaagaagtggcagacttatcgaggaaattccactggaaccttaatggtcttctttggcaacgtggattcatctgggatcaagcacaatatcttcaaccctccaattattgctcgatacatccgtttgcacccaactcattatagcattcgcagcactcttcgcatggagttgatgggctgtgatttaaatagttgcagcatgccattgggaatggagagtaaagcaatatcagacgcacagattactgcttcatcctactttaccaatatgtttgccacctggtccccttccaaggctcgacttcacctccaagggaggagtaacgcctggaggcctcaggtgaataatccgaaggagtggctgcaagtggacttccagaagacaatgaaagtcacaggagtaactactcagggagtcaagtctctgcttaccagcatgtacgtgaaggagttcctcatctccagcagtcaagacggccatcagtggactctcttctttcagaacggcaaagtaaaggtgtttcagggaaatcaagactccttcacacctgtggtgaacagcctggacccaccgttactgactcgctaccttcgaattcatccgcagtcctgggtgcaccagatcgccctcaggatggaggttctgggctgcgaggcacaggacctctac

SEQ ID NO: 205 - RNA, 10 nt, synthetic construct
  source 1..10: mol_type = other RNA
ccrrccatgg

SEQ ID NO: 206 - RNA, 281 nt, synthetic construct
  source 1..281: mol_type = other RNA
tgataataggctggagcctcattaactacatattactcacggtacgatctaaactgaaattctacatattactcacggtacgacaccactataatacctacatattactcacggtacgataataataataatcctacatattactcacggtacgatatatcactcactcatcaccacgctgttctacatattactcacggtacgatacccccgtggtcttggtactcgtctacatattactcacggtacgaggcacattaaataaagtctaagtgggcggc

SEQ ID NO: 207 - RNA, 297 nt, synthetic construct
  source 1..297: mol_type = other RNA
taaagctccccgggggcctcattaagctggagcctcattaactacatattactcacggtacgatctaaactgaaattctacatattactcacggtacgacaccactataatacctacatattactcacggtacgataataataataatcctacatattactcacggtacgatatatcactcactcatcaccacgctgttctacatattactcacggtacgatacccccgtggtcttggtactcgtctacatattactcacggtacgaggcacattaaataaagtctaagtgggcggc

(19) United States
(12) Patent Application Publication
(10) Pub. No.: US 2026/0035714 A1
(43) Pub. Date: Feb. 5, 2026

(54) ENGINEERED POLYNUCLEOTIDES FOR CELL SELECTIVE EXPRESSION

(71) Applicant: ModernaTX, Inc., Cambridge, MA (US)

(72) Inventors: Elizaveta A. Andrianova, Stoneham, MA (US); Ruchi Jain, Chestnut Hill, MA (US); Christopher Pepin, Bedford, MA (US)

(21) Appl. No.: 18/998,251

(22) PCT Filed: Jul. 24, 2023
(86) PCT No.: PCT/US2023/070820

§ 371 Date: Jan. 24, 2025

Related U.S. Application Data
(60) Provisional application No. 63/391,863, filed on Jul. 25, 2022.

(51) Int. Cl. C12N 15/85; A61K 48/00

(52) U.S. Cl. CPC C12N 15/85; A61K 48/0041; A61K 48/0058; A61K 48/0066; C12N 2830/50; C12N 2840/007

(57) ABSTRACT

The disclosure features compositions or systems and uses thereof, comprising a first polynucleotide encoding a target molecule, optionally a second polynucleotide encoding an effector, repressor, or endonuclease molecule; optionally a recognition or cleavage site in the first or second polynucleotide, and optionally a repressor/effector binding site in the first polynucleotide. The disclosure also features compositions or systems and uses thereof, comprising a messenger RNA (mRNA) comprising (i) an open reading frame encoding a polypeptide, and (ii) at least six miR142 target sites.

### CROSS-REFERENCE TO RELATED APPLICATIONS

This application claims priority to U.S. Patent Application No. 63/391,863, filed on Jul. 25, 2022, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Jul. 24, 2023, is named 45817-0112WO1.xml and is 263,327 bytes in size.

### BACKGROUND

Off-target effects are common and limit the utility of many drug candidates (Chartier, M., et al. BMC Pharmacol Toxicol 18, 18 (2017). While target-cell specificity is desirable in general for enhancing the safety of therapeutics, limiting off-target expression is especially important for some applications, e.g., gene editing where the genetic material of cells is permanently altered and/or the expression of toxic cargo in certain cell types, e.g., myeloid cells that release perforin (Dufait I. et al., Cancers (Basel). 2019 Jun. 11; 11(6):808) or cancer cells that make the p53 upregulated modulator of apoptosis (PUMA) (Yu J, Zhang L. Oncogene. 2008; 27 Suppl 1(Suppl 1):S71-S83). This is particularly relevant in the case of gene therapy and mRNA therapeutics which have the potential to revolutionize vaccination, protein replacement therapies, and the treatment of genetic diseases (Kowalski P. S. et al., Mol Ther. 2019 Apr. 10; 27(4): 710-728).

Further, there is a need to better utilize the body's own cells, such as hematopoietic stem cells and other immune cells, to treat a wide range of conditions such as cancers and hematological malignancies, autoimmune disorders, allergies, inherited and acquired disorders, etc. For instance, treatments such as allogeneic hematopoietic stem cell transplantation are limited by the poor availability of matched donors and the mortality associated with the allogenic procedure due to graft vs host disease (GvHD) (Jalapothu D, et al. Front Immunol. 2016; 7:361). As an alternative to allogeneic HCT, an inherited genetic defect can be corrected in the patient's own hematopoietic cells by gene therapy. The isolated hematopoietic stem cell can be genetically modified and returned to the patient as an autologous transplant. However, it is challenging to deliver a relevant gene into all or a fraction of the affected cells of the body and sustain its therapeutic effect.

Thus, there is a need to enable selective expression of therapeutic proteins in specific cells and tissues, as well as tuning that expression to achieve a desired expression profile and to limit off-target effects and enhance safety of treatments, with wide-ranging applications in immunotherapy and immune-oncology therapeutics, in vivo gene editing, stem cell transplantation, and gene therapies for various diseases.

### SUMMARY

The present disclosure provides, inter alia, compositions or systems comprising a polynucleotide (e.g., a messenger RNA or DNA) encoding a polypeptide (e.g., a target molecule) with selective expression in cell types (e.g., hematopoietic stem and progenitor cells or mature immune cells), wherein the expression is dependent on the microRNA composition of the cells.

In one aspect, the disclosure features a composition comprising a messenger RNA (mRNA) comprising (i) an open reading frame encoding a polypeptide, and (ii) one or more microRNA target sites present in hematopoietic stem and progenitor cells (HSPC miRts).

In some embodiments, the one or more HSPC miRts comprise miR-126-3p, miR-130a-3p, miR-10a-5p, miR-29a-3p, miR125a-5p, miR125b-5p, or miR196b-5p.

In some embodiments, the polypeptide is a gene editor, a cytokine, an apoptotic protein, a transcription factor, a DNA-binding protein, a receptor, an enzyme, or a chimeric antigen receptor.

In some embodiments, the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

In some embodiments, the composition comprises a lipid nanoparticle.

In some embodiments, the lipid nanoparticle comprises an ionizable amino lipid of Formula (I):

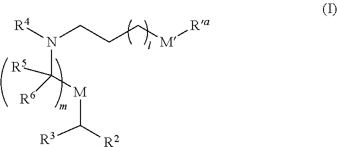

or a salt thereof,
wherein R′^{a} is R′^{branched}; wherein
   R′^{branched} is:

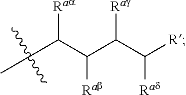

wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

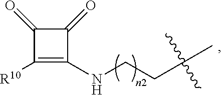

wherein [symbol] denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid has the formula:

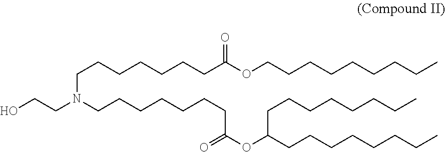 [SMILES: CCCCCCCCCOC(=O)CCCCCCCN(CCO)CCCCCCCC(=O)OC(CCCCCCCC)CCCCCCCC]

or a salt thereof.

In some embodiments, the lipid nanoparticle further comprises a PEG-lipid.

In some embodiments, the PEG-lipid has the formula:

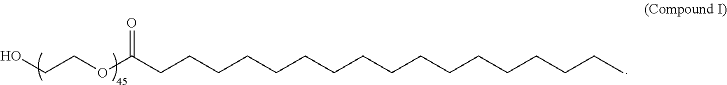

In some embodiments, the one or more HSPC miRts comprise at least one microRNA target site specific for miR126.

In some embodiments, the one or more HSPC miRts comprise at least two microRNA target sites specific for miR126.

In some embodiments, the one or more HSPC miRts comprise at least one microRNA target site specific for miR130a.

In some embodiments, the one or more HSPC miRts comprise at least two microRNA target sites specific for miR130a.

In some embodiments, the one or more HSPC miRts comprise at least two microRNA target sites specific for miR126 and at least two microRNA target sites specific for miR130a.

In some embodiments, the one or more HSPC miRts are in a non-coding region of the mRNA.

In some embodiments, the 3′ untranslated region (UTR) of the mRNA comprises at least one HSPC miRts.

In some embodiments, the 3′ UTR of the mRNA comprises at least two repeats of one HSPC miRts.

In some embodiments, the 3′ UTR of the mRNA comprises six repeats of one HSPC miRts.

In some embodiments, the 5′ UTR of the mRNA comprises at least one HSPC miRts.

In some embodiments, the 5′ UTR of the mRNA comprises at least two repeats of one HSPC miRts.

In some embodiments, the 5′ UTR of the mRNA comprises three repeats of one HSPC miRts.

In some embodiments, the mRNA has one or more HSPC miRts in the 3′ UTR and the 5′ UTR.

In some embodiments, the mRNA has one or more of the following features: (1) an AU-rich element; (2) the one or more HSPC miRts comprise at least one mismatch to the microRNA that binds the one or more HSPC miRts; (3) structurally accessible UTRs; (4) a short polyA tail; and (5) the ability to form microRNA bridges when a microRNA binds to the one or more HSPC miRts.

In some embodiments, the 5′ UTR and/or the 3′ UTR comprises an AU-rich element.

In some embodiments, the 3′ UTR is 60%-90% AU-rich.

In some embodiments, the 3′ UTR is about 70% AU-rich.

In some embodiments, the one or more HSPC miRts comprise one to three mismatches to the microRNA that binds the one or more HSPC miRts.

In some embodiments, the polyA tail is 20-100 nucleotides in length.

In some embodiments, the microRNA bridge is formed by one or more miRts in the 5′ UTR and the 3′ UTR of the mRNA.

In another aspect, the disclosure features a method of preferentially expressing a polypeptide in hematopoietic cell types other than hematopoietic stem and progenitor cells (HSPCs), the method comprising contacting a population of hematopoietic cells with a composition described herein, wherein the population of hematopoietic cells comprises HSPCs and hematopoietic cell types other than HSPCs.

In some embodiments, the contacting of the population of hematopoietic cells occurs ex vivo.

In some embodiments, the contacting of the population of hematopoietic cells occurs in vivo.

In another aspect, the disclosure features a composition comprising:
(a) a first polynucleotide comprising (i) a repressor binding element, (ii) an open reading frame encoding a polypeptide, and (iii) optionally one or more microRNA target sites present in non-hematopoietic stem and progenitor cells (non-HSPC miRts); and
(b) a second polynucleotide comprising (i) a sequence encoding a repressor that binds to the repressor binding element and (ii) one or more microRNA target sites present in hematopoietic stem and progenitor cells (HSPC miRts),
wherein binding of the repressor to the repressor binding element reduces translation of the polypeptide from the first polynucleotide.

In some embodiments, the one or more HSPC miRts comprise miR-126-3p, miR-130a-3p, miR-10a-5p, miR-29a-3p, miR125a-5p, miR125b-5p, or miR196b-5p.

In some embodiments, the one or more non-HSPC miRts comprise miR142-3p, miR150-5p, miR223-3p, or miR122-5p.

In some embodiments, the polypeptide is toxic to HSPCs.

In some embodiments, the polypeptide is a gene editor, a cytokine, an apoptotic protein, a transcription factor, a DNA-binding protein, a receptor, an enzyme, or a chimeric antigen receptor.

In some embodiments, the one or more HSPC miRts comprise at least one microRNA target site specific for miR126.

In some embodiments, the one or more HSPC miRts comprise at least two microRNA target sites specific for miR126.

In some embodiments, the one or more HSPC miRts comprise at least one microRNA target site specific for miR130a.

In some embodiments, the one or more HSPC miRts comprise at least two microRNA target sites specific for miR130a.

In some embodiments, the one or more HSPC miRts comprise at least two microRNA target sites specific for miR126 and at least two microRNA target sites specific for miR130a.

In some embodiments, the one or more microRNA target sites are in the non-coding region of each of the first and second polynucleotides, wherein each of the first and second polynucleotides is an mRNA.

In some embodiments, the 3′ UTR of the first and second polynucleotides each comprises one microRNA target site.

In some embodiments, the 3′ UTR of the first and second polynucleotides each comprises at least two repeats of one microRNA target site.

In some embodiments, the 3′ UTR of the first and second polynucleotides each comprises six repeats of one microRNA target site.

In some embodiments, the 5′ UTR of the first and second polynucleotides each comprises one microRNA target site.

In some embodiments, the 5′ UTR of the first and second polynucleotides each comprises at least two repeats of one microRNA target site.

In some embodiments, the 5′ UTR of the first and second polynucleotides each comprises three repeats of one microRNA target site.

In some embodiments, the mRNA has one or more HSPC miRts in the 3′ UTR and the 5′ UTR.

In some embodiments, the mRNA has one or more of the following features: (1) an AU-rich element; (2) the one or more HSPC miRts comprise at least one mismatch to the microRNA that binds the one or more HSPC miRts; (3) structurally accessible UTRs; (4) a short polyA tail; and (5) the ability to form microRNA bridges when a microRNA binds to the one or more HSPC miRts.

In some embodiments, the 5′ UTR and/or the 3′ UTR comprises an AU-rich element.

In some embodiments, the 3′ UTR is 60%-90% AU-rich.

In some embodiments, the 3′ UTR is about 70% AU-rich.

In some embodiments, the one or more HSPC miRts comprise one to eight mismatches to the microRNA that binds the one or more HSPC miRts.

In some embodiments, the polyA tail is 40-100 nucleotides in length.

In some embodiments, the microRNA bridge is formed by one or more miRts in the 5′ UTR and the 3′ UTR of the mRNA.

In some embodiments, the first and the second polynucleotide each is an mRNA and comprises a polyA tail or is a DNA.

In some embodiments, the one or more HSPC miRts in the second polynucleotide are in the non-coding portion of the second polynucleotide.

In some embodiments, the one or more HSPC miRts in the second polynucleotide are (a) positioned between the sequence encoding the repressor and a polyA tail; or (b) positioned between a 5′ cap and a start codon, wherein the second polynucleotide is an mRNA.

In some embodiments, the repressor binding element comprises a kink-turn forming sequence.

In some embodiments, the repressor binding element is selected from the group consisting of PRE, PRE2, MS2, PP7, BoxB, U1A hairpin, and 7SK.

In some embodiments, the repressor is selected from the group consisting of Snu13, 50S ribosomal L7Ae protein, Pumilio and FBF (PUF) protein, PUF2 protein, MBP-LacZ, MBP, PCP, Lambda N, U1A, 15.5kd, LARP7, L30e, and other RNA-binding proteins.

In some embodiments, the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

In some embodiments, the composition comprises a lipid nanoparticle.

In some embodiments, the lipid nanoparticle comprises an ionizable amino lipid of Formula (I):

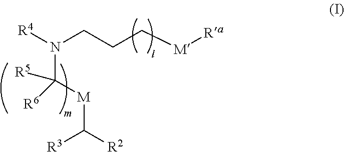

or a salt thereof,
wherein R′^{a} is R′^{branched}; wherein
   R′^{branched} is:

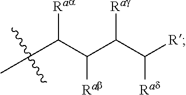

wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

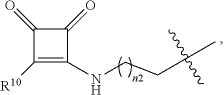

wherein [symbol] denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid has the formula:

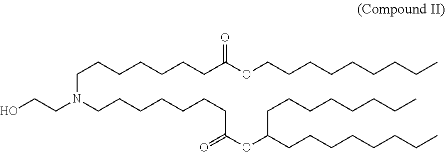 [SMILES: CCCCCCCCCOC(=O)CCCCCCCN(CCO)CCCCCCCC(=O)OC(CCCCCCCC)CCCCCCCC]

or a salt thereof.

In some embodiments, the lipid nanoparticle further comprises a PEG-lipid. In some embodiments, the PEG-lipid has the formula:

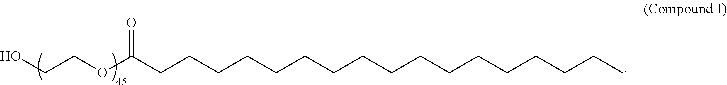

In another aspect, the disclosure features a method of preferentially expressing a polypeptide in hematopoietic stem and progenitor cells (HSPCs), the method comprising contacting a population of hematopoietic cells with a composition described herein, wherein the population of hematopoietic cells comprises HSPCs.

In some embodiments, the contacting of the population of hematopoietic cells occurs ex vivo.

In some embodiments, the contacting of the population of hematopoietic cells occurs in vivo.

In another aspect, the disclosure features a method of expressing a polypeptide in a hematopoietic stem and progenitor cell (HSPC), the method comprising contacting the cell with:
(a) a first polynucleotide comprising (i) a repressor binding element, (ii) an open reading frame encoding a polypeptide, and (iii) optionally one or more non-hematopoietic stem cell-microRNA target sites present in non-hematopoietic stem and progenitor cells (non-HSPC miRts), wherein modification of the one or more non-HSPC miRts reduces translation of the polypeptide from the first polynucleotide; and
(b) a second polynucleotide comprising (i) a sequence encoding a repressor that binds to the repressor binding element and (ii) one or more microRNA target sites present in hematopoietic stem and progenitors (HSPC miRts),
wherein the HSPC expresses one or more microRNAs that bind to the one or more HSPC miRts and reduces translation of the repressor from the second polynucleotide.

In another aspect, the disclosure features a method of expressing a polypeptide in a hematopoietic stem and progenitor cell in a subject, the method comprising administering to the subject:
(a) a first polynucleotide comprising (i) a repressor binding element, (ii) an open reading frame encoding a polypeptide, and (iii) optionally one or more microRNA target sites present in non-hematopoietic stem and progenitor cells (non-HSPC miRts), wherein modification of the one or more non-HSPC miRts reduces translation of the polypeptide from the first polynucleotide; and
(b) a second polynucleotide comprising (i) a sequence encoding a repressor that binds to the repressor binding element and (ii) one or more microRNA target sites present in hematopoietic stem and progenitor cells (HSPC miRts),
wherein the HSPC expresses one or more microRNAs that bind to the one or more HSPC miRts and reduces translation of the repressor from the second polynucleotide.

In another aspect, the disclosure features a composition comprising:
(a) a first polynucleotide comprising (i) a repressor binding element, (ii) an open reading frame encoding a polypeptide, and (iii) optionally one or more microRNA target sites present in hematopoietic stem and progenitor cells (HSPC miRts); and
(b) a second polynucleotide comprising (i) a sequence encoding a repressor that binds to the repressor binding element and (ii) one or more microRNA target sites present in non-hematopoietic stem and progenitor cells (non-HSPC miRts),
wherein binding of the repressor to the repressor binding element reduces translation of the polypeptide from the first polynucleotide.

In some embodiments, the one or more HSPC miRts comprise miR-126-3p, miR-130a-3p, miR-10a-5p, miR-29a-3p, miR125a-5p, miR125b-5p, or miR196b-5p.

In some embodiments, the one or more non-HSPC miRts comprise a microRNA target site present in an immune cell or in a hepatocyte.

In some embodiments, the microRNA target site present in an immune cell is miR142-3p, miR150-5p, or miR223-3p.

In some embodiments, the microRNA target site present in a hepatocyte is miR-122-5p.

In some embodiments, the polypeptide is a gene editor, a cytokine, an apoptotic protein, a transcription factor, a DNA-binding protein, a receptor, an enzyme, or a chimeric antigen receptor.

In some embodiments, the one or more microRNA target sites are in the non-coding region of each of the first and second polynucleotides, wherein each of the first and second polynucleotides is an mRNA.

In some embodiments, the 3′ UTR of the first and second polynucleotides each comprises one microRNA target site.

In some embodiments, the 3′ UTR of the first and second polynucleotides each comprises at least two repeats of one microRNA target site.

In some embodiments, the 3′ UTR of the first and second polynucleotides each comprises six repeats of one microRNA target site.

In some embodiments, the 5′ UTR of the first and second polynucleotides each comprises one microRNA target site.

In some embodiments, the 5′ UTR of the first and second polynucleotides each comprises at least two repeats of one microRNA target site.

In some embodiments, the 5′ UTR of the first and second polynucleotides each comprises three repeats of one microRNA target site.

In some embodiments, the mRNA has one or more HSPC miRts in the 3′ UTR and the 5′ UTR.

In some embodiments, the mRNA has one or more of the following features: (1) an AU-rich element; (2) the one or more HSPC miRts comprise at least one mismatch to the microRNA that binds the one or more HSPC miRts; (3) structurally accessible UTRs; (4) a short polyA tail; and (5) the ability to form microRNA bridges when a microRNA binds to the one or more HSPC miRts.

In some embodiments, the 3′ UTR comprises an AU-rich element.

In some embodiments, the 3′ UTR is 60%-90% AU-rich.

In some embodiments, the 3′ UTR is about 70% AU-rich.

In some embodiments, the one or more HSPC miRts comprise one to three mismatches to the microRNA that binds the one or more HSPC miRts.

In some embodiments, the microRNA bridge is formed by one or more miRts in the 5′ UTR and the 3′ UTR of the mRNA.

In some embodiments, the first and second polynucleotide each is an mRNA and comprises a polyA tail or is a DNA.

In some embodiments, the one or more microRNA target sites in the second polynucleotide are (a) positioned between the sequence encoding the repressor and a polyA tail; or (b) positioned between a 5′ cap and a start codon, wherein the second polynucleotide is an mRNA.

In some embodiments, the repressor binding element comprises a kink-turn forming sequence.

In some embodiments, the repressor binding element is selected from the group consisting of PRE, PRE2, MS2, PP7, BoxB, U1A hairpin, and 7SK.

In some embodiments, the repressor is selected from the group consisting of Snu13, 50S ribosomal L7Ae protein, Pumilio and FBF (PUF) protein, PUF2 protein, MBP-LacZ, MBP, PCP, Lambda N, U1A, 15.5kd, LARP7, L30e, and other RNA-binding proteins.

In some embodiments, the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

In some embodiments, the composition comprises a lipid nanoparticle.

In some embodiments, the lipid nanoparticle comprises an ionizable amino lipid of Formula (I):

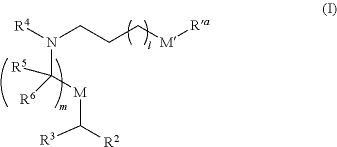

or a salt thereof,
wherein R′^{a} is R′^{branched}; wherein

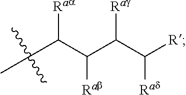

R′^{branched} is:
   wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₄ alkenyl;
R′ is selected from the group consisting of —(CH₂)ₙOH, wherein n is selected from the group consisting of 1, 2, 3, 4, and 5, and

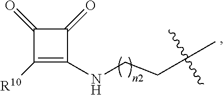

wherein [symbol] denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid has the formula:

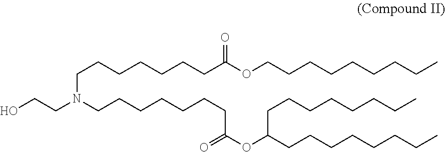 [SMILES: CCCCCCCCCOC(=O)CCCCCCCN(CCO)CCCCCCCC(=O)OC(CCCCCCCC)CCCCCCCC]

or a salt thereof.

In some embodiments, the lipid nanoparticle further comprises a PEG-lipid. In some embodiments, the PEG-lipid has the formula:

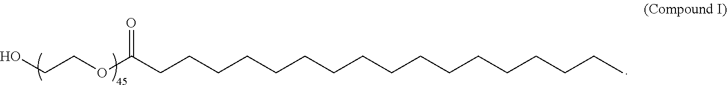

In another aspect, the disclosure features a method of preferentially expressing a polypeptide in hematopoietic cell types other than hematopoietic stem and progenitor cells (HSPCs), the method comprising contacting a population of hematopoietic cells with a composition described herein, wherein the population of hematopoietic cells comprises HSPCs and hematopoietic cell types other than HSPCs.

In some embodiments, the contacting of the population of hematopoietic cells occurs ex vivo.

In some embodiments, the contacting of the population of hematopoietic cells occurs in vivo.

In another aspect, the disclosure features a method of expressing a polypeptide in a hematopoietic cell other than a hematopoietic stem and progenitor cell (HSPC), the method comprising contacting the cell with
(a) a first polynucleotide comprising (i) a repressor binding element, (ii) an open reading frame encoding a polypeptide, and (iii) optionally one or more microRNA target sites present in hematopoietic stem and progenitor cells (HSPC miRts), wherein modification of the one or more HSPC miRts reduces translation of the polypeptide from the polynucleotide; and
(b) an second polynucleotide comprising (i) a sequence encoding a repressor that binds to the repressor binding element and (ii) one or more non-HSPC miRts,
wherein the hematopoietic cell expresses one or more microRNAs that bind to the one or more non-HSPC miRts and reduces translation of the repressor from the second polynucleotide.

In another aspect, the disclosure features a method of expressing a polypeptide in a hematopoietic cell other than a hematopoietic stem and progenitor cell (HSPC) in a subject, the method comprising administering to the subject:
(a) a first polynucleotide comprising (i) a repressor binding element, (ii) an open reading frame encoding a polypeptide, and (iii) optionally one or more microRNA target sites present in hematopoietic stem and progenitor cell (HSPC miRts), wherein modification of the one or more HSPC miRts reduces translation of the polypeptide from the first polynucleotide; and
(b) a second polynucleotide comprising (i) a sequence encoding a repressor that binds to the repressor binding element and (ii) one or more non-HSPC miRts,
wherein the hematopoietic cell expresses one or more microRNAs that bind to the one or more non-HSPC miRts and reduces translation of the repressor from the second polynucleotide.

In another aspect, the disclosure features a composition a first messenger RNA (mRNA) comprising (i) a first open reading frame encoding a first polypeptide, and (ii) at least six miR142 target sites.

In some embodiments, the polypeptide is a secreted protein.

In some embodiments, the at least six miR142 target sites are in the 3′ UTR of the first mRNA.

In some embodiments, the at least six miR142 target sites each comprise the sequence UCCAUAAAGUAGGAAACACUACA (SEQ ID NO:191).

In some embodiments, the at least six miR142 target sites each comprise the sequence UUACAAAAGUAGGAAACACUACA (SEQ ID NO:197).

In some embodiments, the composition further comprises a second mRNA comprising (i) a second open reading frame encoding a second polypeptide, and (ii) at least one miR target site.

In some embodiments, the second mRNA comprises at least two miR target sites.

In some embodiments, the second mRNA comprises at least three miR target sites.

In some embodiments, the second mRNA comprises at least four miR target sites.

In some embodiments, the second mRNA comprises at least five miR target sites.

In some embodiments, the second mRNA comprises at least six miR target sites.

In some embodiments, the at least one, at least two, at least three, at least four, at least five, or at least six miR target sites of the second mRNA are miR142 target sites.

In some embodiments, the at least one, at least two, at least three, at least four, at least five, or at least six miR target sites of the second mRNA each comprise the sequence UCCAUAAAGUAGGAAACACUACA (SEQ ID NO:191).

In some embodiments, the at least one, at least two, at least three, at least four, at least five, or at least six miR target sites of the second mRNA each comprise the sequence UUACAAAAGUAGGAAACACUACA (SEQ ID NO:197).

In some embodiments, the at least one, at least two, at least three, at least four, at least five, or at least six miR target sites of the second mRNA are selected from the group consisting of miR-126-3p, miR-130a-3p, miR-10a-5p, miR-29a-3p, miR125a-5p, miR125b-5p, miR196b-5p, miR150-5p, miR223-3p, and miR-122-5p target sites.

In some embodiments, the second mRNA does not comprise a miR142 target site.

In some embodiments, the first and/or second mRNA comprise a 3′ UTR comprising the sequence

(SEQ ID NO: 154)
UGAUAAUAGGCUGGAGCCUCAUUAAUCCAUAAAGUAGGAAACACUACAUA
UAAAGUAAAAUUUCCAUAAAGUAGGAAACACUACACACCAUUUUAAUUAU
CCAUAAAGUAGGAAACACUACAUAAUAAAAAUAAAGUCCAUAAAGUAGGA
AACACUACAUAUAUAAUUCAUAGUCCAUAAAGUAGGAAACACUACAUACC
CCCGUGGUCUUCCAUAAAGUAGGAAACACUACAUUAAAUAAAGUCUAAGU
GGGCGGC.

In some embodiments, the first and/or second mRNA comprise a 3′ UTR comprising the sequence

(SEQ ID NO: 155)
UGAUAAUAGGCUGGAGCCUCAUUAAUUACAAAAGUAGGAAACACUACAUA
UAAAGUAAAAUUUUACAAAAGUAGGAAACACUACACACCAUUUUAAUUAU
UACAAAAGUAGGAAACACUACAUAAUAAAAAUAAAGUUACAAAAGUAGGA
AACACUACAUAUAUAAUUCAUAGUUACAAAAGUAGGAAACACUACAUACC
CCCGUGGUCUUUACAAAAGUAGGAAACACUACAUUAAAUAAAGUCUAAGU
GGGCGGC.

In some embodiments, the first and/or second mRNA comprise a 3′ UTR comprising the sequence

(SEQ ID NO: 170)
UAAAGCUCCCCGGGGGCUGGAGCCUCAUUAAUUACAAAAGUAGGAAACAC
UACAUAUAAAGUAAAAUUUUACAAAAGUAGGAAACACUACACACCAUUUU
AAUUAUUACAAAAGUAGGAAACACUACAUAAUAAAAAUAAAGUUACAAAA
GUAGGAAACACUACAUAUAUAAUUCAUAGUUACAAAAGUAGGAAACACUA
CAUACCCCCGUGGUCUUUACAAAAGUAGGAAACACUACAUUAAAUAAAGU
CUAAGUGGGCGGC.

In some embodiments, the first and/or second mRNA comprise a 3′ UTR comprising the sequence

(SEQ ID NO: 171)
UAAAGCUCCCCGGGGGCCUCAUUAAUUACAAAAGUAGGAAACACUACAUA
UAAAGUAAAAUUUUACAAAAGUAGGAAACACUACACACCAUUUUAAUUAU
UACAAAAGUAGGAAACACUACAUAAUAAAAAUAAAGUUACAAAAGUAGGA
AACACUACAUAUAUAAUUCAUAGUUACAAAAGUAGGAAACACUACAUACC
CCCGUGGUCUUUACAAAAGUAGGAAACACUACAUUAAAUAAAGUCUAAGU
GGGCGGC.

In some embodiments, the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

In some embodiments, the composition comprises a lipid nanoparticle.

In another aspect, the disclosure features a method of expressing a polypeptide in a subject, the method comprising administering to the subject a composition comprising a messenger RNA (mRNA) comprising (i) an open reading frame encoding a polypeptide, and (ii) at least six miR142 target sites, as described above.

In some embodiments, the method comprises multiple administrations of the composition to the subject.

In some embodiments, the method comprises at least two, at least three, at least four, at least five, at least six, at least seven, at least eight, at least nine, or at least ten administrations of the composition to the subject.

Other features and advantages of the invention will be apparent from the following detailed description, and from the claims.

### BRIEF DESCRIPTION OF THE DRAWINGS

FIGS. 1A-E provide schematics of systems that provide cell-specific expression of target sequences. FIG. 1A shows a system in which cell-specific microRNA binds to microRNA target sites (miRts) on polynucleotide (mRNA), leading to mRNA degradation and suppressing (turning OFF) target protein translation. FIG. 1B shows a schematic of a polynucleotide encoding a target sequence, and having multiple microRNA target sites in the 3′ untranslated region (3′ UTR). FIG. 1C shows a system in which microRNA binding to the miRts of a repressor encoding RNA turns ON expression of target RNA, by reducing expression of the repressor and thereby preventing the repressor from binding to the repressor binding site on the target RNA. FIG. 1D shows a system in which microRNA binding to the microRNA target sites (e.g., miR150-ts or miR142-ts miRts) of a repressor encoding RNA turns ON expression of target RNA in mature immune cells, by reducing expression of the repressor and thereby preventing the repressor from binding to the repressor binding site on the target RNA. FIG. 1E shows a system in which microRNA binding to a microRNA target site of a repressor encoding RNA turns ON expression of target RNA in HSPC, by reducing expression of the repressor and thereby preventing the repressor from binding to the repressor binding site on the target RNA. Alternatively, a pan-immune cell-specific microRNA binding to the microRNA target sites of a repressor encoding RNA, combined with a target that has miR-target sites to turn off expression in mature immune cells, turns ON expression of target RNA in HSC, by i) reducing expression of the repressor in all immune cells and thereby preventing the repressor from binding to the repressor binding site on the target RNA and ii) by further reducing the target RNA in mature immune cells

FIG. 2 depicts OX40L expression in various cell types in the presence of mOX40L reporter mRNA OFF system. C57BL/6 mice were injected intravenously with 0.5 mg/kg mOX40L reporter mRNAs containing three miR-target-sites for the potential HSC specific miR candidates, PBS (control), or mOX40L reporter mRNA with no miR target sites (miRless mOX40L group). mOX40L expression was analyzed in the bone marrow and spleen 24 h post dose. The graphs show the % frequency of OX40L+ cells and geometric MFI of OX40L in LSK: Lin-Sca1+c-Kit+ progenitor cells, hematopoietic stem and progenitor cells (HSPCs) and in mature immune cells (macrophages) in the bone marrow.

FIG. 3 depicts the percentage suppression of mOX40L depicted in FIG. 2 in a tabular form. % suppression is calculated as percentage frequency of mOX40L+ cells in a given cell type for a given miRts vs miRless for the same cell-type. HSCs: hematopoietic stem cells; HSPCs: hematopoietic stem and progenitor cells; Macs: macrophages; cDC1: Conventional type 1 dendritic cells; cDC2: Conventional type 2 dendritic cells; Ly6C-hi mono: monocytes expressing high level of Ly6C; Ly6C-lo mono: monocytes expressing low level of Ly6C; NK: Natural killer cells; CD8 T: CD8+ T cells; Neut: neutrophils; Eos: Eosinophils

FIGS. 4A-B depicts the percentage suppression of mOX40L in various cell types as indicated (HSC, HSPC, Mature BM, Multipotent progenitors (MPP), Common lymphoid progenitors (CLP), Granulocyte Monocyte Progenitors (GMP), Common Myeloid Progenitors (CMP), and Megakaryocyte-Erythrocyte Progenitors (MEP)) in the presence of mOX40L reporter mRNA OFF system. Lonza human bone marrow mononuclear cells were transfected with 500 ng of mOX40L reporter mRNAs containing the indicated miR-target-sites, mOX40L reporter mRNA with no miR target sites (miRless mOX40L group) or PBS (control). mOX40L expression was analyzed 24 h post transfection. Each graph in FIG. 4A shows the geometric MFI of mOX40L+ cells. FIG. 4B shows a summary table of the percentage suppression of OX40L in various cell types as indicated 24 h post transfection.

FIGS. 5A-C depict the percentage suppression of OX40L in various cell types as indicated (Lin+, Lin− CD34+, HSC, MPP, CLP, GMP, and MEP) in the presence of mOX40L reporter mRNA OFF system. Lonza human bone marrow mononuclear cells were transfected with 500 ng of mOX40L reporter mRNAs containing the indicated miR-target-sites, mOX40L reporter mRNA with no miR target sites (miRless mOX40L group) or PBS (control). mOX40L expression was analyzed 24 h post transfection. The graph in FIG. 5A shows the normalized % frequency of OX40L+ cells in the indicated cell types. The graph in FIG. 5B shows the normalized geometric MFI of the mOX40L in the cell types. FIG. 5C shows a summary table of the mOX40L percentage suppression for each cell type.

FIGS. 6A-B depict the percentage frequency of OX40L+ cells (FIG. 6A), MFI of OX40L (FIG. 6A), and the percentage suppression of OX40L (FIG. 6B) in various cell types as indicated in the presence of mOX40L reporter mRNA OFF system. 3M HSPC cells maintained ex vivo were plated in a 24 well plate and transfected with 100 or 500 ng mOX40L reporter mRNAs containing the indicated miR-target-sites, mOX40L reporter mRNA with no miR target sites (miRless mOX40L group) or PBS (control). mOX40L expression and MFI was measured by flow cytometry over a 6 h-48 h timecourse. The graphs in FIG. 6A show the % frequency of OX40L+ cells and MFI of OX40L in the various cell types (HSC, Lin+, Lin− CD34+) at various timepoints as indicated. FIG. 6B shows a summary table of the mOX40L percentage suppression for each cell type at various timepoints as indicated. One-way ANOVA comparisons were made to the miRless mOX40L group. Mature BM: Mature bone marrow cells; HSCs: hematopoietic stem cells; HSPCs: hematopoietic stem and progenitor cells; MPP: multipotential progenitor cells; CLP: common lymphoid progenitor cells; CMP: common myeloid progenitor cells; GMP: granulocyte/monocyte progenitor cells; MEP: megakaryocyte-erythroid progenitor cell

FIGS. 7A-B depicts the % frequency of OX40L+ cells (FIG. 7A) and geometric MFI (FIG. 7B) of mOX40L in stem/progenitor cells in the presence of the mOX40L reporter mRNA OFF system (top panel) or ON system (bottom panel). In the OFF system, human bone marrow mononuclear cells were transfected with 100 ng or 500 ng of mOX40L reporter mRNAs containing the indicated target-sites miR126ts, miR142ts, and miR150ts (Target_3×miRts), mOX40L reporter mRNA with no miR target sites (control) or PBS. mOX40L expression was analyzed 24 h post transfection. In the ON system, human bone marrow mononuclear cells were transfected with 100 ng or 500 ng of the mOX40L_D99K target with miRless repressor RNA (Repressor) or repressor RNA containing target sites miR126ts, miR142ts, and miR150ts (Repressor_3×miRts). mOX40L expression was analyzed 24 h post transfection.

FIGS. 8A-B depict the % frequency of mOX40L+ cells and geometric MFI of mOX40L (total cells in FIG. 8A, and OX40L+ cells in FIG. 8B) in HSCs, HSPCs, and mature bone marrow cells in the presence of the mOX40L reporter mRNA OFF system. Human bone marrow mononuclear cells were transfected with 100 ng of mOX40L_D99K reporter mRNAs containing the indicated target-sites miR126ts, miR142ts, and miR150ts, mOX40L reporter mRNA with no miR target sites (miRless) or PBS. mOX40L expression was analyzed 24 h post transfection. The experiment was run in triplicate.

FIGS. 9A-B depict the % frequency of OX40L+ cells and geometric MFI of OX40L in various cells as indicated (HSCs, HSPCs, and mature bone marrow cells Lin+ cells in FIG. 9A; MPP, CLP, GMP, and MEP in FIG. 9B) in the presence of the mOX40L reporter mRNA OFF system. Lonza bone marrow mononuclear cells were transfected with 100 ng of mOX40L_D99K reporter mRNAs containing the indicated target-sites miR10ats, miR126ats, miR130ats, and miR1542ats, mOX40L reporter mRNA with no miR target sites (miRless) or PBS. mOX40L expression was analyzed 24 h post transfection.

FIG. 10 shows the various design modifications that can be employed for increasing efficacy of the miR target sites in ON/OFF systems in a polynucleotide construct. Ts #: number of target sites; AU=AU-rich element.

FIG. 11 shows the various design modifications that can be employed for increasing efficacy of the miR ON/OFF system in an exemplary polynucleotide construct.

FIG. 12 depicts the % frequency of OX40L+ cells and geometric MFI of OX40L in HSCs and monocytes in the presence of an mOX40L reporter mRNA OFF system. Human bone marrow mononuclear cells were transfected with 1 μg of the mOX40L_D99K target RNA without miR target sites(miRless), or containing 3×miR126ts, or 2×miR126ts+2×miR130ats (AU, mm). mOX40L expression was analyzed 24 h post transfection. AU=70% AU rich UTR; acc=predicted structurally accessible UTRs; mm=mismatch 18-21

FIG. 13 depicts the % frequency of OX40L+ cells and geometric MFI of OX40L in HSCs and monocytes in the presence of an mOX40L reporter mRNA ON system. Human bone marrow mononuclear cells were transfected with 100 ng of the mOX40L_D99K target RNA with non-relevant filler RNA (hEPO control) or with various L7Ae repressor RNA constructs (each 0.5× molar) as follows: L7Ae repressor RNA alone (R_miRless); L7Ae repressor RNA containing miR126ts (R_3×miR126); L7Ae repressor RNA containing miR130a (R_6×miR130a (AU, mm)); and L7Ae repressor RNA containing 2×miR126ts+2×miR130ats (bridge, AU, mm). AU=70% AU rich UTR; mm=mismatch 18-21. mOX40L expression was analyzed 24 h post transfection.

FIG. 14 depicts the % frequency of OX40L+ cells and geometric MFI of OX40L in HSCs and monocytes in the presence of an mOX40L reporter mRNA ON system. Human bone marrow mononuclear cells were transfected with 100 ng of the mOX40L_D99K target RNA with non-relevant filler RNA (control) or with various L7Ae repressor RNA constructs (each 0.5× molar) as follows: L7Ae repressor RNA without miR target sites (R_miRless); L7Ae repressor RNA containing miR142ts (R_3×miR142); L7Ae repressor RNA containing miR150a (R_3×miR150a (AU, mm)); and L7Ae repressor RNA containing 3×miR150 ((R_3×miR150). mOX40L expression was analyzed 24 h post transfection.

FIG. 15 depicts the % frequency of OX40L+ cells and geometric MFI of OX40L in CD8+ T cells and HSCs in the presence of an mOX40L reporter mRNA ON system. Human bone marrow mononuclear cells and PBMCs were transfected with 100 ng of the mOX40L_D99K target RNA with non-relevant filler RNA (control) or with various L7Ae repressor RNA constructs (each 0.5× molar) as follows: L7Ae repressor RNA without miR target sites (R_miRless); L7Ae repressor RNA containing miR150ts (R_3×miR150 (AU, mm)); L7Ae repressor RNA containing miR150 (R_3×miR150 (structure prediction)); and L7Ae repressor RNA containing miR142 (R_3×miR142). mOX40L expression was analyzed 24 h post transfection.

FIG. 16A-D depicts the % frequency of OX40L+ cells and geometric MFI of OX40L in HSCs and monocytes total cells (FIG. 16A), HSCs and monocytes OX40L+ cells (FIG. 16B), in CD34+ HSPCs (FIG. 16C), and in CD4+ T cells (FIG. 16D) in the presence of an mOX40L reporter mRNA dual ON/OFF system. Human bone marrow mononuclear cells (in triplicate) were transfected with 1 μg of the mOX40L_D99K target RNA with non-relevant filler RNA (control) or with various L7Ae repressor RNA constructs (each 0.5× molar) as follows: L7Ae repressor RNA without miR target sites (R_miRless); L7Ae repressor RNA containing miR126ts (R_3×miR126ts); L7Ae repressor RNA containing L7Ae repressor RNA containing both miR126ts and miR130ats (R_2×miR126ts+2×miR130a (bridge, AU, mm)); or L7Ae repressor RNA containing miR142ts (R_3×miR142ts). R_2×miR126ts+2×miR130a (bridge, AU, mm) represents an L7Ae repressor RNA construct with further modifications—AU=70% AU rich UTR; mm=mismatch 18-21. Some combinations further included 0.5× molar L7Ae repressor RNA R_3×miR150-OFF as indicated in the graphs. mOX40L expression was analyzed 24 h post transfection.

FIG. 17 depicts the % frequency of OX40L+ cells and geometric MFI of OX40L in monocytes and HSCs in the presence of an mOX40L reporter mRNA ON system. Human bone marrow mononuclear cells were transfected with 100 ng or 500 ng of the mOX40L_D99K target RNA with non-relevant filler RNA (control) or with various L7Ae repressor RNA constructs (each 0.5× molar) as follows: L7Ae repressor RNA without miR target sites (R_miRless); L7Ae repressor; L7Ae repressor RNA containing miR150ts (R_3×miR150ts (AU, mm)); L7Ae repressor RNA containing miR150ts (R_3×miR150 (structure prediction)); and L7Ae repressor RNA containing miR142ts (R_3×miR142). R_3×miR150ts (AU, mm) and R_3×miR150 (structure prediction) represent L7Ae repressor RNA constructs with further modifications—AU=70% AU rich UTR; mm=mismatch 18-21. mOX40L expression was analyzed 24 h post transfection.

FIG. 18 depict the % frequency of OX40L+ cells in CD8+ T cells and HSCs in the presence of an mOX40L reporter mRNA ON system. Human bone marrow mononuclear cells and PBMCs were transfected with 1 μg or 500 ng of the mOX40L_D99K target RNA with non-relevant filler RNA (control) or with various L7Ae repressor RNA constructs (each 0.5× molar) as follows: L7Ae repressor RNA without miR target sites (R_miRless); L7Ae repressor RNA containing miR150ts (R_3×miR150ts (AU, mm)); L7Ae repressor RNA containing miR150ts (R_3×miR150 (structure prediction)); and L7Ae repressor RNA containing miR142ts (R_3×miR142). R_3×miR150ts (AU, mm) and R_3×miR150 (structure prediction) represent L7Ae repressor RNA constructs with further modifications AU=70% AU rich UTR; mm=mismatch 18-21. mOX40L expression was analyzed 24 h post transfection.

FIG. 19 shows the trends of repression and rescue with miR-OFF and ON systems.

FIG. 20A is a graph depicting GFP protein AUC with mimic as a percentage of expression without mimic. mirVana miR mimic was transfected using lipofectamine 2000 at concentrations listed, along with eGFP reporter mRNAs containing 3×miR150ts, 3×miR150ts (AU, mm), or no miRts (miRless and miRless-AU), in HeLa cells. eGFP reporter expression was analyzed over 48 h using Incucyte live cell imaging. FIG. 20B is a table depicting representative data from the same experiment. FIG. 20C is a graph depicting GFP protein AUC with mimic as a percentage of expression without mimic. mirVana miR mimic was transfected using lipofectamine 2000 at concentrations listed, along with eGFP reporter mRNAs containing 3×miR150ts, 3×miR150ts (AU, mm), 3×miR150ts (accV2), 3×miR150ts (accV1), or no miRts (miRless) in HEP3B cells. eGFP reporter expression was analyzed over 48 h using Incucyte live cell imaging. In FIG. 20D, human PBMCs (n=3 donors) were transfected at 100 ng with lipid nanoparticles containing mOX40L reporter mRNAs with the indicated target sites 3×miR150ts, 3×miR150ts_AU, 3×miR150ts_mm_AU, or mOX40L reporter with no target sites (miRless) or with no target sites in the AU rich UTR (miRless_AU). mOX40L expression was analyzed 24 h post transfection by flow cytometry. AU=70% AU rich UTR; mm=mismatch 18-21. Acc=predicted structurally accessible UTRs.

FIGS. 21A-B depict the AUC of total green fluorescence as a percentage of no mimic (FIG. 21A) and AUC (FIG. 21B). For FIG. 21A, 20 ng eGFP reporter mRNAs containing indicated target sites 1×miR122ts, 3×miR122ts, 1×miR122ts (AU, mm, bridge), 3×miR122ts (AU, mm, bridge), or eGFP reporter with no target sites (miRless), and 10 nM miR122 mimic were administered to HeLa cells using lipofectamine 2000. eGFP expression was analyzed over 48 h using Incucyte live cell imaging. For FIG. 21B, HUH7 cells were transfected with 10 ng eGFP target RNA with non-relevant filler RNA (control), or with various L7Ae repressor RNA constructs (each 0.0625× molar) as follows: L7Ae repressor RNA without miR target sites (R_miRless); L7Ae repressor RNA containing 3×miR122ts; L7Ae repressor RNA containing 3×miR122ts (mm); L7Ae repressor RNA containing 3×miR122ts (AU, mm); L7Ae repressor RNA containing 6×miR122ts (AU, mm, bridge). eGFP expression was analyzed over 48 h using Incucyte live cell imaging. AU=70% AU rich UTR; mm=mismatch 18-21, bridge=miRts in the 5′ and 3′UTR.

FIGS. 22A-C are graphs depicting the AUC of total green fluorescence. In FIG. 22A, RAW264.7 cells were transfected with 10 ng eGFP reporter mRNAs containing indicated target sites 1×miR142ts, 3×miR142ts, 3×miR122ts (AU, mm, bridge) using lipofectamine 2000. eGFP expression was analyzed over 48 h using Incucyte live cell imaging. In FIG. 22B, RAW264.7 cells were transfected with 10 ng eGFP target RNA with non-relevant filler RNA (control), or with various L7Ae repressor RNA constructs (each 0.0625× molar) as follows: L7Ae repressor RNA without miR target sites (R_miRless); L7Ae repressor RNA containing 3×miR142ts; L7Ae repressor RNA containing 6×miR142ts (AU, mm, bridge). eGFP expression was analyzed over 48 h using Incucyte live cell imaging. In FIG. 22C, RAW264.7 cells were transfected with 10 ng eGFP target RNA with non-relevant filler RNA (control), or with various Puf repressor RNA constructs (each 0.2× molar) as follows: Puf repressor RNA with no miR target sites (R_miRless); Puf repressor RNA containing 3×miR142ts (R_3×142ts); Puf repressor RNA containing 6×miR142ts (R_6×miR142ts (AU, mm, bridge)). eGFP expression was analyzed over 48 h using Incucyte live cell imaging. AU=70% AU rich UTR; mm=mismatch 18-21, bridge=miRts in the 5′ and 3′UTR.

FIGS. 23A-B depict % V5 positive cells in mouse spleen (FIG. 23A) and liver (FIG. 23B). CD-1 mice, at 5 mice/group, were administered lipid nanoparticles IV containing 1 mg/kg NPI-Luciferase target mRNA with non-relevant filler mRNA or with various L7Ae repressor RNA constructs (each 0.5× molar) as follows: L7Ae repressor RNA without miR target sites (R_miRless); L7Ae repressor RNA containing 3×miR142ts, (R_3×miR142ts); L7Ae repressor RNA containing 3×miR122ts, (R_3×miR122ts), L7Ae repressor RNA containing 6×miR142ts, (R_6×miR142ts (AU, mm, bridge); L7Ae repressor RNA containing 6×miR122ts, (R_6×miR122ts (AU, mm, bridge). Spleen and liver were collected at 6 h post dose for IHC analysis. % V5 positive Kupffer cells in liver in FIG. 23B were determined by brightfield colocalization.

FIGS. 24A-B depict expression of FVIII. FIG. 24A is a schematic showing the first dosing regimen. FIG. 24B shows that FVIII mRNAs with mir142 targeting sequences show lower FVIII expression.

FIG. 25 shows that FVIII mRNAs with mir142 targeting sequences show low or no FVIII inhibitor formation.

FIGS. 26A-B depict expression of FVIII. FIG. 26A is a schematic showing the second dosing regimen. FIG. 26B shows that FVIII expression was maintained in HemA mice after repeated dosing of hFVIII mRNAs containing 6 mir142 targeting sequences in the 3′UTR.

FIGS. 27A-B depict expression of FVIII. FIG. 27A is a schematic showing the second dosing regimen. FIG. 27B shows FVIII mRNAs with mir142 targeting sequences show low or no FVIII inhibitor formation.

FIG. 28A are graphs showing expression of eGFP in THP-1 non-activated (left) and THP-1 activated (right) monocytes. Cells were transfected with eGFP reporter mRNAs containing indicated miR target sites. eGFP expression was analyzed.

FIG. 28B is a graph depicting the % frequency of OX40L+ cells. Molm-13 cells were transfected with mOX40L reporter mRNA with the indicated target sites. mOX40L expression was analyzed.

FIG. 28C are graphs showing expression of eGFP in HEL cells. Cells were transfected with eGFP reporter mRNAs containing indicated miR target sites. eGFP expression was analyzed.

FIG. 28D is a graph showing expression of eGFP in HeLa cells. Cells were transfected with eGFP reporter mRNAs containing indicated miR target sites. eGFP expression was analyzed.

FIGS. 29A-C are graphs depicting the % frequency of OX40L+/eGFP+ cells in monocytes (FIG. 29A), T cells (FIG. 29B), and dendtrictic cells (FIG. 29C). Human PBMCs, as noted, were transfected with eGFP and mOX40L reporter mRNAs containing indicated miR target sites. eGFP and mOX40L expression was analyzed.

FIG. 29D is a graph showing expression of eGFP in CD11c+DC cells. Cells were transfected with eGFP reporter mRNAs containing indicated miR target sites. eGFP expression was analyzed.

FIG. 30 depicts the eGFP reporter expression from several mRNAs. A mimic experiment was done in which HeLa cells were transfected with a mirVana miR223 mimic across a dose range, along with eGFP reporter mRNAs containing the indicated miR target sites. eGFP reporter expression was analyzed.

FIG. 31 are graphs showing mOX40L expression. Spleens of Sprague Dawley rats were injected with 0.5 mg/kg of mOX40L reporter mRNAs containing the indicated miR-target-sites. mOX40L expression in macrophages (left), T cells (middle), and B cells (right).

FIG. 32 are graphs showing the total green intensity. HeLa cells were transfected using with 10 nM, 1 nM, or 0.2 nM mirVana miR126 mimic or miR150 mimic along with mGreenLantern reporter mRNAs with the indicated miR target sites. Reporter expression was analyzed.

FIG. 33 are graphs showing total green intensity. HeLa cells were transfected using with 10 nM, 1 nM, or 0.2 nM mirVana miR126 mimic along with mGreenLantern reporter mRNAs with the indicated miR target sites. Reporter expression was analyzed.

FIGS. 34A-C show that 6×mm and ‘bridge’ designs perform comparably. The findings of FIG. 34A are depicted in tabular form in FIG. 34B. Similar studies were done in THP1 and Hep3b cells. Degree of knockdown achieved for the noted constructs are shown in FIG. 34C.

### DETAILED DESCRIPTION

Efforts to limit off-target effects of RNA-based therapeutics have focused on turning off expression of the therapeutic RNA in undesired cells and locations. The present disclosure is based on the discovery that RNA expression can be turned on or off in specific cells (ON and/or OFF systems), thereby permitting targeted and precise therapeutic and/or prophylactic action and preventing off-target effects. Further, the present disclosure is based on the discovery that various design modifications can be employed for increasing efficacy of micro RNA target sites in polynucleotide constructs for delivery.

Accordingly, disclosed herein are compositions or systems comprising polynucleotide constructs that are dependent on endogenous microRNAs in specific cells to express the polypeptide of interest, and minimize off-target expression

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean +/−5% of the recited value. For instance, an LNP including a lipid component having about 40% of a given compound may include 30-50% of the compound.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering one or more polynucleotides of this disclosure to a subject may involve administering a composition (e.g., an LNP including the one or more polynucleotides) to the subject (e.g., by an intravenous, intramuscular, intradermal, pulmonary or subcutaneous route). Administration of a composition (e.g., an LNP) to a mammal or mammalian cell may involve contacting one or more cells with the composition.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Modified: As used herein “modified” refers to a changed state or structure of a molecule of the disclosure, e.g., a change in a composition or structure of a polynucleotide (e.g., mRNA). Molecules, e.g., polynucleotides, may be modified in various ways including chemically, structurally, and/or functionally. For example, molecules, e.g., polynucleotides, may be structurally modified by the incorporation of one or more RNA elements, wherein the RNA element comprises a sequence and/or an RNA secondary structure(s) that provides one or more functions (e.g., translational regulatory activity). Accordingly, molecules, e.g., polynucleotides, of the disclosure may be comprised of one or more modifications (e.g., may include one or more chemical, structural, or functional modifications, including any combination thereof). In one embodiment, polynucleotides, e.g., mRNA molecules, of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence. However, in some embodiments the mRNA does not have a polyA tail. In some embodiments, the mRNA is a circular mRNA.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a polynucleotide of the disclosure by a delivery agent (e.g., a nanoparticle) to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of a first and/or second polypeptide in a target cell versus non-target cell to the amount of total first and/or second polypeptide in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Repressor binding element: As used herein, the term “repressor binding element” or “binding element” refers to a nucleic acid sequence, e.g., a DNA or RNA sequence, which is recognized by a repressor molecule. In an embodiment, the binding element forms a structure, e.g., a three-dimensional structure, e.g., a kink-turn, a loop, a stem or other known structure. Exemplary binding elements are provided in Table 4.

Repressor Molecule: As used herein, the term “repressor molecule” or “repressor” refers to a molecule which binds to, e.g., recognizes, a binding element or a fragment thereof. In an embodiment, the repressor binds to, e.g., recognizes, a sequence, e.g., a DNA or RNA sequence, comprising the binding element, or fragment thereof. In an embodiment, the repressor binds to, e.g., recognizes, a structure comprising a sequence, e.g., a DNA or RNA sequence, comprising the binding element, or fragment thereof. In an embodiment, the repressor comprises an RNA-binding protein or a fragment thereof. Exemplary repressors are provided in Table 1.

Therapeutic Agent: The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises or is a therapeutic payload. In some embodiments, the therapeutic agent comprises or is a small molecule or a biologic (e.g., an antibody molecule).

First polypeptide: As used herein, the term “first polypeptide” refers to an agent which elicits a desired biological and/or pharmacological effect. In an embodiment, the first polypeptide has a therapeutic and/or prophylactic effect. In an embodiment, the first polypeptide comprises a protein, a polypeptide, a peptide or a fragment (e.g., a biologically active fragment) thereof. In an embodiment, the first polypeptide includes a sequence encoding a protein, e.g., a therapeutic protein. Some examples of first polypeptides include, but are not limited to a secreted protein, a membrane-bound protein, or an intracellular protein. In an embodiment, the first polypeptide includes a cytokine, an antibody, a vaccine (e.g., an antigen, or an immunogenic epitope), a receptor, an enzyme, a hormone, a transcription factor, a ligand, a membrane transporter, a structural protein, a nuclease, or a component, a variant or a fragment (e.g., a biologically active fragment) thereof. The terms protein, polypeptide and peptide are used interchangeably herein.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

Target Site: As used herein, the term “target site”, “cleavage site”, and binding site are used interchangeably and refer to the recognition sequence within a polynucleotide molecule. A target site in the context of this disclosure can be a microRNA target site, or an endonuclease cleavage site. A target site can be engineered to be in the 5′ UTR or the 3′ UTR of the polynucleotide molecule.

Turning ON expression: As used herein, the term “turning ON expression” or “turning ON translation” refers to refers to increasing the degree of transcription or translation from a particular polynucleotide when the polynucleotide comes into contact with a particular microRNA) in a particular cell/microenvironment and that miRNA binds to and cleaves one or more microRNA target sites (miRts) on the second and optionally the first polynucleotide in a dual polynucleotide system.

In some embodiments, binding of the miRNA (e.g., HSPC-specific miRNA) to the corresponding miRts on the second polynucleotide leads to degradation of the second polynucleotide encoding the repressor, thereby allowing expression of the target protein from the first polynucleotide (turning ON expression) in the desired cells (e.g., HSPC cells). In other embodiments, binding of the miRNA (e.g., non-HSPC-specific miRNA) to the corresponding miRts on the first polynucleotide leads to degradation of the first polynucleotide encoding the target protein, thereby reducing expression of the target protein in undesired cells (e.g., non-HSPC cells). In some embodiments, the second polynucleotide has one or more microRNA target sites that are capable of binding to miRNA present in hematopoietic stem and progenitor cells (HSPC miRts). In other embodiments, the first polynucleotide has one or more microRNA target sites that are capable of binding to miRNA present in non-hematopoietic stem and progenitor cells (non-HSPC miRts).

In some embodiments, binding of the miRNA (e.g., non-HSPC-specific miRNA, such as a mature-immune cell specific miRNA) to the corresponding miRts on the second polynucleotide leads to degradation of the second polynucleotide encoding the repressor, thereby allowing expression of the target protein from the first polypeptide (turning ON expression) in the desired cells (e.g., non-HSPC cells). In other embodiments, binding of the miRNA (e.g., HSPC-specific miRNA) to the corresponding miRts on the first polynucleotide leads to degradation of the first polynucleotide encoding the target protein, thereby reducing expression of the target protein in undesired cells (e.g., HSPC cells), but keeping expression ON in non-HSPC cells. In some embodiments, the second polynucleotide has one or more microRNA target sites that are capable of binding to miRNA present in non-hematopoietic stem and progenitor cells (non-HSPC miRts). In other embodiments, the first polynucleotide has one or more microRNA target sites that are capable of binding to miRNA present in hematopoietic stem and progenitor cells (HSPC miRts).

Binding of the miRNA to the miRts can cause partial or full cleavage of the second polynucleotide, leading to turning ON expression or translation of the first polynucleotide. When the transcription or translation of the first polypeptide is turned ON due to miRNA-miRts binding and cleavage in a specific cell/tissue, it can increase the level of transcription or translation of the first polypeptide in that specific cell/tissue by at least about 20%, 30%, 40%, 50%, 60%, 70%, 80%, 85%, 90%, 95%, 96%, 97%, 98%, 99% or 100% when compared to the level of transcription or translation in the absence of miRNA-miRts binding and cleavage.

Turning OFF expression: As used herein, the term “turning OFF expression” or “turning OFF translation” refers to refers to decreasing the degree of transcription or translation from a particular polynucleotide (e.g., an mRNA) when the polynucleotide comes into contact with a particular microRNA) in a particular cell/microenvironment and that miRNA binds to and cleaves one or more microRNA target sites (miRts) on the mRNA in a single polynucleotide system.

In some embodiments, binding of the miRNA (e.g., HSPC-specific miRNA) to the corresponding miRts on an mRNA encoding a target protein leads to degradation of the mRNA encoding the target, thereby turning OFF expression of the target protein from the mRNA in the cells that express the miRNA specific to the miRts on the mRNA (e.g., HSPC cells).

Binding of the miRNA to the miRts can cause partial or full cleavage of the mRNA, leading to turning OFF expression or translation of the mRNA. When the transcription or translation of the first polypeptide is turned OFF due to miRNA-miRts binding and cleavage in a specific cell/tissue, it can decrease the level of transcription or translation of the target protein in that specific cell/tissue by at least about 20%, 30%, 40%, 50%, 60%, 70%, 80%, 85%, 90%, 95%, 96%, 97%, 98%, 99% or 100% when compared to the level of transcription or translation in the absence of miRNA-miRts binding and cleavage.

Unsuitable for canonical translation: A polynucleotide (e.g., RNA) that is “unsuitable for canonical translation” is a polynucleotide with a nucleotide modification, sequence modification, and/or a structure that is not suitable for translation. In some embodiments, the modification(s) are at the 5′ end, and/or the 3′ end. In some embodiments, the modification(s) stabilize the polynucleotide. In some embodiments, a polynucleotide that is unsuitable for canonical translation (a) does not have a polyA tail; (b) is circular; (c) has no cap; and/or (d) has no cap and no tail.

Polynucleotides of ON and/or OFF Systems

Disclosed herein, inter alia, are compositions and systems that are ON and/or OFF compositions and systems that encode for a polypeptide and optionally, a repressor. In a single polynucleotide system, the compositions and systems encode for a polypeptide, that is a target protein. In a single polynucleotide system, the single polynucleotide is an mRNA of the composition. In a dual polynucleotide system, the first polynucleotide of the composition encodes for a polypeptide, that is a target protein. Additionally, the first polynucleotide also has a repressor binding element. In a dual polynucleotide system, the second polynucleotide of the composition encodes for repressor protein. The single and dual polynucleotide systems and its features are discussed below in greater detail.

### Single Polynucleotide OFF System

Disclosed herein, inter alia, are compositions and systems comprising a single polynucleotide, i.e., a messenger RNA (mRNA).

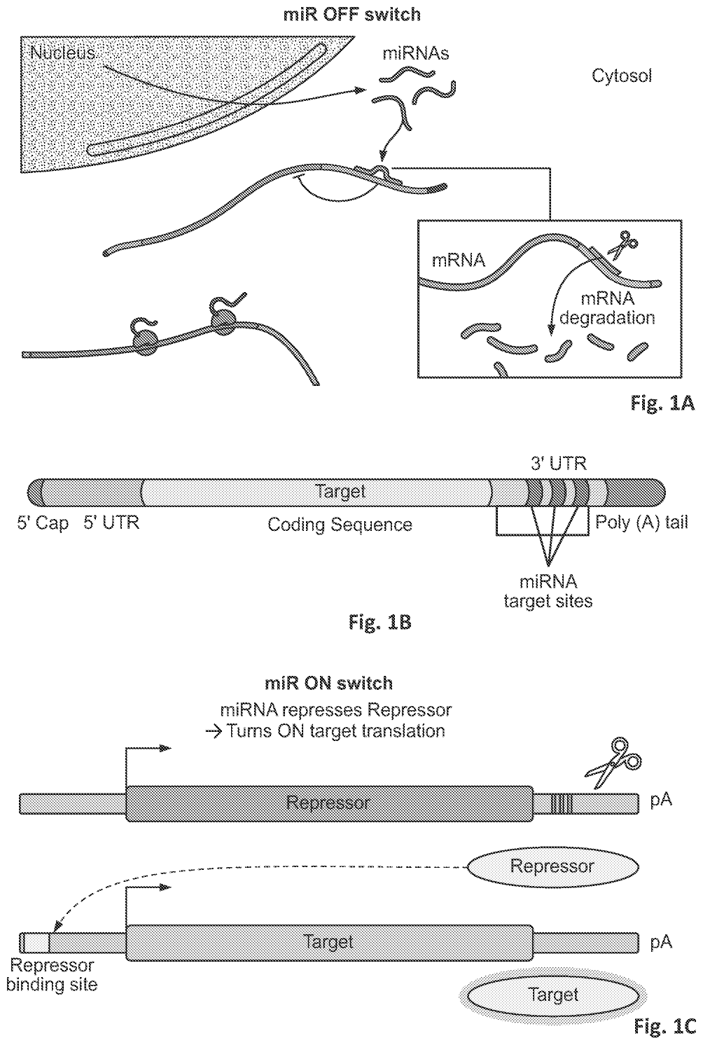

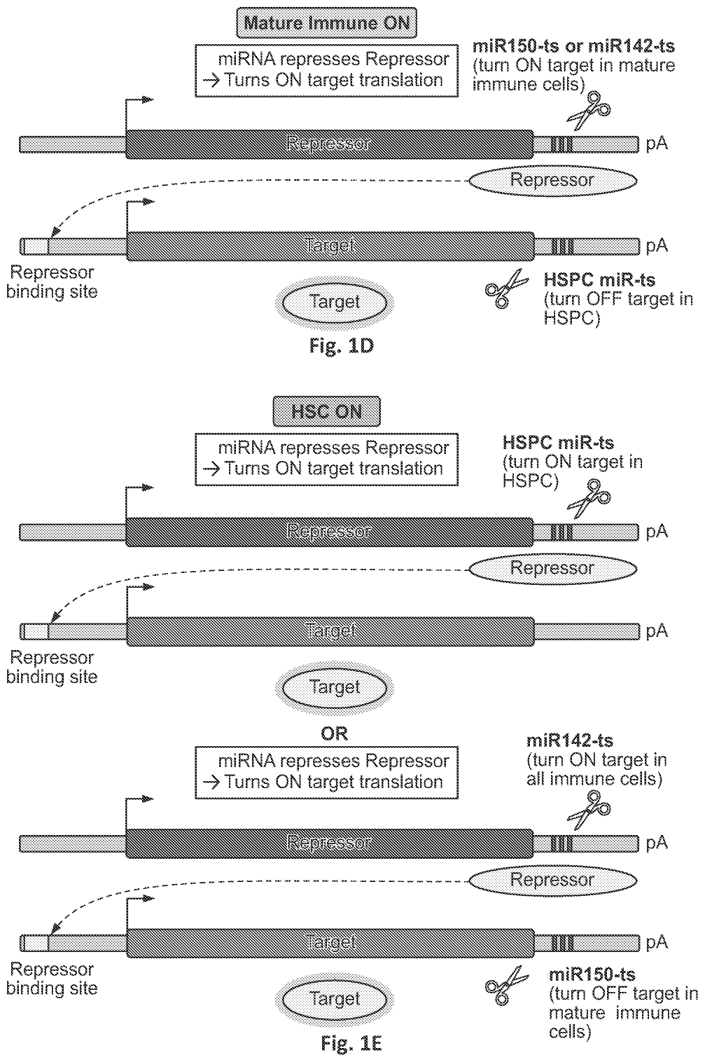

In one aspect, the composition comprises a messenger RNA (mRNA) comprising (i) an open reading frame encoding a polypeptide, and (ii) one or more microRNA target sites present in hematopoietic stem and progenitor cells (HSPC miRts). An example of such a system (a single RNA system) is depicted in FIG. 1A. In this example, the mRNA encodes a target polypeptide and contains microRNA target sites. FIG. 1A shows that in the presence of microRNA binding to the microRNA target sites (miRts), the mRNA is degraded, thereby suppressing (turning OFF) target protein translation. Target expression is turned OFF only in HSPCs that express the particular microRNA which is capable of binding to the miRts on the mRNA. FIG. 1B shows an exemplary mRNA with a 5′ Cap, a 5′ untranslated region (UTR), a target polypeptide encoding region followed by 3 miRts in the 3′ UTR and a poly A tail.

In some embodiments, the methods of this disclosure can be used to turn OFF expression of target polypeptide in particular cell lineages, e.g., hematopoietic progenitor cells in which gene editing is desired, thereby increasing safety of gene-editing technologies, or mature immune cells (e.g., macrophages, T cells, B cells, etc) in which gene expression is not desired. In some embodiments, the single polynucleotide OFF system disclosed herein has immune-oncology applications. For example, the OFF system can be used to turn OFF and reduce expression of mRNAs in HSPCs (HSPC-OFF system) that induce differentiation and polarization in HSPCs to mitigate risk of reprograming bone marrow development. In some embodiments, the HSPC-OFF system can be coupled with a mature immune cell ON system, such that target mRNA expression is turned OFF in HSPCs but turned ON in mature immune cells, thereby providing additional safety and selectivity for target RNA expression in mature (differentiated) immune cells.

### Dual Polynucleotide ON/OFF System

Disclosed herein, inter alia, are compositions and systems comprising two polynucleotides, i.e., (a) a first polynucleotide comprising (i) a repressor binding element, (ii) an open reading frame encoding a polypeptide, and (iii) optionally one or more microRNA target sites present in non-HSPCs; and (b) a second polynucleotide comprising (i) a sequence encoding a repressor that binds to the repressor binding element and (ii) one or more microRNA target sites present in HSPCs, wherein binding of the repressor to the repressor binding element reduces translation of the polypeptide from the first polynucleotide. In some embodiments, both the polynucleotides are RNA molecules (e.g., mRNAs). In some embodiments, both the polynucleotides are DNA molecules. In some embodiments, one polynucleotide is an RNA molecule (e.g., mRNA) and the other polynucleotides is a DNA molecule. An example of such a dual polynucleotide OFF system is depicted in FIG. 1C in which microRNA binding to the miRts of a repressor encoding RNA turns ON expression of target RNA, by reducing expression of the repressor and thereby preventing the repressor from binding to the repressor binding site on the target RNA. FIG. 1D shows a mature immune cell ON system in which microRNA binding to the microRNA target sites (e.g., miR150-ts or miR142-ts miRts) of a repressor encoding RNA turns ON expression of target RNA in mature immune cells, by reducing expression of the repressor and thereby preventing the repressor from binding to the repressor binding site on the target RNA. FIG. 1E shows an HSPC ON system in which microRNA binding to a microRNA target site of a repressor encoding RNA turns ON expression of target RNA in HSPCs, by reducing expression of the repressor and thereby preventing the repressor from binding to the repressor binding site on the target RNA. In some embodiments, the dual ON system comprises different miRts in each polypeptide of the dual ON system to fine-tune expression of the target polypeptide in desired cells. For instance, as shown in FIG. 1E, the repressor encoding RNA contains microRNA target sites that are specific for a pan-immune cell microRNA (e.g., miR142). Binding of the microRNA (e.g., miR142) to the microRNA target sites (e.g., miR142ts) of the repressor encoding RNA turns ON expression of target RNA in all immune cells, by reducing expression of the repressor and thereby preventing the repressor from binding to the repressor binding site on the target RNA. Meanwhile, the target encoding RNA contains microRNA target sites that are specific for another non-HSPC-specific microRNA (e.g., miR150). Binding of the microRNA (e.g., miR150) to the microRNA target sites (e.g., miR150ts) of the target encoding RNA turns OFF expression of target RNA in mature immune cells, by degrading target RNA in those cells.

Polypeptides of ON and/or OFF Systems

In some embodiments of the ON and/or OFF systems disclosed herein, the polypeptide of the ON and/or OFF compositions and systems encodes: a secreted protein; a membrane-bound protein; or an intercellular protein, or peptides, polypeptides or biologically active fragments thereof.

In some embodiments, the polypeptide is a secreted protein, or a peptide, a polypeptide or a biologically active fragment thereof. In some embodiments, the secreted protein comprises a cytokine, or a variant or fragment (e.g., a biologically active fragment) thereof. In some embodiments, the secreted protein comprises an antibody or a variant or fragment (e.g., a biologically active fragment) thereof. In some embodiments, the secreted protein comprises an enzyme or a variant or fragment (e.g., a biologically active fragment) thereof. In some embodiments, the secreted protein comprises a hormone or a variant or fragment (e.g., a biologically active fragment) thereof. In some embodiments, the secreted protein comprises a ligand, or a variant or fragment (e.g., a biologically active fragment) thereof. In some embodiments, the secreted protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the secreted protein comprises a growth factor or a component, variant or fragment (e.g., a biologically active fragment) thereof. In some embodiments, the secreted protein comprises an immune modulator, e.g., an immune checkpoint agonist or antagonist.

In some embodiments, the polypeptide is a membrane-bound protein, or a peptide, a polypeptide or a biologically active fragment thereof. In some embodiments, the membrane-bound protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the membrane-bound protein comprises a ligand, a variant or fragment (e.g., a biologically active fragment) thereof. In some embodiments, the membrane-bound protein comprises a membrane transporter, a variant or fragment (e.g., a biologically active fragment) thereof. In some embodiments, the membrane-bound protein comprises a structural protein, a variant or fragment (e.g., a biologically active fragment) thereof. In some embodiments, the membrane-bound protein comprises an immune modulator, e.g., an immune checkpoint agonist or antagonist.

In some embodiments, the polypeptide is an intracellular protein, or a peptide, a polypeptide or a biologically active fragment thereof. In some embodiments, the intracellular protein comprises an enzyme, or a variant or fragment (e.g., a biologically active fragment) thereof. In some embodiments, the intracellular protein comprises a hormone, or a variant or fragment (e.g., a biologically active fragment) thereof. In some embodiments, the intracellular protein comprises a cytokine, or a variant or fragment (e.g., a biologically active fragment) thereof. In some embodiments, the intracellular protein comprises a transcription factor, or a variant or fragment (e.g., a biologically active fragment) thereof. In some embodiments, the intracellular protein comprises a nuclease, or a variant or fragment (e.g., a biologically active fragment) thereof. In some embodiments, the intracellular protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the intracellular protein comprises a structural protein, or a variant or fragment (e.g., a biologically active fragment) thereof.

In some embodiments, the polypeptide is chosen from a cytokine, an antibody, a vaccine (e.g., an antigen, an immunogenic epitope), a receptor, an enzyme, a hormone, a transcription factor, a ligand, a membrane transporter, a structural protein, a nuclease, a growth factor, an immune modulator, or a component, variant or fragment (e.g., a biologically active fragment) thereof.

In some embodiments, the polypeptide comprises a cytokine, or a variant or fragment (e.g., a biologically active fragment) thereof.

In some embodiments, the polypeptide comprises an antibody or a variant or fragment (e.g., a biologically active fragment) thereof.

In some embodiments, the polypeptide comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine.

In some embodiments, the polypeptide comprises a receptor, or a variant or fragment (e.g., a biologically active fragment) thereof.

In some embodiments, the polypeptide comprises an enzyme, or a variant or fragment (e.g., a biologically active fragment) thereof.

In some embodiments, the polypeptide comprises a hormone, or a variant or fragment (e.g., a biologically active fragment) thereof.

In some embodiments, the polypeptide comprises a growth factor, or a variant or fragment (e.g., a biologically active fragment) thereof.

In some embodiments, the polypeptide comprises a nuclease, or a variant or fragment (e.g., a biologically active fragment) thereof.

In some embodiments, the polypeptide comprises a transcription factor, or a variant or fragment (e.g., a biologically active fragment) thereof.

In some embodiments, the polypeptide comprises a ligand, or a variant or fragment (e.g., a biologically active fragment) thereof.

In some embodiments, the polypeptide comprises a membrane transporter, or a variant or fragment (e.g., a biologically active fragment) thereof.

In some embodiments, the polypeptide comprises a structural protein, or a variant or fragment (e.g., a biologically active fragment) thereof.

In some embodiments, the polypeptide comprises an immune modulator, or a variant or fragment (e.g., a biologically active fragment) thereof. In some embodiments, the immune modulator comprises an immune checkpoint agonist or antagonist.

In some embodiments, the polypeptide comprises a protein or peptide.

In some embodiments, the polypeptide comprises a protein associated with gene editing in a cell (an editor protein). For instance, the protein is a Cas nuclease, a zinc finger nuclease, or a homing endonuclease. In some embodiments, the polypeptide comprises a self-splicing intein, wherein the editor protein is fused to a degron domain that leads to rapid protein degradation.

Repressors of the ON and/or OFF System

In some embodiments of the ON and/or OFF systems disclosed herein, the single polynucleotide of the OFF system, or the second polynucleotide of the ON and/or OFF system encodes an RNA binding protein or a fragment thereof, e.g., a repressor or a biologically active fragment thereof.

In some embodiments, the repressor is chosen from the molecules provided in Table 2, e.g., Snu13, 50S ribosomal L7Ae protein, Pumilio and FBF (PUF) protein, PUF2 protein, MBP-LacZ, MBP, PCP, Lambda N, U1A, 15.5kd, LARP7, L30e, or a variant or fragment thereof.

Snu13 is a human spliceosomal protein which binds U4 snRNA during spliceosomal assembly. (EMBO J. 1999 Nov. 1; 18(21):6119-33.)

L7Ae is an archaeal ribosomal protein which regulates the translation of a designed mRNA in vitro and in human cells (see, e.g., Saito H, et al., Nat Chem Biol. 2010 January; 6(1):71-8); and Wroblewska L, et al. Nat Biotechnol. 2015; 33(8):839-841.

In some embodiments, when the repressor is 50S ribosomal L7Ae protein (e.g., wildtype 50S ribosomal L7Ae protein, a variant or fragment thereof) the binding element is a kink-turn forming sequence (e.g., SEQ ID NO: 7, or a variant or fragment thereof).

PUF is a family of proteins that bind RNA sequence stretches defined by their amino acid identities at specific positions. Some amino acids in the protein can be engineered to change binding to any other RNA sequence. PUF2 is such an engineered protein. Filipovska A, et al. Nat Chem Biol. 2011 May 15; 7(7):425-7.

In some embodiments, when the repressor is PUF (e.g., wildtype PUF, or a variant or fragment thereof) the repressor binding element is PRE (e.g., wildtype PRE, or a variant or fragment thereof).

In some embodiments, when the repressor is PUF2 (e.g., wildtype PUF2, or a variant or fragment thereof) the repressor binding element is PRE2 (e.g., wildtype PRE2, or a variant or fragment thereof).

In some embodiments, when the repressor is MBP (e.g., wildtype MBP, a variant or fragment thereof) the repressor binding element is MS2 (e.g., wildtype MS2, or a variant or fragment thereof). In some instances, MBP predimerizes prior to binding to MS2 hairpins.

In some embodiments, when the repressor is MBP-LacZ or a variant or fragment thereof, the repressor binding element is MS2 (e.g., wildtype MS2, or a variant or fragment thereof).

In some embodiments, when the repressor is PCP (e.g., wildtype PCP, or a variant or fragment thereof) the repressor binding element is PP7 (e.g., wildtype PP7, or a variant or fragment thereof).

In some embodiments, when the repressor is Lambda N (e.g., wildtype Lambda N, or a variant or fragment thereof) the repressor binding element is BoxB (e.g., wildtype BoxB, or a variant or fragment thereof).

In some embodiments, when the repressor is U1A (e.g., wildtype U1A, or a variant or fragment thereof) the repressor binding element is U1A hairpin (e.g., wildtype U1A hairpin, or a variant or fragment thereof).

In some embodiments, when the repressor is 15.5kd (e.g., wildtype 15.5kd, or a variant or fragment thereof) the repressor binding element is a kink-turn forming sequence (e.g., wildtype U1A hairpin, or a variant or fragment thereof).

In some embodiments, when the repressor is LARP7 (e.g., wildtype LARP7, or a variant or fragment thereof) the repressor binding element is 7SK (e.g., wildtype 7SK, or a variant or fragment thereof).

In some embodiments, a repressor comprises an RNA-binding protein or a variant or a fragment thereof. Exemplary RNA-binding proteins are provided in Tables 1 and 2.

TABLE 1
Exemplary repressors and repressor binding elements

| RNA binding | | Recognition |
|---|---|---|
| protein (RBP) | RNA element | basis |
| PUF | PRE | sequence |
| PUF2 | PRE2 | sequence |
| MBP | MS2 | structure |
| MBP-LacZ | MS2 | structure |
| PCP | PP7 | structure |
| Lambda N | BoxB | structure |
| U1A | U1A hairpin | structure |
| 15.5 kd | Kink-turn forming sequence | structure |
| 50S ribosomal | Kink-turn forming sequence | structure |
| L7Ae protein | | |
| LARP7 | 7SK | structure |

Additional exemplary RNA-binding proteins or RNA-binding domains which can be used as repressors are disclosed in Corley et al, Molecular Cell 78:1 pp. 9-29, the entire contents of which are hereby incorporated by reference. For example, Table 2 provides additional exemplary RNA-binding proteins or domains which can be used as repressors. In an embodiment, a repressor disclosed herein comprises a domain (or a variant, or a fragment thereof) or a protein (or a variant or a fragment thereof) listed in Table 2.

TABLE 2
Exemplary RNA-binding proteins and domains

| Domain name | Protein family containing domain |
|---|---|
| Cold shock domain | Cold shock proteins, Y-box proteins |
| Double stranded RNA binding | RNAses, ADARs, DICER |
| domain | |
| Helicase | DExH/D-box, Ski2-like, RIGI-like, |
| | NS3, UPF1-like RNA binding helicases |
| Intrinsically disordered | Most RBPs |
| regions (IDR) | |
| K homology | hnRNPs, translation regulation |
| | proteins, |
| La motif (LAM) | La proteins, La-related proteins |
| | (LARPs) |
| Piwi-Argonaute-Zwille | Argonaute proteins, Dicer |
| (PAZ) | |
| P-element Induced Wimpy | Argonaute proteins, |
| Testis (PIWI) | |
| Pentatricopeptide repeat (PPR) | RNA editing proteins |
| Pseudouridine synthase and | RNA modifying enzymes, metabolic |
| archaeosine transglycoslyase | enzymes |
| (PUA) | |
| Pumillo-like repeat (PUM) | PUF proteins |
| Ribosomal S1-like | Ribosomal proteins, Translation |
| | initiation factors, RNase II, PNPase |
| RNA recognition motif (RRM) | hnRNPs, splicing factors |
| Sm and Like-Sm (Sm/Lsm) | U1 spliceosomal proteins, Hfq |
| thiouridine synthases, RNA | tRNA modifying enzyme |
| methylases and pseudouridine | |
| synthases (THUMP) | |
| YT521-B homology (YTH) | YTH family m6A readers |
| Zn finger | Transcription factors, METTL |
| | enzymes, |

In some embodiments, the repressor comprises MBP. In some embodiments, the repressor comprises an amino acid sequence provided in Table 3 or a sequence with at least 80%, 85%, 90%, 95%, 96%, 97%, 98%, 99% or 100% identity thereof. In some embodiments, the repressor comprises the amino acid sequence of SEQ ID NO: 21, or an amino acid sequence with at least 80%, 85%, 90%, 95%, 96%, 97%, 98%, 99% or 100% identity thereof.

In some embodiments, the repressor is encoded by a nucleotide sequence provided in Table 3 or a sequence with at least 80%, 85%, 90%, 95%, 96%, 97%, 98%, 99% or 100% identity thereof. In some embodiments, the repressor is encoded by the nucleotide sequence of SEQ ID NO: 22, or a sequence with at least 80%, 85%, 90%, 95%, 96%, 97%, 98%, 99% or 100% identity thereof.

TABLE 3
Exemplary sequences of repressor molecules

| | | |
|---|---|---|
| SEQ ID | Sequence | |
| NO | information | Sequence |

Repressor sequence

| | | |
|---|---|---|
| 21 | MBP (aa) | MASNFTQFVLVDNGGTGDVTVAPSNFANGIAEWISSNSRSQAYKVTCS |
| | | VRQSSAQNRKYTIKVEVPKGAWRSYLNMELTIPIFATNSDCELIVKAMQ |
| | | GLLKDGNPIPSAIAANSGIY |
| 22 | MBP (nt) | ATGGCCAGCAACTTCACCCAGTTCGTGCTGGTGGACAACGGCGGTA |
| | | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTAC |
| 25 | MBP- | MASNFTQFVLVDNGGTGDVTVAPSNFANGIAEWISSNSRSQAYKVTCS |
| | eIF4GdN | VRQSSAQNRKYTIKVEVPKGAWRSYLNMELTIPIFATNSDCELIVKAMQ |
| | (aa) | GLLKDGNPIPSAIAANSGIYGGGGSIFASMQKPEGLPHISDVVLDKANKT |
| | | PLRPLDPTRLQGINCGPDFTPSFANLGRTTLSTRGPPRGGPGGELPRGPA |
| | | GLGPRRSQQGPRKEPRKIIATVLMTEDIKLNKAEKAWKPSSKRTAADK |
| | | DRGEEDADGSKTQDLFRRVRSILNKLTPQMFQQLMKQVTQLAIDTEER |
| | | LKGVIDLIFEKAISEPNFSVAYANMCRCLMALKVPTTEKPTVTVNFRKL |
| | | LLNRCQKEFEKDKDDDEVFEKKQKEMDEAATAEERGRLKEELEEARDI |
| | | ARRRSLGNIKFIGELFKLKMLTEAIMHDCVVKLLKNHDEESLECLCRLL |
| | | TTIGKDLDFEKAKPRMDQYFNQMEKIIKEKKTSSRIRFMLQDVLDLRGS |
| | | NWVPRRGDQGPKTIDQIHKEAEMEEHREHIKVQQLMAKGSDKRRGGP |
| | | PGPPISRGLPLVDDGGWNTVPISKGSRPIDTSRLTKITKPGSIDSNNQLFA |
| | | PGGRLSWGKGSSGGSGAKPSDAASEAARPATSTLNRFSALQQAVPTEST |
| | | DNRRVVQRSSLSRERGEKAGDRGDRLERSERGGDRGDRLDRARTPATK |
| | | RSFSKEVEERSRERPSQPEGLRKAASLTEDRDRGRDAVKREAALPPVSP |
| | | LKAALSEEELEKKSKAIIEEYLHLNDMKEAVQCVQELASPSLLFIFVRHG |
| | | VESTLERSAIAREHMGQLLHQLLCAGHLSTAQYYQGLYEILELAEDMEI |
| | | DIPHVWLYLAELVTPILQEGGVPMGELFREITKPLRPLGKAASLLLEILG |
| | | LLCKSMGPKKVGTLWREAGLSWKEFLPEGQDIGAFVAEQKVEYTLGEE |
| | | SEAPGQRALPSEELNRQLEKLLKEGSSNQRVFDWIEANLSEQQIVSNTL |
| | | VRALMTAVCYSAIIFETPLRVDVAVLKARAKLLQKYLCDEQKELQALY |
| | | ALQALVVTLEQPPNLLRMFFDALYDEDVVKEDAFYSWESSKDPAEQQ |
| | | GKGVALKSVTAFFKWLREAEEESDHN |
| 26 | MBP- | ATGGCCAGCAACTTCACCCAGTTCGTGCTGGTGGACAACGGCGGTA |
| | eIF4GdN (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCATA |
| | | TTCGCGAGCATGCAGAAGCCTGAAGGTCTGCCTCACATCAGCGACG |
| | | TGGTGCTGGACAAGGCCAACAAGACCCCTCTTAGACCTCTGGACCCT |
| | | ACCAGACTGCAGGGCATCAACTGCGGCCCTGACTTCACTCCTTCTTT |
| | | CGCAAACCTGGGCAGAACCACCCTGAGCACCAGAGGCCCTCCTAGA |
| | | GGTGGTCCCGGCGGAGAACTCCCCAGGGGTCCTGCCGGCCTGGGCC |
| | | CTAGACGCTCCCAGCAAGGTCCTAGAAAGGAGCCAAGGAAGATCAT |
| | | CGCCACCGTGCTGATGACCGAGGACATCAAGCTGAACAAAGCTGAG |
| | | AAGGCCTGGAAGCCTAGCAGCAAGAGAACCGCCGCCGACAAGGAC |
| | | AGAGGCGAGGAGGACGCCGACGGATCCAAGACCCAGGACCTGTTCA |
| | | GAAGAGTGAGAAGCATCCTCAACAAGCTGACCCCTCAGATGTTCCA |
| | | GCAGCTGATGAAGCAGGTGACGCAGCTCGCCATCGACACCGAGGAG |
| | | AGACTGAAGGGCGTGATCGACCTGATCTTTGAGAAGGCTATCTCAG |
| | | AGCCTAACTTCAGCGTGGCCTACGCCAACATGTGCCGTTGCCTGATG |
| | | GCATTGAAGGTGCCAACCACCGAGAAGCCTACTGTGACCGTCAATTT |
| | | CCGTAAACTGCTGCTGAACCGGTGCCAGAAAGAGTTCGAGAAGGAT |
| | | AAGGACGACGACGAGGTCTTCGAGAAGAAACAGAAAGAAATGGAC |
| | | GAGGCCGCCACCGCAGAGGAAAGGGGCCGATTAAAGGAGGAGCTG |
| | | GAGGAGGCCAGAGACATCGCCAGACGGCGTTCTCTGGGCAACATCA |
| | | AGTTCATAGGTGAGCTGTTCAAGCTAAAGATGCTCACCGAGGCCAT |
| | | AATGCACGACTGCGTGGTGAAGCTACTGAAGAACCACGACGAAGAA |
| | | AGCCTGGAGTGCCTGTGCAGACTGCTGACCACCATCGGCAAGGACC |
| | | TGGACTTCGAGAAGGCAAAGCCTCGAATGGACCAGTACTTCAACCA |
| | | GATGGAGAAGATTATCAAGGAGAAGAAGACCAGCAGCAGAATCAG |
| | | ATTCATGCTGCAGGACGTACTGGACCTGCGCGGAAGCAACTGGGTG |
| | | CCAAGGAGAGGGGACCAAGGACCAAAGACCATCGACCAGATCCAC |
| | | AAGGAAGCGGAGATGGAGGAGCACAGAGAGCACATAAAGGTGCAG |
| | | CAGCTTATGGCCAAGGGCAGCGACAAGCGAAGAGGCGGCCCGCCCG |
| | | GACCTCCTATCAGCAGAGGCCTTCCTCTGGTAGACGACGGCGGCTGG |
| | | AACACCGTGCCTATCTCTAAGGGCTCCAGACCTATCGACACTTCCCG |
| | | TCTTACCAAGATCACCAAGCCAGGATCTATTGACAGCAACAACCAG |
| | | CTGTTCGCCCCAGGAGGAAGACTTAGCTGGGGCAAGGGAAGTTCCG |
| | | GCGGATCCGGCGCCAAGCCTTCCGACGCCGCCAGCGAGGCTGCCAG |
| | | ACCTGCCACCAGCACCTTGAACCGCTTTTCCGCTCTGCAGCAAGCTG |
| | | TGCCGACCGAGAGCACCGACAACCGGCGAGTTGTGCAGAGAAGCAG |
| | | CCTGAGCAGAGAGAGGGGCGAGAAAGCCGGCGATAGAGGTGACCG |
| | | ACTGGAGAGAAGCGAGAGAGGAGGTGATAGAGGCGACCGGCTTGA |
| | | CAGAGCCAGAACCCCTGCCACAAAGCGATCGTTCAGCAAGGAGGTC |
| | | GAAGAGAGGTCCAGGGAGCGCCCTAGCCAGCCGGAAGGACTCAGA |
| | | AAGGCAGCCAGTCTAACAGAGGACCGCGACAGGGGAAGAGACGCC |
| | | GTGAAGAGGGAGGCCGCACTGCCTCCTGTGAGCCCTCTGAAGGCGG |
| | | CACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTCAA |
| | | GGAGGGATCCTCAAATCAGAGAGTGTTCGACTGGATCGAGGCCAAT |
| | | CTCAGCGAGCAGCAAATCGTGAGCAACACGTTGGTTCGCGCTCTCAT |
| | | GACAGCCGTGTGCTACTCAGCCATTATCTTCGAGACGCCTCTCCGCG |
| | | TGGACGTGGCAGTGCTCAAGGCCCGCGCTAAGCTGTTACAGAAGTA |
| | | CCTGTGCGACGAGCAGAAGGAACTGCAGGCCCTGTACGCCCTGCAA |
| | | GCTCTGGTCGTCACACTCGAGCAGCCTCCTAACCTGCTGAGAATGTT |
| | | CTTCGACGCATTGTACGACGAGGACGTTGTGAAGGAAGACGCTTTCT |
| | | ACAGCTGGGAGTCTAGTAAGGATCCTGCGGAACAACAAGGCAAGGG |
| | | CGTTGCTCTAAAGAGCGTGACCGCCTTCTTCAAGTGGCTGCGAGAGG |
| | | CTGAGGAAGAGTCGGACCACAAC |
| 27 | MBP_eIF4G | MASNFTQFVLVDNGGTGDVTVAPSNFANGIAEWISSNSRSQAYKVTCS |
| | (fl) (aa) | GLLKDGNPIPSAIAANSGIYGGGGSNKAPQSTGPPPAPSPGLPQPAFPPG |
| | | VRQSSAQNRKYTIKVEVPKGAWRSYLNMELTIPIFATNSDCELIVKAMQ |
| | | QTAPVVFSTPQATQMNTPSQPRQHFYPSRAQPPSSAASRVQSAAPARPG |
| | | PAAHVYPAGSQVMMIPSQISYPASQGAYYIPGQGRSTYVVPTQQYPVQP |
| | | GAPGFYPGASPTEFGTYAGAYYPAQGVQQFPTGVAPTPVLMNQPPQIA |
| | | PKRERKTIRIRDPNQGGKDITEEIMSGARTASTPTPPQTGGGLEPQANGE |
| | | TPQVAVIVRPDDRSQGAIIADRPGLPGPEHSPSESQPSSPSPTPSPSPVLEP |
| | | GSEPNLAVLSIPGDTMTTIQMSVEESTPISRETGEPYRLSPEPTPLAEPILE |
| | | VEVTLSKPVPESEFSSSPLQAPTPLASHTVEIHEPNGMVPSEDLEPEVESS |
| | | PELAPPPACPSESPVPIAPTAQPEELLNGAPSPPAVDLSPVSEPEEQAKEV |
| | | TASMAPPTIPSATPATAPSATSPAQEEEMEEEEEEEEGEAGEAGEAESEK |
| | | GGEELLPPESTPIPANLSQNLEAAAATQVAVSVPKRRRKIKELNKKEAV |
| | | GDLLDAFKEANPAVPEVENQPPAGSNPGPESEGSGVPPRPEEADETWDS |
| | | KEDKIHNAENIQPGEQKYEYKSDQWKPLNLEEKKRYDREFLLGFQFIFA |
| | | SMQKPEGLPHISDVVLDKANKTPLRPLDPTRLQGINCGPDFTPSFANLG |
| | | RTTLSTRGPPRGGPGGELPRGPAGLGPRRSQQGPRKEPRKIIATVLMTED |
| | | IKLNKAEKAWKPSSKRTAADKDRGEEDADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKFIGELFKLKMLTEAIMH |
| | | DCVVKLLKNHDEESLECLCRLLTTIGKDLDFEKAKPRMDQYFNQMEKII |
| | | KEKKTSSRIRFMLQDVLDLRGSNWVPRRGDQGPKTIDQIHKEAEMEEH |
| | | REHIKVQQLMAKGSDKRRGGPPGPPISRGLPLVDDGGWNTVPISKGSRP |
| | | IDTSRLTKITKPGSIDSNNQLFAPGGRLSWGKGSSGGSGAKPSDAASEAA |
| | | RPATSTLNRFSALQQAVPTESTDNRRVVQRSSLSRERGEKAGDRGDRLE |
| | | RSERGGDRGDRLDRARTPATKRSFSKEVEERSRERPSQPEGLRKAASLT |
| | | EDRDRGRDAVKREAALPPVSPLKAALSEEELEKKSKAIIEEYLHLNDMK |
| | | EAVQCVQELASPSLLFIFVRHGVESTLERSAIAREHMGQLLHQLLCAGH |
| | | LSTAQYYQGLYEILELAEDMEIDIPHVWLYLAELVTPILQEGGVPMGEL |
| | | FREITKPLRPLGKAASLLLEILGLLCKSMGPKKVGTLWREAGLSWKEFL |
| | | PEGQDIGAFVAEQKVEYTLGEESEAPGQRALPSEELNRQLEKLLKEGSS |
| | | NQRVFDWIEANLSEQQIVSNTLVRALMTAVCYSAIIFETPLRVDVAVLK |
| | | ARAKLLQKYLCDEQKELQALYALQALVVTLEQPPNLLRMFFDALYDE |
| | | DVVKEDAFYSWESSKDPAEQQGKGVALKSVTAFFKWLREAEEESDHN |
| 28 | MBP_eIF4G | ATGGCCAGCAACTTCACCCAGTTCGTGCTGGTGGACAACGGCGGTA |
| | (fl) (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAAC |
| | | AAGGCCCCTCAGAGCACCGGCCCTCCTCCTGCCCCTAGCCCTGGCCT |
| | | GCCTCAGCCTGCCTTCCCTCCTGGCCAGACCGCCCCTGTGGTGTTCA |
| | | GCACCCCTCAGGCCACCCAGATGAACACACCTAGCCAGCCTAGACA |
| | | GCACTTCTACCCTAGCAGAGCCCAGCCTCCTAGCAGCGCCGCCAGCA |
| | | GAGTGCAGTCCGCTGCACCTGCCAGACCTGGCCCTGCCGCCCACGTG |
| | | TACCCTGCCGGCAGCCAGGTGATGATGATCCCGAGTCAAATCAGCT |
| | | ACCCTGCAAGCCAGGGCGCCTACTACATCCCGGGCCAGGGCAGAAG |
| | | CACCTACGTGGTGCCTACCCAGCAGTACCCTGTGCAGCCTGGCGCCC |
| | | CTGGTTTCTATCCTGGCGCAAGCCCTACCGAGTTCGGAACTTACGCC |
| | | GGCGCTTACTATCCAGCTCAGGGCGTGCAGCAGTTCCCTACCGGCGT |
| | | GGCCCCTACCCCTGTGCTGATGAATCAGCCACCTCAGATCGCCCCTA |
| | | AGCGCGAGCGCAAGACCATCAGAATCCGCGATCCTAACCAGGGCGG |
| | | CAAGGACATCACCGAGGAGATCATGAGCGGCGCCAGAACAGCAAGT |
| | | ACTCCAACCCCGCCACAAACCGGCGGCGGCCTGGAGCCTCAAGCCA |
| | | ACGGCGAGACGCCACAAGTGGCCGTGATCGTACGCCCTGACGACCG |
| | | GAGCCAAGGTGCAATCATCGCCGATAGGCCTGGCCTCCCAGGTCCG |
| | | GAGCACAGCCCTAGCGAGTCCCAGCCGTCTTCACCATCACCAACCCC |
| | | TAGTCCATCCCCTGTTCTCGAACCAGGCAGCGAGCCTAACCTGGCCG |
| | | TGCTGAGCATACCAGGTGACACCATGACCACCATCCAGATGAGCGT |
| | | GGAGGAGAGCACCCCAATCAGCAGAGAAACTGGAGAGCCTTACAG |
| | | ACTGTCCCCAGAGCCGACCCCACTGGCCGAGCCAATACTGGAGGTG |
| | | GAGGTGACCCTGAGCAAGCCTGTGCCTGAGAGCGAGTTCAGCTCCT |
| | | CTCCACTGCAGGCCCCAACTCCTCTCGCAAGCCACACCGTGGAGATC |
| | | CACGAACCTAACGGCATGGTACCAAGCGAAGATCTTGAGCCAGAGG |
| | | TCGAATCAAGCCCAGAACTGGCCCCTCCACCTGCCTGCCCGTCTGAA |
| | | TCTCCGGTCCCTATCGCTCCTACGGCACAGCCTGAGGAGCTGCTGAA |
| | | CGGTGCCCCGAGCCCTCCAGCAGTGGACTTATCCCCAGTATCAGAGC |
| | | CTGAAGAACAGGCCAAGGAGGTAACTGCCTCTATGGCGCCACCTAC |
| | | CATACCTTCGGCAACACCGGCTACAGCACCATCTGCGACTAGTCCGG |
| | | CTCAGGAGGAGGAGATGGAGGAAGAAGAAGAGGAAGAGGAGGGCG |
| | | AGGCCGGAGAGGCCGGTGAAGCCGAGTCCGAGAAGGGCGGCGAAG |
| | | AACTTCTCCCTCCAGAGTCAACTCCTATCCCTGCCAACCTTAGTCAG |
| | | AATCTGGAGGCCGCCGCCGCTACTCAGGTTGCAGTGAGCGTGCCAA |
| | | AGAGACGTCGCAAGATCAAGGAGCTGAACAAGAAGGAGGCCGTGG |
| | | GCGACCTGCTGGACGCCTTCAAGGAGGCAAACCCGGCGGTGCCTGA |
| | | AGTGGAGAATCAGCCTCCGGCCGGATCAAACCCTGGTCCTGAGAGT |
| | | GAAGGCAGCGGCGTCCCACCAAGACCTGAAGAGGCTGACGAAACTT |
| | | GGGACAGCAAGGAGGACAAGATCCACAACGCCGAGAACATCCAGC |
| | | CAGGCGAGCAGAAGTACGAGTACAAGAGCGACCAGTGGAAGCCTCT |
| | | TAACCTTGAAGAGAAGAAGAGATACGACAGAGAGTTCCTGCTGGGC |
| | | TTCCAGTTCATATTCGCGAGCATGCAGAAGCCTGAAGGTCTGCCTCA |
| | | CATCAGCGACGTGGTGCTGGACAAGGCCAACAAGACCCCTCTTAGA |
| | | CCTCTGGACCCTACCAGACTGCAGGGCATCAACTGCGGCCCTGACTT |
| | | CACTCCTTCTTTCGCAAACCTGGGCAGAACCACCCTGAGCACCAGAG |
| | | GCCCTCCTAGAGGTGGTCCCGGCGGAGAACTCCCCAGGGGTCCTGC |
| | | CGGCCTGGGCCCTAGACGCTCCCAGCAAGGTCCTAGAAAGGAGCCA |
| | | AGGAAGATCATCGCCACCGTGCTGATGACCGAGGACATCAAGCTGA |
| | | ACAAAGCTGAGAAGGCCTGGAAGCCTAGCAGCAAGAGAACCGCCG |
| | | CCGACAAGGACAGAGGCGAGGAGGACGCCGACGGATCCAAGACCC |
| | | AGGACCTGTTCAGAAGAGTGAGAAGCATCCTCAACAAGCTGACCCC |
| | | TCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAG |
| | | TTCGAGAAGGATAAGGACGACGACGAGGTCTTCGAGAAGAAACAG |
| | | AAAGAAATGGACGAGGCCGCCACCGCAGAGGAAAGGGGCCGATTA |
| | | AAGGAGGAGCTGGAGGAGGCCAGAGACATCGCCAGACGGCGTTCTC |
| | | TGGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTGCCGACCGAGAGCACCGACAACCGGCGAGTTGTGC |
| | | AGAGAAGCAGCCTGAGCAGAGAGAGGGGCGAGAAAGCCGGCGATA |
| | | GAGGTGACCGACTGGAGAGAAGCGAGAGAGGAGGTGATAGAGGCG |
| | | ACCGGCTTGACAGAGCCAGAACCCCTGCCACAAAGCGATCGTTCAG |
| | | CAAGGAGGTCGAAGAGAGGTCCAGGGAGCGCCCTAGCCAGCCGGA |
| | | AGGACTCAGAAAGGCAGCCAGTCTAACAGAGGACCGCGACAGGGG |
| | | AAGAGACGCCGTGAAGAGGGAGGCCGCACTGCCTCCTGTGAGCCCT |
| | | CTGAAGGCGGCACTGAGCGAAGAAGAACTTGAGAAGAAGAGTAAG |
| | | GCGATCATCGAGGAGTACCTGCACCTGAACGACATGAAGGAGGCCG |
| | | TGCAGTGCGTGCAAGAGCTCGCGAGCCCATCACTGCTGTTCATCTTC |
| | | GTCCGGCACGGCGTGGAGTCCACACTGGAAAGATCTGCCATTGCTA |
| | | GGGAGCATATGGGCCAGTTGTTGCACCAATTGCTTTGCGCCGGCCAC |
| | | CTAAGTACTGCCCAGTACTATCAGGGTTTATACGAGATCCTCGAACT |
| | | GGCCGAAGACATGGAGATCGACATCCCTCACGTGTGGCTGTACCTC |
| | | GCAGAACTGGTGACCCCTATCCTGCAGGAGGGCGGCGTTCCAATGG |
| | | GTGAGTTGTTCAGAGAAATCACAAAGCCACTGCGCCCACTGGGCAA |
| | | GGCGGCTAGTCTCCTTCTGGAGATTCTCGGCCTGCTCTGTAAGAGCA |
| | | TGGGTCCAAAGAAGGTGGGCACCCTGTGGAGGGAAGCTGGACTCTC |
| | | CTGGAAGGAATTCCTCCCTGAGGGTCAGGACATCGGCGCCTTCGTGG |
| | | CCGAACAGAAGGTTGAGTACACCCTGGGAGAGGAATCGGAAGCGCC |
| | | GGGTCAGCGGGCTCTGCCGAGTGAGGAGCTCAACAGACAACTCGAG |
| | | AAGCTCCTCAAGGAGGGATCCTCAAATCAGAGAGTGTTCGACTGGA |
| | | TCGAGGCCAATCTCAGCGAGCAGCAAATCGTGAGCAACACGTTGGT |
| | | TCGCGCTCTCATGACAGCCGTGTGCTACTCAGCCATTATCTTCGAGA |
| | | CGCCTCTCCGCGTGGACGTGGCAGTGCTCAAGGCCCGCGCTAAGCTG |
| | | TTACAGAAGTACCTGTGCGACGAGCAGAAGGAACTGCAGGCCCTGT |
| | | ACGCCCTGCAAGCTCTGGTCGTCACACTCGAGCAGCCTCCTAACCTG |
| | | CTGAGAATGTTCTTCGACGCATTGTACGACGAGGACGTTGTGAAGG |
| | | AAGACGCTTTCTACAGCTGGGAGTCTAGTAAGGATCCTGCGGAACA |
| | | ACAAGGCAAGGGCGTTGCTCTAAAGAGCGTGACCGCCTTCTTCAAG |
| | | TGGCTGCGAGAGGCTGAGGAAGAGTCGGACCACAAC |
| 29 | MBP_eIF4G- | MASNFTQFVLVDNGGTGDVTVAPSNFANGIAEWISSNSRSQAYKVTCS |
| | dN (623- | VRQSSAQNRKYTIKVEVPKGAWRSYLNMELTIPIFATNSDCELIVKAMQ |
| | 1599, 3A) | GLLKDGNPIPSAIAANSGIYGGGGSIFASMQKPEGLPHISDVVLDKANKT |
| | (aa) | PLRPLDPTRLQGINCGPDFTPSFANLGRTTLSTRGPPRGGPGGELPRGPA |
| | | GLGPRRSQQGPRKEPRKIIATVLMTEDIKLNKAEKAWKPSSKRTAADK |
| | | DRGEEDADGSKTQDLFRRVRSIANKATPQMAQQLMKQVTQLAIDTEER |
| | | LKGVIDLIFEKAISEPNFSVAYANMCRCLMALKVPTTEKPTVTVNFRKL |
| | | LLNRCQKEFEKDKDDDEVFEKKQKEMDEAATAEERGRLKEELEEARDI |
| | | ARRRSLGNIKFIGELFKLKMLTEAIMHDCVVKLLKNHDEESLECLCRLL |
| | | TTIGKDLDFEKAKPRMDQYFNQMEKIIKEKKTSSRIRFMLQDVLDLRGS |
| | | NWVPRRGDQGPKTIDQIHKEAEMEEHREHIKVQQLMAKGSDKRRGGP |
| | | PGPPISRGLPLVDDGGWNTVPISKGSRPIDTSRLTKITKPGSIDSNNQLFA |
| | | PGGRLSWGKGSSGG |
| | | SGAKPSDAASEAARPATSTLNRFSALQQAVPTESTDNRRVVQRSSLSRE |
| | | RGEKAGDRGDRLERSERGGDRGDRLDRARTPATKRSFSKEVEERSRER |
| | | PSQPEGLRKAASLTEDRDRGRDAVKREAALPPVSPLKAALSEEELEKKS |
| | | KAIIEEYLHLNDMKEAVQCVQELASPSLLFIFVRHGVESTLERSAIAREH |
| | | MGQLLHQLLCAGHLSTAQYYQGLYEILELAEDMEIDIPHVWLYLAELV |
| | | TPILQEGGVPMGELFREITKPLRPLGKAASLLLEILGLLCKSMGPKKVGT |
| | | LWREAGLSWKEFLPEGQDIGAFVAEQKVEYTLGEESEAPGQRALPSEEL |
| | | NRQLEKLLKEGSSNQRVFDWIEANLSEQQIVSNTLVRALMTAVCYSAII |
| | | FETPLRVDVAVLKARAKLLQKYLCDEQKELQALYALQALVVTLEQPPN |
| | | LLRMFFDALYDEDVVKEDAFYSWESSKDPAEQQGKGVALK |
| | | SVTAFFKWLREAEEESDHN |
| 30 | MBP_eIF4G- | ATGGCCAGCAACTTCACCCAGTTCGTGCTGGTGGACAACGGCGGTA |
| | dN (623- | CCGGAGACGTGACCGTGGCCCCTTCTAACTTCGCCAACGGCATCGCC |
| | 1599, 3A) | GAGTGGATCAGCAGCAACAGCAGAAGCCAGGCCTACAAGGTGACCT |
| | (nt) | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCATA |
| | | TTCGCGAGCATGCAGAAGCCTGAAGGTCTGCCTCACATCAGCGACG |
| | | TGGTGCTGGACAAGGCCAACAAGACCCCTCTTAGACCTCTGGACCCT |
| | | ACCAGACTGCAGGGCATCAACTGCGGCCCTGACTTCACTCCTTCTTT |
| | | CGCAAACCTGGGCAGAACCACCCTGAGCACCAGAGGCCCTCCTAGA |
| | | GGTGGTCCCGGCGGAGAACTCCCCAGGGGTCCTGCCGGCCTGGGCC |
| | | CTAGACGCTCCCAGCAAGGTCCTAGAAAGGAGCCAAGGAAGATCAT |
| | | CGCCACCGTGCTGATGACCGAGGACATCAAGCTGAACAAAGCTGAG |
| | | AAGGCCTGGAAGCCTAGCAGCAAGAGAACCGCCGCCGACAAGGAC |
| | | AGAGGCGAGGAGGACGCCGACGGATCCAAGACCCAGGACCTGTTCA |
| | | GAAGAGTGAGAAGCATCGCGAACAAGGCGACCCCTCAGATGGCGCA |
| | | GCAGCTGATGAAGCAGGTGACGCAGCTCGCCATCGACACCGAGGAG |
| | | AGACTGAAGGGCGTGATCGACCTGATCTTTGAGAAGGCTATCTCAG |
| | | AGCCTAACTTCAGCGTGGCCTACGCCAACATGTGCCGTTGCCTGATG |
| | | GCATTGAAGGTGCCAACCACCGAGAAGCCTACTGTGACCGTCAATTT |
| | | CCGTAAACTGCTGCTGAACCGGTGCCAGAAAGAGTTCGAGAAGGAT |
| | | AAGGACGACGACGAGGTCTTCGAGAAGAAACAGAAAGAAATGGAC |
| | | GAGGCCGCCACCGCAGAGGAAAGGGGCCGATTAAAGGAGGAGCTG |
| | | GAGGAGGCCAGAGACATCGCCAGACGGCGTTCTCTGGGCAACATCA |
| | | AGTTCATAGGTGAGCTGTTCAAGCTAAAGATGCTCACCGAGGCCAT |
| | | AATGCACGACTGCGTGGTGAAGCTACTGAAGAACCACGACGAAGAA |
| | | AGCCTGGAGTGCCTGTGCAGACTGCTGACCACCATCGGCAAGGACC |
| | | TGGACTTCGAGAAGGCAAAGCCTCGAATGGACCAGTACTTCAACCA |
| | | GATGGAGAAGATTATCAAGGAGAAGAAGACCAGCAGCAGAATCAG |
| | | ATTCATGCTGCAGGACGTACTGGACCTGCGCGGAAGCAACTGGGTG |
| | | CCAAGGAGAGGGGACCAAGGACCAAAGACCATCGACCAGATCCAC |
| | | AAGGAAGCGGAGATGGAGGAGCACAGAGAGCACATAAAGGTGCAG |
| | | CAGCTTATGGCCAAGGGCAGCGACAAGCGAAGAGGCGGCCCGCCCG |
| | | GACCTCCTATCAGCAGAGGCCTTCCTCTGGTAGACGACGGCGGCTGG |
| | | AACACCGTGCCTATCTCTAAGGGCTCCAGACCTATCGACACTTCCCG |
| | | TCTTACCAAGATCACCAAGCCAGGATCTATTGACAGCAACAACCAG |
| | | CTGTTCGCCCCAGGAGGAAGACTTAGCTGGGGCAAGGGAAGTTCCG |
| | | GCGGATCCGGCGCCAAGCCTTCCGACGCCGCCAGCGAGGCTGCCAG |
| | | ACCTGCCACCAGCACCTTGAACCGCTTTTCCGCTCTGCAGCAAGCTG |
| | | TGCCGACCGAGAGCACCGACAACCGGCGAGTTGTGCAGAGAAGCAG |
| | | CCTGAGCAGAGAGAGGGGCGAGAAAGCCGGCGATAGAGGTGACCG |
| | | ACTGGAGAGAAGCGAGAGAGGAGGTGATAGAGGCGACCGGCTTGA |
| | | CAGAGCCAGAACCCCTGCCACAAAGCGATCGTTCAGCAAGGAGGTC |
| | | GAAGAGAGGTCCAGGGAGCGCCCTAGCCAGCCGGAAGGACTCAGA |
| | | AAGGCAGCCAGTCTAACAGAGGACCGCGACAGGGGAAGAGACGCC |
| | | GTGAAGAGGGAGGCCGCACTGCCTCCTGTGAGCCCTCTGAAGGCGG |
| | | CACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTCAA |
| | | GGAGGGATCCTCAAATCAGAGAGTGTTCGACTGGATCGAGGCCAAT |
| | | CTCAGCGAGCAGCAAATCGTGAGCAACACGTTGGTTCGCGCTCTCAT |
| | | GACAGCCGTGTGCTACTCAGCCATTATCTTCGAGACGCCTCTCCGCG |
| | | TGGACGTGGCAGTGCTCAAGGCCCGCGCTAAGCTGTTACAGAAGTA |
| | | CCTGTGCGACGAGCAGAAGGAACTGCAGGCCCTGTACGCCCTGCAA |
| | | GCTCTGGTCGTCACACTCGAGCAGCCTCCTAACCTGCTGAGAATGTT |
| | | CTTCGACGCATTGTACGACGAGGACGTTGTGAAGGAAGACGCTTTCT |
| | | ACAGCTGGGAGTCTAGTAAGGATCCTGCGGAACAACAAGGCAAGGG |
| | | CGTTGCTCTAAAGAGCGTGACCGCCTTCTTCAAGTGGCTGCGAGAGG |
| | | CTGAGGAAGAGTCGGACCACAAC |
| 31 | MBP_eIF4G- | MASNFTQFVLVDNGGTGDVTVAPSNFANGIAEWISSNSRSQAYKVTCS |
| | dN2 (654- | VRQSSAQNRKYTIKVEVPKGAWRSYLNMELTIPIFATNSDCELIVKAMQ |
| | 1599) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAVPTESTDNRRVVQRSSLSRERGEKAGDRGDRLER |
| | | SERGGDRGDRLDRARTPATKRSFSKEVEERSRERPSQPEGLRKAASLTE |
| | | DRDRGRDAVKREAALPPVSPLKAALSEEELEKKSKAIIEEYLHLNDMKE |
| | | AVQCVQELASPSLLFIFVRHGVESTLERSAIAREHMGQLLHQLLCAGHL |
| | | STAQYYQGLYEILELAEDMEIDIPHVWLYLAELVTPILQEGGVPMGELF |
| | | REITKPLRPLGKAASLLLEILGLLCKSMGPKKVGTLWREAGLSWKEFLP |
| | | EGQDIGAFVAEQKVEYTLGEESEAPGQRALPSEELNRQLEKLLKEGSSN |
| | | QRVFDWIEANLSEQQIVSNTLVRALMTAVCYSAIIFETPLRVDVAVLKA |
| | | RAKLLQKYLCDEQKELQALYALQALVVTLEQPPNLLRMFFDALYDED |
| | | VVKEDAFYSWESSKDPAEQQGKGVALKSVTAFFKWLREAEEESDHN |
| 32 | MBP_eIF4G- | ATGGCCAGCAACTTCACCCAGTTCGTGCTGGTGGACAACGGCGGTA |
| | dN2 (654- | CCGGAGACGTGACCGTGGCCCCTTCTAACTTCGCCAACGGCATCGCC |
| | 1599) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTTGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCA |
| | | TCGAGGAGTACCTGCACCTGAACGACATGAAGGAGGCCGTGCAGTG |
| | | CGTGCAAGAGCTCGCGAGCCCATCACTGCTGTTCATCTTCGTCCGGC |
| | | ACGGCGTGGAGTCCACACTGGAAAGATCTGCCATTGCTAGGGAGCA |
| | | TATGGGCCAGTTGTTGCACCAATTGCTTTGCGCCGGCCACCTAAGTA |
| | | CTGCCCAGTACTATCAGGGTTTATACGAGATCCTCGAACTGGCCGAA |
| | | GACATGGAGATCGACATCCCTCACGTGTGGCTGTACCTCGCAGAACT |
| | | GGTGACCCCTATCCTGCAGGAGGGCGGCGTTCCAATGGGTGAGTTGT |
| | | TCAGAGAAATCACAAAGCCACTGCGCCCACTGGGCAAGGCGGCTAG |
| | | TCTCCTTCTGGAGATTCTCGGCCTGCTCTGTAAGAGCATGGGTCCAA |
| | | AGAAGGTGGGCACCCTGTGGAGGGAAGCTGGACTCTCCTGGAAGGA |
| | | ATTCCTCCCTGAGGGTCAGGACATCGGCGCCTTCGTGGCCGAACAGA |
| | | AGGTTGAGTACACCCTGGGAGAGGAATCGGAAGCGCCGGGTCAGCG |
| | | GGCTCTGCCGAGTGAGGAGCTCAACAGACAACTCGAGAAGCTCCTC |
| | | AAGGAGGGATCCTCAAATCAGAGAGTGTTCGACTGGATCGAGGCCA |
| | | ATCTCAGCGAGCAGCAAATCGTGAGCAACACGTTGGTTCGCGCTCTC |
| | | ATGACAGCCGTGTGCTACTCAGCCATTATCTTCGAGACGCCTCTCCG |
| | | CGTGGACGTGGCAGTGCTCAAGGCCCGCGCTAAGCTGTTACAGAAG |
| | | TACCTGTGCGACGAGCAGAAGGAACTGCAGGCCCTGTACGCCCTGC |
| | | AAGCTCTGGTCGTCACACTCGAGCAGCCTCCTAACCTGCTGAGAATG |
| | | TTCTTCGACGCATTGTACGACGAGGACGTTGTGAAGGAAGACGCTTT |
| | | CTACAGCTGGGAGTCTAGTAAGGATCCTGCGGAACAACAAGGCAAG |
| | | GGCGTTGCTCTAAAGAGCGTGACCGCCTTCTTCAAGTGGCTGCGAGA |
| | | GGCTGAGGAAGAGTCGGACCACAAC |
| 33 | MBP_eIF4G- | MASNFTQFVLVDNGGTGDVTVAPSNFANGIAEWISSNSRSQAYKVTCS |
| | dN3 (654- | VRQSSAQNRKYTIKVEVPKGAWRSYLNMELTIPIFATNSDCELIVKAMQ |
| | 1451) (aa) | LSTRGPPRGGPGGELPRGPAGLGPRRSQQGPRKEPRKIIATVLMTEDIKL |
| | | GLLKDGNPIPSAIAANSGIYGGGGSDPTRLQGINCGPDFTPSFANLGRTT |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAVPTESTDNRRVVQRSSLSRERGEKAGDRGDRLER |
| | | SERGGDRGDRLDRARTPATKRSFSKEVEERSRERPSQPEGLRKAASLTE |
| | | DRDRGRDAVKREAALPPVSPLKAALSEEELEKKSKAIIEEYLHLNDMKE |
| | | AVQCVQELASPSLLFIFVRHGVESTLERSAIAREHMGQLLHQLLCAGHL |
| | | STAQYYQGLYEILELAEDMEIDIPHVWLYLAELVTPILQEGGVPMGELF |
| | | REITKPLRPLGKAASLLLEILGLLCKSMGPKKVGTLWREAGLSWKEFLP |
| | | EGQDIGAFVAEQKVEYTLGEESEAPGQRALPSEELNRQLEKLL |
| 34 | MBP_eIF4G- | ATGGCCAGCAACTTCACCCAGTTCGTGCTGGTGGACAACGGCGGTA |
| | dN3 (654- | CCGGAGACGTGACCGTGGCCCCTTCTAACTTCGCCAACGGCATCGCC |
| | 1451) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTTGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCA |
| | | TCGAGGAGTACCTGCACCTGAACGACATGAAGGAGGCCGTGCAGTG |
| | | CGTGCAAGAGCTCGCGAGCCCATCACTGCTGTTCATCTTCGTCCGGC |
| | | ACGGCGTGGAGTCCACACTGGAAAGATCTGCCATTGCTAGGGAGCA |
| | | TATGGGCCAGTTGTTGCACCAATTGCTTTGCGCCGGCCACCTAAGTA |
| | | CTGCCCAGTACTATCAGGGTTTATACGAGATCCTCGAACTGGCCGAA |
| | | GACATGGAGATCGACATCCCTCACGTGTGGCTGTACCTCGCAGAACT |
| | | GGTGACCCCTATCCTGCAGGAGGGCGGCGTTCCAATGGGTGAGTTGT |
| | | TCAGAGAAATCACAAAGCCACTGCGCCCACTGGGCAAGGCGGCTAG |
| | | TCTCCTTCTGGAGATTCTCGGCCTGCTCTGTAAGAGCATGGGTCCAA |
| | | AGAAGGTGGGCACCCTGTGGAGGGAAGCTGGACTCTCCTGGAAGGA |
| | | ATTCCTCCCTGAGGGTCAGGACATCGGCGCCTTCGTGGCCGAACAGA |
| | | AGGTTGAGTACACCCTGGGAGAGGAATCGGAAGCGCCGGGTCAGCG |
| | | GGCTCTGCCGAGTGAGGAGCTCAACAGACAACTCGAGAAGCTCCTC |
| 35 | MBP_eIF4G- | MASNFTQFVLVDNGGTGDVTVAPSNFANGIAEWISSNSRSQAYKVTCS |
| | mid1 (674- | VRQSSAQNRKYTIKVEVPKGAWRSYLNMELTIPIFATNSDCELIVKAMQ |
| | 1079) (aa) | GLLKDGNPIPSAIAANSGIYGGGGSGRTTLSTRGPPRGGPGGELPRGPAG |
| | | LGPRRSQQGPRKEPRKIIATVLMTEDIKLNKAEKAWKPSSKRTAADKDR |
| | | GEEDADGSKTQDLFRRVRSILNKLTPQMFQQLMKQVTQLAIDTEERLK |
| | | GVIDLIFEKAISEPNFSVAYANMCRCLMALKVPTTEKPTVTVNFRKLLL |
| | | NRCQKEFEKDKDDDEVFEKKQKEMDEAATAEERGRLKEELEEARDIAR |
| | | RRSLGNIKFIGELFKLKMLTEAIMHDCVVKLLKNHDEESLECLCRLLTTI |
| | | GKDLDFEKAKPRMDQYFNQMEKIIKEKKTSSRIRFMLQDVLDLRGSNW |
| | | VPRRGDQGPKTIDQIHKEAEMEEHREHIKVQQLMAKGSDKRRGGPPGP |
| | | PISRGLPLVDDGGWNTVPISKGSRPIDTSRLTKITKPGSID |
| 36 | MBP_eIF4G- | ATGGCCAGCAACTTCACCCAGTTCGTGCTGGTGGACAACGGCGGTA |
| | mid1 (674- | CCGGAGACGTGACCGTGGCCCCTTCTAACTTCGCCAACGGCATCGCC |
| | 1079) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGGC |
| | | AGAACCACCCTGAGCACCAGAGGCCCTCCTAGAGGTGGTCCCGGCG |
| | | GAGAACTCCCCAGGGGTCCTGCCGGCCTGGGCCCTAGACGCTCCCA |
| | | GCAAGGTCCTAGAAAGGAGCCAAGGAAGATCATCGCCACCGTGCTG |
| | | ATGACCGAGGACATCAAGCTGAACAAAGCTGAGAAGGCCTGGAAGC |
| | | CTAGCAGCAAGAGAACCGCCGCCGACAAGGACAGAGGCGAGGAGG |
| | | ACGCCGACGGATCCAAGACCCAGGACCTGTTCAGAAGAGTGAGAAG |
| | | CATCCTCAACAAGCTGACCCCTCAGATGTTCCAGCAGCTGATGAAGC |
| | | AGGTGACGCAGCTCGCCATCGACACCGAGGAGAGACTGAAGGGCGT |
| | | GATCGACCTGATCTTTGAGAAGGCTATCTCAGAGCCTAACTTCAGCG |
| | | TGGCCTACGCCAACATGTGCCGTTGCCTGATGGCATTGAAGGTGCCA |
| | | ACCACCGAGAAGCCTACTGTGACCGTCAATTTCCGTAAACTGCTGCT |
| | | GAACCGGTGCCAGAAAGAGTTCGAGAAGGATAAGGACGACGACGA |
| | | GGTCTTCGAGAAGAAACAGAAAGAAATGGACGAGGCCGCCACCGC |
| | | AGAGGAAAGGGGCCGATTAAAGGAGGAGCTGGAGGAGGCCAGAGA |
| | | CATCGCCAGACGGCGTTCTCTGGGCAACATCAAGTTCATAGGTGAGC |
| | | TGTTCAAGCTAAAGATGCTCACCGAGGCCATAATGCACGACTGCGT |
| | | GGTGAAGCTACTGAAGAACCACGACGAAGAAAGCCTGGAGTGCCTG |
| | | TGCAGACTGCTGACCACCATCGGCAAGGACCTGGACTTCGAGAAGG |
| | | CAAAGCCTCGAATGGACCAGTACTTCAACCAGATGGAGAAGATTAT |
| | | CAAGGAGAAGAAGACCAGCAGCAGAATCAGATTCATGCTGCAGGAC |
| | | GTACTGGACCTGCGCGGAAGCAACTGGGTGCCAAGGAGAGGGGACC |
| | | AAGGACCAAAGACCATCGACCAGATCCACAAGGAAGCGGAGATGG |
| | | AGGAGCACAGAGAGCACATAAAGGTGCAGCAGCTTATGGCCAAGG |
| | | GCAGCGACAAGCGAAGAGGCGGCCCGCCCGGACCTCCTATCAGCAG |
| | | AGGCCTTCCTCTGGTAGACGACGGCGGCTGGAACACCGTGCCTATCT |
| | | CTAAGGGCTCCAGACCTATCGACACTTCCCGTCTTACCAAGATCACC |
| | | AAGCCAGGATCTATTGAC |
| 37 | MBP_eIF4G- | MASNFTQFVLVDNGGTGDVTVAPSNFANGIAEWISSNSRSQAYKVTCS |
| | mid2 (654- | VRQSSAQNRKYTIKVEVPKGAWRSYLNMELTIPIFATNSDCELIVKAMQ |
| | 1130) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAV |
| 38 | MBP_eIF4G- | ATGGCCAGCAACTTCACCCAGTTCGTGCTGGTGGACAACGGCGGTA |
| | mid2 (654- | CCGGAGACGTGACCGTGGCCCCTTCTAACTTCGCCAACGGCATCGCC |
| | 1130) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTG |
| 39 | MBP_eIF4G- | MASNFTQFVLVDNGGTGDVTVAPSNFANGIAEWISSNSRSQAYKVTCS |
| | mid3 (712- | VRQSSAQNRKYTIKVEVPKGAWRSYLNMELTIPIFATNSDCELIVKAMQ |
| | 1130) (aa) | GLLKDGNPIPSAIAANSGIYGGGGSPRKIIATVLMTEDIKLNKAEKAWKP |
| | | SSKRTAADKDRGEEDADGSKTQDLFRRVRSILNKLTPQMFQQLMKQVT |
| | | QLAIDTEERLKGVIDLIFEKAISEPNFSVAYANMCRCLMALKVPTTEKPT |
| | | VTVNFRKLLLNRCQKEFEKDKDDDEVFEKKQKEMDEAATAEERGRLK |
| | | EELEEARDIARRRSLGNIKFIGELFKLKMLTEAIMHDCVVKLLKNHDEE |
| | | SLECLCRLLTTIGKDLDFEKAKPRMDQYFNQMEKIIKEKKTSSRIRFMLQ |
| | | DVLDLRGSNWVPRRGDQGPKTIDQIHKEAEMEEHREHIKVQQLMAKG |
| | | SDKRRGGPPGPPISRGLPLVDDGGWNTVPISKGSRPIDTSRLTKITKPGSI |
| | | DSNNQLFAPGGRLSWGKGSSGGSGAKPSDAASEAARPATSTLNRFSAL |
| | | QQAV |
| 40 | MBP_eIF4G- | ATGGCCAGCAACTTCACCCAGTTCGTGCTGGTGGACAACGGCGGTA |
| | mid3 (712- | CCGGAGACGTGACCGTGGCCCCTTCTAACTTCGCCAACGGCATCGCC |
| | 1130) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCCCA |
| | | AGGAAGATCATCGCCACCGTGCTGATGACCGAGGACATCAAGCTGA |
| | | ACAAAGCTGAGAAGGCCTGGAAGCCTAGCAGCAAGAGAACCGCCG |
| | | CCGACAAGGACAGAGGCGAGGAGGACGCCGACGGATCCAAGACCC |
| | | AGGACCTGTTCAGAAGAGTGAGAAGCATCCTCAACAAGCTGACCCC |
| | | TCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAG |
| | | TTCGAGAAGGATAAGGACGACGACGAGGTCTTCGAGAAGAAACAG |
| | | AAAGAAATGGACGAGGCCGCCACCGCAGAGGAAAGGGGCCGATTA |
| | | AAGGAGGAGCTGGAGGAGGCCAGAGACATCGCCAGACGGCGTTCTC |
| | | TGGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTG |
| 41 | MBP_eIF4G- | MASNFTQFVLVDNGGTGDVTVAPSNFANGIAEWISSNSRSQAYKVTCS |
| | C1 (1080- | VRQSSAQNRKYTIKVEVPKGAWRSYLNMELTIPIFATNSDCELIVKAMQ |
| | 1599) (aa) | GLLKDGNPIPSAIAANSGIYGGGGSSNNQLFAPGGRLSWGKGSSGGSGA |
| | | KPSDAASEAARPATSTLNRFSALQQAVPTESTDNRRVVQRSSLSRERGE |
| | | KAGDRGDRLERSERGGDRGDRLDRARTPATKRSFSKEVEERSRERPSQP |
| | | EGLRKAASLTEDRDRGRDAVKREAALPPVSPLKAALSEEELEKKSKAII |
| | | EEYLHLNDMKEAVQCVQELASPSLLFIFVRHGVESTLERSAIAREHMGQ |
| | | LLHQLLCAGHLSTAQYYQGLYEILELAEDMEIDIPHVWLYLAELVTPIL |
| | | QEGGVPMGELFREITKPLRPLGKAASLLLEILGLLCKSMGPKKVGTLWR |
| | | EAGLSWKEFLPEGQDIGAFVAEQKVEYTLGEESEAPGQRALPSEELNRQ |
| | | LEKLLKEGSSNQRVFDWIEANLSEQQIVSNTLVRALMTAVCYSAIIFETP |
| | | LRVDVAVLKARAKLLQKYLCDEQKELQALYALQALVVTLEQPPNLLR |
| | | MFFDALYDEDVVKEDAFYSWESSKDPAEQQGKGVALKSVTAFFKWLR |
| | | EAEEESDHN |
| 42 | MBP_eIF4G- | ATGGCCAGCAACTTCACCCAGTTCGTGCTGGTGGACAACGGCGGTA |
| | C1 (1080- | CCGGAGACGTGACCGTGGCCCCTTCTAACTTCGCCAACGGCATCGCC |
| | 1599) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAGC |
| | | AACAACCAGCTGTTCGCCCCAGGAGGAAGACTTAGCTGGGGCAAGG |
| | | GAAGTTCCGGCGGATCCGGCGCCAAGCCTTCCGACGCCGCCAGCGA |
| | | GGCTGCCAGACCTGCCACCAGCACCTTGAACCGCTTTTCCGCTCTGC |
| | | AGCAAGCTGTGCCGACCGAGAGCACCGACAACCGGCGAGTTGTGCA |
| | | GAGAAGCAGCCTGAGCAGAGAGAGGGGCGAGAAAGCCGGCGATAG |
| | | AGGTGACCGACTGGAGAGAAGCGAGAGAGGAGGTGATAGAGGCGA |
| | | CCGGCTTGACAGAGCCAGAACCCCTGCCACAAAGCGATCGTTCAGC |
| | | AAGGAGGTCGAAGAGAGGTCCAGGGAGCGCCCTAGCCAGCCGGAA |
| | | GGACTCAGAAAGGCAGCCAGTCTAACAGAGGACCGCGACAGGGGA |
| | | AGAGACGCCGTGAAGAGGGAGGCCGCACTGCCTCCTGTGAGCCCTC |
| | | TGAAGGCGGCACTGAGCGAAGAAGAACTTGAGAAGAAGAGTAAGG |
| | | CGATCATCGAGGAGTACCTGCACCTGAACGACATGAAGGAGGCCGT |
| | | GCAGTGCGTGCAAGAGCTCGCGAGCCCATCACTGCTGTTCATCTTCG |
| | | TCCGGCACGGCGTGGAGTCCACACTGGAAAGATCTGCCATTGCTAG |
| | | GGAGCATATGGGCCAGTTGTTGCACCAATTGCTTTGCGCCGGCCACC |
| | | TAAGTACTGCCCAGTACTATCAGGGTTTATACGAGATCCTCGAACTG |
| | | GCCGAAGACATGGAGATCGACATCCCTCACGTGTGGCTGTACCTCGC |
| | | AGAACTGGTGACCCCTATCCTGCAGGAGGGCGGCGTTCCAATGGGT |
| | | GAGTTGTTCAGAGAAATCACAAAGCCACTGCGCCCACTGGGCAAGG |
| | | CGGCTAGTCTCCTTCTGGAGATTCTCGGCCTGCTCTGTAAGAGCATG |
| | | GGTCCAAAGAAGGTGGGCACCCTGTGGAGGGAAGCTGGACTCTCCT |
| | | GGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTCAAGGAGGGATCCTCAAATCAGAGAGTGTTCGACTGGAT |
| | | CGAGGCCAATCTCAGCGAGCAGCAAATCGTGAGCAACACGTTGGTT |
| | | CGCGCTCTCATGACAGCCGTGTGCTACTCAGCCATTATCTTCGAGAC |
| | | GCCTCTCCGCGTGGACGTGGCAGTGCTCAAGGCCCGCGCTAAGCTGT |
| | | TACAGAAGTACCTGTGCGACGAGCAGAAGGAACTGCAGGCCCTGTA |
| | | CGCCCTGCAAGCTCTGGTCGTCACACTCGAGCAGCCTCCTAACCTGC |
| | | TGAGAATGTTCTTCGACGCATTGTACGACGAGGACGTTGTGAAGGA |
| | | AGACGCTTTCTACAGCTGGGAGTCTAGTAAGGATCCTGCGGAACAA |
| | | CAAGGCAAGGGCGTTGCTCTAAAGAGCGTGACCGCCTTCTTCAAGT |
| | | GGCTGCGAGAGGCTGAGGAAGAGTCGGACCACAAC |
| 43 | MBP_eIF4G- | MASNFTQFVLVDNGGTGDVTVAPSNFANGIAEWISSNSRSQAYKVTCS |
| | C2 (1080- | VRQSSAQNRKYTIKVEVPKGAWRSYLNMELTIPIFATNSDCELIVKAMQ |
| | 1451) (aa) | GLLKDGNPIPSAIAANSGIYGGGGSSNNQLFAPGGRLSWGKGSSGGSGA |
| | | KPSDAASEAARPATSTLNRFSALQQAVPTESTDNRRVVQRSSLSRERGE |
| | | KAGDRGDRLERSERGGDRGDRLDRARTPATKRSFSKEVEERSRERPSQP |
| | | EGLRKAASLTEDRDRGRDAVKREAALPPVSPLKAALSEEELEKKSKAII |
| | | EEYLHLNDMKEAVQCVQELASPSLLFIFVRHGVESTLERSAIAREHMGQ |
| | | LLHQLLCAGHLSTAQYYQGLYEILELAEDMEIDIPHVWLYLAELVTPIL |
| | | QEGGVPMGELFREITKPLRPLGKAASLLLEILGLLCKSMGPKKVGTLWR |
| | | EAGLSWKEFLPEGQDIGAFVAEQKVEYTLGEESEAPGQRALPSEELNRQ |
| | | LEKLL |
| 44 | MBP_eIF4G- | ATGGCCAGCAACTTCACCCAGTTCGTGCTGGTGGACAACGGCGGTA |
| | C2 (1080- | CCGGAGACGTGACCGTGGCCCCTTCTAACTTCGCCAACGGCATCGCC |
| | 1451) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAGC |
| | | AACAACCAGCTGTTCGCCCCAGGAGGAAGACTTAGCTGGGGCAAGG |
| | | GAAGTTCCGGCGGATCCGGCGCCAAGCCTTCCGACGCCGCCAGCGA |
| | | GGCTGCCAGACCTGCCACCAGCACCTTGAACCGCTTTTCCGCTCTGC |
| | | AGCAAGCTGTGCCGACCGAGAGCACCGACAACCGGCGAGTTGTGCA |
| | | GAGAAGCAGCCTGAGCAGAGAGAGGGGCGAGAAAGCCGGCGATAG |
| | | AGGTGACCGACTGGAGAGAAGCGAGAGAGGAGGTGATAGAGGCGA |
| | | CCGGCTTGACAGAGCCAGAACCCCTGCCACAAAGCGATCGTTCAGC |
| | | AAGGAGGTCGAAGAGAGGTCCAGGGAGCGCCCTAGCCAGCCGGAA |
| | | GGACTCAGAAAGGCAGCCAGTCTAACAGAGGACCGCGACAGGGGA |
| | | AGAGACGCCGTGAAGAGGGAGGCCGCACTGCCTCCTGTGAGCCCTC |
| | | TGAAGGCGGCACTGAGCGAAGAAGAACTTGAGAAGAAGAGTAAGG |
| | | CGATCATCGAGGAGTACCTGCACCTGAACGACATGAAGGAGGCCGT |
| | | GCAGTGCGTGCAAGAGCTCGCGAGCCCATCACTGCTGTTCATCTTCG |
| | | TCCGGCACGGCGTGGAGTCCACACTGGAAAGATCTGCCATTGCTAG |
| | | GGAGCATATGGGCCAGTTGTTGCACCAATTGCTTTGCGCCGGCCACC |
| | | TAAGTACTGCCCAGTACTATCAGGGTTTATACGAGATCCTCGAACTG |
| | | GCCGAAGACATGGAGATCGACATCCCTCACGTGTGGCTGTACCTCGC |
| | | AGAACTGGTGACCCCTATCCTGCAGGAGGGCGGCGTTCCAATGGGT |
| | | GAGTTGTTCAGAGAAATCACAAAGCCACTGCGCCCACTGGGCAAGG |
| | | CGGCTAGTCTCCTTCTGGAGATTCTCGGCCTGCTCTGTAAGAGCATG |
| | | GGTCCAAAGAAGGTGGGCACCCTGTGGAGGGAAGCTGGACTCTCCT |
| | | GGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTC |
| 45 | MBP-CTIF | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSENSSAASASSEAGSSRSQEIEELERF |
| | | IDSYVLEYQVQGLLADKTEGDGESERTQSHISQWTADCSEPLDSSCSFS |
| | | RGRAPPQQNGSKDNSLDMLGTDIWAANTFDSFSGATWDLQPEKLDFTQ |
| | | FHRKVRHTPKQPLPHIDREGCGKGKLEDGDGINLNDIEKVLPAWQGYH |
| | | PMPHEVEIAHTKKLFRRRRNDRRRQQRPPGGNKPQQHGDHQPGSAKH |
| | | NRDHQKSYQGGSAPHPSGRPTHHGYSQNRRWHHGNMKHPPGDKGEA |
| | | GAHRNAKETMTIENPKLEDTAGDTGHSSLEAPRSPDTLAPVASERLPPQ |
| | | QSGGPEVETKRKDSILPERIGERPKITLLQSSKDRLRRRLKEKDEVAVET |
| | | TTPQQNKMDKLIEILNSMRNNSSDVDTKLTTFMEEAQNSTNSEEMLGEI |
| | | VRTIYQKAVSDRSFAFTAAKLCDKMALFMVEGTKFRSLLLNMLQKDFT |
| | | VREELQQQDVERWLGFITFLCEVFGTMRSSTGEPFRVLVCPIYTCLRELL |
| | | QSQDVKEDAVLCCSMELQSTGRLLEEQLPEMMTELLASARDKMLCPSE |
| | | SMLTRSLLLEVIELHANSWNPLTPPITQYYNRTIQKLTA |
| 46 | MBP-CTIF | ATGGCCAGCAACTTCACCCAGTTCGTGCTGGTGGACAACGGCGGTA |
| | (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAG |
| | | AACAGTAGCGCCGCTAGCGCGAGCAGCGAAGCCGGGAGCAGCCGGT |
| | | CTCAGGAGATCGAGGAGCTGGAGCGGTTCATCGACAGCTACGTGCT |
| | | GGAGTACCAGGTGCAGGGCCTGCTGGCCGACAAGACCGAGGGCGAC |
| | | GGCGAGAGCGAGCGGACCCAGAGCCACATCAGCCAGTGGACCGCCG |
| | | ACTGCAGCGAGCCCCTGGACTCTAGCTGCTCATTCAGTCGTGGACGG |
| | | GCCCCTCCACAGCAGAACGGCAGCAAGGACAACAGCCTGGACATGC |
| | | TGGGCACCGACATCTGGGCCGCCAACACCTTCGACAGCTTCAGCGG |
| | | CGCCACCTGGGATCTGCAGCCCGAGAAGCTGGACTTTACCCAGTTCC |
| | | ACCGGAAGGTGCGGCACACTCCCAAGCAGCCCCTGCCCCACATCGA |
| | | TCGGGAGGGCTGCGGCAAGGGCAAGCTGGAAGACGGCGACGGCAT |
| | | CAACCTGAACGACATCGAGAAGGTGCTGCCTGCCTGGCAGGGCTAC |
| | | CACCCCATGCCCCACGAGGTGGAGATCGCCCACACCAAGAAGCTGT |
| | | TCCGGCGACGACGCAACGACCGGCGTAGGCAGCAACGGCCGCCTGG |
| | | AGGGAACAAGCCCCAGCAGCACGGAGACCACCAGCCCGGTAGCGCC |
| | | AAGCACAACCGGGACCACCAGAAGAGCTACCAGGGCGGAAGCGCA |
| | | CCACACCCCTCGGGCAGACCCACCCACCACGGCTACAGCCAGAACC |
| | | GGCGGTGGCATCACGGTAACATGAAGCACCCACCCGGCGACAAAGG |
| | | AGAGGCCGGCGCTCACCGTAACGCCAAGGAGACCATGACCATCGAG |
| | | AACCCCAAGCTGGAGGATACCGCCGGCGATACGGGTCACAGCAGCC |
| | | TGGAGGCACCGCGGTCTCCCGACACCCTGGCACCCGTGGCCAGCGA |
| | | ACGGCTGCCACCCCAACAGAGCGGCGGCCCTGAGGTTGAGACCAAG |
| | | CGGAAGGACAGCATCCTGCCCGAACGGATCGGTGAGCGGCCCAAGA |
| | | TCACCTTACTGCAGAGTAGCAAGGACCGGCTGAGACGGCGGCTGAA |
| | | GGAGAAGGACGAGGTGGCCGTGGAGACAACCACTCCCCAGCAGAA |
| | | CAAGATGGACAAGCTGATCGAGATCCTGAACAGCATGCGGAACAAC |
| | | AGCAGCGACGTGGACACCAAGCTGACCACCTTCATGGAGGAGGCCC |
| | | AGAACAGCACCAACAGCGAGGAGATGCTGGGCGAGATCGTGCGGA |
| | | CCATCTACCAGAAGGCCGTGAGCGACCGGAGCTTCGCCTTCACCGCC |
| | | GCCAAGCTGTGCGACAAGATGGCCCTGTTCATGGTGGAGGGCACCA |
| | | AGTTCCGGAGCTTACTGCTGAATATGCTGCAGAAGGACTTCACCGTG |
| | | CGGGAGGAGCTGCAGCAGCAGGACGTGGAGCGGTGGCTGGGCTTCA |
| | | TCACCTTCCTGTGCGAGGTGTTCGGCACCATGCGGAGCAGCACCGGC |
| | | GAACCCTTCCGGGTGCTGGTGTGCCCCATCTACACCTGCCTGCGGGA |
| | | GTTGCTGCAGAGCCAGGACGTGAAGGAGGACGCCGTGCTGTGCTGC |
| | | AGCATGGAACTGCAGAGCACTGGCCGGCTGCTGGAGGAGCAGCTGC |
| | | CCGAGATGATGACCGAGCTGCTCGCTAGCGCCCGGGACAAGATGCT |
| | | GTGCCCCAGCGAGAGCATGCTGACCCGGAGCCTGCTTCTGGAGGTG |
| | | ATCGAGCTGCACGCCAACAGCTGGAATCCCCTGACCCCTCCCATCAC |
| | | CCAGTACTACAACCGGACCATCCAGAAGCTGACCGCC |
| 47 | MBP- | MASNFTQFVLVDNGGTGDVTVAPSNFANGIAEWISSNSRSQAYKVTCS |
| | CTIF(379- | VRQSSAQNRKYTIKVEVPKGAWRSYLNMELTIPIFATNSDCELIVKAMQ |
| | 579) (aa) | GLLKDGNPIPSAIAANSGIYGGGGSLNSMRNNSSDVDTKLTTFMEEAQN |
| | | STNSEEMLGEIVRTIYQKAVSDRSFAFTAAKLCDKMALFMVEGTKFRSL |
| | | LLNMLQKDFTVREELQQQDVERWLGFITFLCEVFGTMRSSTGEPFRVL |
| | | VCPIYTCLRELLQSQDVKEDAVLCCSMELQSTGRLLEEQLPEMMTELLA |
| | | SARDKMLCPSESMLTRSLLLEVIELHANSWN |
| 48 | MBP- | ATGGCCAGCAACTTCACCCAGTTCGTGCTGGTGGACAACGGCGGTA |
| | CTIF(379- | CCGGAGACGTGACCGTGGCCCCTTCTAACTTCGCCAACGGCATCGCC |
| | 579) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCCTG |
| | | AACAGCATGAGAAACAACAGCAGCGACGTGGACACCAAGCTGACC |
| | | ACCTTCATGGAGGAGGCCCAGAACAGCACCAACAGCGAGGAGATGC |
| | | TGGGCGAGATCGTGAGAACCATCTACCAGAAGGCCGTGAGCGACAG |
| | | AAGCTTCGCCTTCACCGCCGCCAAGCTGTGCGACAAGATGGCCCTGT |
| | | TCATGGTGGAGGGCACCAAGTTCAGAAGCCTGCTGCTGAACATGCT |
| | | GCAGAAGGACTTCACCGTGAGAGAGGAGCTGCAGCAGCAGGACGTG |
| | | GAGCGATGGCTGGGCTTCATCACCTTCCTGTGCGAGGTGTTCGGCAC |
| | | CATGAGAAGCAGCACCGGCGAGCCTTTCAGAGTGCTGGTGTGCCCT |
| | | ATCTACACCTGCCTGAGAGAGCTGCTGCAGAGCCAGGACGTGAAGG |
| | | AGGACGCCGTGCTGTGCTGCAGCATGGAGCTGCAGAGCACCGGCAG |
| | | ACTGCTGGAGGAGCAGCTGCCTGAGATGATGACCGAGCTGCTGGCC |
| | | AGCGCCAGAGACAAGATGCTGTGCCCTAGCGAGAGCATGCTGACTA |
| | | GATCCCTACTGTTGGAGGTGATCGAGCTGCACGCCAACAGCTGGAA |
| | | C |
| 49 | MBP- | MASNFTQFVLVDNGGTGDVTVAPSNFANGIAEWISSNSRSQAYKVTCS |
| | CTIF(365- | VRQSSAQNRKYTIKVEVPKGAWRSYLNMELTIPIFATNSDCELIVKAMQ |
| | 598) (aa) | GLLKDGNPIPSAIAANSGIYGGGGSTTPQQNKMDKLIEILNSMRNNSSD |
| | | VDTKLTTFMEEAQNSTNSEEMLGEIVRTIYQKAVSDRSFAFTAAKLCDK |
| | | MALFMVEGTKFRSLLLNMLQKDFTVREELQQQDVERWLGFITFLCEVF |
| | | GTMRSSTGEPFRVLVCPIYTCLRELLQSQDVKEDAVLCCSMELQSTGRL |
| | | LEEQLPEMMTELLASARDKMLCPSESMLTRSLLLEVIELHANSWNPLTP |
| | | PITQYYNRTIQKLTA |
| 50 | MBP- | ATGGCCAGCAACTTCACCCAGTTCGTGCTGGTGGACAACGGCGGTA |
| | _CTIF(365- | CCGGAGACGTGACCGTGGCCCCTTCTAACTTCGCCAACGGCATCGCC |
| | 598) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCACC |
| | | ACCCCTCAGCAGAACAAGATGGACAAGCTGATCGAGATCCTGAACA |
| | | GCATGAGAAACAACAGCAGCGACGTGGACACCAAGCTGACCACCTT |
| | | CATGGAGGAGGCCCAGAACAGCACCAACAGCGAGGAGATGCTGGG |
| | | CGAGATCGTGAGAACCATCTACCAGAAGGCCGTGAGCGACAGAAGC |
| | | TTCGCCTTCACCGCCGCCAAGCTGTGCGACAAGATGGCCCTGTTCAT |
| | | GGTGGAGGGCACCAAGTTCAGAAGCCTGCTGCTGAACATGCTGCAG |
| | | AAGGACTTCACCGTGAGAGAGGAGCTGCAGCAGCAGGACGTGGAG |
| | | AGGTGGCTGGGCTTCATCACCTTCCTGTGCGAGGTGTTCGGCACCAT |
| | | GAGAAGCAGCACCGGCGAGCCTTTCAGAGTGCTGGTGTGCCCTATCT |
| | | ACACCTGCCTGAGAGAGCTGCTGCAGAGCCAGGACGTGAAGGAGGA |
| | | CGCCGTGCTGTGCTGCAGCATGGAGCTGCAGAGCACCGGCAGACTG |
| | | CTGGAGGAGCAGCTGCCTGAGATGATGACCGAGCTGCTGGCCAGCG |
| | | CCAGAGACAAGATGCTGTGCCCTAGCGAGAGCATGCTGACGCGCAG |
| | | CCTCCTGCTGGAGGTGATCGAGCTGCACGCCAACAGCTGGAACCCTC |
| | | TGACCCCTCCTATCACCCAGTACTACAACAGAACCATCCAGAAGCTG |
| | | ACCGCC |
| 51 | MBP-eIF4G- | MASNFTQFVLVDNGGTGDVTVAPSNFANGIAEWISSNSRSQAYKVTCS |
| | mid4 (752- | VRQSSAQNRKYTIKVEVPKGAWRSYLNMELTIPIFATNSDCELIVKAMQ |
| | 993) (aa) | GLLKDGNPIPSAIAANSGIYGGGGSADGSKTQDLFRRVRSILNKLTPQMF |
| | | QQLMKQVTQLAIDTEERLKGVIDLIFEKAISEPNFSVAYANMCRCLMAL |
| | | KVPTTEKPTVTVNFRKLLLNRCQKEFEKDKDDDEVFEKKQKEMDEAA |
| | | TAEERGRLKEELEEARDIARRRSLGNIKFIGELFKLKMLTEAIMHDCVV |
| | | KLLKNHDEESLECLCRLLTTIGKDLDFEKAKPRMDQYFNQMEKIIKEKK |
| | | TSSRIRFMLQDVLDLRGSNWVPR |
| 52 | MBP-eIF4G- | ATGGCCAGCAACTTCACCCAGTTCGTGCTGGTGGACAACGGCGGTA |
| | mid4 (752- | CCGGAGACGTGACCGTGGCCCCTTCTAACTTCGCCAACGGCATCGCC |
| | 993) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGCC |
| | | GACGGCAGCAAGACCCAGGACCTGTTCAGAAGAGTGAGAAGCATCC |
| | | TGAACAAGCTGACCCCTCAGATGTTCCAGCAGCTGATGAAGCAGGT |
| | | GACCCAGCTGGCCATCGACACCGAGGAGAGACTGAAGGGCGTGATC |
| | | GACCTGATCTTCGAGAAGGCCATCAGCGAGCCTAACTTCAGCGTGG |
| | | CCTACGCCAACATGTGCCGGTGCCTGATGGCCCTGAAGGTGCCTACC |
| | | ACCGAGAAGCCTACCGTGACCGTGAACTTCAGAAAGCTGCTGCTGA |
| | | ACCGGTGCCAGAAGGAGTTCGAGAAGGACAAGGACGACGACGAGG |
| | | TGTTCGAGAAGAAGCAGAAGGAGATGGACGAGGCCGCCACCGCCG |
| | | AGGAGAGAGGCAGACTGAAGGAGGAGCTGGAGGAGGCCAGAGACA |
| | | TCGCCAGAAGAAGAAGCCTGGGCAACATCAAGTTCATCGGCGAGCT |
| | | GTTCAAGCTGAAGATGCTGACCGAGGCCATCATGCACGACTGCGTG |
| | | GTGAAGCTGCTGAAGAACCACGACGAGGAGAGCCTGGAGTGCCTGT |
| | | GCAGACTGCTGACCACCATCGGCAAGGACCTGGATTTCGAGAAGGC |
| | | GAAGCCTAGAATGGACCAGTACTTCAACCAGATGGAGAAGATCATC |
| | | AAGGAGAAGAAGACCAGCAGCAGAATCAGATTCATGCTGCAGGAC |
| | | GTGCTGGACCTGAGAGGCAGCAACTGGGTGCCTAGA |
| 53 | MBP-LacZ | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSSFTLTNKNVIFVAGLGGIGLDTSKE |
| | | LLKRDPVVLQRRDWENPGVTQLNRLAAHPPFASWRNSEEARTDRPSQQ |
| | | LRSLNGEWRFAWFPAPEAVPESWLECDLPEADTVVVPSNWQMHGYDA |
| | | PIYTNVTYPITVNPPFVPTENPTGCYSLTFNVDESWLQEGQTRIIFDGVNS |
| | | AFHLWCNGRWVGYGQDSRLPSEFDLSAFLRAGENRLAVMVLRWSDGS |
| | | YLEDQDMWRMSGIFRDVSLLHKPTTQISDFHVATRENDDFSRAVLEAE |
| | | VQMCGELRDYLRVTVSLWQGETQVASGTAPFGGEIIDERGGYADRVTL |
| | | RLNVENPKLWSAEIPNLYRAVVELHTADGTLIEAEACDVGFREVRIENG |
| | | LLLLNGKPLLIRGVNRHEHHPLHGQVMDEQTMVQDILLMKQNNFNAV |
| | | RCSHYPNHPLWYTLCDRYGLYVVDEANIETHGMVPMNRLTDDPRWLP |
| | | AMSERVTRMVQRDRNHPSVIIWSLGNESGHGANHDALYRWIKSVDPSR |
| | | PVQYEGGGADTTATDIICPMYARVDEDQPFPAVPKWSIKKWLSLPGET |
| | | RPLILCEYAHAMGNSLGGFAKYWQAFRQYPRLQGGFVWDWVDQSLIK |
| | | YDENGNPWSAYGGDFGDTPNDRQFCMNGLVFADRTPHPALTEAKHQQ |
| | | QFFQFRLSGQTIEVTSEYLFRHSDNELLHWMVALDGKPLASGEVPLDV |
| | | APQGKQLIELPELPQPESAGQLWLTVRVVQPNATAWSEAGHISAWQQ |
| | | WRLAENLSVTLPAASHAIPHLTTSEMDFCIELGNKRWQFNRQSGFLSQ |
| | | MWIGDKKQLLTPLRDQFTRAPLDNDIGVSEATRIDPNAWVERWKAAG |
| | | HYQAEAALLQCTADTLADAVLITTAHAWQHQGKTLFISRKTYRIDGSG |
| | | QMAITVDVEVASDTPHPARIGLNCQLAQVAERVNWLGLGPQENYPDRL |
| | | TAACFDRWDLPLSDMYTPYVFPSENGLRCGTRELNYGPHQWRGDFQF |
| | | NISRYSQQQLMETSHRHLLHAEEGTWLNIDGFHMGIGGDDSWSPSVSA |
| | | ELQLSAGRYHYQLVWCQK |
| 54 | MBP-LacZ | ATGGCCAGCAACTTCACCCAGTTCGTGCTGGTGGACAACGGCGGTA |
| | (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCTCG |
| | | TTCACGCTAACGAACAAGAACGTCATCTTCGTAGCGGGACTTGGCG |
| | | GTATCGGCCTAGACACGTCGAAGGAACTACTAAAGCGTGACCCGGT |
| | | AGTCCTCCAACGTCGCGATTGGGAGAACCCGGGCGTAACGCAACTA |
| | | AACCGTCTTGCGGCGCACCCGCCGTTTGCGTCGTGGCGTAACTCGGA |
| | | GGAGGCGCGAACGGATCGTCCGTCGCAACAACTACGTTCGCTCAAC |
| | | GGGGAGTGGCGCTTCGCGTGGTTCCCGGCGCCGGAGGCGGTACCGG |
| | | AGTCGTGGCTCGAGTGCGATCTACCGGAGGCGGACACGGTCGTCGT |
| | | ACCGTCGAACTGGCAAATGCACGGTTACGACGCGCCGATATACACG |
| | | AACGTCACGTACCCGATAACGGTAAACCCGCCGTTCGTCCCGACGG |
| | | AGAACCCGACGGGGTGCTACTCGCTAACGTTCAACGTTGACGAGTC |
| | | GTGGTTGCAAGAGGGTCAAACGCGTATCATATTCGACGGTGTAAAC |
| | | TCGGCGTTCCACCTGTGGTGCAACGGGCGCTGGGTAGGGTACGGCC |
| | | AAGACTCGCGTCTACCGTCGGAGTTCGACCTATCGGCGTTCCTACGA |
| | | GCGGGTGAGAACCGGCTAGCGGTCATGGTCCTACGTTGGTCGGACG |
| | | GTTCGTACCTCGAGGACCAAGACATGTGGCGAATGTCGGGTATCTTC |
| | | CGCGACGTATCGCTCCTACACAAGCCGACGACGCAAATCTCGGACTT |
| | | CCACGTCGCGACGCGTTTCAACGACGATTTCTCGCGGGCAGTCCTAG |
| | | AGGCGGAGGTCCAAATGTGCGGGGAGCTACGTGACTACCTCCGTGT |
| | | CACGGTATCGCTCTGGCAAGGTGAGACGCAAGTAGCGTCGGGTACG |
| | | GCGCCGTTCGGCGGTGAGATCATCGACGAGCGTGGTGGGTACGCGG |
| | | ACCGTGTAACGCTACGTCTAAACGTCGAGAACCCGAAGCTCTGGTC |
| | | GGCGGAGATCCCGAACCTATACCGTGCGGTCGTCGAGCTACATACG |
| | | GCGGACGGGACGCTAATAGAGGCGGAAGCGTGCGACGTCGGGTTTC |
| | | GAGAGGTTCGTATAGAGAACGGGCTGCTACTTCTAAACGGGAAGCC |
| | | GTTGCTCATACGTGGTGTCAACCGTCACGAGCACCACCCGCTACACG |
| | | GTCAAGTAATGGACGAGCAAACGATGGTACAAGACATCCTACTAAT |
| | | GAAGCAGAACAACTTCAACGCGGTACGCTGTTCGCATTACCCGAAC |
| | | CATCCGTTGTGGTACACGCTTTGCGACCGATACGGTCTATACGTCGT |
| | | AGACGAGGCGAACATAGAGACGCACGGGATGGTACCGATGAATCGC |
| | | CTAACGGACGACCCGCGTTGGCTACCGGCGATGTCGGAGCGAGTCA |
| | | CGCGTATGGTCCAACGGGACCGTAACCACCCGTCGGTAATAATCTG |
| | | GTCGCTAGGCAACGAATCGGGGCACGGGGCGAACCACGACGCGCTA |
| | | TACCGTTGGATCAAGTCGGTAGACCCGTCGCGTCCGGTACAATACGA |
| | | AGGTGGCGGTGCGGACACGACGGCGACGGACATCATCTGCCCGATG |
| | | TACGCGCGCGTCGACGAAGACCAACCGTTCCCGGCGGTACCGAAGT |
| | | GGTCGATCAAGAAGTGGCTCTCGTTGCCGGGTGAAACGCGTCCGTTG |
| | | ATACTTTGCGAGTACGCGCACGCGATGGGCAACTCGTTGGGTGGGTT |
| | | CGCGAAGTACTGGCAGGCGTTCCGTCAATACCCGCGTCTACAGGGT |
| | | GGGTTCGTCTGGGACTGGGTAGACCAATCGCTAATCAAGTACGACG |
| | | AGAACGGCAACCCGTGGTCGGCGTACGGTGGGGACTTCGGGGACAC |
| | | GCCGAACGACCGCCAATTCTGTATGAACGGCCTAGTCTTCGCGGACC |
| | | GAACGCCGCACCCGGCGTTGACGGAGGCGAAGCATCAACAACAATT |
| | | CTTCCAATTCCGTCTATCGGGGCAAACGATCGAGGTAACGTCGGAGT |
| | | ACTTGTTCCGGCACTCGGACAACGAGCTACTACACTGGATGGTAGCA |
| | | CTAGACGGCAAGCCGCTAGCGTCGGGAGAAGTCCCTTTGGACGTCG |
| | | CGCCGCAAGGTAAGCAACTAATCGAGCTACCGGAGCTACCGCAACC |
| | | GGAGTCGGCGGGTCAACTGTGGTTGACGGTCCGTGTCGTTCAACCGA |
| | | ACGCGACGGCGTGGTCGGAGGCGGGTCACATCTCGGCGTGGCAGCA |
| | | GTGGCGTCTAGCGGAGAACCTCTCGGTCACGCTACCGGCGGCGTCG |
| | | CACGCGATACCGCATCTAACGACGTCGGAGATGGACTTCTGCATCG |
| | | AGTTGGGGAACAAGAGGTGGCAGTTCAACCGTCAATCGGGATTCCT |
| | | ATCGCAAATGTGGATAGGTGACAAGAAGCAACTACTAACGCCGCTA |
| | | CGTGATCAGTTCACGCGTGCTCCGCTAGACAACGACATAGGTGTTTC |
| | | GGAGGCGACGCGTATAGACCCGAACGCGTGGGTGGAGCGGTGGAA |
| | | GGCGGCGGGGCACTACCAAGCGGAGGCGGCGCTACTACAGTGCACG |
| | | GCGGACACGCTAGCGGACGCGGTATTGATCACGACGGCGCACGCGT |
| | | GGCAACACCAGGGGAAGACGCTATTCATCTCGCGTAAGACGTACCG |
| | | TATCGACGGTTCGGGCCAAATGGCGATCACGGTCGACGTAGAGGTA |
| | | GCGTCGGACACGCCGCATCCGGCGCGCATCGGTCTAAACTGCCAAC |
| | | TAGCGCAAGTAGCGGAGCGTGTAAACTGGCTAGGGCTAGGGCCGCA |
| | | AGAGAACTATCCGGACCGCCTAACGGCGGCGTGCTTCGACCGTTGG |
| | | GACCTACCGCTTTCGGACATGTATACCCCGTACGTCTTCCCGTCGGA |
| | | GAACGGGTTGAGGTGCGGGACGCGCGAGCTAAACTACGGGCCGCAC |
| | | CAGTGGCGAGGGGACTTCCAATTCAACATATCGCGTTACTCGCAACA |
| | | ACAACTAATGGAGACGTCGCACCGTCACCTACTACACGCGGAGGAG |
| | | GGGACGTGGCTAAACATCGACGGGTTCCACATGGGCATAGGTGGGG |
| | | ACGACTCGTGGTCGCCGTCGGTCTCGGCGGAGCTCCAACTCTCGGCG |
| | | GGTCGTTACCATTACCAACTAGTTTGGTGCCAGAAG |
| 55 | 50S | MYVRFEVPEDMQNEALSLLEKVRESGKVKKGTNETTKAVERGLAKLV |
| | ribosomal | YIAEDVDPPEIVAHLPLLCEEKNVPYIYVKSKNDLGRAVGIEVPCASAAI |
| | L7Ae protein | INEGELRKELGSLVEKIKGLQK |
| | (aa) | |
| 56 | 50S | ATGTACGTGAGATTTGAGGTTCCTGAGGACATGCAGAACGAAGCTC |
| | ribosomal | TGAGTCTGCTGGAGAAGGTTAGGGAGAGCGGTAAGGTAAAGAAAG |
| | L7Ae protein | GTACCAACGAGACGACAAAGGCTGTGGAGAGGGGACTGGCAAAGC |
| | (nt) | TCGTTTACATCGCAGAGGATGTTGACCCGCCTGAGATCGTTGCTCAT |
| | | CTGCCCCTCCTCTGCGAGGAGAAGAATGTGCCGTACATTTACGTTAA |
| | | AAGCAAGAACGACCTTGGAAGGGCTGTGGGCATTGAGGTGCCATGC |
| | | GCTTCGGCAGCGATAATCAACGAGGGAGAGCTGAGAAAGGAGCTTG |
| | | GAAGCCTTGTGGAGAAGATTAAAGGCCTTCAGAAGAGATCTCATAT |
| | | GCATCTCGAG |
| 57 | Pre- | MASNFTQFVLVDNGGTGDVTVAPSNFANGVAEWISSNSRSQAYKVTCS |
| | dimerized | VRQSSAQNRKYTIKVEVPKVATQTVGGVELPVAAWRSYLNMELTIPIF |
| | MBP (aa) | ATNSDCELIVKAMQGLLKDGNPIPSAIAANSGIYGGGGSASNFTQFVLV |
| | (linker | DNGGTGDVTVAPSNFANGVAEWISSNSRSQAYKVTCSVRQSSAQNRK |
| | sequence | YTIKVEVPKVATQTVGGVELPVAAWRSYLNMELTIPIFATNSDCELIVK |
| | underlined) | AMQGLLKDGNPIPSAIAANSGIY |
| 58 | Pre- | ATGGCCAGCAATTTTACCCAGTTCGTGCTGGTCGACAACGGCGG |
| | dimerized | GACCGGTGACGTTACCGTGGCCCCTAGCAATTTCGCTAACGGCGTGG |
| | MBP (nt) | CTGAGTGGATCTCCAGCAACAGCCGGAGCCAAGCTTACAAGGTGAC |
| | (linker | CTGTAGCGTGCGGCAGAGCAGCGCCCAGAACCGGAAGTACACCATC |
| | sequence | AAGGTAGAAGTGCCTAAGGTGGCCACACAGACTGTGGGCGGTGTCG |
| | underlined) | AGCTGCCCGTGGCAGCTTGGAGGAGCTATCTGAATATGGAACTGAC |
| | | CATCCCCATCTTCGCCACTAACAGCGATTGTGAGCTGATTGTTAAGG |
| | | CCATGCAAGGCCTGCTAAAGGACGGGAACCCCATTCCCAGCGCCAT |
| | | CGCTGCCAACAGCGGCATCTACGGAGGAGGCGGAAGCGCTAGCAAC |
| | | TTCACCCAGTTTGTGCTGGTGGACAACGGTGGCACCGGCGACGTGAC |
| | | CGTGGCTCCCAGCAACTTTGCCAACGGTGTGGCCGAGTGGATCAGC |
| | | AGCAATTCTCGGAGCCAGGCCTACAAGGTCACGTGCAGCGTGCGCC |
| | | AAAGCAGCGCTCAGAATCGGAAGTATACTATCAAGGTGGAGGTGCC |
| | | CAAAGTGGCCACCCAGACCGTGGGTGGCGTGGAGCTGCCTGTGGCC |
| | | GCTTGGCGATCATACCTGAACATGGAGCTCACCATCCCTATCTTCGC |
| | | AACCAACAGCGACTGCGAGCTGATCGTGAAAGCCATGCAGGGCCTG |
| | | CTGAAGGACGGCAATCCCATCCCCAGCGCTATCGCCGCCAATAGCG |
| | | GAATCTAC |
| 59 | MBP-mid2- | MASNFTQFVLVDNGGTGDVTVAPSNFANGIAEWISSNSRSQAYKVTCS |
| | mut (aa) | VRQSSAQNRKYTIKVEVPKGAWRSYLNMELTIPIFATNSDCELIVKAMQ |
| | | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKAIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAV |
| 60 | MBP-mid2- | ATGGCCAGCAACTTCACCCAGTTCGTGCTGGTGGACAACGGCGGTA |
| | mut (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGGCGATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTG |
| 61 | PCP (aa) | MSKTIVLSVGEATRTLTEIQSTADRQIFEEKVGPLVGRLRLTASLRQNGA |
| | | KTAYRVNLKLDQADVVDCSTSVCGELPKVRYTQVWSHDVTIVANSTE |
| | | ASRKSLYDLTKSLVVQATSEDLVVNLVPLGR |
| 62 | PCP (nt) | ATGAGCAAGACCATCGTGCTGAGCGTGGGCGAGGCCACCAGAACCC |
| | | TGACCGAGATCCAGAGCACCGCCGACAGACAGATCTTCGAGGAGAA |
| | | GGTGGGCCCTCTGGTGGGCAGACTGAGACTGACCGCCAGCCTGAGA |
| | | CAGAACGGCGCCAAGACCGCCTACAGAGTGAACCTGAAGCTGGACC |
| | | AGGCCGACGTGGTGGACTGCAGCACCAGCGTGTGCGGCGAGCTGCC |
| | | TAAGGTGAGATACACCCAGGTGTGGAGCCACGACGTGACCATCGTG |
| | | GCCAACAGCACCGAGGCCAGCAGAAAGAGCCTGTACGACCTGACCA |
| | | AGAGCCTGGTGGTGCAGGCCACCAGCGAGGACCTGGTGGTGAACCT |
| | | GGTGCCTCTGGGCAGA |
| 63 | PUF (aa) | MGSSHHHHHHSQDLEVLFQGPHMGRSRLLEDFRNNRYPNLQLREIAGH |
| | | IMEFSQDQHGSRFIQLKLERATPAERQLVFNEILQAAYQLMVDVFGSYV |
| | | IEKFFEFGSLEQKLALAERIRGHVLSLALQMYGSRVIEKALEFIPSDQQN |
| | | EMVRELDGHVLKCVKDQNGCHVVQKCIECVQPQSLQFIIDAFKGQVFA |
| | | LSTHPYGSRVIERILEHCLPDQTLPILEELHQHTEQLVQDQYGCYVIQHV |
| | | LEHGRPEDKSKIVAEIRGNVLVLSQHKFASNVVEKCVTHASRTERAVLI |
| | | DEVCTMNDGPHSALYTMMKDQYASYVVEKMIDVAEPGQRKIVMHKIR |
| | | PHIMEFSQDQHGSRFIELKLERATPAERQLVFNEILQAAYQLMVDVFGC |
| | | YVIQKFFEFGSLEQKLALAERIRGHVLSLALQMYGSRVIEKALEFIPSDQ |
| | | QNEMVRELDGHVLKCVKDQNGCHVVQKCIECVQPQSLQFIIDAFKGQV |
| | | FALSTHPYGSRVIERILEHCLPDQTLPILEELHQHTEQLVQDQYGCYVIQ |
| | | HVLEHGRPEDKSKIVAEIRGNVLVLSQHKFASNVVEKCVTHASRTERA |
| | | VLIDEVCTMNDGPHSALYTMMKDQYACYVVQKMIDVAEPGQRKIVM |
| | | HKIRPHIATLRKYTYGKHILAKLEKYYMKNGVDLG |
| 64 | PUF (nt) | ATGGGCAGCAGCCACCATCACCACCACCACAGCCAGGACCTGGAGG |
| | | TGCTGTTCCAGGGCCCACACATGGGCCGTAGCCGGCTGCTGGAGGA |
| | | CTTCCGGAACAACCGGTACCCCAACCTGCAGCTGCGGGAGATCGCC |
| | | GGCCACATCATGGAGTTCAGCCAGGACCAGCACGGCAGCCGCTTCA |
| | | TCCAGCTGAAGTTGGAGCGGGCTACACCCGCTGAGCGGCAGCTGGT |
| | | GTTCAACGAGATCCTGCAGGCCGCCTATCAACTGATGGTAGACGTGT |
| | | TCGGCAGCTACGTGATCGAGAAGTTCTTTGAGTTCGGCAGTCTGGAG |
| | | CAGAAGCTCGCCCTGGCCGAACGGATACGGGGCCACGTGCTTAGCC |
| | | TGGCCCTACAGATGTACGGCAGCAGGGTGATCGAGAAAGCCCTGGA |
| | | GTTCATCCCCAGCGACCAACAGAACGAAATGGTGCGGGAGCTCGAC |
| | | GGCCACGTCCTGAAGTGCGTGAAGGACCAGAACGGTTGCCACGTGG |
| | | TGCAGAAGTGCATCGAGTGCGTCCAGCCCCAGTCTCTGCAGTTCATT |
| | | ATCGACGCCTTCAAGGGCCAAGTGTTCGCCCTGAGTACCCACCCCTA |
| | | CGGAAGCCGGGTGATCGAACGGATTCTGGAGCATTGCCTGCCCGAT |
| | | CAGACCCTGCCCATCCTGGAGGAGCTGCATCAACACACCGAACAGC |
| | | TTGTGCAAGACCAGTACGGCTGCTACGTGATCCAGCACGTGTTGGAG |
| | | CACGGACGGCCCGAGGACAAGAGCAAGATTGTGGCCGAGATCAGG |
| | | GGTAACGTGCTGGTGCTGTCTCAGCACAAATTCGCCAGCAACGTGGT |
| | | AGAGAAGTGCGTGACCCACGCCAGCCGGACTGAACGGGCCGTGCTG |
| | | ATCGACGAGGTGTGCACTATGAACGACGGGCCCCACTCTGCCCTGTA |
| | | CACAATGATGAAAGACCAATACGCCAGCTACGTGGTGGAGAAGATG |
| | | ATCGACGTGGCCGAGCCAGGCCAACGGAAGATCGTGATGCATAAGA |
| | | TCCGGCCTCATATCATGGAGTTTAGCCAGGATCAACACGGCAGCCG |
| | | GTTCATCGAGCTGAAGCTGGAACGGGCCACCCCTGCCGAACGGCAG |
| | | CTGGTCTTTAACGAGATACTGCAAGCCGCCTACCAGCTGATGGTGGA |
| | | CGTGTTTGGTTGCTACGTTATCCAGAAGTTCTTCGAGTTCGGAAGCC |
| | | TGGAGCAGAAACTGGCCCTGGCCGAGCGTATCCGGGGCCACGTTCT |
| | | GAGCTTAGCCCTGCAGATGTACGGGAGCCGGGTTATCGAGAAGGCC |
| | | CTGGAATTCATCCCTAGCGATCAGCAGAACGAGATGGTCAGAGAGC |
| | | TGGACGGCCACGTGCTCAAGTGCGTGAAAGACCAGAACGGCTGCCA |
| | | CGTGGTACAGAAGTGTATCGAGTGTGTGCAGCCCCAGAGCCTCCAG |
| | | TTTATCATTGACGCCTTTAAGGGCCAGGTGTTTGCCCTGAGCACACA |
| | | CCCCTACGGCAGCCGGGTAATCGAGCGGATCCTGGAGCACTGCCTG |
| | | CCCGACCAGACCTTGCCCATCCTCGAGGAACTGCACCAGCACACCG |
| | | AGCAGCTGGTGCAGGACCAATACGGCTGTTACGTCATCCAGCACGTT |
| | | CTGGAGCACGGCCGGCCTGAGGACAAGTCTAAGATCGTGGCCGAAA |
| | | TCCGGGGCAACGTGTTGGTGCTGAGCCAGCACAAGTTCGCCTCAAA |
| | | CGTCGTGGAGAAGTGTGTGACTCACGCCTCTCGGACCGAGCGGGCA |
| | | GTGCTGATTGACGAGGTGTGTACCATGAACGACGGCCCACACAGCG |
| | | CCCTGTACACCATGATGAAGGACCAGTACGCCTGCTACGTTGTGCAG |
| | | AAGATGATTGACGTGGCCGAACCCGGTCAGCGCAAGATTGTGATGC |
| | | ACAAGATCCGGCCCCACATCGCCACCCTGCGGAAGTACACCTACGG |
| | | CAAGCACATCCTGGCCAAGCTGGAGAAGTACTACATGAAGAACGGC |
| | | GTGGACCTGGGC |
| 65 | PUF2 (aa) | MGSSHHHHHHSQDLEVLFQGPHMGRSRLLEDFRNNRYPNLQLREIAGH |
| | | IMEFSQDQHGSRFIQLKLERATPAERQLVFNEILQAAYQLMVDVFGNYV |
| | | IQKFFEFGSLEQKLALAERIRGHVLSLALQMYGNRVIQKALEFIPSDQQN |
| | | EMVRELDGHVLKCVKDQNGCHVVQKCIECVQPQSLQFIIDAFKGQVFA |
| | | LSTHPYGNRVIQRILEHCLPDQTLPILEELHQHTEQLVQDQYGCYVIQHV |
| | | LEHGRPEDKSKIVAEIRGNVLVLSQHKFASNVVEKCVTHASRTERAVLI |
| | | DEVCTMNDGPHSALYTMMKDQYANYVVQKMIDVAEPGQRKIVMHKI |
| | | RPHIMEFSQDQHGNRFIQLKLERATPAERQLVFNEILQAAYQLMVDVFG |
| | | NYVIQKFFEFGSLEQKLALAERIRGHVLSLALQMYGNRVIQKALEFIPSD |
| | | QQNEMVRELDGHVLKCVKDQNGNHVVQKCIECVQPQSLQFIIDAFKGQ |
| | | VFALSTHPYGNRVIQRILEHCLPDQTLPILEELHQHTEQLVQDQYGNYVI |
| | | QHVLEHGRPEDKSKIVAEIRGNVLVLSQHKFANNVVQKCVTHASRTER |
| | | AVLIDEVCTMNDGPHSALYTMMKDQYANYVVQKMIDVAEPGQRKIV |
| | | MHKIRPHIATLRKYTYGKHILAKLEKYYMKNGVDLG |
| 66 | PUF2 (nt) | ATGGGCAGCTCCCATCACCACCACCATCATTCGCAGGACCTTGAAGT |
| | | CCTGTTCCAAGGTCCTCATATGGGTAGATCAAGGCTTTTGGAAGATT |
| | | TTCGAAATAACCGGTATCCTAACCTGCAGCTCAGGGAGATAGCAGG |
| | | ACACATCATGGAGTTCAGCCAGGACCAACACGGAAGCAGGTTTATC |
| | | CAACTGAAACTCGAGAGAGCAACGCCCGCAGAGCGTCAGCTGGTGT |
| | | TTAACGAGATCCTGCAGGCAGCCTACCAGCTGATGGTTGACGTGTTC |
| | | GGTAACTACGTAATTCAGAAGTTCTTTGAGTTCGGTTCTCTGGAACA |
| | | GAAACTCGCTCTGGCTGAGCGAATTCGGGGTCACGTGCTCTCACTGG |
| | | CGCTCCAGATGTACGGCAATAGAGTGATTCAGAAGGCTCTGGAATTT |
| | | ATTCCATCGGACCAGCAGAACGAAATGGTACGAGAACTGGACGGTC |
| | | ACGTCCTTAAGTGTGTCAAAGATCAGAACGGCTGTCACGTAGTACA |
| | | GAAGTGCATCGAGTGTGTGCAGCCGCAGAGCCTCCAGTTTATCATCG |
| | | ACGCCTTTAAAGGGCAGGTCTTCGCCTTGTCCACCCATCCGTACGGA |
| | | AACCGAGTCATTCAGCGCATCCTGGAACATTGCCTCCCGGATCAGAC |
| | | CCTGCCGATTCTGGAGGAGCTTCACCAGCACACCGAGCAACTAGTG |
| | | CAGGATCAGTACGGCTGTTACGTGATACAGCACGTCCTGGAGCACG |
| | | GTAGACCAGAGGACAAATCGAAGATTGTGGCCGAAATCAGAGGAA |
| | | ACGTGCTGGTCCTTTCCCAGCACAAGTTCGCTTCCAACGTGGTGGAG |
| | | AAGTGCGTGACACACGCAAGCCGCACCGAGAGGGCAGTCTTAATCG |
| | | ACGAAGTGTGCACAATGAACGACGGTCCACACTCTGCCTTATACAC |
| | | AATGATGAAGGACCAGTACGCAAACTACGTGGTGCAGAAGATGATT |
| | | GACGTAGCTGAGCCTGGCCAGCGAAAGATTGTCATGCATAAGATCA |
| | | GGCCTCATATTATGGAGTTTTCTCAGGATCAACACGGCAATCGGTTC |
| | | ATCCAATTAAAGCTGGAGAGGGCCACACCTGCGGAGAGGCAGTTGG |
| | | TCTTTAACGAAATTCTACAAGCCGCTTACCAATTAATGGTGGACGTC |
| | | TTTGGCAACTACGTGATCCAGAAATTCTTCGAGTTTGGAAGTCTTGA |
| | | ACAGAAGCTGGCGCTGGCTGAAAGAATCAGAGGTCACGTCCTCAGC |
| | | CTGGCATTGCAAATGTACGGAAATCGAGTCATCCAGAAAGCCCTTG |
| | | AGTTTATCCCCAGCGATCAACAGAACGAGATGGTTCGCGAATTGGA |
| | | CGGGCACGTGCTGAAGTGTGTTAAGGACCAGAACGGGAACCACGTC |
| | | GTGCAGAAGTGCATAGAGTGCGTCCAGCCACAGTCCCTGCAGTTTAT |
| | | TATTGACGCATTTAAGGGACAAGTGTTCGCCTTATCTACCCACCCTT |
| | | ACGGCAATAGGGTCATTCAGAGGATCTTGGAACACTGTCTTCCCGAC |
| | | CAGACACTGCCAATATTGGAAGAACTGCATCAACATACCGAACAGC |
| | | TCGTACAAGATCAGTACGGTAATTACGTCATTCAACACGTTCTCGAA |
| | | CACGGAAGGCCCGAAGACAAGTCTAAGATCGTCGCAGAGATACGTG |
| | | GGAACGTCCTCGTCTTAAGTCAGCATAAGTTCGCAAACAACGTTGTG |
| | | CAGAAGTGTGTAACACACGCGAGCAGAACAGAGAGAGCCGTGCTGA |
| | | TCGACGAGGTGTGTACCATGAACGACGGGCCACACAGCGCCCTCTA |
| | | CACCATGATGAAAGACCAATACGCTAACTACGTGGTTCAGAAGATG |
| | | ATCGACGTCGCCGAACCAGGTCAACGGAAGATCGTGATGCACAAGA |
| | | TCAGGCCACACATAGCCACCCTGAGAAAGTATACGTACGGTAAACA |
| | | TATCCTGGCAAAGTTAGAGAAGTACTATATGAAGAACGGCGTGGAC |
| | | TTAGGA |
| 67 | Lambda (aa) | MGNARTRRRERRAEKQAQWKAAN |
| 68 | Lambda (nt) | ATGGGCAACGCCCGGACCAGGAGAAGAGAGCGGCGGGCCGAGAAG |
| | | CAGGCCCAGTGGAAGGCCGCCAAC |
| 69 | U1A (aa) | MAVPETRPNHTIYINNLNEKIKKDELKKSLYAIFSQFGQILDILVSRSLK |
| | | MRGQAFVIFKEVSSATNALRSMQGFPFYDKPMRIQYAKTDSDIIAKMK |
| | | GTF |
| 70 | U1A (nt) | ATGGCCGTGCCTGAGACCAGACCTAACCACACCATCTACATCAACA |
| | | ACCTGAACGAGAAGATCAAGAAGGACGAGCTGAAGAAGAGCCTGT |
| | | ACGCCATCTTCAGCCAGTTCGGCCAGATCCTGGACATCCTGGTGAGC |
| | | AGAAGCCTGAAGATGAGAGGCCAGGCCTTCGTGATCTTCAAGGAGG |
| | | TGAGCAGCGCCACCAACGCCCTGAGAAGCATGCAGGGCTTCCCTTTC |
| | | TACGACAAGCCTATGAGAATCCAGTACGCCAAGACCGACAGCGACA |
| | | TCATCGCCAAGATGAAGGGCACCTTC |
| 71 | 15.5kd (aa) | MTEADVNPKAYPLADAHLTKKLLDLVQQSCNYKQLRKGANEATKTLN |
| | | RGISEFIVMAADAEPLEIILHLPLLCEDKNVPYVFVRSKQALGRACGVSR |
| | | PVIACSVTIKEGSQLKQQIQSIQQSIERLLV |
| 72 | 15.5kd (nt) | ATGACCGAGGCCGACGTGAACCCTAAGGCCTACCCTCTGGCCGACG |
| | | CCCACCTGACCAAGAAGCTGCTGGACCTGGTGCAGCAGAGCTGCAA |
| | | CTACAAGCAGCTGAGAAAGGGCGCCAACGAGGCCACCAAGACCCTG |
| | | AACAGAGGCATCAGCGAGTTCATCGTGATGGCCGCCGACGCCGAGC |
| | | CTCTGGAGATCATCCTGCACCTGCCTCTGCTGTGCGAGGACAAGAAC |
| | | GTGCCTTACGTGTTCGTGAGAAGCAAGCAGGCCCTGGGCAGAGCCT |
| | | GCGGCGTGAGCAGACCTGTGATCGCCTGCAGCGTGACCATCAAGGA |
| | | GGGCAGCCAGCTGAAGCAGCAGATCCAGAGCATCCAGCAGAGCATC |
| | | GAGAGACTGCTGGTG |
| 73 | LARP7 (aa) | METESGNQEKVMEEESTEKKKEVEKKKRSRVKQVLADIAKQVDFWFG |
| | | DANLHKDRFLREQIEKSRDGYVDISLLVSFNKMKKLTTDGKLIARALRS |
| | | SAVVELDLEGTRIRRKKPLGERPKDEDERTVYVELLPKNVNHSWIERVF |
| | | GKCGNVVYISIPHYKSTGDPKGFAFVEFETKEQAAKAIEFLNNPPEEAPR |
| | | KPGIFPKTVKNKPIPALRVVEEKKKKKKKKGRMKKEDNIQAKEENMDT |
| | | SNTSISKMKRSRPTSEGSDIESTEPQKQCSKKKKKRDRVEASSLPEVRTG |
| | | KRKRSSSEDAESLAPRSKVKKIIQKDIIKEASEASKENRDIEISTEEEKDT |
| | | GDLKDSSLLKTKRKHKKKHKERHKMGEEVIPLRVLSKSEWMDLKKEY |
| | | LALQKASMASLKKTISQIKSESEMETDSGVPQNTGMKNEKTANREECR |
| | | TQEKVNATGPQFVSGVIVKIISTEPLPGRKQVRDTLAAISEVLYVDLLEG |
| | | DTECHARFKTPEDAQAVINAYTEINKKHCWKLEILSGDHEQRYWQKIL |
| | | VDRQAKLNQPREKKRGTEKLITKAEKIRLAKTQQASKHIRFSEYD |
| 74 | LARP7 (nt) | ATGGAGACCGAGAGCGGCAACCAGGAGAAGGTGATGGAGGAGGAG |
| | | AGCACCGAGAAGAAGAAGGAGGTGGAGAAGAAGAAGAGAAGCAG |
| | | AGTGAAGCAGGTGCTGGCCGACATCGCCAAGCAGGTGGACTTCTGG |
| | | TTCGGCGACGCCAACCTGCACAAGGACAGATTCCTGAGAGAGCAGA |
| | | TCGAGAAGAGCAGAGACGGCTACGTGGACATCAGCCTGCTGGTGAG |
| | | CTTCAACAAGATGAAGAAGCTGACCACCGACGGCAAGCTGATCGCC |
| | | AGAGCCCTGAGAAGCAGCGCCGTGGTGGAGCTGGACCTGGAGGGCA |
| | | CCAGAATCAGAAGAAAGAAGCCTCTGGGCGAGAGACCTAAGGACG |
| | | AGGACGAGAGAACCGTGTACGTGGAGCTGCTGCCTAAGAACGTGAA |
| | | CCACAGCTGGATCGAGAGAGTGTTCGGCAAGTGCGGCAACGTGGTG |
| | | TACATCAGCATCCCTCACTACAAGAGCACCGGCGACCCTAAGGGCTT |
| | | CGCCTTCGTGGAGTTCGAGACCAAGGAGCAGGCCGCCAAGGCCATC |
| | | GAGTTCCTGAACAACCCTCCTGAGGAGGCCCCTAGAAAGCCTGGCA |
| | | TCTTCCCTAAGACCGTGAAGAACAAGCCTATCCCTGCCCTGAGAGTG |
| | | GTGGAGGAGAAGAAGAAGAAGAAGAAGAAGAAGGGCAGAATGAA |
| | | GAAGGAGGACAACATCCAGGCCAAGGAGGAGAACATGGACACCAG |
| | | CAACACCAGCATCAGCAAGATGAAGAGAAGCAGACCTACCAGCGA |
| | | GGGCAGCGACATCGAGAGCACCGAGCCTCAGAAGCAGTGCAGCAA |
| | | GAAGAAGAAGAAGAGAGACAGAGTGGAGGCCAGCAGCCTGCCTGA |
| | | GGTGAGAACCGGCAAGAGAAAGAGAAGCAGCAGCGAGGACGCCGA |
| | | GAGCCTGGCCCCTAGAAGCAAGGTGAAGAAGATCATCCAGAAGGAC |
| | | ATCATCAAGGAGGCCAGCGAGGCCAGCAAGGAGAACAGAGACATC |
| | | GAGATCAGCACCGAGGAGGAGAAGGACACCGGCGACCTGAAGGAC |
| | | AGCAGCCTGCTGAAGACCAAGAGAAAGCACAAGAAGAAGCACAAG |
| | | GAGAGACACAAGATGGGCGAGGAGGTGATCCCTCTGAGAGTGCTGA |
| | | GCAAGAGCGAGTGGATGGACCTGAAGAAGGAGTACCTGGCCCTGCA |
| | | GAAGGCCAGCATGGCCAGCCTGAAGAAGACCATCAGCCAGATCAAG |
| | | AGCGAGAGCGAGATGGAGACCGACAGCGGCGTGCCTCAGAACACC |
| | | GGCATGAAGAACGAGAAGACCGCCAACAGAGAGGAGTGCAGAACC |
| | | CAGGAGAAGGTGAACGCCACCGGCCCTCAGTTCGTGAGCGGCGTGA |
| | | TCGTGAAGATCATCAGCACCGAGCCTCTGCCTGGCAGAAAGCAGGT |
| | | GAGAGACACCCTGGCCGCCATCAGCGAGGTGCTGTACGTGGACCTG |
| | | CTGGAGGGCGACACCGAGTGCCACGCCAGATTCAAGACCCCTGAGG |
| | | ACGCCCAGGCCGTGATCAACGCCTACACCGAGATCAACAAGAAGCA |
| | | CTGCTGGAAGCTGGAGATCCTGAGCGGCGACCACGAGCAGAGATAC |
| | | TGGCAGAAGATCCTGGTGGACAGACAGGCCAAGCTGAACCAGCCTA |
| | | GAGAGAAGAAGAGAGGCACCGAGAAGCTGATCACCAAGGCCGAGA |
| | | AGATCAGACTGGCCAAGACCCAGCAGGCCAGCAAGCACATCAGATT |
| | | CAGCGAGTACGAC |
| 187 | L7Ae | MYVRFEVPEDMQNEALSLLEKVRESGKVKKGTNETTKAVERGLAKLV |
| | | YIAEDVDPPEIVAHLPLLCEEKNVPYIYVKSKNDLGRAVGIEVPCASAAI |
| | | INEGELRKELGSLVEKIKGLQKRSHMHLE |
| 188 | Snu13 | MTEADVNPKAYPLADAHLTKKLLDLVQQSCNYKQLRKGANEATKTLN |
| | | RGISEFIVMAADAEPLEIILHLPLLCEDKNVPYVFVRSKQALGRACGVSR |
| | | PVIACSVTIKEGSQLKQQIQSIQQSIERLLV |

### Repressor Binding Element

In some embodiments of the ON and/or OFF systems disclosed herein, the first polynucleotide of the ON and/or OFF dual polynucleotide system has a repressor binding element. A repressor binding element comprises a sequence, e.g., a DNA or RNA sequence, which is bound, e.g., recognized by a repressor described elsewhere in this disclosure. In some embodiments, the repressor binds to a sequence comprising the binding element, or a fragment thereof. In some embodiments, the repressor binds to a structure comprising the binding element, or a fragment thereof.

In some embodiments, the composition or system comprises a repressor that binds to e.g., recognizes, the repressor binding element of the first polynucleotide in a dual polynucleotide system.

In some embodiments, the repressor binding element of the first polynucleotide is situated upstream (5′) or downstream (3′), or in the open reading frame of the sequence encoding the polypeptide.

In some embodiments, the repressor binding element of the first polynucleotide is situated upstream (5′) or downstream (3′) of a 5′ UTR of the first polynucleotide. In some embodiments, the binding element of the first polynucleotide is situated upstream (5′) or downstream (3′) of a 3′ UTR of the first polynucleotide. In some embodiments, the repressor binding element of the first polynucleotide is situated in the 5′ UTR of the first polynucleotide. In some embodiments, the repressor binding element of the first polynucleotide is situated downstream of a 3′ UTR of the first polynucleotide. In some embodiments, the repressor binding element of the first polynucleotide is situated adjacent, e.g., next to, a Poly A tail.

In some embodiments, the repressor binding element is MS2. In some embodiments, the repressor binding element is PP7. In some embodiments, the repressor binding element is BoxB. In some embodiments, the repressor binding element is U1A hairpin. In some embodiments, the repressor binding element is PRE. In some embodiments, the repressor binding element is PRE2. In some embodiments, the repressor binding element is a kink-turn forming sequence. In some embodiments, the repressor binding element is 7SK. In some embodiments, the repressor binding element is an RNA sequence/structure element that binds to a protein.

In some embodiments, when the binding element is MS2 (e.g., wildtype MS2, or a variant or fragment thereof) the repressor is MBP (e.g., wildtype MBP, a variant or fragment thereof).

In some embodiments, when the repressor binding element is PP7 (e.g., wildtype PP7, or a variant or fragment thereof) the repressor is PCP (e.g., wildtype PCP, or a variant or fragment thereof). PP7 can comprise the sequence of any one of the PP7 and variants thereof described in Lim F, and Peabody D S. Nucleic Acids Res. 2002; 30(19):4138-4144, and U.S. Pat. No. 9,365,831, incorporated by reference herein in its entirety.

In some embodiments, when the repressor binding element is BoxB (e.g., wildtype BoxB, or a variant or fragment thereof) the repressor is Lambda N (e.g., wildtype Lambda N, or a variant or fragment thereof).

In some embodiments, when the repressor binding element is U1A hairpin (e.g., wildtype U1A hairpin, or a variant or fragment thereof) the repressor is U1A (e.g., wildtype U1A, or a variant or fragment thereof).

In some embodiments, when the repressor binding element is PRE (e.g., wildtype PRE, or a variant or fragment thereof) the repressor is PUF (e.g., wildtype PUF, or a variant or fragment thereof).

In some embodiments, when the repressor binding element is a kink-turn forming sequence the repressor is 15.5kd (e.g., wildtype 15.5kd, or a variant or fragment thereof).

In some embodiments, when the repressor binding element is a 7SK sequence repressor is LARP7 (e.g., wildtype LARP7, or a variant or fragment thereof).

In some embodiments, the repressor binding element comprises a sequence comprising 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the repressor binding element comprises a sequence comprising about 5-100, about 5-90, about 5-80, about 5-70, about 5-60, about 5-50, about 5-40, about 5-30, about 5-25, about 5-20, about 5-19, about 5-18, about 5-17, about 5-16, about 5-15, about 5-14, about 5-13, about 5-12, about 5-11, about 5-10, about 5-9, about 5-8, about 5-7 or about 5-6 nucleotides. In some embodiments, the repressor binding element comprises a sequence comprising about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the repressor binding element comprises a sequence comprising about 5-100, about 6-90, about 7-80, about 8-70, about 9-60, about 10-50, about 11-40, about 12-30, about 13-25, about 14-24, about 15-23, about 16-22, about 17-21, or about 18-20 nucleotides. In some embodiments, the repressor binding element comprises a sequence comprising 19 nucleotides.

In some embodiments, the repressor binding element comprises a binding element nucleotide sequence provided in Table 4 or a sequence with at least 80%, 85%, 90%, 95%, 96%, 97%, 98%, 99% or 100% identity thereof. In some embodiments, the repressor binding element comprises a binding element sequence provided in SEQ ID NO: 1, SEQ ID NO: 2, SEQ ID NO: 3, or a sequence with at least 80%, 85%, 90%, 95%, 96%, 97%, 98%, 99% or 100% identity thereof.

In some embodiments of any of the compositions, systems, methods or uses disclosed herein, the repressor binding element comprises at least 1, 2, 3, 4, 5, 6, 7, 8, 9, 10, 11, 12, 13, 14, 15, 20 or 30 repeats of the sequence bound by the second polypeptide.

In some embodiments, the binding element comprises no more than 80, 70, 60, 50, 40 or 30 repeats of the sequence bound by the second polypeptide. In some embodiments, the repressor binding element comprises about 1-30, about 1-20, about 1-10, about 1-9, about 1-8, about 1-7, about 1-6, about 1-5, about 1-4, about 1-3, or about 1-2 repeats of the sequence bound by the second polypeptide. In some embodiments, the repressor binding element comprises about 1-30, about 2-30, about 3-30, about 4-30 about, 5-30 about, 6-30, about 7-30, about 8-30, about 9-30, about 10-30, about 11-30, about 12-30, about 13-30, about 14-30, about 15-30, or about 20-30 repeats of the sequence bound by the second polypeptide. In some embodiments, the repressor binding element comprises about 1-30, about 2-20, about 3-15, about 4-14, about 5-13, about 6-12, about 7-11, or about 8-10 repeats of the sequence bound by the second polypeptide. In some embodiments, the repressor binding element comprises 6 repeats of the sequence bound by the second polypeptide.

In some embodiments of any of the compositions, systems, methods or uses disclosed herein, each repeat is separated by a spacer sequence comprising 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the spacer sequence comprises about 1-100, about 1-90, about 1-80, about 1-70, about 1-60, about 1-50, about 1-40, about 1-30, about 1-25, about 1-20, about 1-19, about 1-18, about 1-17, about 1-16, about 1-15, about 1-14, about 1-13, about 1-12, about 1-11, about 1-10, about 1-9, about 1-8, about 1-7, about 1-6, about 1-5, about 1-4, about 1-3, or about 1-2 nucleotides. In some embodiments, the spacer sequence comprises about 1-100, about 2-100, about 3-100, about 4-100, about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the spacer sequence comprises about 1-100, about 2-90, about 3-80, about 4-70, about 5-60, about 6-50, about 7-40, about 8-40, about 9-30, about 10-25, about 11-24, about 12-23, about 13-22, about 14-21, about 15-20, about 16-19, about 17-18 nucleotides. In some embodiment, the spacer sequence comprises 20 nucleotides.

In some embodiments, the spacer sequence comprises a spacer sequence provided in Table 1 or a sequence with at least 80%, 85%, 90%, 95%, 96%, 97%, 98%, 99% or 100% identity thereof.

In some embodiments, the repressor binding element is an MS2 dimer which comprises monomers linked by a linker sequence. The linker sequence could be any peptide sequence known to link two protein sequences, including but not limited to those known in the art (See, e.g., Chen et al., Adv Drug Deliv Rev(2013) October 15; 65(10): 1357-1369).

TABLE 4
Exemplary sequences of a repressor binding element

| | | |
|---|---|---|
| SEQ ID | | |
| NO | Sequence | Sequence |

3′UTR sequences

| | | |
|---|---|---|
| 1 | 3′v1.1_6xMS2 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCCC |
| | | CGUGGUCUUUGAAUAAAGUCUGAGUGGGCGGCCAGGUAUGAU |
| | | UACAACCUGUGCUACUUUAGAUGACUAUUGAAAGACCAUUAG |
| | | GCUUUCUAACGGACGCUCACGAGAGAGGGGCACCAAUAGGGCC |
| | | CCACGGCACUUCAAAGGUUUGGCUUGGAGUAGUAACCCAAGCA |
| | | GCAACAGUUUUGACUUUCGGACCACCAUCAGGGGUCCCACGUU |
| | | GGGAACACGUAACUCUCCUACUAACAAGAGGAG |
| 2 | 3′6xMS2_3′v1.1 | UGAUAAUAGCAGGUAUGAUUACAACCUGUGCUACUUUAGAUG |
| | | ACUAUUGAAAGACCAUUAGGCUUUCUAACGGACGCUCACGAGA |
| | | GAGGGGCACCAAUAGGGCCCCACGGCACUUCAAAGGUUUGGCU |
| | | UGGAGUAGUAACCCAAGCAGCAACAGUUUUGACUUUCGGACCA |
| | | CCAUCAGGGGUCCCACGUUGGGAACACGUAACUCUCCUACUAA |
| | | CAAGAGGAGGCUGGAGCCUCGGUGGCCUAGCUUCUUGCCCCUU |
| | | GGGCCUCCCCCCAGCCCCUCCUCCCCUUCCUGCACCCGUACCCC |
| | | CGUGGUCUUUGAAUAAAGUCUGAGUGGGCGGC |
| 3 | 3′_6xMS2 | UGAUAAUAGCAGGUAUGAUUACAACCUGUGCUACUUUAGAUG |
| | (MS2 sequences | ACUAUUGAAAGACCAUUAGGCUUUCUAACGGACGCUCACGAG |
| | in bold and | AGAGGGGCACCAAUAGGGCCCCACGGCACUUCAAAGGUUUGG |
| | underline) | CUUGGAGUAGUAACCCAAGCAGCAACAGUUUUGACUUUCGGA |
| | | CCACCAUCAGGGGUCCCACGUUGGGAACACGUAACUCUCCUA |
| | | CUAACAAGAGGAG |
| 4 | 3′v1.1 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCCC |
| | | CGUGGUCUUUGAAUAAAGUCUGAGUGGGCGGC |

5′ UTR sequence*

| | | |
|---|---|---|
| 6 | 5′ v1.1 | GGGAAAUAAGAGAGAAAAGAAGAGUAAGAAGAAAUAUAAGAC |
| | | CCCGGCGCCGCCACC |
| 7 | 5′kink turn (5′kt) | GGAUCCGUGAUCGGAAACGUGAGAUCCACCUCAGAUCCGCUAG |
| | | GACACCCGCAGAUCGAGAAGAAGGCGAAUUAAGAGAGAAAAG |
| | | AAGAGUA AGAAGAAAUAUAAGACACCGGUCGC CACC |
| 8 | 5′_6xMS2 | GGGAAACGGCAAGCAGCAGCUGCCGCUGUUUUGUCUUAACCGG |
| | (underlined | UUGAAAGACCAUUAGGCUUUCUAACGGACGCUCACGAGAGAG |
| | sequence indicates | GGGCACCAAUAGGGCCCCACGGCACUUCAAAGGUUUGGCUUGG |
| | the region MBP | AGUAGUAACCCAAGCAGCAACAGUUUUGACUUUCGGACCACCA |
| | binds to) | UCAGGGGUCCCACGUUGGGAACACGUAACUCUCCUACUAACAA |
| | | GAGGAGCCCCGGCGCCGCCACC |
| 9 | PRE_p2 | GGGAAAUAAGAGAGAAAAAGAGAGAGGGAGAGGAGAAGAGUA |
| | | AGAAGAAAUAUAAGACCCCGGCGCCGCCACC |
| 10 | PRE_p3 | GGGAAAUAAGAGAGAAAAGAAGAGUAAGAAAGAGAGAGGGAG |
| | | AGGAGAAAUAUAAGACCCCGGCGCCGCCACC |
| 11 | PRE_p4 | GGGAAAUAAGAGAGAAAAGAAGAGUAAGAAGAAAUAUAA |
| | | GACAGAGAGAGGGAGAGGACCCGGCGCCGCCACC |
| 12 | PRE2_p1_3x | GGGAAAUUUUUUUUUGAUAUUAUAAGAGUUUUUUUUUGA |
| | | UAUUAAGAAAAUUUUUUUUUGAUAUUAGAAGAGUAAGAAGAA |
| | | AUAUAAGACCCCGGCGCCGCCACC |
| 13 | PRE2_p2_3x | GGGAAAUAAGAGUUUUUUUUUGAUAUUAAGAAAAUUUUU |
| | | UUUUGAUAUUAGAAGAGUUUUUUUUUGAUAUUAUAAGAAGAA |
| | | AUAUAAGACCCCGGCGCCGCCACC |
| 14 | PRE2_p3_3x | GGGAAAUAAGAGAGAAAAUUUUUUUUUGAUAUUAGAAGA |
| | | GUUUUUUUUUGAUAUUAUAAGAAUUUUUUUUUGAUAUUAGAA |
| | | AUAUAAGACCCCGGCGCCGCCACC |
| 15 | PRE2_p4_3x | GGGAAAUAAGAGAGAAAAGAAGAGUUUUUUUUUGAUAUU |
| | | AUAAGAAUUUUUUUUUGAUAUUAGAAAUAUUUUUUUUUGAUA |
| | | UUAUAAGACCCCGGCGCCGCCACC |
| 16 | PRE2_p5_3x | GGGAAAUAAGAGAGAAAAGAAGAGUAAGAAUUUUUUUUU |
| | | GAUAUUAGAAAUAUUUUUUUUUGAUAUUAUAAGACUUUUUUU |
| | | UUGAUAUUACCCGGCGCCGCCACC |
| 17 | PRE2_p6_3x | GGGAAAUAAGAGAGAAAAGAAGAGUAAGAAGAAAUAUUU |
| | | UUUUUUGAUAUUAUAAGACUUUUUUUUUGAUAUUACCCGGCU |
| | | UUUUUUUUGAUAUUAGCCGCCACC |

Miscellaneous sequences

| | | |
|---|---|---|
| 18 | BoxB | GGGCCCUGAAGAAGGGCCC |
| 19 | U1A hairpin | AAUCCAUUGCACUCCGGAUUU |
| 20 | 7SK | GGAUGUGUGGCCAGUUGGUGCUGUCUGUCUUCUUGGUGAC |
| | | CAGGAGUAAUUUGGACCAUCUGGGAAACCAGACCUCUUUUCAC |
| | | CCAGGCUGUUUCGUGUGCAUCACUUCUGAGGCUGUGUGCUCAG |
| | | UCAAGACCUUGCCAGAGAGUUGGGGGAUUAGCCUUAGGUCGA |
| | | GAAUAUUCUGAGCAGUAGUAAGAAAUUUAAAAAUCAUCCAUA |
| | | AUUUCAUCACUCUUAUUUUAAGAGAUUAGUUUAU |

*underlined sequence indicates the region the RBP binds to

miRNA Target Site

In some embodiments of the ON and/or OFF systems disclosed herein, the polynucleotide of the single polynucleotide system, the second polynucleotide of the ON and/or OFF dual polynucleotide system, and optionally the first polynucleotide of the ON and/or OFF dual polynucleotide system has one or more microRNA target sites (miRts). In some embodiments, the polynucleotide of the single polynucleotide system has one or more microRNA target sites (miRts). In some embodiments, the first polynucleotide of the dual polynucleotide system has one or more miRts. In some embodiments, the second polynucleotide of the dual polynucleotide system has one or more miRts. In some embodiments, the one or more miRts is on the second polynucleotide but not on the first polynucleotide of the dual polynucleotide system. In some embodiments, the one or more miRts is on the first polynucleotide and on the second polynucleotide of the dual polynucleotide system.

In some embodiments, the one or more miRts are situated on the polynucleotide (e.g., any of the polynucleotides of the single or dual polynucleotide systems) upstream (5′) or downstream (3′) of the open reading frame encoding the polypeptide (i.e. the target protein) or the repressor. In some embodiments, the one or more miRts are situated on the polynucleotide downstream of the open reading frame encoding the polypeptide or the repressor (e.g., in the 3′ UTR). In some embodiments, the one or more miRts are situated on the polynucleotide upstream of the open reading frame encoding the polypeptide or the repressor (e.g., in the 5′ UTR). In some embodiments, the one or more miRts are situated on the polynucleotide between the repressor binding site and upstream of the open reading frame encoding the polypeptide. In some embodiments, the one or more miRts are situated on the polynucleotide downstream of the poly A tail.

In some embodiments, the one or more miRts are situated on the second polynucleotide upstream (5′) of the open reading frame encoding the repressor. In some embodiments, the one or more miRts are situated on the second polynucleotide downstream (3′) of the open reading frame encoding the repressor. In some embodiments, the one or more miRts are situated on the second polynucleotide between the open reading frame encoding the repressor and upstream of the poly A tail.

In some embodiments, the one or more miRts are in a non-coding region of the polynucleotide. In some embodiments, the 3′ untranslated region (UTR) of the polynucleotide (e.g., an mRNA) comprises at least one miRts (e.g., an HSPC miRts). In some embodiments, the 3′ UTR of the mRNA comprises at least two repeats of one miRts (e.g., one HSPC miRts). In some embodiments, the 3′ UTR of the mRNA comprises six repeats of one miRts (e.g., one HSPC miRts).

In some embodiments, the 5′ UTR of the polynucleotide (e.g., an mRNA) comprises at least one miRts. In some embodiments, the 5′ UTR of the polynucleotide (e.g., an mRNA) comprises at least two repeats of one miRts. In some embodiments, the 5′ UTR of the polynucleotide (e.g., an mRNA) comprises three repeats of one miRts. In some embodiments, the polynucleotide (e.g., an mRNA) has one or more miRts in the 3′ UTR and the 5′ UTR.

In some embodiments, the miRNA target sequence is 100% complementary to the miRNA and selected from, but not limited to, the sequences in Table 5 below:

TABLE 5a
miRNA target sequences

| SEQ | | |
|---|---|---|
| ID | Sequence | |
| NO | information | Sequence |
| 75 | miR126-3p | CGCAUUAUUACUCACGGUACGA |
| 76 | miR-130a-3p | AUGCCCUUUUAACAUUGCACUG |
| 77 | miR-10a-5p | CACAAAUUCGGAUCUACAGGGUA |
| 78 | miR-29a-3p | UAACCGAUUUCAGAUGGUGCUA |
| 79 | miR125a-5p | UCACAGGUUAAAGGGUCUCAGGGA |
| 80 | miR125b-5p | AGCUCCCAAGAGCCUAACCCGU |
| 189 | miR196b-5p | CCCAACAACAGGAAACUACCUA |
| 190 | miR150-5p | CACUGGUACAAGGGUUGGGAGA |
| 191 | miR142-3p | UCCAUAAAGUAGGAAACACUACA |
| 192 | miR122-5p | CAAACACCAUUGUCACACUCCA |
| 193 | miR223-5p | UGGGGUAUUUGACAAACUGACA |

For example, the microRNA target site is a miR122 target site or a miR142 target site.

In some embodiments, the miRNA target sequence has mismatches anywhere from position 16 in the miRNA target sequence and o towards the 3′ end of miRNA. For instance, the miRNA target sequence is selected from, but not limited to, the sequences in Table below:

TABLE 5b
miRNA target sequences with mismatches

| SEQ | | |
|---|---|---|
| ID | Sequence | |
| NO | information | Sequence |
| 194 | miR126-3p | CUACAUAUUACUCACGGUACGA |
| 195 | miR-130a-3p | AUACACUUUUAACAUUGCACUG |
| 196 | miR150-5p | CUACAGUACAAGGGUUGGGAGA |
| 197 | miR142-3p | UUACAAAAGUAGGAAACACUACA |
| 198 | miR122-5p | CUACAACCAUUGUCACACUCCA |
| 199 | miR233-5p | UUACAUAUUUGACAAACUGACA |

microRNAs

In some embodiments of the ON and/or OFF systems disclosed herein, the expression of the polypeptide or repressor from the polynucleotides of the single or dual polynucleotide systems is influenced by the levels of one or more miRNAs present in the cell-type that comes in contact with the polynucleotide that has one or more microRNA target sites that are complementary to the one or more miRNAs.

In some embodiments, miRNAs of interest can be determined from miRNA databases as referenced in sources known in the art, e.g., Kozomara A, et al., Nucleic Acids Res 2019 47:D155-D162; Kozomara A, Griffiths-Jones S., Nucleic Acids Res 2014 42:D68-D73; Kozomara A, Griffiths-Jones S. Nucleic Acids Res 2011 39:D152-D157; Griffiths-Jones S, Saini H K, van Dongen S, Enright A J. Nucleic Acids Res 2008 36:D154-D158; Griffiths-Jones S, Grocock R J, van Dongen S, Bateman A, Enright A J. Nucleic Acids Res 2006 34:D140-D144; Griffiths-Jones S. Nucleic Acids Res 2004 32:D109-D111; Ambros V, et al., RNA 2003 9(3):277-279; and Meyers B C, et al., Plant Cell. 2008 20(12):3186-3190. A nucleotide sequence that is a reverse complement of a miRNA sequence selected from such a database would be the mi microRNA expression can vary across different hematopoietic cells and at different stages of development. For instance, miR142 is specific to cells of the hematopoietic lineage. miR-150 is abundantly expressed in mature immune cells.

In some instances, miRNAs of interest such as miR126-3p, miR130a-3p, miR125b-5p, miR196b-5p, and miR10a-5p are abundant in CD34+ bone marrow cells but not present at high levels in more differentiated immune cells where expression of a target is desired. miR126-3p, miR130a-3p, miR196b-5p are have been described as expressed in hematopoietic stem cells (HSCs) and early progenitors in both mice and humans, but not in more differentiated progeny. These miRs were shown to effectively repress reporter lentiviral vectors containing their target sites in both mouse and human hematopoietic stem and progenitor cells (HSPCs) (Gentner B. et al., Sci Trans. Med. 2010; 2(58) p. 58ra84).

In some instances, miR126-3p, miR130a-3p, and miR196b-5p, miR29a-3p, miR125b-5p, miR125a-5p, are known to be enriched in hematopoietic stem cells profiled in mouse and human stem cell and progenitor cell populations by RT-qPCR, bead-based miR detection, and miR arrays. See e.g., Petriv O. I. et al., Proc. Natl. Acad. Sci, 2010; 107 (35) 15443-15448; Chung S S, et al., Ther Adv Hematol. 2011 October; 2(5):317-34; Bissels U, et al., Haematologica. 2012 February; 97(2):160-7. Epub 2011 Nov. 4.).

### Modifications of Polynucleotides

In some embodiments of the ON and/or OFF systems disclosed herein, the design of the polynucleotides can be modified in order to increase efficacy of the outcome of miR target sites-miRNA interaction in an ON/OFF systems. For instance, a polynucleotide design modification described herein can enhance expression of the target protein in a desired cell type. In another instance, a polynucleotide design modification described herein can suppress expression of the target protein in a particular cell type, while enhancing expression of the target protein in another cell type.

In some embodiments, the polynucleotide of the system (e.g., an mRNA) has one or more of the following design modifications: (1) an AU-rich element; (2) the one or more miRts comprise at least one mismatch to the microRNA that binds the one or more miRts; (3) structurally accessible UTRs; (4) a short polyA tail; and (5) the ability to form microRNA bridges when a microRNA binds to the one or more miRts. The 3′ UTR comprises an AU-rich element, which can be 60%-90% AU-rich, for example, about 70% AU-rich. The one or more miRts comprise one to three mismatches to the microRNA that binds the one or more miRts.

In some embodiments, the polyA tail of any of the polynucleotides described herein is 40-100 nucleotides in length. In some embodiments, when one or more miRts are included in both the 5′ and the 3′ UTRs of the polynucleotide, a microRNA bridge is formed by the one or more miRts in the 5′ UTR and the 3′ UTR.

### Cell-Specific Expression

In some embodiments of the ON and/or OFF systems disclosed herein, the polypeptide and/or repressor of the ON and/or OFF compositions and systems are expressed in cells of the hematopoietic cell lineage. Cells of the hematopoietic cell lineage include, but are not limited to, hematopoietic stem cells (HSCs), hematopoietic stem and progenitor cells (HSPCs), mature bone marrow cells, multipotent progenitors (MPP), common lymphoid progenitors (CLP), granulocyte monocyte progenitors (GMP), common myeloid progenitors (CMP), and megakaryocyte-erythrocyte progenitors (MEP). In some embodiments, the expression of the polypeptide of interest is selectively turned OFF in HSCs. In some embodiments, the expression of the polypeptide of interest is selectively turned OFF in HSPCs. In some embodiments, the expression of the polypeptide of interest is selectively turned ON in mature immune cells (such as T cells, B cells, neutrophils, and macrophages). The selectivity of expression is the cell-type of interest is determined by the presence of the microRNAs in the particular cell type. For instance, miR142 is present in all immune cells. In an OFF system, when hematopoietic cells that express miR142 are exposed to target mRNA constructs with miR142ts, the expression of the target will be turned off in all those cells.

A “hematopoietic stem cell” or “HSC” is an immature cell that can develop into all types of blood cells, including white blood cells, red blood cells, and platelets. Hematopoietic stem cells are found in the peripheral blood and the bone marrow. The defining property of a HSC is its ability to reconstitute hematopoiesis following transplantation. “Hematopoietic stem and progenitor cells” “or “HSPCs” are a rare population of precursor cells that possess the capacity for self-renewal and multilineage differentiation. In the bone marrow (BM), HSPCs warrant blood cell homeostasis. In addition, they may also replenish tissue-resident myeloid cells and directly participate in innate immune responses once they home to peripheral tissues. See e.g., Schulz C, et al. Immunol Res. 2009; 44(1-3):160-8. HSPCs isolated from bone marrow have been successfully used for hematological transplantations.

The different types of blood cell and their lineage relationships are summarized in FIG. 1.3 in Janeway C A Jr, et al., Immunobiology: The Immune System in Health and Disease. 5th edition. New York: Garland Science; (2001). The myeloid progenitor is the precursor of the granulocytes, macrophages, dendritic cells, and mast cells of the immune system. The common lymphoid progenitor gives rise to the lymphocytes (T cells, and B cells). A third lineage of lymphoid cells, called natural killer cells, lack antigen-specific receptors and are part of the innate immune system.

In some embodiments, the CMP cells are determined to be CD45RA-CD123+/lo); GMP cells are CD45RA+ CD123+); MEP cells are CD45RA− CD123−; MPP cells are CD45+ CD34+ CD90− CD133+ CD45RA−); CLP cells are CD45+ CD34+CD90− CD10+ CD45RA_; and mature bone marrow cells are CD45+ Lin+.

A “mature immune cell” includes mature cells of the adaptive and/or innate immune systems. These include lymphocytes that mature in the bone marrow or thymus. Mature granulocytes include neutrophils, eosinophils, basophils, and mast cells, which all develop from GMP. Fang, P., et al. J Hematol Oncol 11, 97 (2018)

In some embodiments, expression of certain immune therapies in HSCs may not be desirable e.g. targets that may induce monocyte differentiation or macrophage polarization whose expression in HSCs may reprogram bone marrow development. For such immune therapies, an miR-OFF system disclosed herein is desirable to limit off-target effects in HSCs/HSPCs. In some embodiments, an miR-ON system disclosed herein is desirable for in vivo gene editing of HSPCs, thereby allowing editing only in cells of interest, (e.g., HSCs), while reducing expression of a gene editor in all other cells.

In some embodiments, endogenous miRNA in specific cell types can be harnessed at multiple levels to allow cell-type-selective gene editing from systemic delivery of editing components. For instance, the disclosed systems can be used to control protein output, protein stability, or control guide RNA activation in specific cells. An HSC miR-ON system can be used to preferentially allow mRNA encoding an editor to express protein appreciably in HSC cells only. In another instance, using self-splicing inteins, an editor protein can be fused to a degron domain in most cells that leads to rapid protein degradation. The degron domain expression can be turned OFF in select cells only (eg. HSC cells here) to allow maintenance of the editor protein. In another instance, single guide RNA can be embedded between miR-target-sites to allow miR-dependent cleavage and selective activation in miR-containing cells only.

In one instance of the HSC miR-ON system, the system can be used to bias expression of target proteins in HSCs only. In one instance, the system can be used to selectively kill HSCs as a replacement of chemotherapy/radiation for bone marrow conditioning prior to stem cell transplant.

### Methods of Using the Systems or Compositions

The present disclosure provides compositions, which can be delivered to cells, e.g., target cells, e.g., in vitro or in vivo. For in vitro protein expression, the cell is contacted with the composition by incubating the composition and the cell ex vivo. Such cells may subsequently be introduced in vivo. For in vivo protein expression, the cell is contacted with the composition by administering the composition to a subject to thereby induce protein expression in or on the desired cells within the subject. For example, in one embodiment, the composition is administered intravenously. In another embodiment, the composition is administered intramuscularly. In yet other embodiment, the composition is administered by a route selected from the group consisting of subcutaneously, intranodally and intratumorally.

For in vitro delivery, in one embodiment the cell is contacted with the composition by incubating the composition and the target cell ex vivo. In one embodiment, the cell is a human cell. Various types of cells have been demonstrated to be transfectable by the composition (e.g., the LNP).

In another embodiment, the cell is contacted with the composition for, e.g., at least 30 minutes, at least 1 hour, at least 2 hours, at least 3 hours, at least 4 hours, at least 5 hours, at least 6 hours, at least 12 hours or at least 24 hours.

In one embodiment, the cell is contacted with the composition for a single treatment/transfection. In another embodiment, the cell is contacted with the composition for multiple treatments/transfections (e.g., two, three, four or more treatments/transfections of the same cells).

In another embodiment, for in vivo delivery, the cell is contacted with the composition by administering the composition to a subject to thereby deliver the polynucleotide(s) to cells within the subject. For example, in one embodiment, the composition is administered intravenously. In another embodiment, the composition is administered intramuscularly. In yet other embodiment, the composition is administered by a route selected from the group consisting of subcutaneously, intranodally and intratumorally.

In an aspect, provided herein is a method of expressing a polypeptide in a cell, comprising administering to the cell a composition disclosed herein.

In a related aspect, provided herein is a composition or system for use in a method of expressing a polypeptide in a cell in a cell.

In another aspect, the disclosure provides a method of expressing a polypeptide in a cell in a subject, comprising administering to the subject an effective amount of a composition disclosed herein.

In a related aspect, provided herein is a composition or system for use in a method of expressing a polypeptide in a cell in a subject.

In yet another aspect, provided herein is a method of delivering a composition disclosed herein.

In a related aspect, provided herein is a composition or system for use in a method of delivering the composition to a cell.

In an embodiment, the method or use, comprises contacting the cell in vitro, in vivo or ex vivo with the composition or system.

In an embodiment, the composition or system formulated as an LNP, a liposome composition, a lipoplex composition, or a polyplex composition of the present disclosure is contacted with cells, e.g., ex vivo or in vivo and can be used to deliver a secreted polypeptide, an intracellular polypeptide, a transmembrane polypeptide, or peptides, polypeptides or biologically active fragments thereof to a subject.

In an aspect, the disclosure provides a method of delivering a composition or system disclosed herein to a subject having a disease or disorder, e.g., as described herein.

In a related aspect, provided herein is a composition or system for use in a method of delivering the composition or system to a subject having a disease or disorder, e.g., as described herein.

In another aspect, provided herein is a method of modulating an immune response in a subject, comprising administering to the subject in need thereof an effective amount of a composition or system disclosed herein.

In a related aspect, provided herein is a composition or system for use in a method of modulating an immune response in a subject, comprising administering to the subject an effective amount of the composition or system.

In another aspect, provided herein is a method of delivering a secreted polypeptide, an intracellular polypeptide, a transmembrane polypeptide, or peptides, polypeptides or biologically active fragments thereof to a subject.

In an aspect, provided herein is a method of treating, preventing, or preventing a symptom of, a disease or disorder comprising administering to a subject in need thereof an effective amount of a composition or system disclosed herein.

In a related aspect, provided herein is a composition or system for use in a method of treating, preventing, or preventing a symptom of, a disease or disorder in a subject, comprising administering to the subject in need thereof an effective amount of the composition or system.

In an embodiment, the first polynucleotide and/or the second polynucleotide of the system is formulated as an LNP. In an embodiment, the first polynucleotide of the system is formulated as an LNP. In an embodiment, the second polynucleotide of the system is formulated as an LNP. In an embodiment, both the first and the second polynucleotides of the system are formulated as LNPs.

In an embodiment, the LNP comprising the first polynucleotide is the same as the LNP comprising the second polynucleotide. In an embodiment, the LNP comprising the first polynucleotide is different from the LNP comprising the second polynucleotide.

In an embodiment, the LNP comprising the first polynucleotide is in a composition. In an embodiment, the LNP comprising the second polynucleotide is in a separate composition. In an embodiment, the LNP comprising the first polynucleotide and the LNP comprising the second polynucleotide are in the same composition. In some embodiments, the first and second polynucleotides are in separate dosage forms packaged together. In some embodiments, the first and second polynucleotides are in a unit dosage form.

In an embodiment, the LNP comprising the first polynucleotide and the LNP comprising the second polynucleotide are administered simultaneously, e.g., substantially simultaneously. In some embodiments, the LNP comprising the first polynucleotide and the LNP comprising the second polynucleotide are co-delivered.

In an embodiment, the LNP comprising the first polynucleotide and the LNP comprising the second polynucleotide are administered sequentially.

In an embodiment, the LNP comprising the first polynucleotide is administered first.

In an embodiment, the LNP comprising the first polynucleotide is administered first followed by administration of the LNP comprising the second polynucleotide.

In an embodiment, the LNP comprising the second polynucleotide is administered first.

In an embodiment, the LNP comprising the second polynucleotide is administered first followed by administration of the LNP comprising the first polynucleotide.

In some embodiments of the disclosed methods, the method comprises contacting the cell with a composition of the disclosure (for example a composition according to FIGS. 1A-1B), wherein the cell expresses a microRNA or endonuclease that binds to the recognition site or cleavage site and reduces translation of the repressor from the second polynucleotide. In some embodiments, the method comprising contacting the cell with a composition of the disclosure, wherein the cell expresses a microRNA or endonuclease that binds to the recognition site or cleavage site, cleaves the repressor binding element from the first polynucleotide, and enhances translation of the first polypeptide from the first polynucleotide.

In some embodiments of the disclosed methods, the method comprises contacting the cell with (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site or cleavage site, wherein the cell expresses a microRNA or an endonuclease that binds to the recognition site or cleavage site and reduces translation of the repressor from the second polynucleotide.

In some embodiments of the disclosed methods, the method comprises expressing a polypeptide in a cell, the method comprising contacting the cell with (a) a first polynucleotide comprising (i) a repressor binding element, (ii) a recognition site or cleavage site, and (iii) an open reading frame encoding a polypeptide; and (b) a second polynucleotide comprising a sequence encoding a repressor that binds to the repressor binding element, wherein the cell expresses a microRNA or endonuclease that binds to the recognition site or cleavage site, cleaves the repressor binding element from the first polynucleotide, and enhances translation of the polypeptide from the first polynucleotide.

In some embodiments of the disclosed methods, the method comprises expressing a polypeptide in a cell in a subject, the method comprising administering to the subject: (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site or cleavage site, wherein the cell expresses a microRNA or an endonuclease that binds to the recognition site or cleavage site and reduces translation of the repressor from the second polynucleotide.

In some embodiments of the disclosed methods, the method comprises expressing a polypeptide in a cell in a subject, the method comprising administering to the subject: (a) a first polynucleotide comprising (i) a repressor binding element, (ii) a recognition site or cleavage site, and (iii) an open reading frame encoding a polypeptide; and (b) a second polynucleotide comprising a sequence encoding a repressor that binds to the repressor binding element, wherein the cell expresses a microRNA or endonuclease that binds to the recognition site or cleavage site, cleaves the repressor binding element from the first polynucleotide, and enhances translation of the polypeptide from the first polynucleotide.

### Sequence Optimization and Methods Thereof

In some embodiments, a polynucleotide of the disclosure comprises a sequence-optimized nucleotide sequence encoding a polypeptide disclosed herein, e.g., a polynucleotide encoding a polypeptide (e.g., a therapeutic or prophylactic protein), or a repressore. In some embodiments, the polynucleotide of the disclosure comprises an open reading frame (ORF) encoding a polypeptide or a repressor, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics. In some embodiments, the percentage of uracil or thymine nucleobases in a sequence-optimized nucleotide sequence (e.g., encoding a polypeptide or a repressor, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response in desired cells and/or microenvironments when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine (T) is present instead of uracil (U), and one would substitute T where U appears. For RNA it is recognized that uracil (U) is present instead of thymine (T). One of skill in the art could readily obtain an RNA sequence when the DNA sequence is provided by substituting thymine in the DNA sequence to uracil. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence encoding a polypeptide, or a repressor of the disclosure has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding a polypeptide, or a repressor of the disclosure has a reduced number of uracil triplets (UUU) with respect to the wild-type nucleic acid sequence. In some embodiments, a uracil-modified sequence encoding a polypeptide, or a repressor has a reduced number of uracil pairs (UU) with respect to the number of uracil pairs (UU) in the wild-type nucleic acid sequence. In some embodiments, a uracil-modified sequence encoding olypeptide, or a repressor of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence”, refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence encoding a polypeptide, or a repressor has a % UUwt between below 100%.

In some embodiments, the polynucleotide of the disclosure comprises a uracil-modified sequence encoding an encoding a polypeptide, or a repressor disclosed herein. In some embodiments, the uracil-modified sequence encoding a polypeptide, or a repressor comprises at least one chemically modified nucleobase, e.g., 5-methoxyuracil. In some embodiments, at least 95% of a nucleobase (e.g., uracil) in a uracil-modified sequence encoding a polypeptide, or a repressor of the disclosure are modified nucleobases. In some embodiments, at least 95% of uracil in a uracil-modified sequence encoding a polypeptide, or a repressor is 5-methoxyuracil.

In some embodiments, a polynucleotide of the disclosure (e.g., a polynucleotide comprising a nucleotide sequence encoding a polypeptide, or a repressor (e.g., the wild-type sequence, functional fragment, or variant thereof) is sequence optimized.

A sequence optimized nucleotide sequence (nucleotide sequence is also referred to as “nucleic acid” herein) comprises at least one codon modification with respect to a reference sequence (e.g., a wild-type sequence encoding a polypeptide, or a repressor. Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence optimized nucleic acids (e.g., a RNA, e.g., an mRNA) can be administered to a subject in need thereof to facilitate in vivo expression of functionally active encoding a polypeptide, or a repressor.

Additional and exemplary methods of sequence optimization are disclosed in International PCT application WO 2017/201325, filed on 18 May 2017, the entire contents of which are hereby incorporated by reference.

Micro RNA (miRNA) and Endonuclease Recognition Sites

Nucleic acid molecules (e.g., RNA, e.g., mRNA) of the disclosure include regulatory elements, for example, microRNA (miRNA) binding sites, endonuclease cleavage sites, structured mRNA sequences and/or motifs, artificial binding sites engineered to act as pseudo-receptors for endogenous nucleic acid binding molecules, and combinations thereof. A regulatory element on an RNA molecule of the disclosure regulates translation of the RNA molecule. In some embodiments, binding of the miRNA or endonuclease to the recognition site within the regulatory element results in cleavage of the RNA molecule at the site of recognition, thereby enhancing or suppressing translation. In other embodiments, binding of the miRNA to the regulatory element on an RNA molecule suppressing translation of the RNA molecule without cleavage. The recognition site may be bound by an miRNA or an endonuclease in a cell-type-specific manner. In some embodiments, the term “modification of the recognition site” refers to the binding of miRNA or endonuclease to the recognition site within the regulatory element, which results in cleavage or non-cleavage based translation repression. In some embodiments, a nucleic acid molecule (e.g., RNA, e.g., mRNA) of the disclosure comprises an open reading frame (ORF) encoding a first polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific, cell-type specific, and/or microenvironment specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site”, “miR target site” (miRts) or “miR recognition site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprises an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion of the full length of a naturally-occurring miRNA sequence that is at least 15 nucleotides in length. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs may still be capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is only intended to be delivered to a specific tissue or cell, then a miRNA abundant in that tissue or cell can inhibit the repression of the expression of the gene of interest on a first polynucleotide if one or multiple binding sites of the miRNA are engineered into the 5′UTR and/or 3′UTR of a second polynucleotide (e.g., an mRNA encoding a repressor). The expression of the gene of interest (e.g., a gene encoding a first polypeptide) would be suppressed in tissues/cells that do not express the miRNA.

For example, one of skill in the art would understand that one or more miR binding sites can be included in a nucleic acid molecule (e.g., an RNA, e.g., mRNA) to minimize expression of the gene of interest in cell types other than liver cells (e.g., cells in the lymphoid, myeloid, endothelial, epithelial, or hematopoietic lineages). In one embodiment, a miR122 binding site can be used. In another embodiment, a miR126 binding site can be used. In another embodiment, a miR142 binding site can be used. In still another embodiment, multiple copies of these miR binding sites or combinations may be used.

Regulation of expression of the gene of interest in specific tissues can be accomplished through introduction one or more miRNA binding sites, e.g., one or more distinct miRNA binding sites. The decision on which miRNA binding site to insert can be made based on miRNA expression patterns and/or their profiling in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), spleen (miR142), lymphoid cells (miR150) and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

In one example, a nucleic acid molecule (e.g., RNA, e.g., mRNA) of the disclosure can comprise a repressor, such as L7Ae under the control of miR142 or miR223 which are selectively abundant in immune cells such as APCs. This would lead to target RNA expression being selectively turned on in APCs (possibly other immune cells such as T cells as well), while target expression would be suppressed in cells (e.g., hepatocytes) that do not express these miRNAs or express them at a negligible level.

In another example, the methods of this disclosure can be used to turn ON expression of a target gene only in APCs. For example, the expression of a target gene may be desired specifically in APCs post vaccination. This could help minimize any unintended events in bystander cells after e.g., intramuscular dosing. In some embodiments, the methods of this disclosure can be used in other immune applications where ON switches could be enabling/increase safety. For example, for Chimeric antigen receptor (CAR) T-cell therapy, it is detrimental to have the CAR expressed in tumor samples since it can mask the target epitope. It can also be detrimental to express the CAR in regulatory T cells (Treg cells). The methods of this disclosure can be used to turn ON expression only in specific T cells.

In some embodiments, the methods of this disclosure can be used to turn ON expression in certain differentiated immune cells for use in immune-oncology applications. In some embodiments, the methods of this disclosure can be used to turn ON expression in particular cell lineages, e.g., hematopoietic progenitor cells in which gene editing is desired, thereby increasing safety of gene-editing technologies.

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11, 288, the content of each of which is incorporated herein by reference in its entirety.)

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA encoding a repressor) of the disclosure, the degree of expression of the gene of interest in specific cell types (e.g., hepatocytes, myeloid cells, endothelial cells, cancer cells, etc.) can be enhanced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR. In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can comprise at least one miRNA binding site in the 3′UTR in order to selectively inhibit repression of mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure that is encoding the repressor to target RNA, more unstable in antigen presenting cells. Non-limiting examples of these miRNAs include mir-142-5p, mir-142-3p, mir-146a-5p, and mir-146-3p.

An endonuclease for use in the present disclosure could be effectively any known RNA endonuclease that is sequence or structure specific. See e.g., Tomecki R and Dziembowski A., RNA 2010. 16: 1692-1724; and Schoenberg D R., Wiley Interdiscip Rev RNA. 2011; 2(4): 582-600. In some embodiments, an endonuclease could be an engineered nuclease where a non-specific endonuclease is fused to a specific RNA recognition element (See eg. Choudhary et al. Nature Comm, 2012; 3:1147.) An endonuclease cleavage site can be any site known in the art. See e.g., Mendez A S et al, Nucleic Acids Research, (2018), 46(22): 11968-11979; Zhou W et al Proceedings of the National Academy of Sciences February 2017, 114 (8) E1554-E1563; Floyd-Smith G et al., Science (1981) 212: 4498: 1030-1032.

### IVT Polynucleotide Architecture

In some embodiments, the polynucleotide of the present disclosure comprising an mRNA encoding a polypeptide, or a repressor is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded polypeptide, or repressor. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding the polypeptide, or the repressor can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of the polypeptide, or the repressor or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### 5′UTR and 3′ UTR

A UTR can be homologous or heterologous to the coding region in a polynucleotide. In some embodiments, the UTR is homologous to the ORF encoding the polypeptide, or the repressor. In some embodiments, the UTR is heterologous to the ORF encoding the polypeptide, or the repressor.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO: 205), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or p actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a 3-F1-ATPase, a creatine kinase, a myoglobin, a granulocyte-colony stimulating factor (G-CSF); a collagen (e.g., collagen type I, alpha 2 (CollA2), collagen type I, alpha 1 (CollA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the disclosure comprise a 5′ UTR and/or a 3′ UTR selected from any of the UTRs disclosed herein, e.g., in Table 6.

TABLE 6
Exemplary 5′ UTR and 3′ UTR sequences

| SEQ ID NO: | Sequence |
|---|---|
| 3′ UTR sequences | |
| 81 | GCUGGAGCCUCGGUGGCCAUGCUUCUUGCCCCUUGGGCCU |
| | CCCCCCAGCCCCUCCUCCCCUUCCUGCACCCGUACCCCCUC |
| | CAUAAAGUAGGAAACACUACAGUGGUCUUUGAAUAAAGU |
| | CUGAGUGGGCGGC |
| | (3′ UTR with miR 142-3p binding site) |
| 82 | 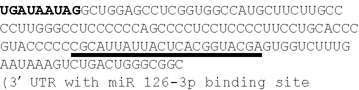 |
| 83 | UGAUAAUAGGCUGGAGCCUCGGUGGCCAUGCUUCUUGCC |
| | CCUUGGGCCUCCCCCCAGCCCCUCCUCCCCUUCCUGCACCC |
| | GUACCCCCGUGGUCUUUGAAUAAAGUCUGAGUGGGCGGC |
| | (3′ UTR, no miR binding sites) |
| 84 | UGAUAAUAGGCUGGAGCCUCGGUGGCCAUGCUUCUUGCC |
| | CCUUGGGCCUCCCCCCAGCCCCUCCUCCCCUUCCUGCACCC |
| | GUACCCCCUCCAUAAAGUAGGAAACACUACAGUGGUCUUU |
| | GAAUAAAGUCUGAGUGGGCGGC |
| | (3′ UTR with miR 142-3p binding site) |
| 85 | 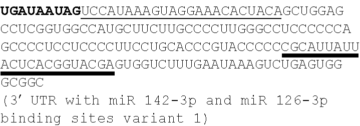 |
| 86 | 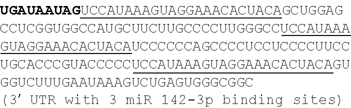 |
| 87 | 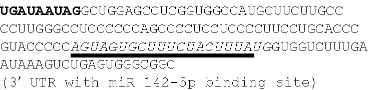 |
| 88 | 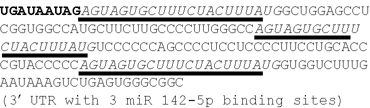 |
| 89 | 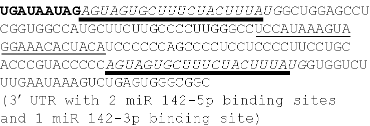 |
| 90 | UGAUAAUAGGCUGGAGCCUCGGUGGCCAUGCUUCUUGCC |
| | CCUUGGGCCUCCCCCCAGCCCCUCCUCCCCUUCCUGCACCC |
| | GUACCCCCACCCCUAUCACAAUUAGCAUUAAGUGGUCUUUG |
| | AAUAAAGUCUGAGUGGGCGGC |
| | (3′ UTR with miR 155-5p binding site) |
| 91 | UGAUAAUAGACCCCUAUCACAAUUAGCAUUAAGCUGGAGCC |
| | UCGGUGGCCAUGCUUCUUGCCCCUUGGGCCACCCCUAUCAC |
| | AAUUAGCAUUAAUCCCCCCAGCCCCUCCUCCCCUUCCUGCA |
| | CCCGUACCCCCACCCCUAUCACAAUUAGCAUUAAGUGGUCU |
| | UUGAAUAAAGUCUGAGUGGGCGGC |
| | (3′ UTR with 3 miR 155-5p binding sites) |
| 92 | UGAUAAUAGACCCCUAUCACAAUUAGCAUUAAGCUGGAGCC |
| | UCGGUGGCCAUGCUUCUUGCCCCUUGGGCCUCCAUAAAGU |
| | AGGAAACACUACAUCCCCCCAGCCCCUCCUCCCCUUCCUG |
| | CACCCGUACCCCCACCCCUAUCACAAUUAGCAUUAAGUGGU |
| | CUUUGAAUAAAGUCUGAGUGGGCGGC |
| | (3′ UTR with 2 miR 155-5p binding sites |
| | and 1 miR 142-3p binding site) |
| 93 | UGAUAAUAGUCCAUAAAGUAGGAAACACUACAGCUGGAG |
| | CCUCGGUGGCCAUGCUUCUUGCCCCUUGGGCCUCCCCCCA |
| | GCCCCUCCUCCCCUUCCUGCACCCGUACCCCCGUGGUCUU |
| | UGAAUAAAGUCUGAGUGGGCGGC |
| | (3′ UTR with miR 142-3p binding site, |
| | Pl insertion) |
| 94 | UGAUAAUAGGCUGGAGCCUCGGUGGCUCCAUAAAGUAGG |
| | AAACACUACACAUGCUUCUUGCCCCUUGGGCCUCCCCCCA |
| | GCCCCUCCUCCCCUUCCUGCACCCGUACCCCCGUGGUCUU |
| | UGAAUAAAGUCUGAGUGGGCGGC |
| | (3′ UTR with miR 142-3p binding site, |
| | P2 insertion) |
| 95 | UGAUAAUAGGCUGGAGCCUCGGUGGCCAUGCUUCUUGCC |
| | CCUUGGGCCUCCAUAAAGUAGGAAACACUACAUCCCCCCA |
| | GCCCCUCCUCCCCUUCCUGCACCCGUACCCCCGUGGUCUU |
| | UGAAUAAAGUCUGAGUGGGCGGC |
| | (3′ UTR with miR 142-3p binding site, |
| | P3 insertion) |
| 96 | 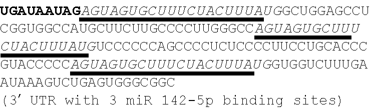 |
| 97 | UGAUAAUAGGCUGGAGCCUCGGUGGCCAUGCUUCUUGCC |
| | CCUUCCAUAAAGUAGGAAACACUACAUGGGCCUCCCCCCA |
| | GCCCCUCCUCCCCUUCCUGCACCCGUACCCCCGUGGUCUU |
| | UGAAUAAAGUCUGAGUGGGCGGC |
| | (3′ UTR including miR142-3p binding |
| | site) |
| 98 | UGAUAAUAGGCUGGAGCCUCGGUGGCCAUGCUUCUUGCC |
| | CCUUGGGCCUCCCCCCAGUCCAUAAAGUAGGAAACACUAC |
| | ACCCCUCCUCCCCUUCCUGCACCCGUACCCCCGUGGUCUU |
| | UGAAUAAAGUCUGAGUGGGCGGC |
| | (3′ UTR including miR142-3p binding |
| | site) |
| 99 | UGAUAAUAGGCUGGAGCCUCGGUGGCCAUGCUUCUUGCC |
| | CCUUGGGCCUCCCCCCAGCCCCUCCUCCCCUUCUCCAUAAA |
| | GUAGGAAACACUACACUGCACCCGUACCCCCGUGGUCUUU |
| | GAAUAAAGUCUGAGUGGGCGGC |
| | (3′ UTR including miR142-3p binding |
| | site) |
| 100 | UGAUAAUAGGCUGGAGCCUCGGUGGCCAUGCUUCUUGCC |
| | CCUUGGGCCUCCCCCCAGCCCCUCCUCCCCUUCCUGCACCC |
| | GUACCCCCGUGGUCUUUGAAUAAAGUUCCAUAAAGUAGG |
| | AAACACUACACUGAGUGGGCGGC |
| | (3′ UTR including miR142-3p binding |
| | site) |
| 101 | 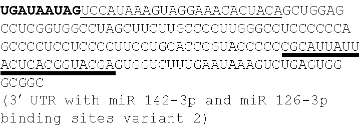 |
| 103 | UGAUAAUAGGCUGGAGCCUCGGUGGCCUAGCUUCUUGCC |
| | CCUUGGGCCUCCCCCCAGCCCCUCCUCCCCUUCCUGCACCC |
| | GUACCCCCUCCAUAAAGUAGGAAACACUACAGUGGUCUUU |
| | GAAUAAAGUCUGAGUGGGCGGC |
| | (3′ UTR with miR 142-3p binding site |
| | variant 3) |
| 104 | 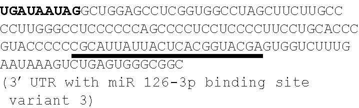 |
| 105 | UGAUAAUAGUCCAUAAAGUAGGAAACACUACAGCUGGAG |
| | CCUCGGUGGCCUAGCUUCUUGCCCCUUGGGCCUCCAUAAA |
| | GUAGGAAACACUACAUCCCCCCAGCCCCUCCUCCCCUUCC |
| | UGCACCCGUACCCCCUCCAUAAAGUAGGAAACACUACAGU |
| | GGUCUUUGAAUAAAGUCUGAGUGGGCGGC |
| | (3′ UTR with 3 miR 142-3p binding sites |
| | variant 2) |
| 106 | UGAUAAUAGUCCAUAAAGUAGGAAACACUACAGCUGGAG |
| | CCUCGGUGGCCUAGCUUCUUGCCCCUUGGGCCUCCCCCCA |
| | GCCCCUCCUCCCCUUCCUGCACCCGUACCCCCGUGGUCUU |
| | UGAAUAAAGUCUGAGUGGGCGGC |
| | (3′ UTR with miR 142-3p binding site, |
| | Pl insertion variant 2) |
| 107 | UGAUAAUAGGCUGGAGCCUCGGUGGCUCCAUAAAGUAGG |
| | AAACACUACACUAGCUUCUUGCCCCUUGGGCCUCCCCCCA |
| | GCCCCUCCUCCCCUUCCUGCACCCGUACCCCCGUGGUCUU |
| | UGAAUAAAGUCUGAGUGGGCGGC |
| | (3′ UTR with miR 142-3p binding site, |
| | P2 insertion variant 2) |
| 108 | UGAUAAUAGGCUGGAGCCUCGGUGGCCUAGCUUCUUGCC |
| | CCUUGGGCCUCCAUAAAGUAGGAAACACUACAUCCCCCCA |
| | GCCCCUCCUCCCCUUCCUGCACCCGUACCCCCGUGGUCUU |
| | UGAAUAAAGUCUGAGUGGGCGGC |
| | (3′ UTR with miR 142-3p binding site, |
| | P3 insertion variant 2) |
| 109 | UGAUAAUAGGCUGGAGCCUCGGUGGCCUAGCUUCUUGCC |
| | CCUUGGGCCUCCCCCCAGCCCCUCCUCCCCUUCCUGCACCC |
| | GUACCCCCACCCCUAUCACAAUUAGCAUUAAGUGGUCUUUG |
| | AAUAAAGUCUGAGUGGGCGGC |
| | (3′ UTR with miR 155-5p binding site |
| | variant 2) |
| 110 | UGAUAAUAGACCCCUAUCACAAUUAGCAUUAAGCUGGAGCC |
| | UCGGUGGCCUAGCUUCUUGCCCCUUGGGCCACCCCUAUCAC |
| | AAUUAGCAUUAAUCCCCCCAGCCCCUCCUCCCCUUCCUGCA |
| | CCCGUACCCCCACCCCUAUCACAAUUAGCAUUAAGUGGUCU |
| | UUGAAUAAAGUCUGAGUGGGCGGC |
| | (3′ UTR with 3 miR 155-5p binding sites |
| | variant 2) |
| 111 | UGAUAAUAGACCCCUAUCACAAUUAGCAUUAAGCUGGAGCC |
| | UCGGUGGCCUAGCUUCUUGCCCCUUGGGCCUCCAUAAAGU |
| | AGGAAACACUACAUCCCCCCAGCCCCUCCUCCCCUUCCUG |
| | CACCCGUACCCCCACCCCUAUCACAAUUAGCAUUAAGUGGU |
| | CUUUGAAUAAAGUCUGAGUGGGCGGC |
| | (3′ UTR with 2 miR 155-5p binding sites |
| | and 1 miR 142-3p binding site variant 2) |
| 112 | UGAUAAUAGGCUGGAGCCUCGGUGGCCAUGCUUCUUGCCC |
| | CUUGGGCCUCCCCCCAGCCCCUCCUCCCCUUCCUGCACCCG |
| | UACCCCCCAAACACCAUUGUCACACUCCAGUGGUCUUUGA |
| | AUAAAGUCUGAGUGGGCGGC |
| | (3′ UTR (miR122)) |
| 113 | UGAUAAUAGGCUGGAGCCUCGGUGGCCUAGCUUCUUGCCC |
| | CUUGGGCCUCCCCCCAGCCCCUCCUCCCCUUCCUGCACCCG |
| | UACCCCCCAAACACCAUUGUCACACUCCAGUGGUCUUUGA |
| | AUAAAGUCUGAGUGGGCGGC |
| | (3′ UTR (mir122-TAG) |
| 114 | UGAUAAUAGGCUGGAGCCUCGGUGGCCUAGCUUCUUGCCC |
| | CUUGGGCCUCCCCCCAGCCCCUCCUCCCCUUCCUGCACCCG |
| | UACCCCCCGCAUUAUUACUCACGGUACGAGUGGUCUUUGA |
| | AUAAAGUCUGAGUGGGCGGC |
| | (3′ UTR v1.1 (miR126-3p)) |
| 143 | UGAUAAUAGGCUGGAGCCUCGGUGCGCAUUAUUACUCAC |
| | GGUACGAGCCUAGCUUCUUGCCCCUUGGGCCCGCAUUAUU |
| | ACUCACGGUACGAUCCCCCCAGCCCCUCCUCCCCUUCCUGC |
| | ACCCGUACCCCCCGCAUUAUUACUCACGGUACGAGUGGUC |
| | UUUGAAUAAAGUCUGAGUGGGCGGC |
| | (3xmiR126ts) |
| 144 | UGAUAAUAGCGCAUUAUUACUCACGGUACGAGCUGGAGC |
| | CUCGGUGGCCUAGCUUCUUGCCCCUUGGGCCCGCAUUAUU |
| | ACUCACGGUACGAUCCCCCCAGCCCCUCCUCCCCUUCCUGC |
| | ACCCGUACCCCCCGCAUUAUUACUCACGGUACGAGUGGUC |
| | UUUGAAUAAAGUCUGAGUGGGCGGC |
| | (3xmiR126ts with mismatches) |
| 145 | UGAUAAUAGCAAACACCAUUGUCACACUCCAGCUGGAGCC |
| | UCGGUGGCCUAGCUUCUUGCCCCUUGGGCCCAAACACCAU |
| | UGUCACACUCCAUCCCCCCAGCCCCUCCUCCCCUUCCUGCA |
| | CCCGUACCCCCCAAACACCAUUGUCACACUCCAGUGGUCU |
| | UUGAAUAAAGUCUGAGUGGGCGGC |
| | (3xmiR122ts) |
| 146 | UGAUAAUAGCACUGGUACAAGGGUUGGGAGAGCUGGAGC |
| | CUCGGUGGCCUAGCUUCUUGCCCCUUGGGCCCACUGGUAC |
| | AAGGGUUGGGAGAUCCCCCCAGCCCCUCCUCCCCUUCCUG |
| | CACCCGUACCCCCCACUGGUACAAGGGUUGGGAGAGUGGU |
| | CUUUGAAUAAAGUCUGAGUGGGCGGC |
| | (3xmiR150ts) |
| 147 | UGAUAAUAGGCUGGAGCCUCGGUGAUGCCCUUUUAACAU |
| | UGCACUGGCCUAGCUUCUUGCCCCUUGGGCCAUGCCCUUU |
| | UAACAUUGCACUGUCCCCCCAGCCCCUCCUCCCCUUCCUG |
| | CACCCGUACCCCCAUGCCCUUUUAACAUUGCACUGGUGGU |
| | CUUUGAAUAAAGUCUGAGUGGGCGGC |
| | (3xmiR130ats) |
| 148 | UGAUAAUAGGCUGGAGCCUCGGUGCACAAAUUCGGAUCU |
| | ACAGGGUAGCCUAGCUUCUUGCCCCUUGGGCCCACAAAUU |
| | CGGAUCUACAGGGUAUCCCCCCAGCCCCUCCUCCCCUUCC |
| | UGCACCCGUACCCCCCACAAAUUCGGAUCUACAGGGUAGU |
| | GGUCUUUGAAUAAAGUCUGAGUGGGCGGC |
| | (3xmiR10ats) |
| 151 | UGAUAAUAGGCUGGAGCCUCAUUAACACUGGUACAAGGG |
| | UUGGGAGAUAUAAAGUAAAAUUCACCAUUUUAAUUACAC |
| | UGGUACAAGGGUUGGGAGAUAAUAAAAAUAAAGUAUAUA |
| | AUUCACUGGUACAAGGGUUGGGAGACAAUGUACCCCCGU |
| | GGUCUUUAAAUAAAGUCUAAGUGGGCGGC |
| | (AU rich_3xmiR150ts) |
| 152 | UGAUAAUAGGCUGGAGCCUCAUUAACUACAGUACAAGGG |
| | UUGGGAGAUAUAAAGUAAAAUUCACCAUUUUAAUUACUA |
| | CAGUACAAGGGUUGGGAGAUAAUAAAAAUAAAGUAUAUA |
| | AUUCUACAGUACAAGGGUUGGGAGACAAUGUACCCCCGU |
| | GGUCUUUAAAUAAAGUCUAAGUGGGCGGC |
| | (AU rich_3xmiR150ts_mm) |
| 153 | UGAUAAUAGGCUGGAGCCUCAUUACUACAGUACAAGGGU |
| | UGGGAGAAUAUAAAGUAAACUACAGUACAAGGGUUGGGA |
| | GAAUUCACCAUUUUCUACAGUACAAGGGUUGGGAGAAAU |
| | UAUAAUAAAAAUAAAGUAUAUAAUCUACAGUACAAGGGU |
| | UGGGAGAUCAAUGUACCCCCUACAGUACAAGGGUUGGGA |
| | GACGUGGUCUUUAACUACAGUACAAGGGUUGGGAGAAUA |
| | AAGUCUAAGUGGGCGGC |
| | (AU rich_6xmiR150ts_mm) |
| 154 | UGAUAAUAGGCUGGAGCCUCAUUAAUCCAUAAAGUAGGA |
| | AACACUACAUAUAAAGUAAAAUUUCCAUAAAGUAGGAAA |
| | CACUACACACCAUUUUAAUUAUCCAUAAAGUAGGAAACAC |
| | UACAUAAUAAAAAUAAAGUCCAUAAAGUAGGAAACACUA |
| | CAUAUAUAAUUCAUAGUCCAUAAAGUAGGAAACACUACA |
| | UACCCCCGUGGUCUUCCAUAAAGUAGGAAACACUACAUUA |
| | AAUAAAGUCUAAGUGGGCGGC |
| | (AU rich_6xmiR142ts) |
| 155 | UGAUAAUAGGCUGGAGCCUCAUUAAUUACAAAAGUAGGA |
| | AACACUACAUAUAAAGUAAAAUUUUACAAAAGUAGGAAA |
| | CACUACACACCAUUUUAAUUAUUACAAAAGUAGGAAACA |
| | CUACAUAAUAAAAAUAAAGUUACAAAAGUAGGAAACACU |
| | ACAUAUAUAAUUCAUAGUUACAAAAGUAGGAAACACUAC |
| | AUACCCCCGUGGUCUUUACAAAAGUAGGAAACACUACAUU |
| | AAAUAAAGUCUAAGUGGGCGGC |
| | (AU rich_6xmiR142ts_mm) |
| 156 | UGAUAAUAGGCUGGAGCCUCAUUAACUACAUAUUACUCA |
| | CGGUACGAUAUAAAGUAAAAUUCUACAUAUUACUCACGG |
| | UACGACACCAUUUUAAUUACUACAUAUUACUCACGGUACG |
| | AUAAUAAAAAUAAAGCUACAUAUUACUCACGGUACGAUA |
| | UAUAAUUCAUAGCUACAUAUUACUCACGGUACGAUACCCC |
| | CGUGGUCUCUACAUAUUACUCACGGUACGAUUAAAUAAA |
| | GUCUAAGUGGGCGGC |
| | (AU rich_6xmiR126ts_mm) |
| 157 | UGAUAAUAGGCUGGAGCCUCGGUGCGCAUUAUUACUCAC |
| | GGUACGAGCCUAGCUUCUUGCCCCUUGGGCCCGCAUUAUU |
| | ACUCACGGUACGAUCCCCCCAGCCCCUCCUCCCAUGCCCU |
| | UUUAACAUUGCACUGCUUCCUGCACCCGUACCCCCAUGCC |
| | CUUUUAACAUUGCACUGGUGGUCUUUGAAUAAAGUCUGA |
| | GUGGGCGGC |
| | (AU rich_2xmiR126ts + 2xmiR130ats) |
| 158 | UGAUAAUAGGCUGGAGCCUCAUUAACUACAUAUUACUCA |
| | CGGUACGAUAUAAAGUAAAAUUCACCAUUUUCUACAUAU |
| | UACUCACGGUACGAAAUUAUAAUAAAAAUAAAGUAUAUA |
| | UACACUUUUAACAUUGCACUGAAUUCAUAGUACCCCCGUG |
| | GUCUAUACACUUUUAACAUUGCACUGUUAAAUAAAGUCU |
| | AAGUGGGCGGC |
| | (AU rich_2xmiR126ts_mm + 2xmiR130ats_mm) |
| 159 | UGAUAAUAGGCUGGAGCCUCAUUAAAUACACUUUUAACA |
| | UUGCACUGUAUAAAGUAAAAUUAUACACUUUUAACAUUG |
| | CACUGCACCAUUUUAAUUAAUACACUUUUAACAUUGCACU |
| | GUAAUAAAAAUAAAGAUACACUUUUAACAUUGCACUGUA |
| | UAUAAUUCAUAGAUACACUUUUAACAUUGCACUGUACCCC |
| | CGUGGUCUAUACACUUUUAACAUUGCACUGUUAAAUAAA |
| | GUCUAAGUGGGCGGC |
| | (AU rich_6xmiR130ats_mm) |
| 160 | UGAUAAUAGGCUGGAGCCUCAUUAACUUUUACCAUUGUC |
| | ACACUCCAUAUAAAGUAAAAUUCACCAUUUUAAUUACUU |
| | UUACCAUUGUCACACUCCAUAAUAAAAAUAAAGUAUAUA |
| | AUUCUUUUACCAUUGUCACACUCCACAAUGUACCCCCGUG |
| | GUCUUUAAAUAAAGUCUAAGUGGGCGGC |
| | (AU rich_3xmiR122ts_mm) |
| 161 | UGAUAAUAGGCUGGAGCCUCAUUAACUACAACCAUUGUC |
| | ACACUCCAUAUAAAGUAAAAUUCUACAACCAUUGUCACAC |
| | UCCACACCAUUUUAAUUACUACAACCAUUGUCACACUCCA |
| | UAAUAAAAAUAAAGCUACAACCAUUGUCACACUCCAUAU |
| | AUAAUUCAUAGCUACAACCAUUGUCACACUCCAUACCCCC |
| | GUGGUCUCUACAACCAUUGUCACACUCCAUUAAAUAAAGU |
| | CUAAGUGGGCGGC |
| | (AU rich_6xmiR122ts_mm) |
| 162 | UGAUAAUAGCACUGGUACAAGGGUUGGGAGAGCUGGAGC |
| | CUCGGUGGCCUAGCUUCUUGCCCCUUGCACUGGUACAAGG |
| | GUUGGGAGAAUUGUAUAACGAGCCCCUCCUCCCCUUCCUG |
| | CACCCGUAAUAACCACUGGUACAAGGGUUGGGAGAGUGG |
| | UCUUUGAAUAAAGUCUGAGUGGGCGGC |
| | (3xmiR150ts_sp(structural prediction/ |
| | CP3X2)) |
| 163 | UAAAGCUCCCCGGGG UCCAUAAAGUAGGAAACACUACA |
| | GCCUCGGUGGCCUAGCUUCUUGCCCCUUGGGCCUCCAUAA |
| | AGUAGGAAACACUACAUCCCCCCAGCCCCUCCUCCCCUUC |
| | CUGCACCCGUACCCCCUCCAUAAAGUAGGAAACACUACAG |
| | UGGUCUUUGAAUAAAGUCUGAGUGGGCGGC(v2.0 3′ |
| | UTR + 3XmiR142-3p |
| 164 | UAAAGCUCCCCGGGGCGCAUUAUUACUCACGGUACGAGCC |
| | UCGGUGGCCUAGCUUCUUGCCCCUUGGGCCCGCAUUAUUA |
| | CUCACGGUACGAUCCCCCCAGCCCCUCCUCCCCUUCCUGCA |
| | CCCGUACCCCCCGCAUUAUUACUCACGGUACGAGUGGUCU |
| | UUGAAUAAAGUCUGAGUGGGCGGC(3′v2_3xmiR126ts) |
| 165 | UAAAGCUCCCCGGGGCAAACACCAUUGUCACACUCCAGCC |
| | UCGGUGGCCUAGCUUCUUGCCCCUUGGGCCCAAACACCAU |
| | UGUCACACUCCAUCCCCCCAGCCCCUCCUCCCCUUCCUGCA |
| | CCCGUACCCCCCAAACACCAUUGUCACACUCCAGUGGUCU |
| | UUGAAUAAAGUCUGAGUGGGCGGC(3′v2_3xmiR122ts) |
| 166 | UAAAGCUCCCCGGGGCACUGGUACAAGGGUUGGGAGAGCC |
| | UCGGUGGCCUAGCUUCUUGCCCCUUGGGCCCACUGGUACA |
| | AGGGUUGGGAGAUCCCCCCAGCCCCUCCUCCCCUUCCUGC |
| | ACCCGUACCCCCCACUGGUACAAGGGUUGGGAGAGUGGUC |
| | UUUGAAUAAAGUCUGAGUGGGCGGC(3′v2_3xmiR150ts) |
| 167 | UAAAGCUCCCCGGGGAUGCCCUUUUAACAUUGCACUGGCC |
| | UCGGUGGCCUAGCUUCUUGCCCCUUGGGCCAUGCCCUUUU |
| | AACAUUGCACUGUCCCCCCAGCCCCUCCUCCCCUUCCUGC |
| | ACCCGUACCCCCAUGCCCUUUUAACAUUGCACUGGUGGUC |
| | UUUGAAUAAAGUCUGAGUGGGCGGC(3′v2_3xmiR130ats) |
| 168 | UAAAGCUCCCCGGGGCACAAAUUCGGAUCUACAGGGUAGC |
| | CUCGGUGGCCUAGCUUCUUGCCCCUUGGGCCCACAAAUUC |
| | GGAUCUACAGGGUAUCCCCCCAGCCCCUCCUCCCCUUCCU |
| | GCACCCGUACCCCCCACAAAUUCGGAUCUACAGGGUAGUG |
| | GUCUUUGAAUAAAGUCUGAGUGGGCGGC |
| | (3′v2_3xmiR10ats) |
| 169 | UAAAGCUCCCCGGGGUGGGGUAUUUGACAAACUGACAGCC |
| | UCGGUGGCCUAGCUUCUUGCCCCUUGGGCCUGGGGUAUUU |
| | GACAAACUGACAUCCCCCCAGCCCCUCCUCCCCUUCCUGC |
| | ACCCGUACCCCCUGGGGUAUUUGACAAACUGACAGUGGUC |
| | UUUGAAUAAAGUCUGAGUGGGCGGC(3′v2_3xmiR223) |
| 170 | UAAAGCUCCCCGGGGGCUGGAGCCUCAUUAAUUACAAAAG |
| | UAGGAAACACUACAUAUAAAGUAAAAUUUUACAAAAGUA |
| | GGAAACACUACACACCAUUUUAAUUAUUACAAAAGUAGG |
| | AAACACUACAUAAUAAAAAUAAAGUUACAAAAGUAGGAA |
| | ACACUACAUAUAUAAUUCAUAGUUACAAAAGUAGGAAAC |
| | ACUACAUACCCCCGUGGUCUUUACAAAAGUAGGAAACACU |
| | ACAUUAAAUAAAGUCUAAGUGGGCGGC |
| | (3′v2cc_6XmiR142_mm) |
| 171 | UAAAGCUCCCCGGGGGCCUCAUUAAUUACAAAAGUAGGA |
| | AACACUACAUAUAAAGUAAAAUUUUACAAAAGUAGGAAA |
| | CACUACACACCAUUUUAAUUAUUACAAAAGUAGGAAACA |
| | CUACAUAAUAAAAAUAAAGUUACAAAAGUAGGAAACACU |
| | ACAUAUAUAAUUCAUAGUUACAAAAGUAGGAAACACUAC |
| | AUACCCCCGUGGUCUUUACAAAAGUAGGAAACACUACAUU |
| | AAAUAAAGUCUAAGUGGGCGGC |
| | (3′kappa_cc6XmiR142mm) |
| 172 | UAAAGCUCCCCGGGGGCUGGAGCCUCAUUAAUUACAAAAG |
| | UAGGAAACACUACAUAUAAAGUAAAAUUCACCAUUUUAA |
| | UUAUUACAAAAGUAGGAAACACUACAUAAUAAAAAUAAA |
| | GUAUAUAAUUUUACAAAAGUAGGAAACACUACACAUAGU |
| | ACCCCCGUGGUCUUUAAAUAAAGUCUAAGUGGGCGGC |
| | (3′v2cc_3XmiR142_mm) |
| 173 | UAAAGCUCCCCGGGGGCCUCAUUAACACUGGUACAAGGGU |
| | UGGGAGAUAUAAAGUAAAAUUCACCAUUUUAAUUACACU |
| | GGUACAAGGGUUGGGAGAUAAUAAAAAUAAAGUAUAUAA |
| | UUCACUGGUACAAGGGUUGGGAGACAAUGUACCCCCGUG |
| | GUCUUUAAAUAAAGUCUAAGUGGGCGGC(3′v2_AU |
| | rich_3xmiR150ts) |
| 174 | UAAAGCUCCCCGGGGGCCUCAUUAACUACAGUACAAGGGU |
| | UGGGAGAUAUAAAGUAAAAUUCACCAUUUUAAUUACUAC |
| | AGUACAAGGGUUGGGAGAUAAUAAAAAUAAAGUAUAUAA |
| | UUCUACAGUACAAGGGUUGGGAGACAAUGUACCCCCGUG |
| | GUCUUUAAAUAAAGUCUAAGUGGGCGGC(3′v2_AU |
| | rich_3xmiR150ts_mm) |
| 175 | UAAAGCUCCCCGGGGGCCUCAUUAACUACAGUACAAGGGU |
| | UGGGAGAAUAUAAAGUAAACUACAGUACAAGGGUUGGGA |
| | GAAUUCACCAUUUUCUACAGUACAAGGGUUGGGAGAAAU |
| | UAUAAUAAAAAUAAAGUAUAUAAUCUACAGUACAAGGGU |
| | UGGGAGAUCAAUGUACCCCCUACAGUACAAGGGUUGGGA |
| | GACGUGGUCUUUAACUACAGUACAAGGGUUGGGAGAAUA |
| | AAGUCUAAGUGGGCGGC |
| | (3′v2_AU rich_6xmiR150ts_mm) |
| 176 | UAAAGCUCCCCGGGGGCCUCAUUAACGCAUUAUUACUCAC |
| | GGUACGAUAUAAAGUAAAAUUCGCAUUAUUACUCACGGU |
| | ACGACACCAUUUUAAUUACGCAUUAUUACUCACGGUACGA |
| | UAAUAAAAAUAAAGCGCAUUAUUACUCACGGUACGAUAU |
| | AUAAUUCAUAGCGCAUUAUUACUCACGGUACGAUACCCCC |
| | GUGGUCUCGCAUUAUUACUCACGGUACGAUUAAAUAAAG |
| | UCUAAGUGGGCGGC(3′v2_AU rich_6xmiR126ts) |
| 177 | UAAAGCUCCCCGGGGGCCUCAUUAACUACAUAUUACUCAC |
| | GGUACGAUAUAAAGUAAAAUUCUACAUAUUACUCACGGU |
| | ACGACACCAUUUUAAUUACUACAUAUUACUCACGGUACGA |
| | UAAUAAAAAUAAAGCUACAUAUUACUCACGGUACGAUAU |
| | AUAAUUCAUAGCUACAUAUUACUCACGGUACGAUACCCCC |
| | GUGGUCUCUACAUAUUACUCACGGUACGAUUAAAUAAAG |
| | UCUAAGUGGGCGGC(3′v2_AU rich_6xmiR126ts_mm) |
| 178 | UAAAGCUCCCCGGGGGCCUCAUUAACGCAUUAUUACUCAC |
| | GGUACGAGCCUAGCUUCUUGCCCCUUGGGCCCGCAUUAUU |
| | ACUCACGGUACGAUCCCCCCAGCCCCUCCUCCCAUGCCCU |
| | UUUAACAUUGCACUGCUUCCUGCACCCGUACCCCCAUGCC |
| | CUUUUAACAUUGCACUGGUGGUCUUUGAAUAAAGUCUGA |
| | GUGGGCGGC(3′v2_AU rich_2xmiR126ts + |
| | 2xmiR130ats) |
| 179 | UAAAGCUCCCCGGGGGCCUCAUUAACUACAUAUUACUCAC |
| | GGUACGAUAUAAAGUAAAAUUCACCAUUUUCUACAUAUU |
| | ACUCACGGUACGAAAUUAUAAUAAAAAUAAAGUAUAUAU |
| | ACACUUUUAACAUUGCACUGAAUUCAUAGUACCCCCGUGG |
| | UCUAUACACUUUUAACAUUGCACUGUUAAAUAAAGUCUA |
| | AGUGGGCGGC(3′v2_AU rich_2xmiR126ts_mm + |
| | 2xmiR130ats_mm) |
| 180 | UAAAGCUCCCCGGGGGCCUCAUUAAAUACACUUUUAACAU |
| | UGCACUGUAUAAAGUAAAAUUAUACACUUUUAACAUUGC |
| | ACUGCACCAUUUUAAUUAAUACACUUUUAACAUUGCACU |
| | GUAAUAAAAAUAAAGAUACACUUUUAACAUUGCACUGUA |
| | UAUAAUUCAUAGAUACACUUUUAACAUUGCACUGUACCCC |
| | CGUGGUCUAUACACUUUUAACAUUGCACUGUUAAAUAAA |
| | GUCUAAGUGGGCGGC |
| | (3′v2_AU rich_6xmiR130ats_mm) |
| 181 | UAAAGCUCCCCGGGGGCCUCAUUAACUACAACCAUUGUCA |
| | CACUCCAUAUAAAGUAAAAUUCACCAUUUUAAUUACUAC |
| | AACCAUUGUCACACUCCAUAAUAAAAAUAAAGUAUAUAA |
| | UUCUACAACCAUUGUCACACUCCACAAUGUACCCCCGUGG |
| | UCUUUAAAUAAAGUCUAAGUGGGCGGC(3′v2_AU |
| | rich_3xmiR122ts_mm) |
| 182 | UAAAGCUCCCCGGGGGCCUCAUUAACUACAACCAUUGUCA |
| | CACUCCAUAUAAAGUAAAAUUCUACAACCAUUGUCACACU |
| | CCACACCAUUUUAAUUACUACAACCAUUGUCACACUCCAU |
| | AAUAAAAAUAAAGCUACAACCAUUGUCACACUCCAUAUA |
| | UAAUUCAUAGCUACAACCAUUGUCACACUCCAUACCCCCG |
| | UGGUCUCUACAACCAUUGUCACACUCCAUUAAAUAAAGUC |
| | UAAGUGGGCGGC(3′v2_AU rich_6xmiR122ts_mm) |
| 183 | UAAAGCUCCCCGGGGGCCUCAUUAACACUGGUACAAGGGU |
| | UGGGAGAGCUGGAGCCUCGGUGGCCUAGCUUCUUGCCCCU |
| | UGCACUGGUACAAGGGUUGGGAGAAUUGUAUAACGAGCC |
| | CCUCCUCCCCUUCCUGCACCCGUAAUAACCACUGGUACAA |
| | GGGUUGGGAGAGUGGUCUUUGAAUAAAGUCUGAGUGGGC |
| | GGC(3′v2_3xmiR150ts_sp (structural |
| | prediction/CP3X2)) |
| 184 | UAAAGCUCCCCGGGGGCUGGAGCCUCAUUAAUUACAUAUU |
| | UGACAAACUGACAUAUAAAGUAAAAUUCACCAUUUUAAU |
| | UAUUACAUAUUUGACAAACUGACAUAAUAAAAAUAAAGU |
| | AUAUAAUUUUACAUAUUUGACAAACUGACACAUAGUACC |
| | CCCGUGGUCUUUAAAUAAAGUCUAAGUGGGCGGC |
| | (3′v2_AU rich_3xmiR223ts_mm) |
| 185 | UAAAGCUCCCCGGGGGCCUCAUUAAUUACAUAUUUGACAA |
| | ACUGACAUAUAAAGUAAAAUUUUACAUAUUUGACAAACU |
| | GACACACCAUUUUAAUUAUUACAUAUUUGACAAACUGAC |
| | AUAAUAAAAAUAAAGUUACAUAUUUGACAAACUGACAUA |
| | UAUAAUUCAUAGUUACAUAUUUGACAAACUGACAUACCC |
| | CCGUGGUCUUUACAUAUUUGACAAACUGACAUUAAAUAA |
| | AGUCUAAGUGGGCGGC |
| | (3′v2_AU rich_6xmiR223ts_mm) |
| 186 | UAAAGCUCCCCGGGGGCUGGAGCCUCAUUAAUUACAAAAG |
| | UAGGAAACACUACAUAUAAAGUAAAAUUUUACAAAAGUA |
| | GGAAACACUACACACCAUUUUAAUUAUUACAAAAGUAGG |
| | AAACACUACAUAAUAAAAAUAAAGUUACAUAUUUGACAA |
| | ACUGACAUAUAUAAUUCAUAGUUACAUAUUUGACAAACU |
| | GACAUACCCCCGUGGUCUUUACAUAUUUGACAAACUGACA |
| | UUAAAUAAAGUCUAAGUGGGCGGC(3′v2_AU |
| | rich_3xmiR223ts_3xmiR223ts_mm) |
| 206 | UGAUAAUAGGCUGGAGCCUCAUUAACUACAUAUUACUCA |
| | CGGUACGAUCUAAACUGAAAUUCUACAUAUUACUCACGG |
| | UACGACACCACUAUAAUACCUACAUAUUACUCACGGUACG |
| | AUAAUAAUAAUAAUCCUACAUAUUACUCACGGUACGAUA |
| | UAUCACUCACUCAUCACCACGCUGUUCUACAUAUUACUCA |
| | CGGUACGAUACCCCCGUGGUCUUGGUACUCGUCUACAUAU |
| | UACUCACGGUACGAGGCACAUUAAAUAAAGUCUAAGUGG |
| | GCGGC(3′accV3_AU_6XmiR126mm) |
| 207 | UAAAGCUCCCCGGGGGCCUCAUUAAGCUGGAGCCUCAUUA |
| | ACUACAUAUUACUCACGGUACGAUCUAAACUGAAAUUCU |
| | ACAUAUUACUCACGGUACGACACCACUAUAAUACCUACAU |
| | AUUACUCACGGUACGAUAAUAAUAAUAAUCCUACAUAUU |
| | ACUCACGGUACGAUAUAUCACUCACUCAUCACCACGCUGU |
| | UCUACAUAUUACUCACGGUACGAUACCCCCGUGGUCUUGG |
| | UACUCGUCUACAUAUUACUCACGGUACGAGGCACAUUAAA |
| | UAAAGUCUAAGUGGGCGGC |
| | (3′v2accV3_AU_6XmiR126mm) |
| 5′ UTR SEQUENCES | |
| 115 | GGGAAAUAAGAGAGAAAAGAAGAGUAAGAAGAAAUAUAA |
| | GAGCCACC |
| | (5′ UTR) |
| 116 | GGGAAAUAAGAGUCCAUAAAGUAGGAAACACUACAAGAA |
| | AAGAAGAGUAAGAAGAAAUAUAAGAGCCACC |
| | (5′ UTR with miR142-3p binding site at |
| | position p1) |
| 117 | GGGAAAUAAGAGAGAAAAGAAGAGUAAUCCAUAAAGUAG |
| | GAAACACUACAGAAGAAAUAUAAGAGCCACC |
| | (5′ UTR with miR142-3p binding site at |
| | position p2) |
| 118 | GGGAAAUAAGAGAGAAAAGAAGAGUAAGAAGAAAUAUAA |
| | UCCAUAAAGUAGGAAACACUACAGAGCCACC |
| | (5′ UTR with miR142-3p binding site at |
| | position p3) |
| 119 | AGGAAAUAAGAGAGAAAAGAAGAGUAAGAAGAAAUAUAA |
| | GAGCCACC |
| | (5′-UTR (v1 A-start)) |
| 120 | GGGAAAUAAGAGAGAAAAGAAGAGUAAGAAGAAAUAUAA |
| | GACCCCGGCGCCGCCACC |
| | (5′ UTR v1.1) |
| 121 | AGGAAAUAAGAGAGAAAAGAAGAGUAAGAAGAAAUAUAA |
| | GACCCCGGCGCCGCCACC |
| | (5′ UTR v1.1 A-start) |
| 122 | GGGAGAUCAGAGAGAAAAGAAGAGUAAGAAGAAAUAUAA |
| | GAGCCACC |
| | 5′ UTR 002 (upstream UTR) |
| 123 | GGGAGACAAGCUUGGCAUUCCGGUACUGUUGGUAAAGCC |
| | ACC |
| | 5′ UTR-004 (Upstream UTR) |
| 124 | GGGAAUUAACAGAGAAAAGAAGAGUAAGAAGAAAUAUAA |
| | GAGCCACC |
| | 5′ UTR-008 (Upstream UTR) |
| 125 | GGGAAAUUAGACAGAAAAGAAGAGUAAGAAGAAAUAUAA |
| | GAGCCACC |
| | 5′ UTR-009 (Upstream UTR) |
| 126 | GGGAAAUAAGAGAGUAAAGAACAGUAAGAAGAAAUAUAA |
| | GAGCCACC |
| | 5′ UTR-010, Upstream |
| 127 | GGGAAAAAAGAGAGAAAAGAAGACUAAGAAGAAAUAUAA |
| | GAGCCACC |
| | 5′ UTR-011 (Upstream UTR) |
| 128 | GGGAAAUAAGAGAGAAAAGAAGAGUAAGAAGAUAUAUAA |
| | GAGCCACC |
| | 5′ UTR-012 (Upstream UTR) |
| 129 | GGGAAAUAAGAGACAAAACAAGAGUAAGAAGAAAUAUAA |
| | GAGCCACC |
| | 5′ UTR-013 (Upstream UTR) |
| 130 | GGGAAAUUAGAGAGUAAAGAACAGUAAGUAGAAUUAAAA |
| | GAGCCACC |
| | 5′ UTR-014 (Upstream UTR) |
| 131 | GGGAAAUAAGAGAGAAUAGAAGAGUAAGAAGAAAUAUAA |
| | GAGCCACC |
| | 5′ UTR-015 (Upstream UTR) |
| 132 | GGGAAAUAAGAGAGAAAAGAAGAGUAAGAAGAAAAUUAA |
| | GAGCCACC |
| | 5′ UTR-016 (Upstream UTR) |
| 133 | GGGAAAUAAGAGAGAAAAGAAGAGUAAGAAGAAAUUUAA |
| | GAGCCACC |
| | 5′ UTR-017 (Upstream UTR) |
| 134 | UCAAGCUUUUGGACCCUCGUACAGAAGCUAAUACGACUCA |
| | CUAUAGGGAAAUAAGAGAGAAAAGAAGAGUAAGAAGAAA |
| | UAUAAGAGCCACC |
| | 5′ UTR-018 (Upstream UTR) |
| 135 | GGGAAACGGCAAGCAGCAGCUGCCGCUGUUUUGUCUUAAC |
| | CGGUUGAAAGACCAUUAGGCUUUCUAACGGACGCUCACGA |
| | GAGAGGGGCACCAAUAGGGCCCCACGGCACUUCAAAGGUU |
| | UGGCUUGGAGUAGUAACCCAAGCAGCAACAGUUUUGACU |
| | UUCGGACCACCAUCAGGGGUCCCACGUUGGGAACACGUAA |
| | CUCUCCUACUAACAAGAGGAGCCCCGGCGCCGCCACC |
| | 5′ UTR with 6XMS2 sites |
| 137 | GGGAAAUCGCAAAAUUUGCUCUUCGCGUUAGAUUUCUUU |
| | UAGUUUUCUCGCAACUAGCAAGCUUUUUGUUCUCGCC |
| | (v2miRless 5′ UTR) |
| 138 | GGAUCCGUGAUCGGAAACGUGAGAUCCACCUCAGAUCCGC |
| | UAGGACACCCGCAGAUCGAGAAGAAGGCGAAUUCGCAAA |
| | AUUUGCUCUUCGCGUUAGAUUUCUUUUAGUUUUCUCGCA |
| | ACUAGCAAGCUUUUUGUUCUCGCC |
| | (5′ KT_v2(kink turn containing for |
| | target RNA)) |
| 139 | GGGAAAUAAGAGAGUCCAUAAAGUAGGAAACACUACAAA |
| | AAGAAGAGUAAUCCAUAAAGUAGGAAACACUACAGAAGA |
| | AAUAUAAUCCAUAAAGUAGGAAACACUACAGACCCCGGCG |
| | CCGCCACC |
| | (3xmiR142ts) |
| 140 | GGGAAAUAAGAGAGCGCAUUAUUACUCACGGUACGAAAA |
| | AGAAGAGUAACGCAUUAUUACUCACGGUACGAGAAGAAA |
| | UAUAACGCAUUAUUACUCACGGUACGAGACCCCGGCGCCG |
| | CCACC |
| | (3xmiR126ts) |
| 141 | GGGAAAUAAGAGAGCAAACACCAUUGUCACACUCCAAAA |
| | AGAAGAGUAACAAACACCAUUGUCACACUCCAGAAGAAA |
| | UAUAACAAACACCAUUGUCACACUCCAGACCCCGGCGCCG |
| | CCACC |
| | (3xmiR122ts) |

Stop codon = bold
miR 142-3p binding site = underline
miR 126-3p binding site = bold underline
miR 155-5p binding site = italicized
miR 142-5p binding site = italicized and bold underline

In certain embodiments, the 5′ UTR and/or 3′ UTR sequence of the disclosure comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 6.

In some embodiments, the 5′ UTR comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 6.

In some embodiments, the 3′ UTR comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 6.

In some embodiments, the polynucleotide disclosed herein, e.g., the polynucleotide encoding a polypeptide, or a repressor, comprises a 5′ UTR having the sequence of a 5′ UTR provided in Table 6, or a sequence with at least about 60%, at least about 70%, at least about 80%, at least about 90%, at least about 95%, at least about 96%, at least about 97%, at least about 98%, at least about 99%, or about 100% identity thereto. In some embodiments, the polynucleotide comprises a 5′ UTR comprising the sequence of any one of SEQ ID NOs: 115-135, or a sequence with at least about 60%, at least about 70%, at least about 80%, at least about 90%, at least about 95%, at least about 96%, at least about 97%, at least about 98%, at least about 99%, or about 100% identity thereto.

In some embodiments, the polynucleotide disclosed herein, e.g., the polynucleotide encoding a polypeptide, or a repressor comprises a 3′ UTR having the sequence of a 3′ UTR provided in Table 6, or a sequence with at least about 60%, at least about 70%, at least about 80%, at least about 90%, at least about 95%, at least about 96%, at least about 97%, at least about 98%, at least about 99%, or about 100% identity thereto. In some embodiments, the polynucleotide comprises a 3′ UTR comprising the sequence of any one of SEQ ID NOs: 81-114, or a sequence with at least about 60%, at least about 70%, at least about 80%, at least about 90%, at least about 95%, at least about 96%, at least about 97%, at least about 98%, at least about 99%, or about 100% identity thereto.

The polynucleotides of the disclosure can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the disclosure. For example, introns or portions of intron sequences can be incorporated into the polynucleotides of the disclosure. Incorporation of intronic sequences can increase protein production as well as polynucleotide expression levels. In some embodiments, the polynucleotide of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

### Regions Having a 5′ Cap

The disclosure also includes a polynucleotide that comprises both a 5′ Cap and a polynucleotide of the present disclosure (e.g., a polynucleotide comprising a nucleotide sequence encoding a polypeptide, or a repressor).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present disclosure (e.g., a polynucleotide comprising a nucleotide sequence encoding a first polypeptide, a polypeptide, or a repressor) incorporate a cap moiety.

In some embodiments, polynucleotides of the present disclosure (e.g., a polynucleotide comprising a nucleotide sequence encoding a polypeptide, or a repressor) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the disclosure (e.g., a polynucleotide comprising a nucleotide sequence encoding a polypeptide, or a repressor), can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N, pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly A Tails

In some embodiments, the polynucleotides of the present disclosure (e.g., a polynucleotide comprising a nucleotide sequence encoding a polypeptide, or a repressor) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO: 149). aaaaaaaaaa aaaaaaaaaa aaaaaaaaaa aaaaaaaaaa aaaaaaaaaa aaaaaaaaaa aaaaaaaaaa aaaaaaaaaa aaaaaaaaaa aaaaaaaaaa (SEQ ID NO: 149)

PolyA tails can also be added after the construct is exported from the nucleus.

According to the present disclosure, terminal groups on the poly A tail can be incorporated for stabilization. Polynucleotides of the present disclosure can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present disclosure can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs (Norbury, Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present disclosure. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO: 150).

(SEQ ID NO: 150)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

### Start Codon Region

The disclosure also includes a polynucleotide that comprises both a start codon region and the polynucleotide described herein (e.g., a polynucleotide comprising a nucleotide sequence encoding a polypeptide or a repressor). In some embodiments, the polynucleotides of the present disclosure can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The disclosure also includes a polynucleotide that comprises both a stop codon region and the polynucleotide described herein (e.g., a polynucleotide comprising a nucleotide sequence encoding a polypeptide or a repressor). In some embodiments, the polynucleotides of the present disclosure can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present disclosure include the stop codon TGA in the case or DNA, or the stop codon UGA in the case of RNA, and one additional stop codon. In a further embodiment the addition stop codon can be TAA or UAA. In another embodiment, the polynucleotides of the present disclosure include three consecutive stop codons, four stop codons, or more.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (mil) substitutions at one or more or all uridine positions of the nucleic acid.

In some embodiments, a RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions

The present disclosure provides pharmaceutical formulations comprising any of the systems, or compositions disclosed herein. In some embodiments, the pharmaceutical formulation comprises a messenger RNA (mRNA) comprising (i) an open reading frame encoding a polypeptide, and (ii) one or more microRNA target sites present in hematopoietic stem and progenitor cells (HSPC miRts).

In some embodiments, the pharmaceutical formulation comprises (a) a first polynucleotide comprising (i) a repressor binding element, (ii) an open reading frame encoding a polypeptide, and (iii) optionally one or more microRNA target sites present in non-hematopoietic stem and progenitor cells (non-HSPC miRts); and (b) a second polynucleotide comprising (i) a sequence encoding a repressor that binds to the repressor binding element and (ii) one or more microRNA target sites present in hematopoietic stem and progenitor cells (HSPC miRts), wherein binding of the repressor to the repressor binding element reduces translation of the polypeptide from the first polynucleotide.

In some embodiments, the pharmaceutical formulation comprises (a) a first polynucleotide comprising (i) a repressor binding element, (ii) an open reading frame encoding a polypeptide, and (iii) optionally one or more microRNA target sites present in hematopoietic stem and progenitor cells (HSPC miRts); and (b) a second polynucleotide comprising (i) a sequence encoding a repressor that binds to the repressor binding element and (ii) one or more microRNA target sites present in non-hematopoietic stem and progenitor cells (non-HSPC miRts), wherein binding of the repressor to the repressor binding element reduces translation of the polypeptide from the first polynucleotide.

In some embodiments of the disclosure, the polynucleotide are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intrasynovial, intrasternal, intrathecal, intrahepatic, intralesional, intracranial, intraventricular, oral, inhalation spray, pulmonary, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations and Delivery

The polynucleotide comprising an mRNA of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

A polynucleotide comprising an mRNA of the disclosure can be delivered to a cell using any method known in the art. For example, the polynucleotide comprising an mRNA of the disclosure can be delivered to a cell by a lipid-based delivery, e.g., transfection, or by electroporation.

### Delivery Agents

The compositions and systems disclosed herein further comprises a delivery agent. The delivery agent of the present disclosure can include, without limitation, liposomes, lipid nanoparticles, lipidoids, polymers, lipoplexes, polyplexes, microvesicles, exosomes, peptides, proteins, cells transfected with polynucleotides, hyaluronidase, nanoparticle mimics, nanotubes, conjugates, and combinations thereof.

a. Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and one or more polynucleotides disclosed herein, e.g., mRNA, has an increased therapeutic index as compared to a corresponding formulation which comprises a reference lipid (e.g., MC3, KC2, or DLinDMA) and the same one or more polyucleotides.

In some embodiments, the pharmaceutical composition comprises a messenger RNA (mRNA) comprising (i) an open reading frame encoding a polypeptide, and (ii) one or more microRNA target sites present in hematopoietic stem and progenitor cells (HSPC miRts).

In some embodiments, the pharmaceutical composition comprises (a) a first polynucleotide comprising (i) a repressor binding element, (ii) an open reading frame encoding a polypeptide, and (iii) optionally one or more microRNA target sites present in non-hematopoietic stem and progenitor cells (non-HSPC miRts); and (b) a second polynucleotide comprising (i) a sequence encoding a repressor that binds to the repressor binding element and (ii) one or more microRNA target sites present in hematopoietic stem and progenitor cells (HSPC miRts),
wherein binding of the repressor to the repressor binding element reduces translation of the polypeptide from the first polynucleotide.

In some embodiments, the pharmaceutical composition comprises (a) a first polynucleotide comprising (i) a repressor binding element, (ii) an open reading frame encoding a polypeptide, and (iii) optionally one or more microRNA target sites present in hematopoietic stem and progenitor cells (HSPC miRts); and (b) a second polynucleotide comprising (i) a sequence encoding a repressor that binds to the repressor binding element and (ii) one or more microRNA target sites present in non-hematopoietic stem and progenitor cells (non-HSPC miRts), wherein binding of the repressor to the repressor binding element reduces translation of the polypeptide from the first polynucleotide.

### (I) Lipid Nanoparticle Compositions

In some embodiments, the nucleic acids of the disclosure are formulated as lipid nanoparticle (LNP) compositions. The LNPs disclosed herein comprise an (i) ionizable lipid; (ii) sterol or other structural lipid; (iii) a non-cationic helper lipid or phospholipid; and, optionally a (iv) PEG lipid. These categories of lipids are set forth in more detail below. Lipid nanoparticles typically comprise amino lipid, phospholipid, structural lipid and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entireties.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### (I)(a) Amino Lipids

In some aspects, the amino lipids of a lipid nanoparticle composition disclosed herein may be one or more of compounds of Formula (I):

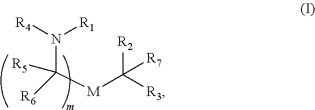

or their N-oxides, or salts or isomers thereof, wherein:
R1 is selected from the group consisting of C5-30 alkyl, C5-20 alkenyl, —R*YR″, —YR″, and —R″M′R′;
R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle;
R4 is selected from the group consisting of hydrogen, a C3-6 carbocycle, —(CH2)nQ, —(CH2)nCHQR, —CHQR, —CQ(R)2, and unsubstituted C1-6 alkyl, where Q is selected from a carbocycle, heterocycle, —OR, —O(CH2)nN(R)2, —C(O)OR, —OC(O)R, —CX3, —CX2H, —CXH2, —CN, —N(R)2, —C(O)N(R)2, —N(R)C(O)R, —N(R)S(O)2R, —N(R)C(O)N(R)2, —N(R)C(S)N(R)2, —N(R)R8, —N(R)S(O)2R8, —O(CH2)nOR, —N(R)C(═NR9)N(R)2, —N(R)C(═CHR9)N(R)2, —OC(O)N(R)2, —N(R)C(O)OR, —N(OR)C(O)R, —N(OR)S(O)2R, —N(OR)C(O)OR, —N(OR)C(O)N(R)2, —N(OR)C(S)N(R)2, —N(OR)C(═NR9)N(R)2, —N(OR)C(═CHR9)N(R)2, —C(═NR9)N(R)2, —C(═NR9)R, —C(O)N(R)OR, and —C(R)N(R)2C(O)OR, and each n is independently selected from 1, 2, 3, 4, and 5;
each R5 is independently selected from the group consisting of C1-3 alkyl, C2-3 alkenyl, and H;
each R6 is independently selected from the group consisting of C1-3 alkyl, C2-3 alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)2-, —S—S—, an aryl group, and a heteroaryl group, in which M″ is a bond, C1-13 alkyl or C2-13 alkenyl;
R7 is selected from the group consisting of C1-3 alkyl, C2-3 alkenyl, and H;
R8 is selected from the group consisting of C3-6 carbocycle and heterocycle;
R9 is selected from the group consisting of H, CN, NO2, C1-6 alkyl, —OR, —S(O)2R, —S(O)2N(R)2, C2-6 alkenyl, C3-6 carbocycle and heterocycle;
each R is independently selected from the group consisting of C1-3 alkyl, C2-3 alkenyl, and H;
each R′ is independently selected from the group consisting of C1-18 alkyl, C2-18 alkenyl, —R*YR″, —YR″, and H;
each R″ is independently selected from the group consisting of C3-15 alkyl and C3-15 alkenyl;
each R* is independently selected from the group consisting of C1-12 alkyl and C2-12 alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R⁴ is —(CH2)nQ, —(CH2)nCHQR, —CHQR, or —CQ(R)2, then (i) Q is not —N(R)2 when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

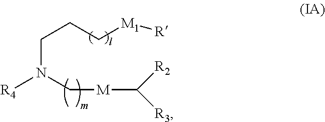

or its N-oxide, or a salt or isomer thereof, wherein l is selected from 1, 2, 3, 4, and 5; m is selected from 5, 6, 7, 8, and 9; M1 is a bond or M′; R4 is hydrogen, unsubstituted C1-3 alkyl, or —(CH2)nQ, in which Q is OH, —NHC(S)N(R)₂, —NHC(O)N(R)₂, —N(R)C(O)R, —N(R)S(O)2R, —N(R)R⁸, —NHC(═NR9)N(R)2, —NHC(═CHR9)N(R)2, —OC(O)N(R)2, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, m is 5, 7, or 9. For example, Q is OH, —NHC(S)N(R)2, or —NHC(O)N(R)2. For example, Q is —N(R)C(O)R, or —N(R)S(O)2R.

In certain embodiments, a subset of compounds of Formula (I) includes those of Formula (IB):

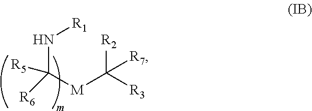

or its N-oxide, or a salt or isomer thereof in which all variables are as defined herein. For example, m is selected from 5, 6, 7, 8, and 9; R4 is hydrogen, unsubstituted C1-3 alkyl, or —(CH2)nQ, in which Q is OH, —NHC(S)N(R)2, —NHC(O)N(R)2, —N(R)C(O)R, —N(R)S(O)2R, —N(R)R⁸, —NHC(═NR9)N(R)2, —NHC(═CHR9)N(R)2, —OC(O)N(R)2, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, m is 5, 7, or 9. For example, Q is OH, —NHC(S)N(R)2, or —NHC(O)N(R)2. For example, Q is —N(R)C(O)R, or —N(R)S(O)2R.

In certain embodiments, a subset of compounds of Formula (I) includes those of Formula (II):

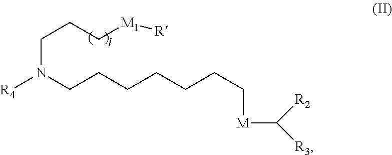

or its N-oxide, or a salt or isomer thereof, wherein l is selected from 1, 2, 3, 4, and 5; M1 is a bond or M′; R4 is hydrogen, unsubstituted C1-3 alkyl, or —(CH2)nQ, in which n is 2, 3, or 4, and Q is OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C1-14 alkyl, and C2-14 alkenyl.

In one embodiment, the compounds of Formula (I) are of Formula (IIa),

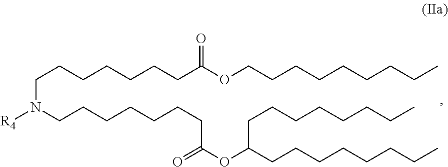

or their N-oxides, or salts or isomers thereof, wherein R4 is as described herein.

In another embodiment, the compounds of Formula (I) are of Formula (IIb),

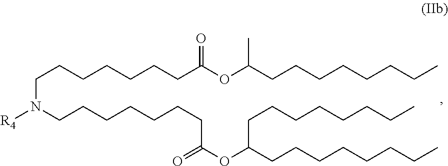

or their N-oxides, or salts or isomers thereof, wherein R4 is as described herein.

In another embodiment, the compounds of Formula (I) are of Formula (IIc) or (IIe):

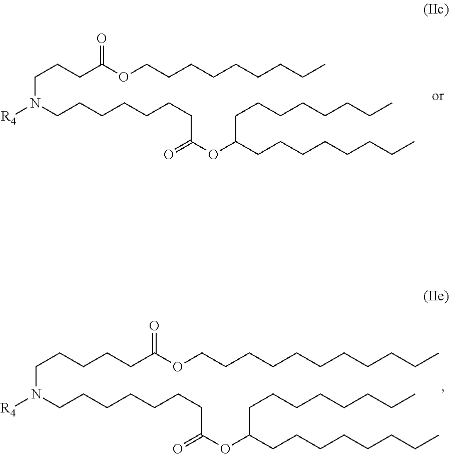

or their N-oxides, or salts or isomers thereof, wherein R4 is as described herein.

In another embodiment, the compounds of Formula (I) are of Formula (IIf):

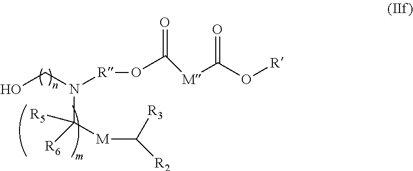

or their N-oxides, or salts or isomers thereof,
wherein M is —C(O)O— or —OC(O)—, M″ is C1-6 alkyl or C2-6 alkenyl, R2 and R3 are independently selected from the group consisting of C5-14 alkyl and C5-14 alkenyl, and n is selected from 2, 3, and 4.

In a further embodiment, the compounds of Formula (I) are of Formula (IId),

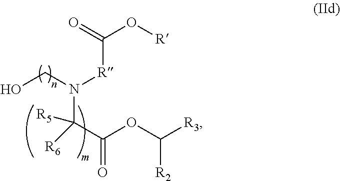

or their N-oxides, or salts or isomers thereof, wherein n is 2, 3, or 4; and m, R′, R″, and R2 through R6 are as described herein. For example, each of R2 and R3 may be independently selected from the group consisting of C5-14 alkyl and C5-14 alkenyl.

In a further embodiment, the compounds of Formula (I) are of Formula (IIg),

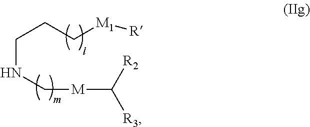

or their N-oxides, or salts or isomers thereof, wherein l is selected from 1, 2, 3, 4, and 5; m is selected from 5, 6, 7, 8, and 9; M1 is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C1-14 alkyl, and C2-14 alkenyl. For example, M″ is C1-6 alkyl (e.g., C1-4 alkyl) or C2-6 alkenyl (e.g. C2-4 alkenyl). For example, R2 and R3 are independently selected from the group consisting of C5-14 alkyl and C5-14 alkenyl.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is

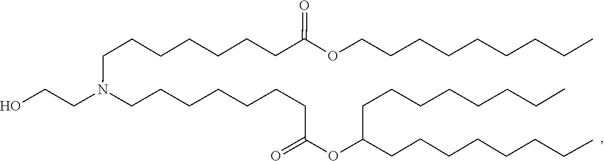 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is

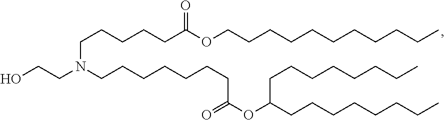 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (III),

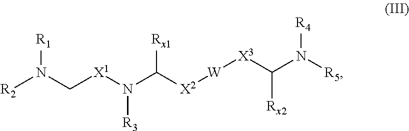

or salts or isomers thereof, wherein
W is

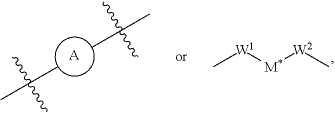

ring A is

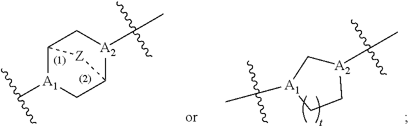

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —S C(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

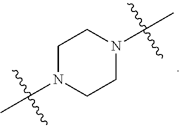 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (IIIa1)-(IIIa8):

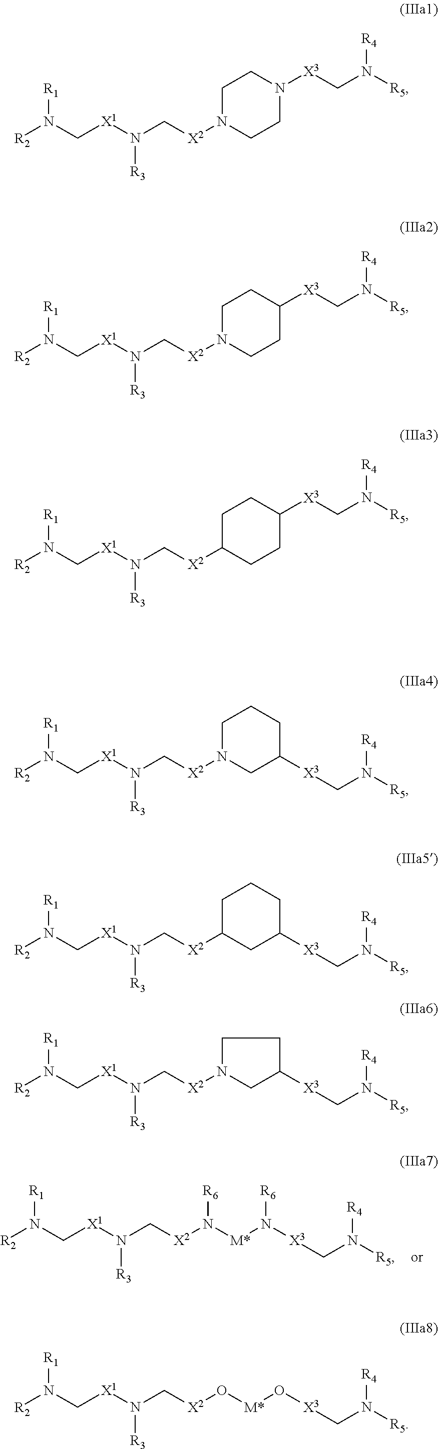

In some embodiments, the amino lipid is

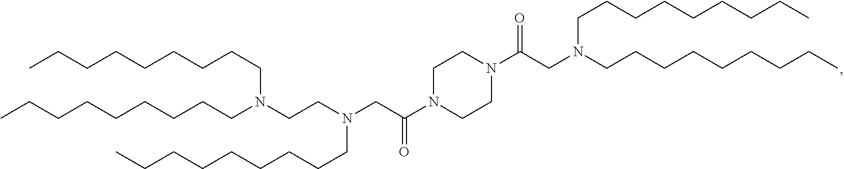 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH Thus, a lipid may have a positive or partial positive charge at physiological pH.

### (I)(b) Phospholipids

The lipid composition of a lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 DietherPC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present disclosure is a compound of Formula (IV):

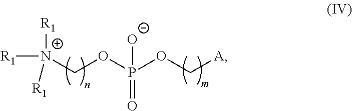

or a salt thereof, wherein:
each R1 is independently optionally substituted alkyl; or optionally two R1 are joined together with the intervening atoms to form optionally substituted monocyclic carbocyclyl or optionally substituted monocyclic heterocyclyl; or optionally three R1 are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
   A is of the formula:

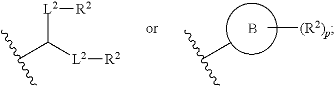

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), —OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N} NR^{N}C(NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

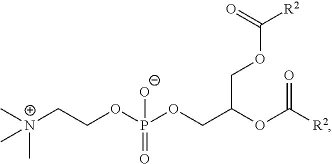

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530, or in International Application PCT/US2018/037922 filed on 15 Jun. 2018, the entire contents of each of which is hereby incorporated by reference in its entirety.

### i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

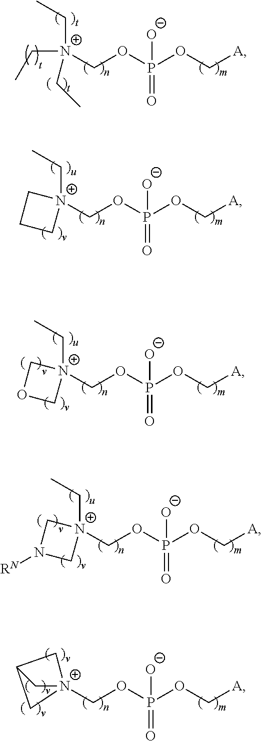

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

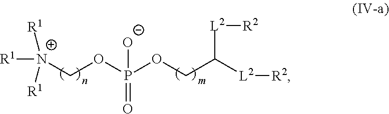

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

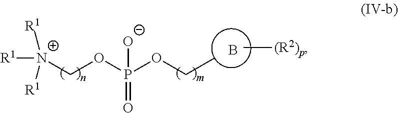

or a salt thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}) NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, —N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (IV) is of Formula (IV-c):

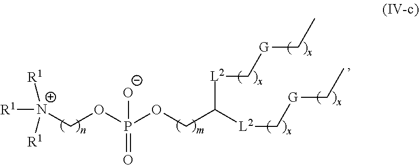

or a salt thereof, wherein:
each x is independently an integer between 0-30, inclusive; and each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O) NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}) NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following formulae:

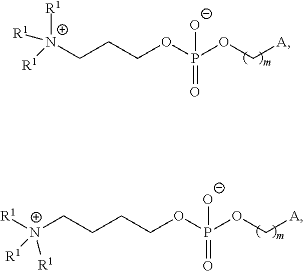

or a salt thereof.
   (iii) Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, an alternative lipid is useful.

In certain embodiments, an alternative lipid is used in place of a phospholipid of the present disclosure. In certain embodiments, an alternative lipid of the invention is oleic acid. In certain embodiments, the alternative lipid is one of the following:

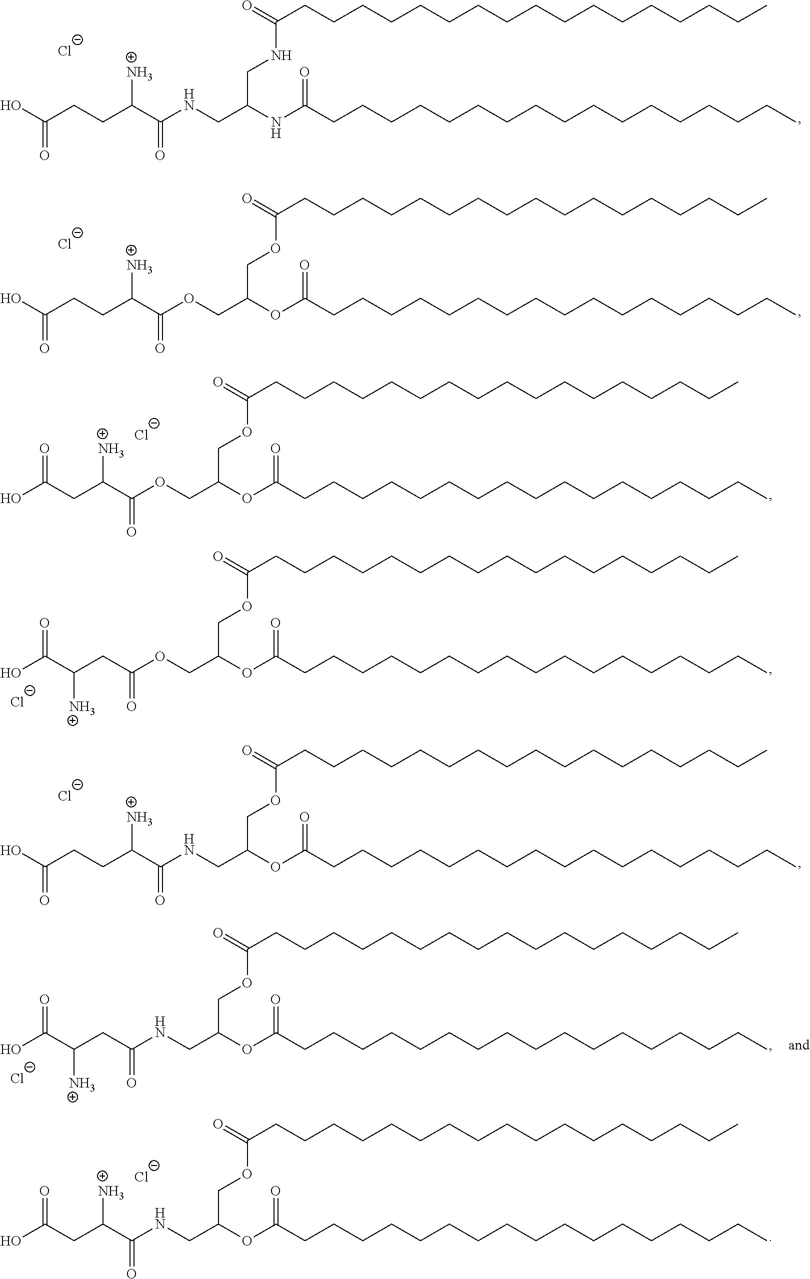

### (I)(c) Structural Lipids

The lipid composition of a lipid nanoparticle composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### (I)(d) Polyethylene Glycol (PEG)-Lipids

The lipid composition of a lipid nanoparticle composition disclosed herein disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C14 to about C22, preferably from about C14 to about C16. In some embodiments, a PEG moiety, for example an mPEG-NH2, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

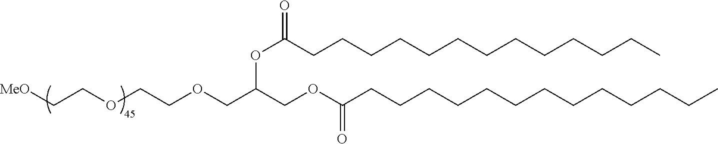

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

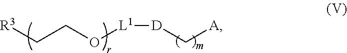

or salts thereof, wherein:
R³ is —ORO;
RO is hydrogen, optionally substituted alkyl, or an oxygen protecting group;
r is an integer between 1 and 100, inclusive;
L1 is optionally substituted C1-10 alkylene, wherein at least one methylene of the optionally substituted C1-10 alkylene is independently replaced with optionally substituted carbocyclylene, optionally substituted heterocyclylene, optionally substituted arylene, optionally substituted heteroarylene, O, N(RN), S, C(O), C(O)N(RN), —NRNC(O), C(O)O, OC(O), OC(O)O, OC(O)N(RN), NRNC(O)O, or NRNC(O)N(R^{N});
D is a moiety obtained by click chemistry or a moiety cleavable under physiological conditions;
m is 0, 1, 2, 3, 4, 5, 6, 7, 8, 9, or 10;
A is of the formula:

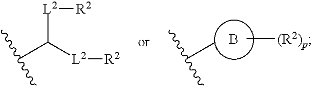

each instance of L2 is independently a bond or optionally substituted C1-6 alkylene, wherein one methylene unit of the optionally substituted C1-6 alkylene is optionally replaced with O, N(RN), S, C(O), C(O)N(RN), NRNC(O), C(O)O, OC(O), OC(O)O, —OC(O)N(RN), NRNC(O)O, or NRNC(O)N(RN);
each instance of R2 is independently optionally substituted C1-30 alkyl, optionally substituted C1-30 alkenyl, or optionally substituted C1-30 alkynyl; optionally wherein one or more methylene units of R2 are independently replaced with optionally substituted carbocyclylene, optionally substituted heterocyclylene, optionally substituted arylene, optionally substituted heteroarylene, N(RN), O, S, C(O), C(O)N(RN), —NRNC(O), NRNC(O)N(RN), C(O)O, OC(O), OC(O)O, OC(O)N(RN), NRNC(O)O, —C(O)S, SC(O), C(═NRN), C(═NRN)N(RN), NRNC(═NRN), NRNC(═NRN)N(RN), —C(S), C(S)N(RN), NRNC(S), NRNC(S)N(RN), S(O), OS(O), S(O)O, OS(O)O, OS(O)2, S(O)2O, OS(O)2O, N(RN)S(O), S(O)N(RN), N(RN)S(O)N(RN), OS(O)N(RN), —N(RN)S(O)O, S(O)2, N(RN)S(O)2, S(O)2N(RN), N(RN)S(O)2N(RN), OS(O)2N(RN), or N(RN)S(O)2O;
each instance of RN is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R3 is —ORO, and RO is hydrogen). In certain embodiments, the compound of Formula (V) is of Formula (V-OH):

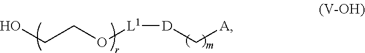

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI-A):

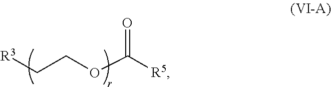

or a salts thereof, wherein:
R³ is —ORO;
RO is hydrogen, optionally substituted alkyl or an oxygen protecting group;
r is an integer between 1 and 100, inclusive;
R5 is optionally substituted C10-40 alkyl, optionally substituted C10-40 alkenyl, or optionally substituted C10-40 alkynyl; and optionally one or more methylene groups of R5 are replaced with optionally substituted carbocyclylene, optionally substituted heterocyclylene, optionally substituted arylene, optionally substituted heteroarylene, —N(RN), O, S, C(O), C(O)N(RN), NRNC(O), NRNC(O)N(RN), C(O)O, OC(O), OC(O)O, OC(O)N(RN), NRNC(O)O, C(O)S, SC(O), C(═NRN), C(═NRN)N(RN), NRNC(═NRN), NRNC(═NRN)N(RN), C(S), C(S)N(RN), NRNC(S), NRNC(S)N(RN), S(O), OS(O), —S(O)O, OS(O)O, OS(O)2, S(O)2O, OS(O)2O, N(RN)S(O), S(O)N(RN), —N(RN)S(O)N(RN), OS(O)N(RN), N(RN)S(O)O, S(O)2, N(RN)S(O)2, S(O)2N(RN), —N(RN)S(O)2N(RN), OS(O)2N(RN), or N(RN)S(O)2O; and
each instance of RN is independently hydrogen, optionally substituted alkyl, or a nitrogen protecting group.

In certain embodiments, the compound of Formula (VI) is of Formula (VI-OH):

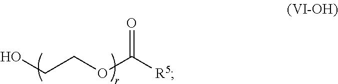

also referred to as (VI-B),
or a salt thereof. In some embodiments, r is 40-50.

In yet other embodiments the compound of Formula (VI-C) is:

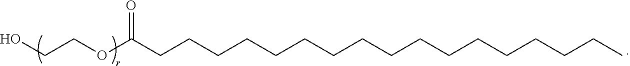

or a salt thereof.

In one embodiment, the compound of Formula (VI-D) is

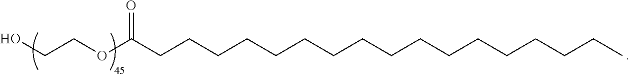

In some aspects, the lipid composition of the pharmaceutical compositions disclosed herein does not comprise a PEG-lipid. In some embodiments, the PEG-lipids may be one or more of the PEG lipids described in U.S. Application No. U.S. Ser. No. 15/674,872.

In some embodiments, a LNP of the disclosure comprises an amino lipid of any of Formula I, II or III, a phospholipid comprising DSPC, a structural lipid, and a PEG lipid comprising PEG-DMG. In some embodiments, a LNP of the disclosure comprises an amino lipid of any of Formula I, II or III, a phospholipid comprising DSPC, a structural lipid, and a PEG lipid comprising a compound having Formula VI. In some embodiments, a LNP of the disclosure comprises an amino lipid of Formula I, II or III, a phospholipid comprising a compound having Formula IV, a structural lipid, and the PEG lipid comprising a compound having Formula V or VI. In some embodiments, a LNP of the disclosure comprises an amino lipid of Formula I, II or III, a phospholipid comprising a compound having Formula IV, a structural lipid, and the PEG lipid comprising a compound having Formula V or VI. In some embodiments, a LNP of the disclosure comprises an amino lipid of Formula I, II or III, a phospholipid having Formula IV, a structural lipid, and a PEG lipid comprising a compound having Formula VI.

In some embodiments, a LNP of the disclosure comprises an N:P ratio of from about 2:1 to about 30:1. In some embodiments, a LNP of the disclosure comprises an N:P ratio of about 6:1. In some embodiments, a LNP of the disclosure comprises an N:P ratio of about 3:1, 4:1, or 5:1. In some embodiments, a LNP of the disclosure comprises a wt/wt ratio of the amino lipid component to the RNA of from about 10:1 to about 100:1. In some embodiments, a LNP of the disclosure comprises a wt/wt ratio of the amino lipid component to the RNA of about 20:1. In some embodiments, a LNP of the disclosure comprises a wt/wt ratio of the amino lipid component to the RNA of about 10:1.

In some embodiments, a LNP of the disclosure has a mean diameter from about 30 nm to about 150 nm. In some embodiments, a LNP of the disclosure has a mean diameter from about 60 nm to about 120 nm.

b. Nanoparticle Compositions

In some embodiments, the pharmaceutical compositions disclosed herein are formulated as lipid nanoparticles (LNP). Accordingly, the present disclosure also provides nanoparticle compositions comprising (I) a lipid composition comprising a delivery agent such as compound as described herein, and (II) (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide.

In some embodiments, the present disclosure also provides nanoparticle compositions comprising (I) a lipid composition comprising a delivery agent such as compound as described herein, and (II)(a) a first polynucleotide comprising (i) an open reading frame encoding a first polypeptide, (ii) an effector binding element, and (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element increases translation of the first polypeptide from the first polynucleotide.

In some embodiments, the present disclosure also provides nanoparticle compositions comprising (I) a lipid composition comprising a delivery agent such as compound as described herein, and (II) (a) an RNA molecule comprising in order from the 5′ to 3′ end of the RNA (i) an open reading frame encoding a polypeptide, (ii) a polyA tail, (iii) a cleavage site, and (iv) a destabilizing sequence; and (b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.

In such nanoparticle compositions described above, the lipid composition disclosed herein can encapsulate the polynucleotide encoding a first polypeptide and, when present, the polynucleotide encoding the second polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid:about 5-25% structural lipid:about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group.

In one embodiment, the ionizable lipid may be selected from, but not limited to, an ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a first polypeptide, and optionally in combination with the polynucleotide encoding a second polypeptide, are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 m or shorter (e.g., 1 m, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

c. Liposomes, Lipoplexes, and Lipid Nanoparticles

In some embodiments, the nucleic acids of the disclosure are formulated as liposome compositions, lipoplex compositions, and/or polyplex compositions. Such compositions, and methods are generally known in the art, see for example Itziar Gomez-Aguado I. et al., Nanomaterials 2020, 10, 364; Kowalski P. S. et al., Mol Ther. 2019 Apr. 10; 27(4): 710-728; ur Rehman Z, et al. ACS Nano. 2013 May 28; 7(5):3767-77; and U.S. Patent Application Publication US20160367702, incorporated by reference herein in its entirety.

In some embodiments, the compositions or formulations of the present disclosure comprise a delivery agent, e.g., a liposome, a lioplexes, a lipid nanoparticle, or any combination thereof. The polynucleotides described herein (e.g., a polynucleotide comprising first polypeptide) can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MVLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the polynucleotides described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the polynucleotides described herein can be formulated in a cationic oil-in-water emulsion where the emulsion particle comprises an oil core and a cationic lipid that can interact with the polynucleotide anchoring the molecule to the emulsion particle. In some embodiments, the polynucleotides described herein can be formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the polynucleotides described herein can be formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the polynucleotides described herein can be formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)-N,N-dimethyl-3-nonyldocosa-13,16-dien-1-amine (L608), (20Z,23Z)-N,N-dimethylnonacosa-20,23-dien-10-amine, (17Z,20Z)-N,N-dimemylhexacosa-17,20-dien-9-amine, (16Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)-N,N-dimethylnonacosa-11,20,2-trien-10-amine, and any combination thereof. Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.
The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.
The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin P4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the polynucleotide described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the polynucleotides described herein are formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the polynucleotides described herein can be formulated for controlled release and/or targeted delivery. As used herein, “controlled release” refers to a pharmaceutical composition or compound release profile that conforms to a particular pattern of release to effect a therapeutic outcome. In one embodiment, the polynucleotides can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the polynucleotides described herein can be encapsulated in a therapeutic nanoparticle, referred to herein as “therapeutic nanoparticle polynucleotides.” Therapeutic nanoparticles can be formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle polynucleotide can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the polynucleotides described herein can be formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle polynucleotide can be formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsevet al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM,) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the polynucleotides described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the polynucleotides can be formulated in lipid nanoparticles using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In some embodiments, the polynucleotides described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, or less than 975 μm.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

d. Lipidoids

In some embodiments, the compositions or formulations of the present disclosure comprise a delivery agent, e.g., a lipidoid. The polynucleotides described herein (e.g., a polynucleotide comprising a nucleotide sequence encoding a first polypeptide) can be formulated with lipidoids. Complexes, micelles, liposomes or particles can be prepared containing these lipidoids and therefore to achieve an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C12-200 (including derivatives and variants), and MD1, can be tested for in vivo activity. The lipidoid “98N12-5” is disclosed by Akinc et al., Mol Ther. 2009 17:872-879. The lipidoid “C12-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the polynucleotides described herein can be formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to polynucleotides. Lipidoids and polynucleotide formulations comprising lipidoids are described in Intl. Pub. No. WO 2015051214 (herein incorporated by reference in its entirety.

e. Hyaluronidase

In some embodiments, the polynucleotides described herein (e.g., a polynucleotide comprising a nucleotide sequence encoding a first polypeptide) and hyaluronidase for injection (e.g., intramuscular or subcutaneous injection). Hyaluronidase catalyzes the hydrolysis of hyaluronan, which is a constituent of the interstitial barrier. Hyaluronidase lowers the viscosity of hyaluronan, thereby increases tissue permeability (Frost, Expert Opin. Drug Deliv. (2007) 4:427-440). Alternatively, the hyaluronidase can be used to increase the number of cells exposed to the polynucleotides administered intramuscularly, or subcutaneously.

f. Nanoparticle Mimics

In some embodiments, the polynucleotides described herein (e.g., a polynucleotide comprising a nucleotide sequence encoding a first polypeptide) is encapsulated within and/or absorbed to a nanoparticle mimic. A nanoparticle mimic can mimic the delivery function organisms or particles such as, but not limited to, pathogens, viruses, bacteria, fungus, parasites, prions and cells. As a non-limiting example, the polynucleotides described herein can be encapsulated in a non-viron particle that can mimic the delivery function of a virus (see e.g., Intl. Pub. No. WO2012006376 and U.S. Pub. Nos. US20130171241 and US20130195968, each of which is herein incorporated by reference in its entirety).

g. Self-Assembled Nanoparticles, or Self-Assembled Macromolecules

In some embodiments, the compositions or formulations of the present disclosure comprise the polynucleotides described herein (e.g., a polynucleotide comprising a nucleotide sequence encoding a first polypeptide) in self-assembled nanoparticles, or amphiphilic macromolecules (AMs) for delivery. AMs comprise biocompatible amphiphilic polymers that have an alkylated sugar backbone covalently linked to poly(ethylene glycol). In aqueous solution, the AMs self-assemble to form micelles. Nucleic acid self-assembled nanoparticles are described in Intl. Appl. No. PCT/US2014/027077, and AMs and methods of forming AMs are described in U.S. Pub. No. US20130217753, each of which is herein incorporated by reference in its entirety.

h. Cations and Anions

In some embodiments, the compositions or formulations of the present disclosure comprise the polynucleotides described herein (e.g., a polynucleotide comprising a nucleotide sequence encoding a first polypeptide) and a cation or anion, such as Zn2+, Ca2+, Cu2+, Mg2+ and combinations thereof. Exemplary formulations can include polymers and a polynucleotide complexed with a metal cation as described in, e.g., U.S. Pat. Nos. 6,265,389 and 6,555,525, each of which is herein incorporated by reference in its entirety. In some embodiments, cationic nanoparticles can contain a combination of divalent and monovalent cations. The delivery of polynucleotides in cationic nanoparticles or in one or more depot comprising cationic nanoparticles can improve polynucleotide bioavailability by acting as a long-acting depot and/or reducing the rate of degradation by nucleases.

i. Amino Acid Lipids

In some embodiments, the compositions or formulations of the present disclosure comprise the polynucleotides described herein (e.g., a polynucleotide comprising a nucleotide sequence encoding a first polypeptide) that is formulation with an amino acid lipid. Amino acid lipids are lipophilic compounds comprising an amino acid residue and one or more lipophilic tails. Non-limiting examples of amino acid lipids and methods of making amino acid lipids are described in U.S. Pat. No. 8,501,824. The amino acid lipid formulations can deliver a polynucleotide in releasable form that comprises an amino acid lipid that binds and releases the polynucleotides. As a non-limiting example, the release of the polynucleotides described herein can be provided by an acid-labile linker as described in, e.g., U.S. Pat. Nos. 7,098,032, 6,897,196, 6,426,086, 7,138,382, 5,563,250, and 5,505,931, each of which is herein incorporated by reference in its entirety.

j. Interpolyelectrolyte Complexes

In some embodiments, the compositions or formulations of the present disclosure comprise the polynucleotides described herein (e.g., a polynucleotide comprising a nucleotide sequence encoding a first polypeptide) in an interpolyelectrolyte complex. Interpolyelectrolyte complexes are formed when charge-dynamic polymers are complexed with one or more anionic molecules. Non-limiting examples of charge-dynamic polymers and interpolyelectrolyte complexes and methods of making interpolyelectrolyte complexes are described in U.S. Pat. No. 8,524,368, herein incorporated by reference in its entirety.

k. Crystalline Polymeric Systems

In some embodiments, the compositions or formulations of the present disclosure comprise the polynucleotides described herein (e.g., a polynucleotide comprising a nucleotide sequence encoding a first polypeptide) in crystalline polymeric systems. Crystalline polymeric systems are polymers with crystalline moieties and/or terminal units comprising crystalline moieties. Exemplary polymers are described in U.S. Pat. No. 8,524,259 (herein incorporated by reference in its entirety).

l. Polymers, Biodegradable Nanoparticles, and Core-Shell Nanoparticles

In some embodiments, the compositions or formulations of the present disclosure comprise the polynucleotides described herein (e.g., a polynucleotide comprising a nucleotide sequence encoding a first polypeptide) and a natural and/or synthetic polymer. The polymers include, but not limited to, polyethenes, polyethylene glycol (PEG), poly(1-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, elastic biodegradable polymer, biodegradable copolymer, biodegradable polyester copolymer, biodegradable polyester copolymer, multiblock copolymers, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyureas, polystyrenes, polyamines, polylysine, poly(ethylene imine), poly(serine ester), poly(L-lactide-co-L-lysine), poly(4-hydroxy-L-proline ester), amine-containing polymers, dextran polymers, dextran polymer derivatives or combinations thereof.

Exemplary polymers include, DYNAMIC POLYCONJUGATE® (Arrowhead Research Corp., Pasadena, CA) formulations from MIRUS® Bio (Madison, WI) and Roche Madison (Madison, WI), PHASERX™ polymer formulations such as, without limitation, SMARTT POLYMER TECHNOLOGY™ (PHASERX®, Seattle, WA), DMRI/DOPE, poloxamer, VAXFECTIN® adjuvant from Vical (San Diego, CA), chitosan, cyclodextrin from Calando Pharmaceuticals (Pasadena, CA), dendrimers and poly(lactic-co-glycolic acid) (PLGA) polymers. RONDEL™ (RNAi/Oligonucleotide Nanoparticle Delivery) polymers (Arrowhead Research Corporation, Pasadena, CA) and pH responsive co-block polymers such as PHASERX® (Seattle, WA).

The polymer formulations allow a sustained or delayed release of the polynucleotide (e.g., following intramuscular or subcutaneous injection). The altered release profile for the polynucleotide can result in, for example, translation of an encoded protein over an extended period of time. The polymer formulation can also be used to increase the stability of the polynucleotide. Sustained release formulations can include, but are not limited to, PLGA microspheres, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc. Deerfield, IL), PEG-based sealants, and COSEAL® (Baxter International, Inc. Deerfield, IL).

As a non-limiting example modified mRNA can be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradable, biocompatible polymers that are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C.

As a non-limiting example, the polynucleotides described herein can be formulated with the polymeric compound of PEG grafted with PLL as described in U.S. Pat. No. 6,177,274. As another non-limiting example, the polynucleotides described herein can be formulated with a block copolymer such as a PLGA-PEG block copolymer (see e.g., U.S. Pub. No. US20120004293 and U.S. Pat. Nos. 8,236,330 and 8,246,968), or a PLGA-PEG-PLGA block copolymer (see e.g., U.S. Pat. No. 6,004,573). Each of the references is herein incorporated by reference in its entirety.

In some embodiments, the polynucleotides described herein can be formulated with at least one amine-containing polymer such as, but not limited to polylysine, polyethylene imine, poly(amidoamine) dendrimers, poly(amine-co-esters) or combinations thereof. Exemplary polyamine polymers and their use as delivery agents are described in, e.g., U.S. Pat. Nos. 8,460,696, 8,236,280, each of which is herein incorporated by reference in its entirety.

In some embodiments, the polynucleotides described herein can be formulated in a biodegradable cationic lipopolymer, a biodegradable polymer, or a biodegradable copolymer, a biodegradable polyester copolymer, a biodegradable polyester polymer, a linear biodegradable copolymer, PAGA, a biodegradable cross-linked cationic multi-block copolymer or combinations thereof as described in, e.g., U.S. Pat. Nos. 6,696,038, 6,517,869, 6,267,987, 6,217,912, 6,652,886, 8,057,821, and 8,444,992; U.S. Pub. Nos. US20030073619, US20040142474, US20100004315, US2012009145 and US20130195920; and Intl Pub. Nos. WO2006063249 and WO2013086322, each of which is herein incorporated by reference in its entirety.

In some embodiments, the polynucleotides described herein can be formulated in or with at least one cyclodextrin polymer as described in U.S. Pub. No. US20130184453. In some embodiments, the polynucleotides described herein can be formulated in or with at least one crosslinked cation-binding polymers as described in Intl. Pub. Nos. WO2013106072, WO2013106073 and WO2013106086. In some embodiments, the polynucleotides described herein can be formulated in or with at least PEGylated albumin polymer as described in U.S. Pub. No. US20130231287. Each of the references is herein incorporated by reference in its entirety.

In some embodiments, the polynucleotides disclosed herein can be formulated as a nanoparticle using a combination of polymers, lipids, and/or other biodegradable agents, such as, but not limited to, calcium phosphate. Components can be combined in a core-shell, hybrid, and/or layer-by-layer architecture, to allow for fine-tuning of the nanoparticle for delivery (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in their entireties). As a non-limiting example, the nanoparticle can comprise a plurality of polymers such as, but not limited to hydrophilic-hydrophobic polymers (e.g., PEG-PLGA), hydrophobic polymers (e.g., PEG) and/or hydrophilic polymers (Intl. Pub. No. WO20120225129, herein incorporated by reference in its entirety).

The use of core-shell nanoparticles has additionally focused on a high-throughput approach to synthesize cationic cross-linked nanogel cores and various shells (Siegwart et al., Proc Natl Acad Sci USA. 2011 108:12996-13001; herein incorporated by reference in its entirety). The complexation, delivery, and internalization of the polymeric nanoparticles can be precisely controlled by altering the chemical composition in both the core and shell components of the nanoparticle. For example, the core-shell nanoparticles can efficiently deliver siRNA to mouse hepatocytes after they covalently attach cholesterol to the nanoparticle.

In some embodiments, a hollow lipid core comprising a middle PLGA layer and an outer neutral lipid layer containing PEG can be used to delivery of the polynucleotides as described herein. In some embodiments, the lipid nanoparticles can comprise a core of the polynucleotides disclosed herein and a polymer shell, which is used to protect the polynucleotides in the core. The polymer shell can be any of the polymers described herein and are known in the art. The polymer shell can be used to protect the polynucleotides in the core.

Core-shell nanoparticles for use with the polynucleotides described herein are described in U.S. Pat. No. 8,313,777 or Intl. Pub. No. WO2013124867, each of which is herein incorporated by reference in their entirety.

m. Peptides and Proteins

In some embodiments, the compositions or formulations of the present disclosure comprise the polynucleotides described herein (e.g., a polynucleotide comprising a nucleotide sequence encoding a first polypeptide) that is formulated with peptides and/or proteins to increase transfection of cells by the polynucleotide, and/or to alter the biodistribution of the polynucleotide (e.g., by targeting specific tissues or cell types), and/or increase the translation of encoded protein (e.g., Intl. Pub. Nos. WO2012110636 and WO2013123298. In some embodiments, the peptides can be those described in U.S. Pub. Nos. US20130129726, US20130137644 and US20130164219. Each of the references is herein incorporated by reference in its entirety.

n. Conjugates
In some embodiments, the compositions or formulations of the present disclosure comprise the polynucleotides described herein (e.g., a polynucleotide comprising a nucleotide sequence encoding a first polypeptide) that is covalently linked to a carrier or targeting group, or including two encoding regions that together produce a fusion protein (e.g., bearing a targeting group and therapeutic protein or peptide) as a conjugate. The conjugate can be a peptide that selectively directs the nanoparticle to neurons in a tissue or organism, or assists in crossing the blood-brain barrier.

The conjugates include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid, an oligonucleotide (e.g., an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some embodiments, the conjugate can function as a carrier for the polynucleotide disclosed herein. The conjugate can comprise a cationic polymer such as, but not limited to, polyamine, polylysine, polyalkylenimine, and polyethylenimine that can be grafted to with poly(ethylene glycol). Exemplary conjugates and their preparations are described in U.S. Pat. No. 6,586,524 and U.S. Pub. No. US20130211249, each of which herein is incorporated by reference in its entirety.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as an endothelial cell or bone cell. Targeting groups can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent frucose, or aptamers. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein. As a non-limiting example, the targeting group can be a glutathione receptor (GR)-binding conjugate for targeted delivery across the blood-central nervous system barrier as described in, e.g., U.S. Pub. No. US2013021661012 (herein incorporated by reference in its entirety).

In some embodiments, the conjugate can be a synergistic biomolecule-polymer conjugate, which comprises a long-acting continuous-release system to provide a greater therapeutic efficacy. The synergistic biomolecule-polymer conjugate can be those described in U.S. Pub. No. US20130195799. In some embodiments, the conjugate can be an aptamer conjugate as described in Intl. Pat. Pub. No. WO2012040524. In some embodiments, the conjugate can be an amine containing polymer conjugate as described in U.S. Pat. No. 8,507,653. Each of the references is herein incorporated by reference in its entirety. In some embodiments, the polynucleotides can be conjugated to SMARTT POLYMER TECHNOLOGY® (PHASERX®, Inc. Seattle, WA).

In some embodiments, the polynucleotides described herein are covalently conjugated to a cell penetrating polypeptide, which can also include a signal sequence or a targeting sequence. The conjugates can be designed to have increased stability, and/or increased cell transfection; and/or altered the biodistribution (e.g., targeted to specific tissues or cell types).

In some embodiments, the polynucleotides described herein can be conjugated to an agent to enhance delivery. In some embodiments, the agent can be a monomer or polymer such as a targeting monomer or a polymer having targeting blocks as described in Intl. Pub. No. WO2011062965. In some embodiments, the agent can be a transport agent covalently coupled to a polynucleotide as described in, e.g., U.S. Pat. Nos. 6,835,393 and 7,374,778. In some embodiments, the agent can be a membrane barrier transport enhancing agent such as those described in U.S. Pat. Nos. 7,737,108 and 8,003,129. Each of the references is herein incorporated by reference in its entirety.

### Exemplary Additional Components of Compositions

### Surfactants

In certain embodiments, a composition of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10-4 M in water at about 30° C. and atmospheric pressure.

For example, amphiphilic polymer has a critical micelle concentration (CMC) ranging between about 0.1×10-4 M and about 1.3×10-4 M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

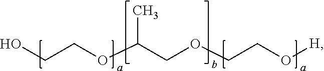

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

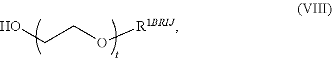

or a salt or isomer thereof, wherein:
t is an integer between 1 and 100;
R1BRIJ independently is C10-40 alkyl, C10-40 alkenyl, or C10-40 alkynyl; and optionally one or more methylene groups of R5PEG are independently replaced with C3-10 carbocyclylene, 4 to 10 membered heterocyclylene, C6-10 arylene, 4 to 10 membered heteroarylene, —N(RN)—, —O—, —S—, —C(O)—, —C(O)N(RN)—, —NRNC(O)—, —NRNC(O)N(RN)—, —C(O)O—, —OC(O)—, —OC(O)O—, —OC(O)N(RN)—, —NRNC(O)O—, —C(O)S—, —SC(O)—, —C(═NRN)—, —C(═NRN)N(RN)—, —NRNC(═NRN)—, —NRNC(═NRN)N(RN)—, —C(S)—, —C(S)N(RN)—, —NRNC(S)—, —NRNC(S)N(RN)—, —S(O)—, —OS(O)—, —S(O)O—, —OS(O)O—, —OS(O)2-, —S(O)2O-, —OS(O)2O-, —N(RN)S(O)—, —S(O)N(RN)—, —N(RN)S(O)N(RN)—, —OS(O)N(RN)—, —N(RN)S(O)O—, —S(O)2-, —N(RN)S(O)2-, —S(O)2N(RN)—, —N(RN)S(O)2N(RN)—, —OS(O)2N(RN)—, or —N(RN)S(O)2O-; and
each instance of RN is independently hydrogen, C1-6 alkyl, or a nitrogen protecting group

In some embodiment, R1BRIJ is C18 alkyl. For example, the polyethylene glycol ether is a compound of Formula (VIII-a):

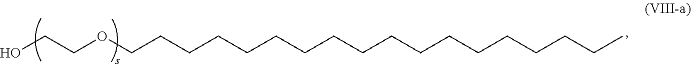

or a salt or isomer thereof.

In some embodiments, R1BRIJ is C18 alkenyl. For example, the polyethylene glycol ether is a compound of Formula (VIII-b):

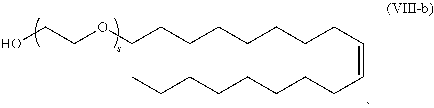

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

### Adjuvants

In some embodiments, a lipid nanoparticle composition of the disclosure optionally includes one or more adjuvants, e.g., Glucopyranosyl Lipid Adjuvant (GLA), CpG oligodeoxynucleotides (e.g., Class A or B), poly(I.C), aluminum hydroxide, and Pam3CSK4.

### Other Components

In some embodiments, a lipid nanoparticle composition of the disclosure may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 04, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

### Pharmaceutically Acceptable Excipients

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R.

Gennaro; Lippincott, Williams & Wilkins, Baltimore, MD, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof. A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Production of Lipid Nanoparticle Compositions

### A. Production of Nanoparticle Compositions

In order to investigate safe and efficacious nanoparticle compositions for use in the delivery of polynucleotides of the disclosure to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the polynucleotides of the disclosure and the other has the lipid components.

Lipid compositions are prepared by combining a lipid according to Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8) or a non-cationic helper lipid (such as DOPE, or DSPC obtainable from Avanti Polar Lipids, Alabaster, AL), a PEG lipid (such as 1,2 dimyristoyl sn glycerol methoxypolyethylene glycol, also known as PEG-DMG, obtainable from Avanti Polar Lipids, Alabaster, AL), and a phytosterol (optionally including a structural lipid such as cholesterol) at concentrations of about, e.g., 50 mM in a solvent, e.g., ethanol. Solutions should be refrigerated for storage at, for example, −20° C. Lipids are combined to yield desired molar ratios (see, for example, Table 7 below) and diluted with water and ethanol to a final lipid concentration of e.g., between about 5.5 mM and about 25 mM. Phytosterol* in Table 7 refers to phytosterol or optionally a combination of phytosterol and structural lipid such as beta-phytosterol and cholesterol.

TABLE 7
Exemplary formulations of Lipid Nanoparticle compositions

| Composition | |
|---|---|
| (mol %) | Components |
| 40:20:38.5:1.5 | Ionizable lipid:Phospholipid:Phytosterol*:PEG-DMG |
| 45:15:38.5:1.5 | Ionizable lipid:Phospholipid:Phytosterol*:PEG-DMG |
| 50:10:38.5:1.5 | Ionizable lipid:Phospholipid:Phytosterol*:PEG-DMG |
| 55:5:38.5:1.5 | Ionizable lipid:Phospholipid:Phytosterol*:PEG-DMG |
| 60:5:33.5:1.5 | Ionizable lipid:Phospholipid:Phytosterol*:PEG-DMG |
| 45:20:33.5:1.5 | Ionizable lipid:Phospholipid:Phytosterol*:PEG-DMG |
| 50:20:28.5:1.5 | Ionizable lipid:Phospholipid:Phytosterol*:PEG-DMG |
| 55:20:23.5:1.5 | Ionizable lipid:Phospholipid:Phytosterol*:PEG-DMG |
| 60:20:18.5:1.5 | Ionizable lipidd:Phospholipid:Phytosterol*:PEG-DMG |
| 40:15:43.5:1.5 | Ionizable lipid:Phospholipid:Phytosterol*:PEG-DMG |
| 50:15:33.5:1.5 | Ionizable lipid:Phospholipid:Phytosterol*:PEG-DMG |
| 55:15:28.5:1.5 | Ionizable lipid:Phospholipid:Phytosterol*:PEG-DMG |
| 60:15:23.5:1.5 | Ionizable lipid:Phospholipid:Phytosterol*:PEG-DMG |
| 40:10:48.5:1.5 | Ionizable lipid:Phospholipid:Phytosterol*:PEG-DMG |
| 45:10:43.5:1.5 | Ionizable lipid:Phospholipid:Phytosterol*:PEG-DMG |
| 55:10:33.5:1.5 | Ionizable lipid:Phospholipid:Phytosterol*:PEG-DMG |
| 60:10:28.5:1.5 | Ionizable lipidd:Phospholipid:Phytosterol*:PEG-DMG |
| 40:5:53.5:1.5 | Ionizable lipid:Phospholipid:Phytosterol*:PEG-DMG |
| 45:5:48.5:1.5 | Ionizable lipid:Phospholipid:Phytosterol*:PEG-DMG |
| 50:5:43.5:1.5 | Ionizable lipid:Phospholipid:Phytosterol*:PEG-DMG |
| 40:20:40:0 | Ionizable lipid:Phospholipid:Phytosterol*:PEG-DMG |
| 45:20:35:0 | Ionizable lipid:Phospholipid:Phytosterol*:PEG-DMG |
| 50:20:30:0 | Ionizable lipidd:Phospholipid:Phytosterol*:PEG-DMG |
| 55:20:25:0 | Ionizable lipid:Phospholipid:Phytosterol*:PEG-DMG |
| 60:20:20:0 | Ionizable lipid:Phospholipid:Phytosterol*:PEG-DMG |
| 40:15:45:0 | Ionizable lipid:Phospholipid:Phytosterol*:PEG-DMG |
| 45:15:40:0 | Ionizable lipid:Phospholipid:Phytosterol*:PEG-DMG |
| 50:15:35:0 | Ionizable lipid:Phospholipid:Phytosterol*:PEG-DMG |
| 55:15:30:0 | Ionizable lipidPhospholipid:Phytosterol*:PEG-DMG |
| 60:15:25:0 | Ionizable lipid:Phospholipid:Phytosterol*:PEG-DMG |
| 40:10:50:0 | Ionizable lipid:Phospholipid:Phytosterol*:PEG-DMG |
| 45:10:45:0 | Ionizable lipidPhospholipid:Phytosterol*:PEG-DMG |
| 50:0:48.5:1.5 | Ionizable lipid:Phospholipid:Phytosterol*:PEG-DMG |
| 50:10:40:0 | Ionizable lipid:Phospholipid:Phytosterol*:PEG-DMG |
| 55:10:35:0 | Ionizable lipid:Phospholipid:Phytosterol*:PEG-DMG |
| 60:10:30:0 | Ionizable lipidPhospholipid:Phytosterol*:PEG-DMG |

Nanoparticle compositions including polynucleotide(s) of the disclosure and a lipid component are prepared by combining the lipid solution with a solution including the polynucleotides of the disclosure at lipid component to polynucleotides wt:wt ratios between about 5:1 and about 50:1. The lipid solution is rapidly injected using a NanoAssemblr microfluidic based system at flow rates between about 10 ml/min and about 18 ml/min into the polynucleotides solution to produce a suspension with a water to ethanol ratio between about 1:1 and about 4:1.

For nanoparticle compositions including an RNA, solutions of the RNA at concentrations of 0.1 mg/ml in deionized water are diluted in a buffer, e.g., 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa. The first dialysis is carried out at room temperature for 3 hours. The formulations are then dialyzed overnight at 4° C. The resulting nanoparticle suspension is filtered through 0.2 m sterile filters (Sarstedt, Nümbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, may be used to achieve the same nano-precipitation.

### B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a polynucleotide (e.g., RNA) in nanoparticle compositions. 100 μL of the diluted formulation in 1×PBS is added to 900 L of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of the polynucleotides of the disclosure in the nanoparticle composition can be calculated based on the extinction coefficient of the polynucleotides used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, CA) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 L of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### C. In Vivo Formulation Studies

In order to monitor how effectively various nanoparticle compositions deliver polynucleotides of the disclosure to targeted cells, different nanoparticle compositions including a particular polynucleotide of the disclosure (for example, an mRNA) are prepared and administered to rodent populations. Mice are intravenously, intramuscularly, subcutaneously, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a polynucleotide of the disclosure in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA are useful in the evaluation of the efficacy and usefulness of various formulations for the delivery of polynucleotides. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the polynucleotides by a given nanoparticle composition relative to other nanoparticle compositions or the absence thereof.

### Example 2: HSPC miR Candidates can Suppress Reporter mRNA Expression in Mouse HSPCs In Vivo

This example describes how to achieve OFF target expression of a target mRNA in HSPCs in vivo using the system described in FIGS. 1A-B.

C57BL/6 mice (n=6 per group) were injected intravenously with 0.5 mg/kg mOX40L reporter mRNAs containing three miR-target-sites for the HSPC miR candidates (miR126-ts, miR142-ts, miR125ts, miR196ts, miR29ts), PBS (control) or mOX40L reporter mRNA with no miR target sites (miRless mOX40L group). mOX40L reporter mRNAs were formulated in lipid nanoparticles containing Compound II and Compound I. mOX40L expression was analyzed in the spleen and bone marrow 24 h post dose using flow cytometry.

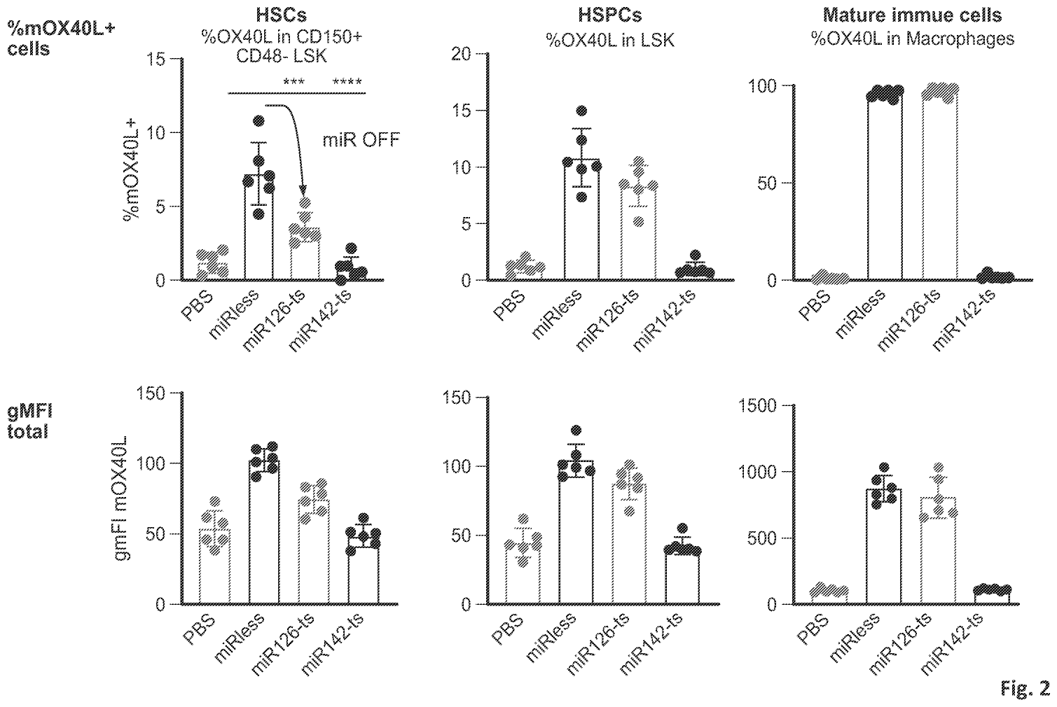

The graphs in FIG. 2 show the % frequency of OX40L+ cells and geometric MFI of OX40L in LSK: Lin-Sca1+c-Kit+ progenitor cells, hematopoietic stem and progenitor cells (HSPCs) and in mature immune cells (macrophages). The expression of mOX40L (percentage of OX40L+ cells) was significantly suppressed in CD150+ CD48− LSK cells (HSCs) in cells in vivo exposed to miR126ts-containing mOX40L constructs, suppressed in HSPCs, but not suppressed in mature immune cells (FIG. 2). By contrast, the expression of mOX40L (percentage of OX40L+ cells) was significantly suppressed in cells exposed in vivo to miR142ts-containing mOX40L constructs to a higher extent in all cell types (HSCs, HSPCs, and mature immune cells). The mean fluorescence intensity (MFI) of OX40L was also lower in cells exposed to miR142ts-containing mOX40L constructs compared to miRtsless constructs (FIG. 2).

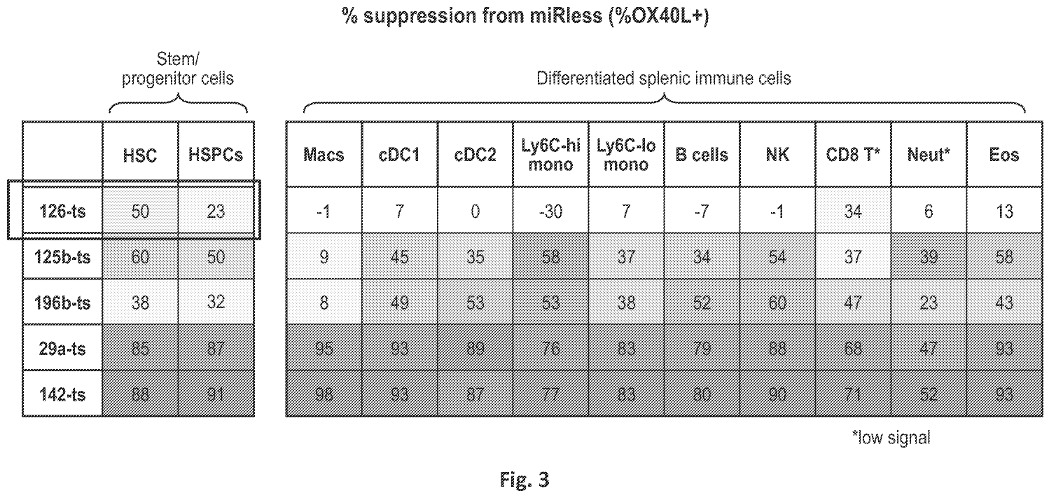

The percentage of suppression was determined by obtaining the percentage of OX40L+ Lin-Sca1+c-Kit+ hematopoietic stem cells obtained in each group administered the indicated miRts-containing mOX40L reporter constructs and comparing that percentage of the same cell phenotype obtained in the group administered the mOX40L reporter constructs with no miRts. FIG. 3 summarizes the results obtained in this in vivo study.

The data shows that the expression of mOX40L was suppressed selectively in stem/progenitor cell types that were exposed in vivo to miR126ts containing mOX40L constructs but not in differentiated splenic immune cells (MACS, cDC1, cDC2, Ly6C-hi, Ly6C-lo, neutrophils, eosinophils, NK cells, B cells, and CD4 T cells). By contrast, the expression of mOX40L was suppressed in all cell types (HSCs, HSPCs, and differentiated splenic immune cells) that were exposed in vivo to miR29ats and miR142ts containing mOX40L constructs. Differential suppression of mOX40L expression was seen in cells exposed in vivo to 125b-ts and 196b-ts containing mOX40L constructs across the various cell types analyzed.

Collectively, this data indicates that differential suppression of OX40L expression depends on the type of miR present in the mouse cells. miR142-ts suppress mRNA expression in all mouse hematopoietic cells. miR29a-ts suppresses mRNA expression in all mouse hematopoietic cells. miR126-ts suppresses mRNA expression preferentially in mouse HSCs (though not completely) compared to mature immune cells.

### Example 3: Constructs with Different miR Target Sites can Suppress Reporter mRNA Expression in Different Human Hematopoietic Cells Ex Vivo

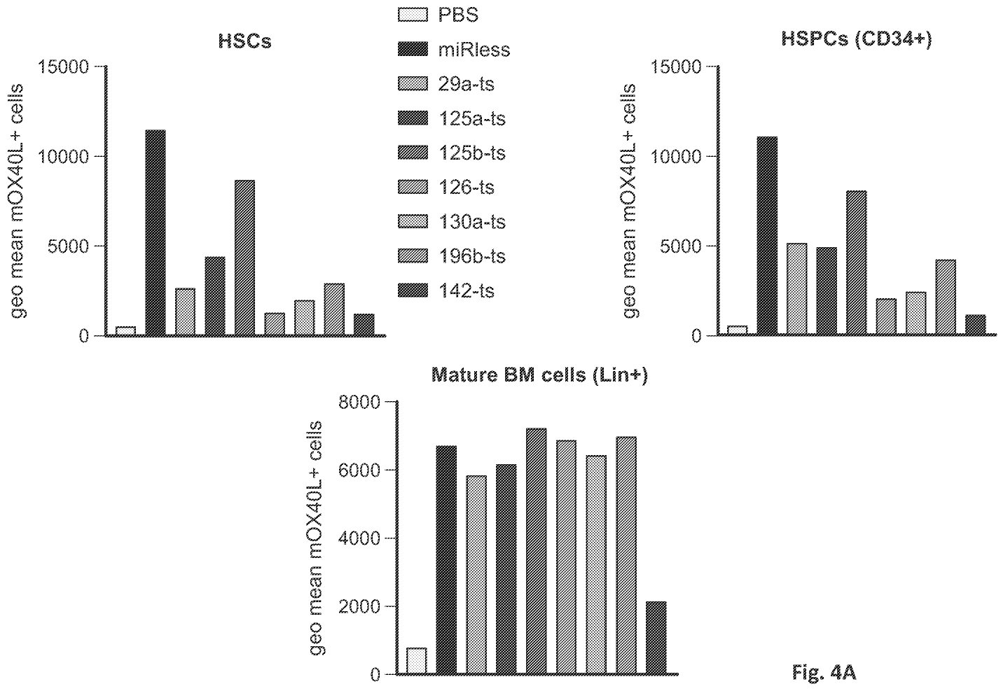

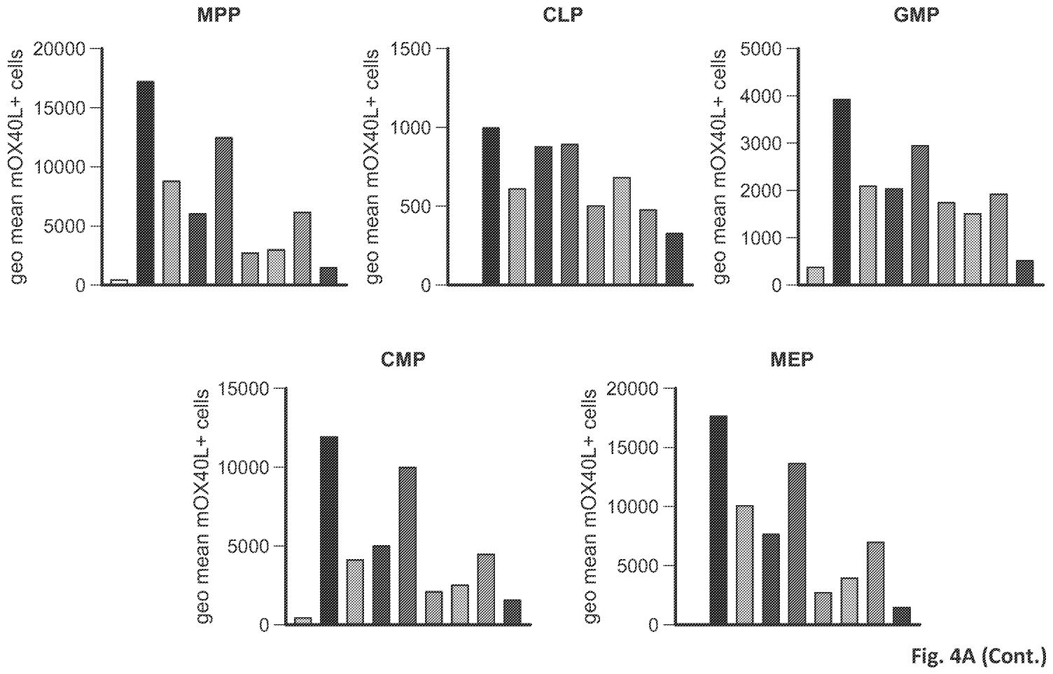

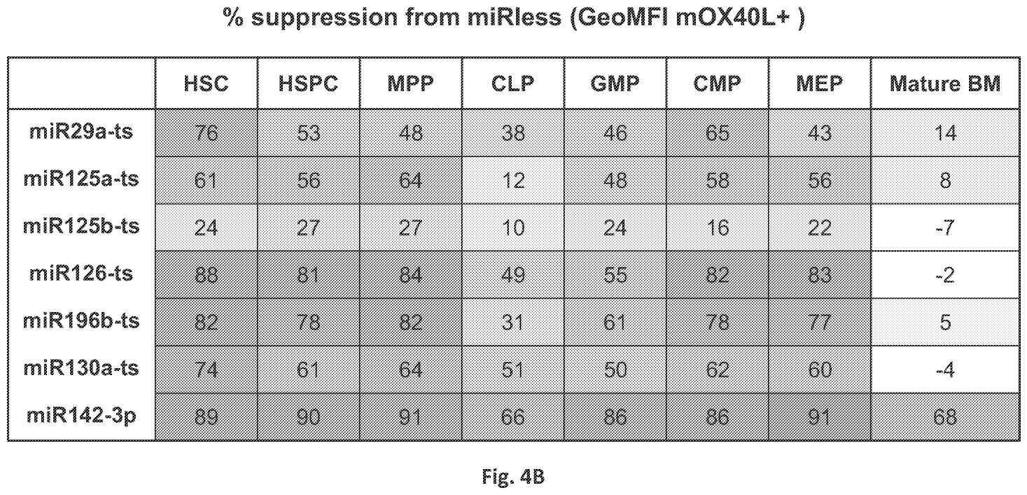

This example describes how to achieve OFF target expression of a target mRNA in HSPCs ex vivo using the system described in FIGS. 1A-B. In the first study, Lonza human bone marrow mononuclear cells were transfected with 500 ng of mOX40L reporter mRNAs containing the indicated miR-target-sites (miR29a-ts, miR29a-ts, miR125-ts, miR125b-ts, miR126-ts, miR196b-ts, miR130a-ts, miR142-ts) or mOX40L reporter mRNA with no miR target sites (miRless mOX40L group) or PBS (control). mOX40L reporter mRNAs were formulated in lipid nanoparticles containing Compound II. mOX40L expression was analyzed 24 h post transfection. Each graph in FIG. 4A shows the geometric MFI of mOX40L+ cells. FIG. 4B shows a summary table of the percentage suppression of OX40L in various cell types as indicated 24 h post transfection.

The data shows that the expression of mOX40L (geometric MFI) was suppressed selectively in HSCs, HSPCs, Multipotent progenitors (MPP), Common lymphoid progenitors (CLP), Granulocyte Monocyte Progenitors (GMP), Common Myeloid Progenitors (CMP), and Megakaryocyte-Erythrocyte Progenitors (MEP) cell types that were exposed ex vivo to miR126ts containing mOX40L constructs but not in mature bone marrow (BM) cells. By contrast, the expression of mOX40L was suppressed in all cell types that were exposed ex vivo to miR142ts containing mOX40L constructs, including mature BM cells. Further, the level of suppression of expression of mOX40L was different in varying cell-types exposed to different miRts containing constructs (miR29a-ts, miR29a-ts, miR125-ts, miR125b-ts, miR126-ts, miR196b-ts, miR130a-ts, or miR142-ts).

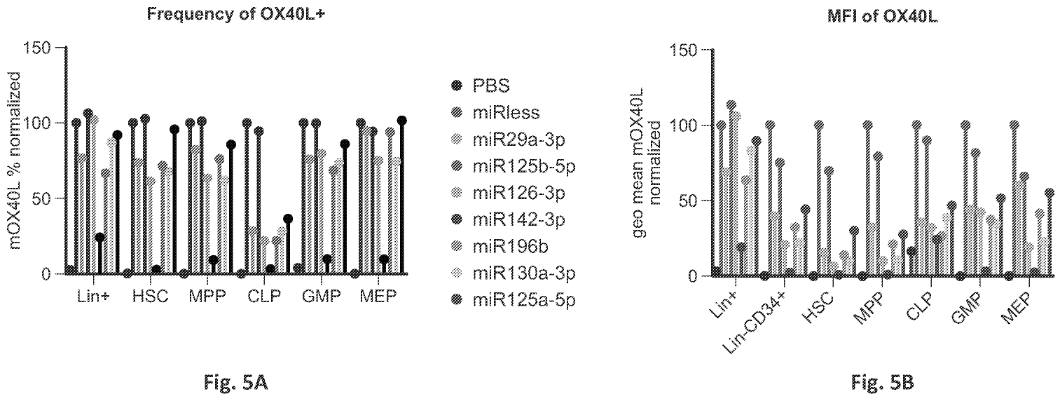

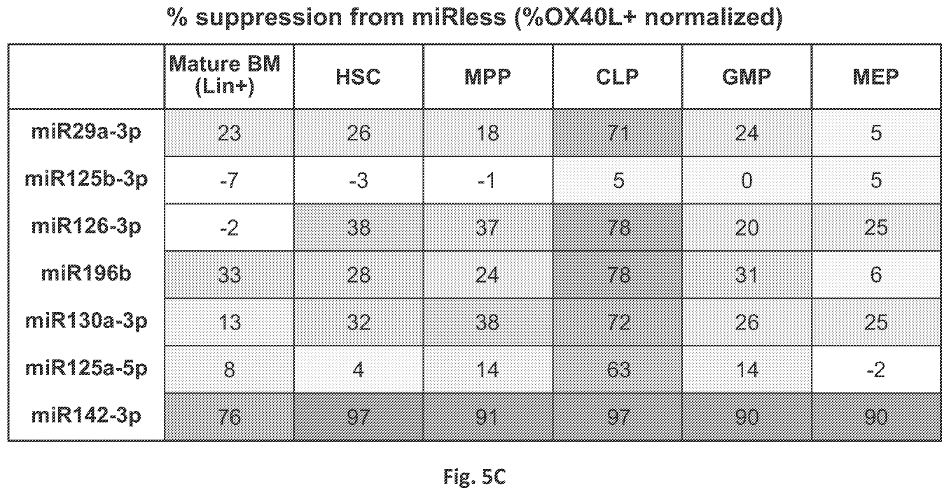

In a second study, Lonza human bone marrow mononuclear cells were transfected with 500 ng of mOX40L reporter mRNAs containing the indicated miR-target-sites (miR29a-3p, miR125b-5p, miR126-3p, miR142-3p, miR196b, miR130a-3p, miR125a-5p), mOX40L reporter mRNA with no miR target sites (miRless mOX40L group) or PBS (control). mOX40L reporter mRNAs were formulated in lipid nanoparticles containing Compound II. mOX40L expression was analyzed 24 h post transfection. FIGS. 5A-B show the % mOX40L normalized expression level and gMFI for the various cell types indicated, which vary depending on the mOX40L reporter construct used (miR29a-ts, miR29a-ts, miR125-ts, miR125b-ts, miR126-ts, miR196b-ts, miR130a-ts, or miR142-ts). FIG. 5C summarizes the percentage suppression of OX40L in cells exposed to the reporter constructs with the various miRts as indicated.

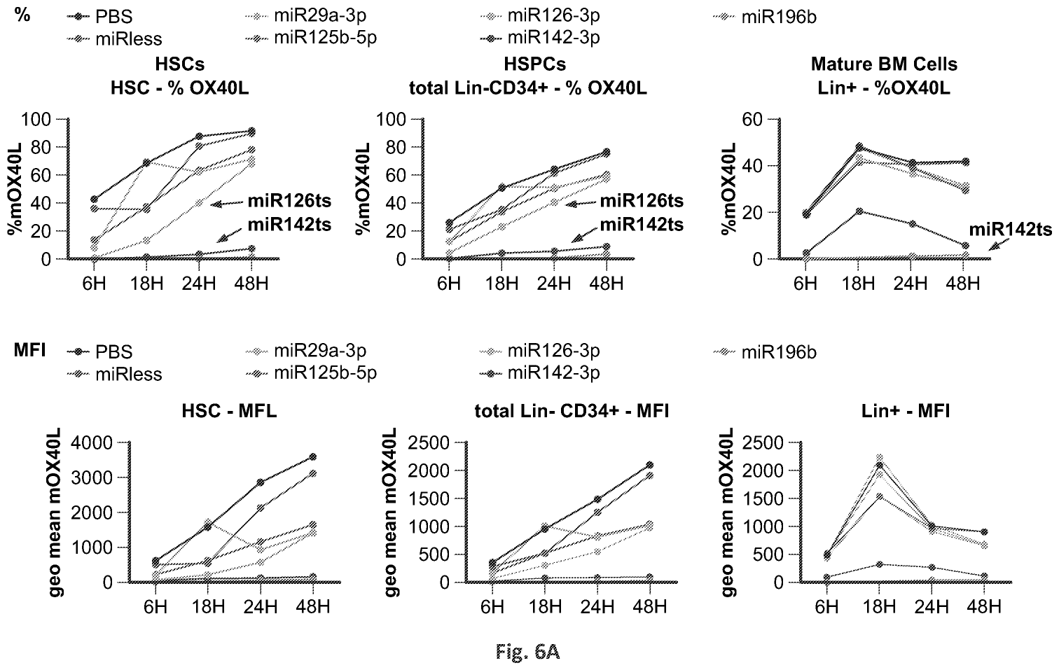

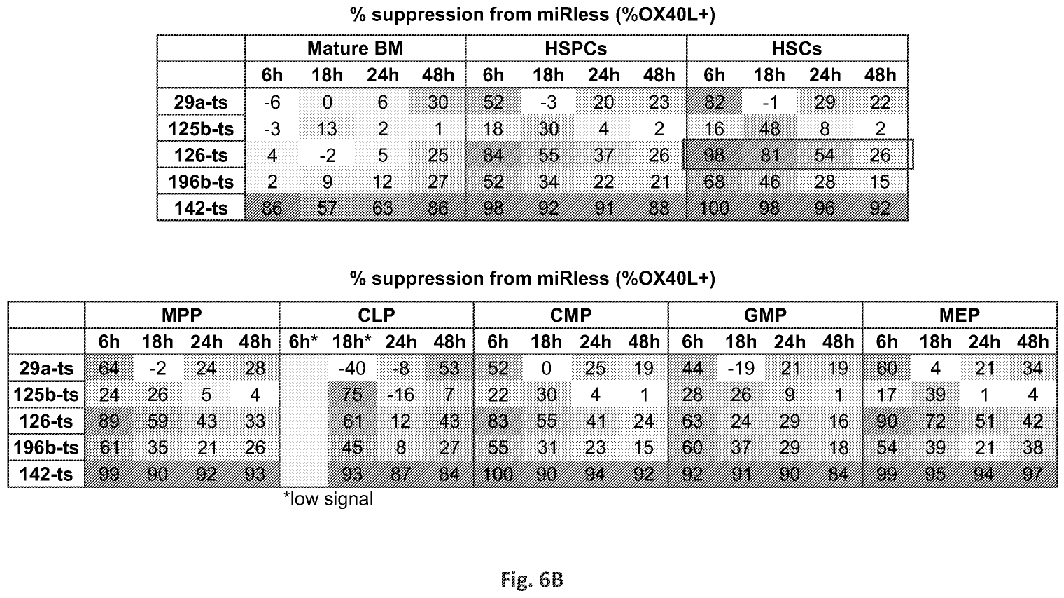

In a third study, human BMMCs were transfected with lipid nanoparticles containing Compound II and mOX40L-encoding mRNAs. Lipid nanoparticles were pre-opsonized in 5% human serum (BioIVT) for 10 minutes then added to the cells at 100 or 500 ng/well in a 24-well plate. Treatments included mOX40L reporter mRNAs containing the indicated miR-target-sites, mOX40L reporter mRNA with no miR target sites (miRless mOX40L group) or PBS (control). Cells were maintained ex vivo overnight in Peprotech human hematopoietic stem cell expansion cytokines containing IL-3 (60 ng/ml), Thrombopoietin (TPO, 100 ng/ml), stem cell factor (SCF, 300 ng/ml), and FLT3-Ligand (300 ng/ml). Cells were harvested at timepoints as indicated, and analyzed by flow cytometry. mOX40L expression and MFI was measured by flow cytometry over a 6 h-48 h timecourse. As seen in FIG. 6A the % frequency of OX40L+ cells and MFI of OX40L in the various cell types differs across time depending on the mOX40L constructs that the cells were exposed to ex vivo. As seen in FIG. 6B, the percentage suppression of OX40L in cells exposed to miR126ts containing mOX40L constructs decreased over the course of 48 hrs.

These data indicate that miR142-ts suppresses mRNA expression in all human hematopoietic cells. miR126-ts suppresses mRNA expression preferentially in human HSCs (though not completely) compared to mature immune cells. miR126-ts also shows some suppression in human progenitor cells. miR130a-ts shows a partial, but potentially preferential, suppression in human HSCs/HSPCs. Collectively, this data indicates that differential suppression of OX40L expression depends on the type of miR present in the human cells, and that the suppression of the OX40L can change over time for certain miRts but not for others.

### Example 4: a miR Target Site can be Used in ON Switch and OFF Switch Systems

This example describes how the same miRts can achieve ON or OFF target expression of a target mRNA in cells ex vivo using the system described in FIGS. 1A-C.

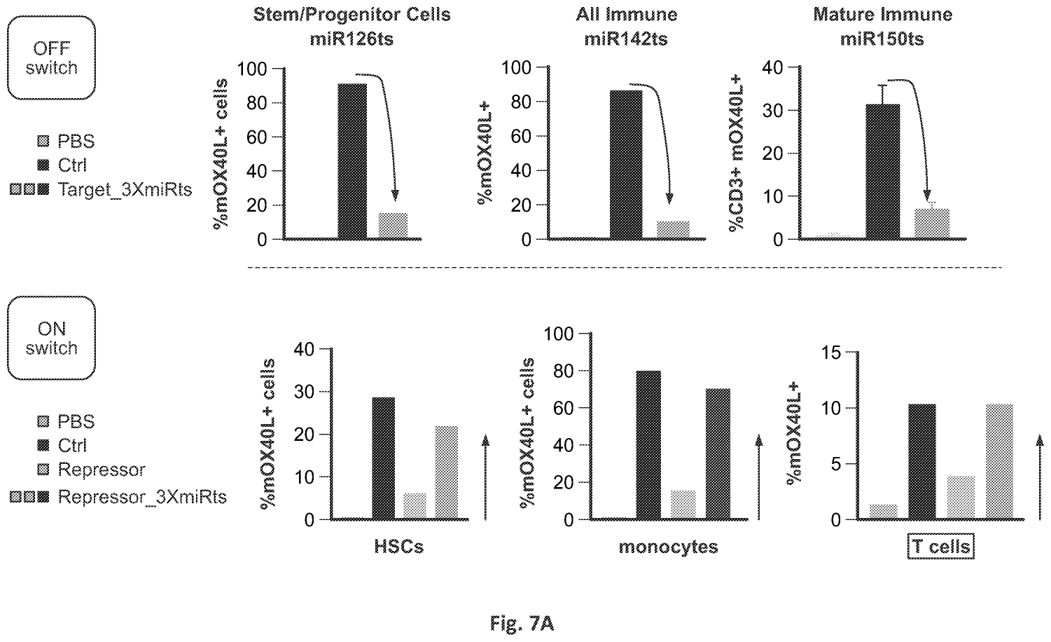

In the OFF system, human bone marrow mononuclear cells were transfected with 100 ng or 500 ng of mOX40L reporter mRNAs containing the indicated target-sites miR126ts, miR142ts, and miR150ts (Target_3×miRts), mOX40L reporter mRNA with no miR target sites (control) or PBS. mOX40L reporter mRNAs were formulated in lipid nanoparticles containing Compound II. mOX40L expression was analyzed 24 h post transfection. In the ON system, human bone marrow mononuclear cells were transfected with 100 ng or 500 ng of the mOX40L_D99K target with miRless repressor RNA without miR target sites (Repressor) or repressor RNA containing target sites miR126ts, miR142ts, and miR150ts (Repressor_3×miRts). The specific doses used for the OFF/ON studies is as follows: 126—OFF: 100 ng; 142—OFF: 100 ng; 150—OFF: 100 ng; 126—ON: 100 ng; 142—ON: 100 ng; 150—ON: 500 ng. The D99K mutation reduces biological activity. mOX40L expression was analyzed by flow cytometry 24 h post transfection. As seen in FIG. 7A, the % frequency of OX40L+ cells decreased in stem/progenitor cells (HSCs) using an miR126ts OFF system (top panel), while it increased in HSCs using an miR126ts ON system (bottom panel). Similarly, the % frequency of OX40L+ cells decreased in monocytes using an miR142ts OFF system (top panel), while it increased in monocytes using an miR142ts ON system (bottom panel). Further, the % frequency of OX40L+ cells decreased in T cells using an miR152ts OFF system (top panel), while it increased in T cells using an miR150ts ON system (bottom panel). FIG. 7B shows similar trends in gMFI expression of mOX40L as that seen in FIG. 7A.

These data indicate that a particular miRts can be used to turn on or off expression of a target selectively in different immune cells.

### Example 5: Specific HSC-OFF miRs Selectively Suppress Expression Ex Vivo Human HSCs

This example describes OFF target expression of a target mRNA in cells ex vivo using the system described in FIGS. 1A-B.

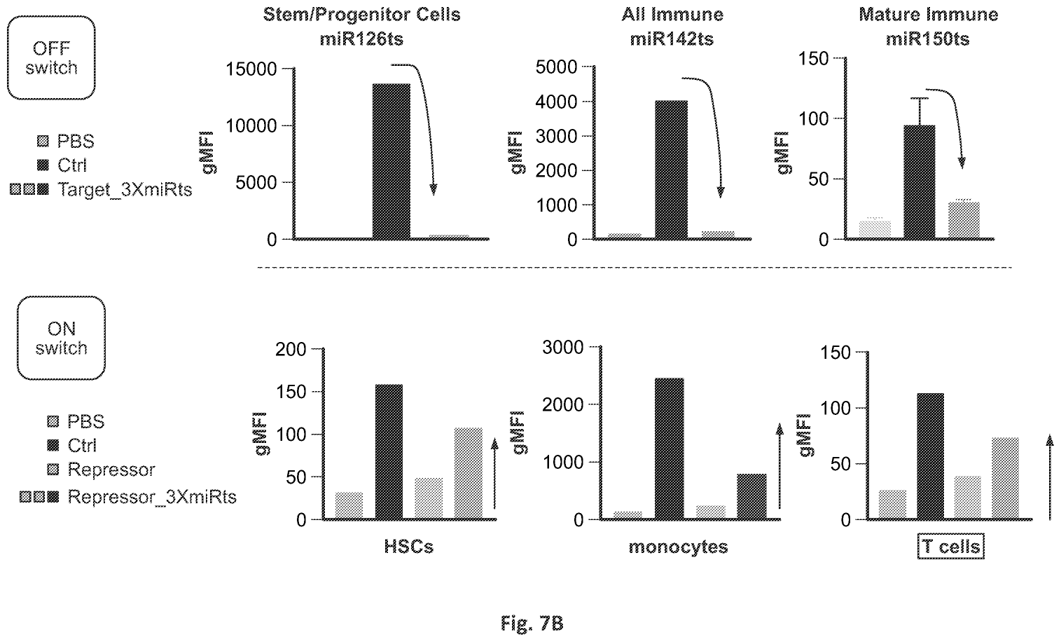

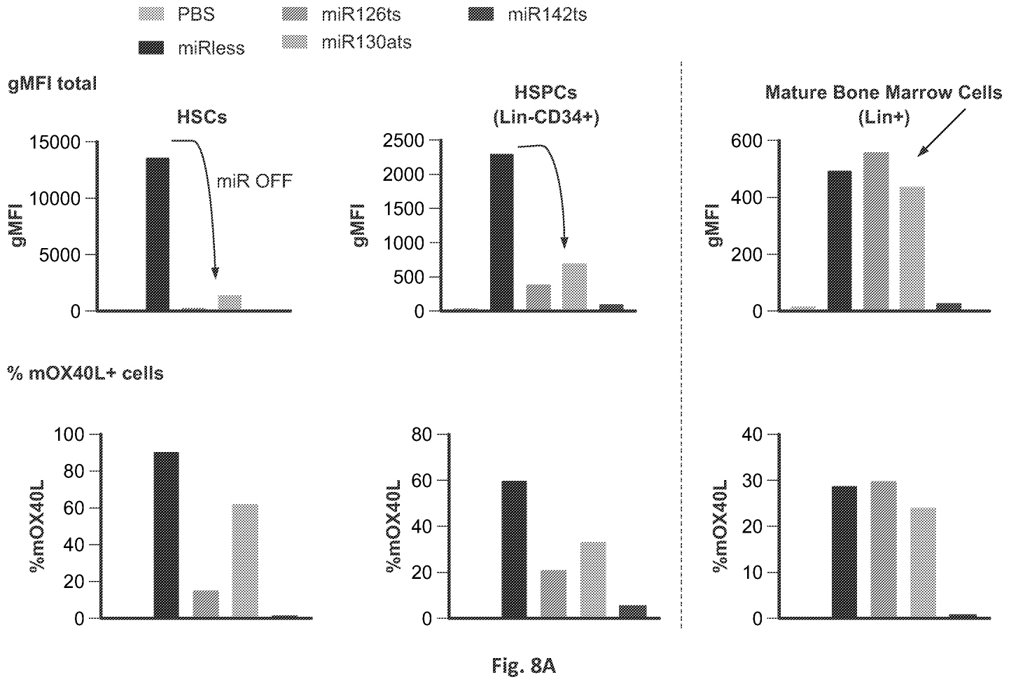

In the first study, human bone marrow mononuclear cells were transfected with 100 ng of mOX40L_D99K reporter mRNAs containing the indicated target-sites miR126ts, miR142ts, and miR150ts, mOX40L reporter mRNA with no miR target sites (miRless) or PBS. mOX40L reporter mRNAs were formulated in lipid nanoparticles containing Compound II. mOX40L expression was analyzed 24 h post transfection. The experiment was run in triplicate. As seen in FIGS. 8A-B, HSC-OFF miR126 and miR130a selectively suppress expression (% of cells expressing mOX40L as well as gMFI of mOX40L) in ex vivo human HSCs and HSPCs, but not in mature bone marrow (Lin+) cells. Similar trends as those seen for HSCs and HSPCs were observed in MPP, CMP, CLP, GMP, and MEP cells. On the other hand, HSC-OFF miR142 suppresses expression of mOX40L in all cell types.

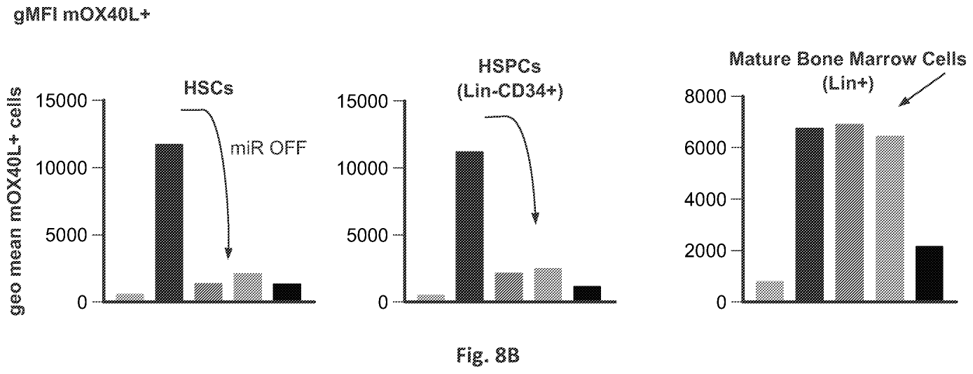

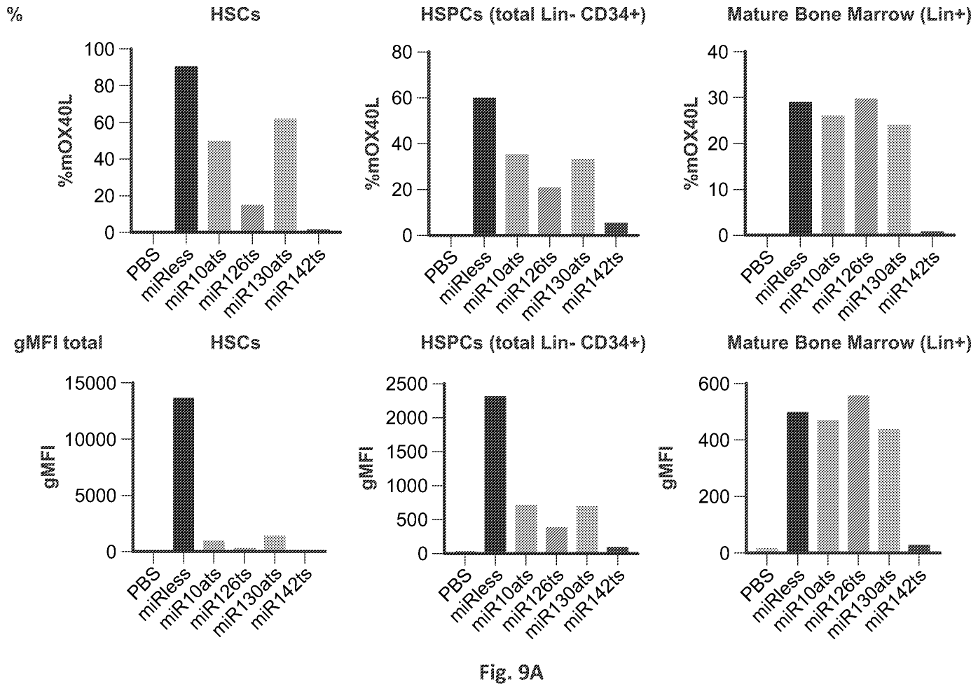

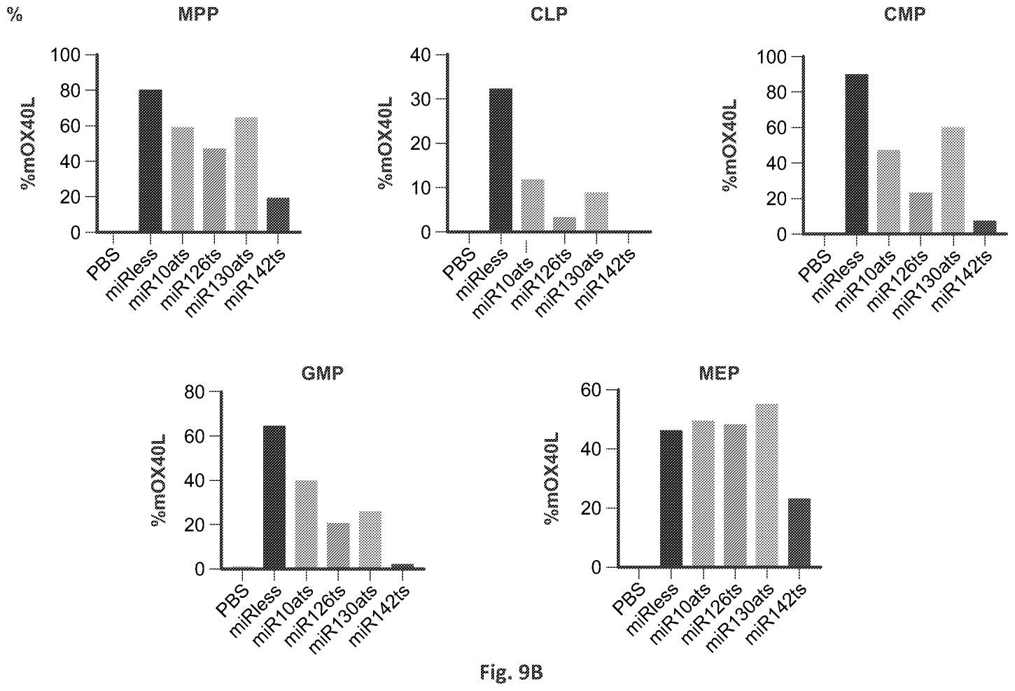

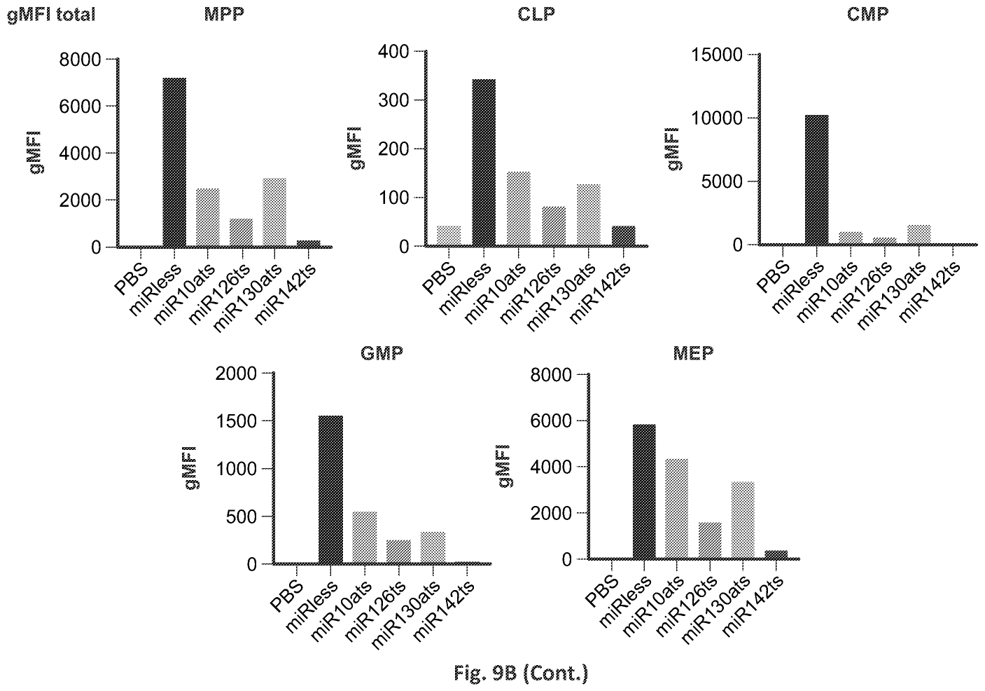

In the second study, Lonza bone marrow mononuclear cells were transfected with 100 ng of mOX40L_D99K reporter mRNAs containing the indicated target-sites miR10ats, miR126ats, miR130ats, and miR142ats, mOX40L reporter mRNA with no miR target sites (miRless) or PBS. mOX40L reporter mRNAs were formulated in lipid nanoparticles containing Compound II. mOX40L expression was analyzed 24 h post transfection. As seen in FIGS. 9A-B, HSC-OFF miR10a, miR126a and miR130a selectively suppress expression (% of cells expressing mOX40L as well as gMFI of mOX40L) in ex vivo human HSCs and HSPCs, but not in mature bone marrow (Lin+) cells. Similar trends as those seen for HSCs and HSPCs were observed in MPP, CMP, CLP, and GMP cells but not in MEP cells where there was no significant different in % expression of mOX40L but gMFI was suppressed. On the other hand, HSC-OFF miR142 suppresses expression of mOX40L in all cell types.

Collectively, this data shows that HSC-OFF miR126 and miR130a selectively suppress expression ex vivo human HSCs in one system. HSC-OFF miR10, miR126, and miR130a selectively suppress expression ex vivo human HSCs in another system.

### Example 6: Modifications of Constructs with miRts can Improve the Efficacy of ON/OFF Switches

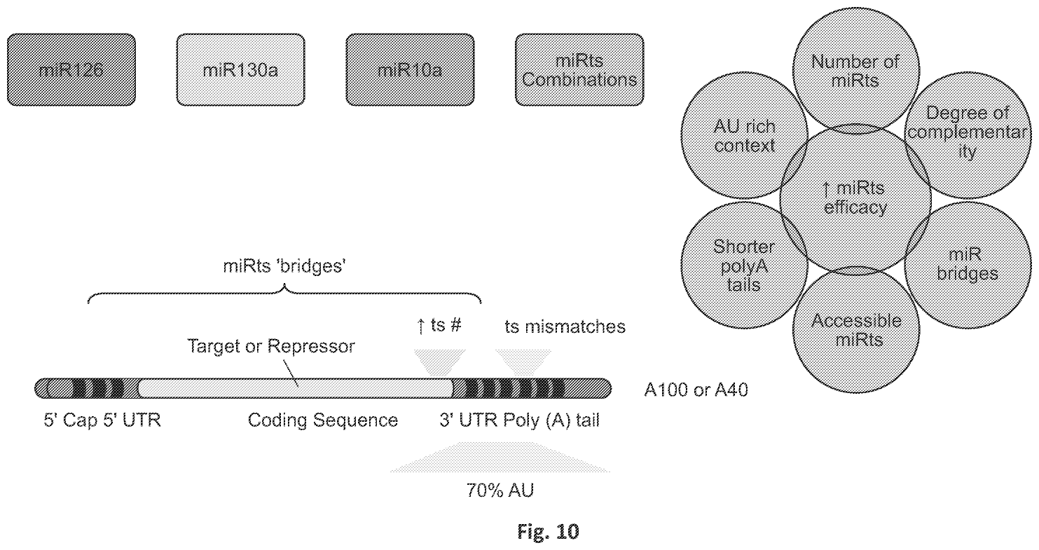

FIG. 10 shows various design modifications that can be employed for increasing efficacy of the miR target sites in ON/OFF systems in a polynucleotide construct. Design modifications include using various miRts combinations in the same construct, increasing the number of miR target sites in the 3′ and/or 5′ UTR of the construct, altering the degree of complementarity of a particular target site with the respective miRNA (e.g., include target site mismatches), including an miR bridge that spans across the 5′ and 3′ UTR, improving the accessibility of the miRts, having a shorter polyA tail (e.g., A40), and/or including AU rich sequences in the constructs. FIG. 11 shows various specific design modifications that can be employed for increasing efficacy of the miR ON/OFF system in an exemplary polynucleotide construct which employs the use of 2×miR126ts plus 2×miR130ats in the 3′ UTR of the target RNA, use of a 3′ UTR enriched with 70% AU nucleotides, and/or use of microRNA target sites having mismatches at positions 18-21 with the corresponding miR at the 5′ target site end.

Target RNA constructs with design modifications have been tested as shown in the following examples.

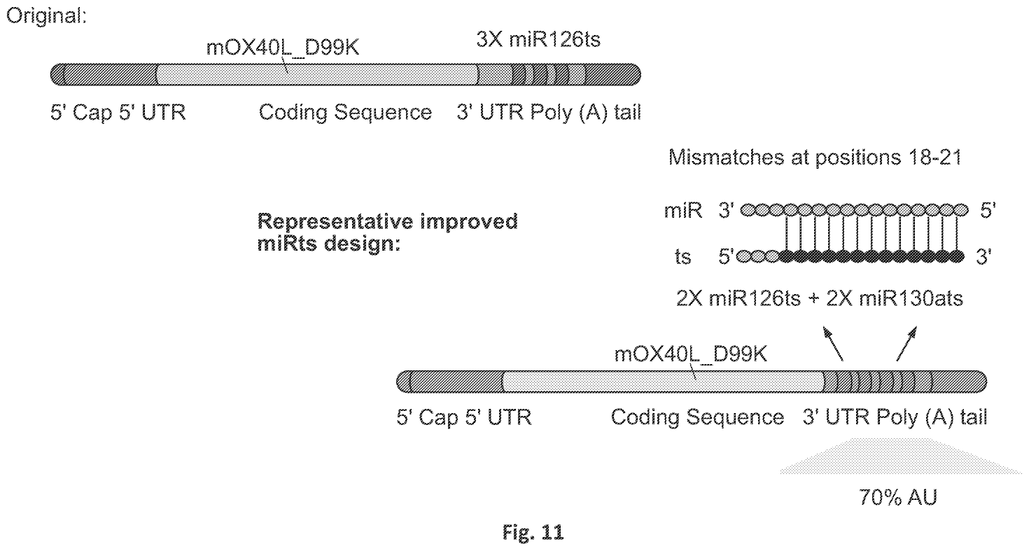

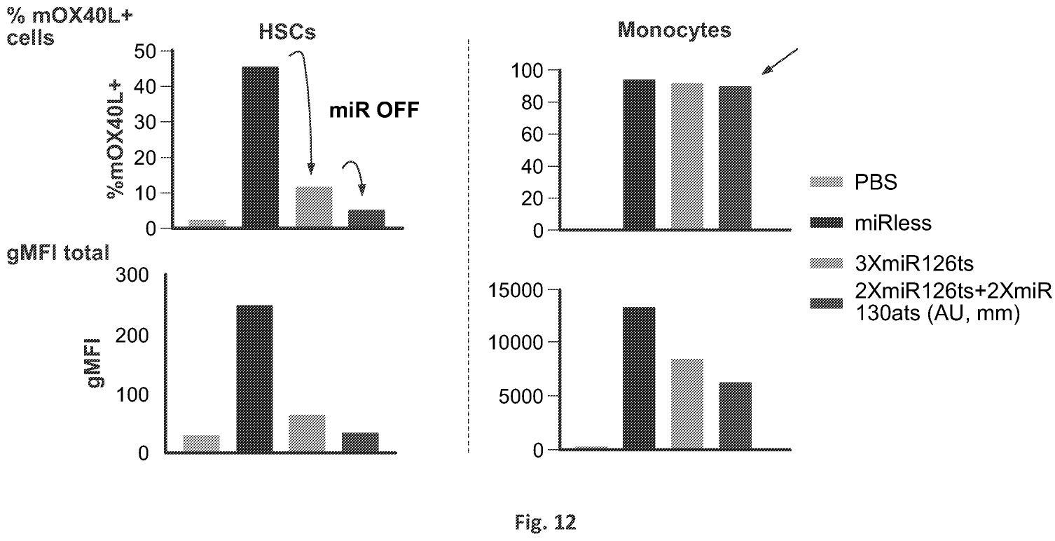

miRts design improvements were shown to selectively increase suppression in HSCs in an miR OFF system. In one study, human bone marrow mononuclear cells were transfected with 1 μg of the mOX40L_D99K target RNA without miR target sites (miRless), or with 3×miR126ts, or 2×miR126ts+2×miR130ats (AU, mm). mOX40L expression was analyzed 24 h post transfection. mOX40L reporter mRNAs were formulated in lipid nanoparticles containing Compound II. As shown in FIG. 12, the expression of mOX40L was turned off selectively in HSCs (FIG. 12 left panel) but not in monocytes (FIG. 12 right panel) in an miR OFF system for HSCs. Similar trends to those seen in HSCs were observed in MPP, CMP, MEP, and GMP cells.

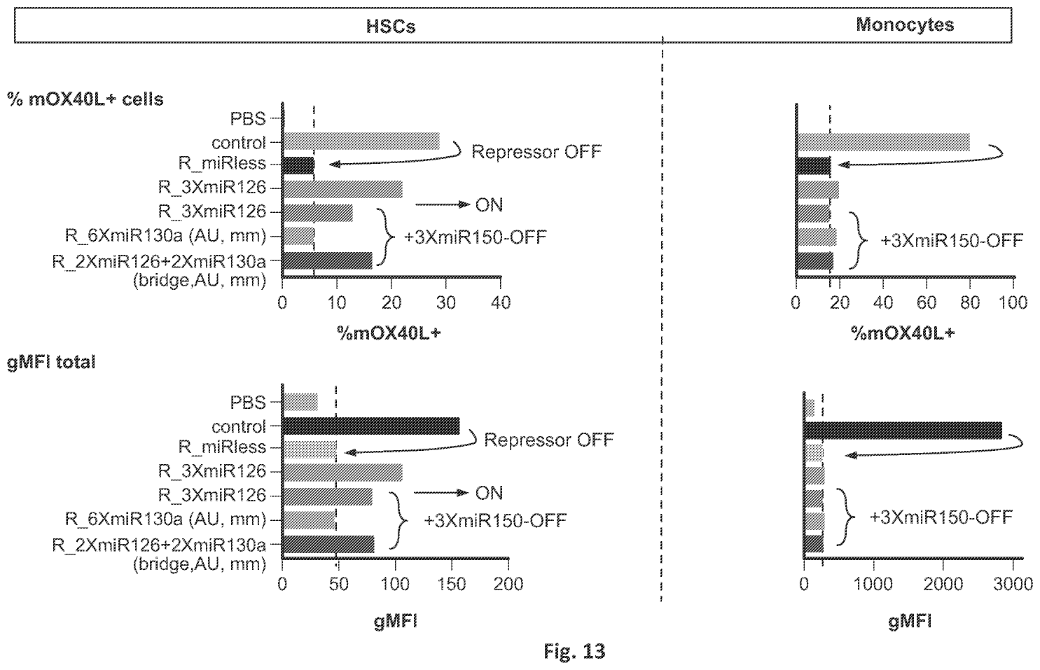

In another study, human bone marrow mononuclear cells were transfected with 100 ng of the mOX40L_D99K target RNA with non-relevant filler mRNA (control) or with various L7Ae repressor RNA constructs (each 0.5× molar) as follows: L7Ae repressor RNA without miR target sites (R_miRless); L7Ae repressor RNA containing miR126ts (R_3×miR126); L7Ae repressor RNA containing miR130a (R_6×miR130a (AU, mm)); and L7Ae repressor RNA containing 2×miR126ts+2×miR130ats (bridge, AU, mm). mOX40L reporter mRNAs were formulated in lipid nanoparticles containing Compound II. As shown in FIG. 13, the expression of mOX40L was turned on selectively in HSCs (FIG. 13 left panel) but not in monocytes (FIG. 13 right panel) in an miR ON system for HSCs. Similar trends to those seen in HSCs were observed in MPP, CMP, MEP, and GMP. Similar trends to those seen in monocytes were observed in CD4+, CD8+ T cells, BM cells & hPBMCs.

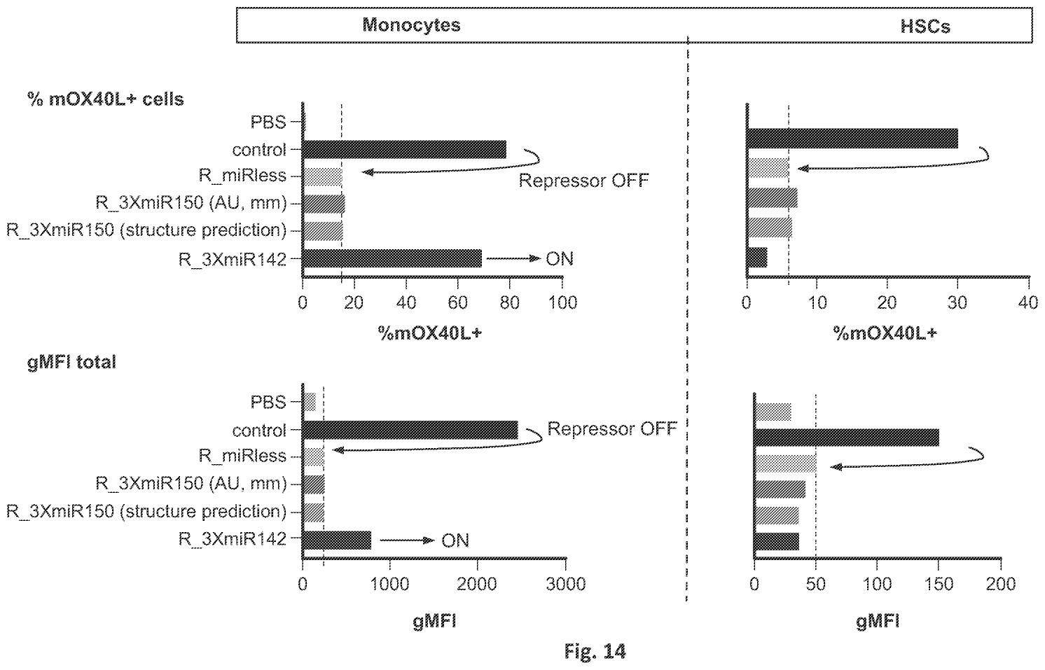

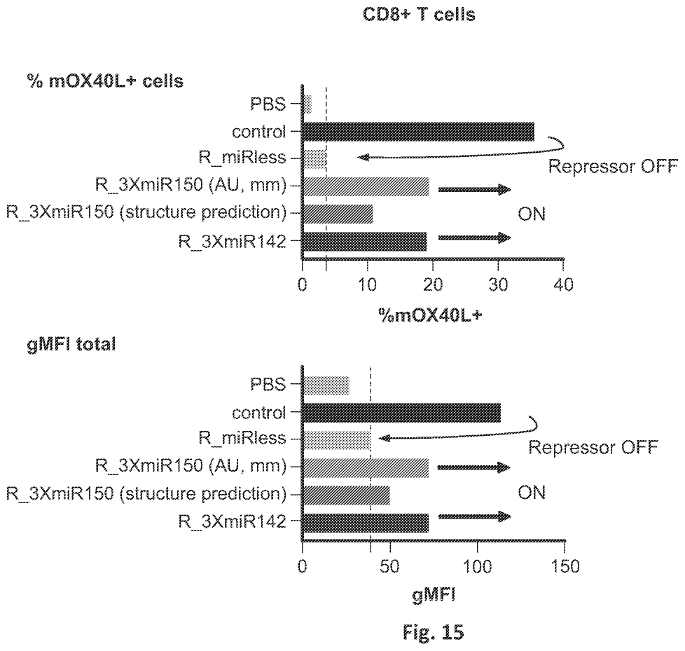

In yet another study, human bone marrow mononuclear cells were transfected with 100 ng of the mOX40L_D99K target RNA with non-relevant filler mRNA (control) or with various L7Ae repressor RNA constructs (each 0.5× molar) as follows: L7Ae repressor RNA without miR target sites (R_miRless); L7Ae repressor RNA containing miR142ts (R_3×miR142); L7Ae repressor RNA containing miR150a (R_3×miR150a (AU, mm)); and L7Ae repressor RNA containing 3×miR150 ((R_3×miR150). mOX40L reporter mRNAs were formulated in lipid nanoparticles containing Compound II. mOX40L expression was analyzed 24 h post transfection. As shown in FIG. 14, the expression of mOX40L was turned on selectively in monocytes (FIG. 14 left panel) and CD8+ T cells (FIG. 15) but not in HSCs (FIG. 14 right panel) in an miR ON system for mature immune cells. Similar trends to those seen in HSCs were observed in MPP, CMP, MEP, and GMP. Similar trends to those seen in CD8+ T cells were observed in CD4+, CD8+ T cells, BM cells & hPBMCs.

Collectively, these results demonstrate that the designs of the miRts ON/OFF constructs can be altered to modulate target expression in specific cell types. miR150, miR122, and miR142 repression has benefited from AU-rich 3′UTR context, 3′ end mismatches in target sites, increased number of target sites, ‘bridges’, increased accessibility, shorter polyA tails, and combining these miR-ts designs increases miR-ON and OFF switches.

### Example 7: Expression of a Target can be Turned ON Selectively in HSC/HSPCs

This example describes ON target expression of a target mRNA in cells ex vivo using the system described in FIG. 1E.

In the first study, human bone marrow mononuclear cells (in triplicate) were transfected with 1 μg of the mOX40L_D99K target RNA with non-relevant filler mRNA (control) or with various L7Ae repressor RNA constructs (each 0.5× molar) as follows: L7Ae repressor RNA without miR target sites (R_miRless); L7Ae repressor RNA containing miR126ts (R_3×miR126ts); L7Ae repressor RNA containing L7Ae repressor RNA containing both miR126ts and miR130ats (R_2×miR126ts+2×miR130a (bridge, AU, mm)); or L7Ae repressor RNA containing miR142ts (R_3×miR142ts). R_2×miR126ts+2×miR130a (bridge, AU, mm) represents an L7Ae repressor RNA construct with further modifications—AU=70% AU rich UTR; mm=mismatch 18-21. Some combinations further included 0.5× molar L7Ae repressor RNA R_3×miR150-OFF as indicated in the graphs. mOX40L reporter mRNAs were formulated in lipid nanoparticles containing Compound II. mOX40L expression was analyzed 24 h post transfection.

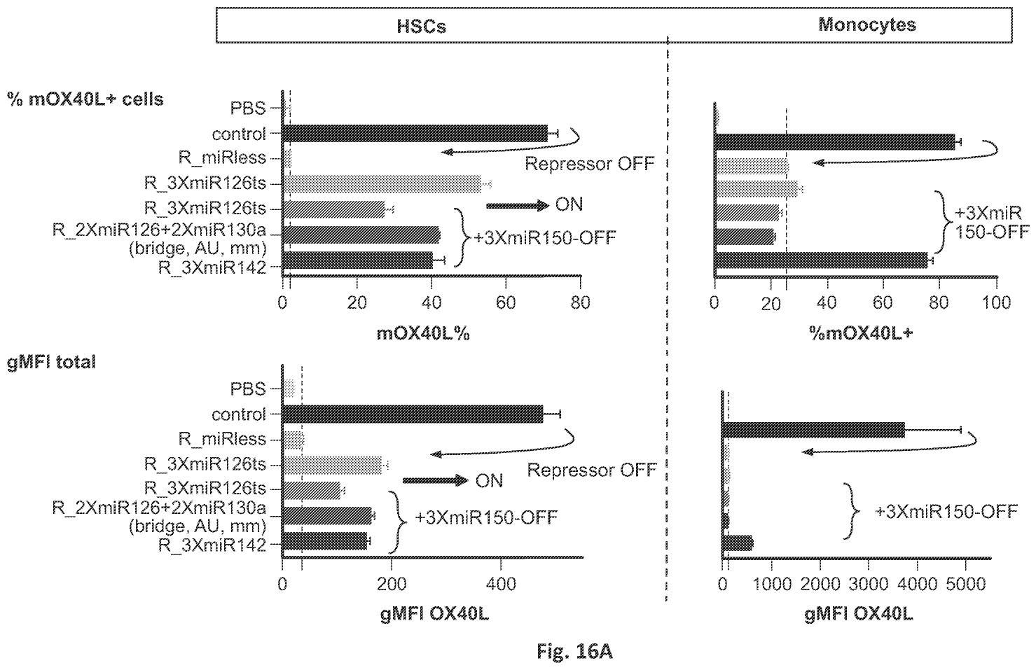

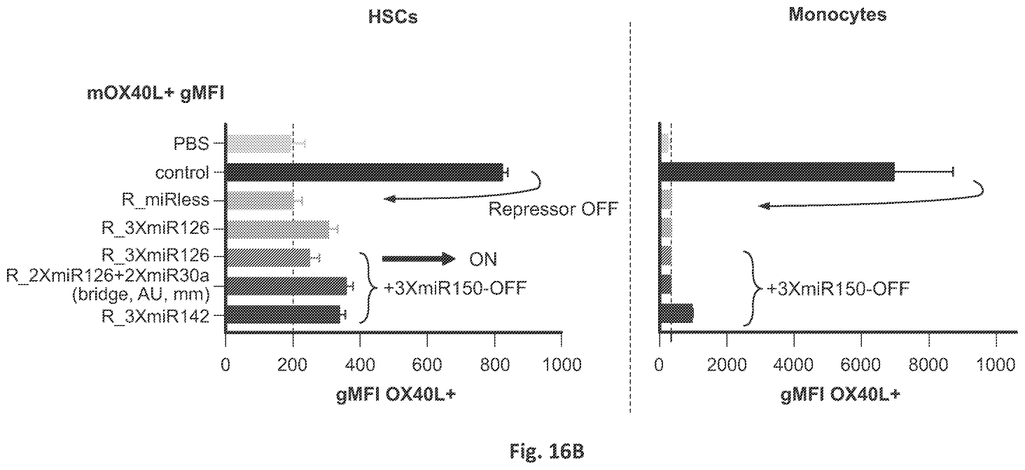

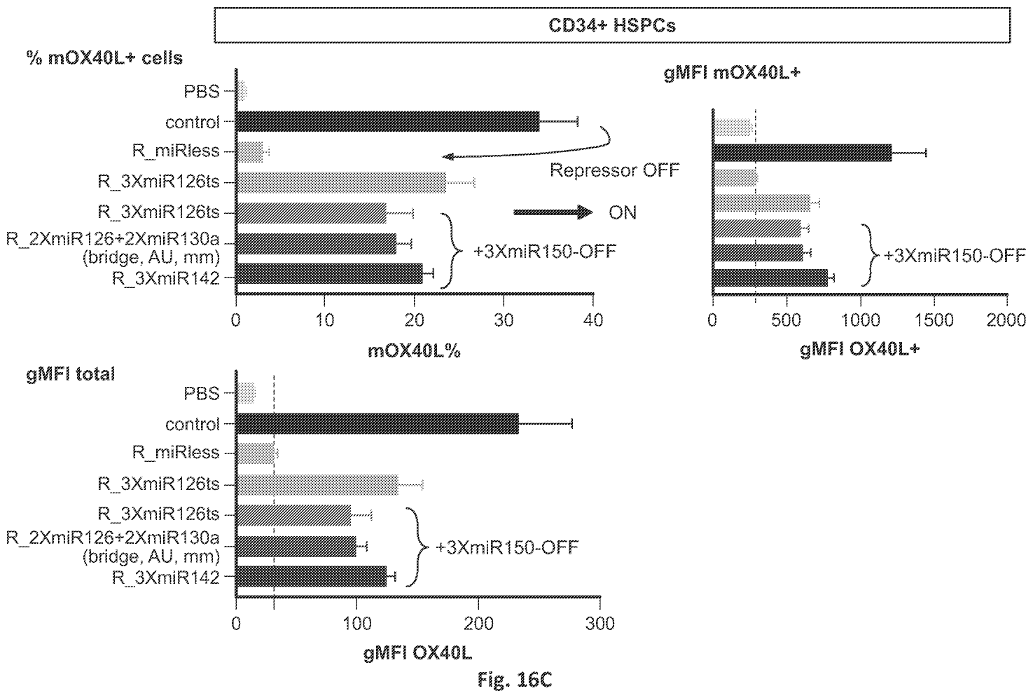

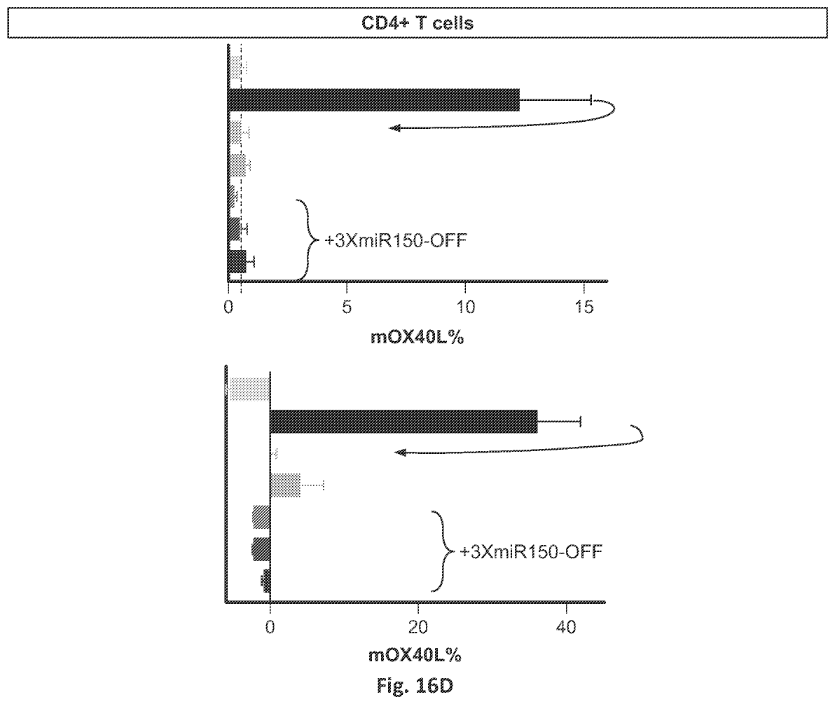

As shown in FIGS. 16A-D, the expression of mOX40L was turned on selectively in HSCs (FIGS. 16A-C) but not in monocytes (FIG. 16C) or CD4+ T cells (FIG. 16D). Similar trends to those seen in HSCs were observed in BM cells and hPBMCs. Similar trends to those seen in monocytes were observed in MPP, CMP, MEP, and GMP cells. Further, using an miR150—OFF system along with the miR126—ON system can further reduce expression of mCD40L in mature immune cells such as CD4+ T cells as shown in FIG. 16D. Similar trends to those seen in CD4+ T cells were observed in CD8+ T cells.

In a further study, human bone marrow mononuclear cells were transfected with 100 ng of the mOX40L_D99K target RNA with non-relevant filler mRNA (control) or with various L7Ae repressor RNA constructs (each 0.5× molar) as follows: L7Ae repressor RNA without miR target sites (R_miRless); L7Ae repressor RNA containing miR126ts (R_3×miR126); L7Ae repressor RNA containing miR130a (R_6×miR130a (AU, mm)); and L7Ae repressor RNA containing 2×miR126ts+2×miR130ats (bridge, AU, mm). The results of that study are described in Example 6.

Collectively, these results demonstrate that the expression of a target can be turned ON selectively in HSCs using an miRts ON system and further modulated using a dual miRts ON/OFF system. Further, miR126-ts, combinations of miR126 plus miR130a-ts, and miR142-ts can turn ON expression selectively in HSCs/HSPCs.

### Example 8: Mature Immune Cell-ON L7Ae with or without Specific miRts Selectively Turn on Expression in Mature Immune Cells

This example describes ON target expression of a target mRNA in cells ex vivo using the system described in FIG. 1D.

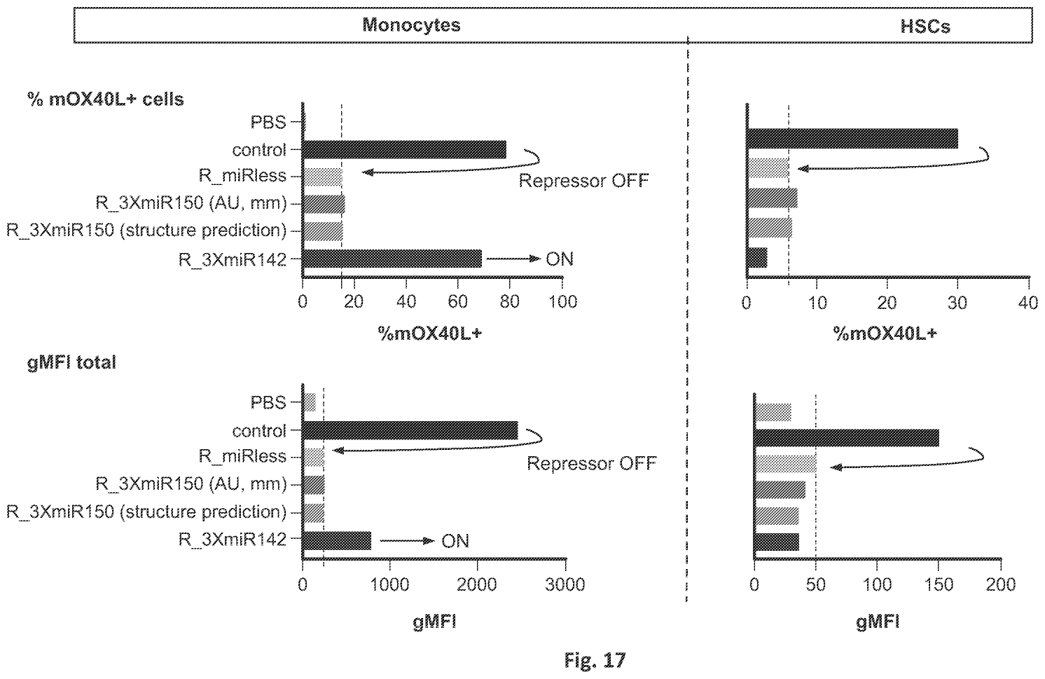

In the first study, human bone marrow mononuclear cells were transfected with 100 ng or 500 ng of the mOX40L_D99K target mRNA with non-relevant filler mRNA (control) or with various L7Ae repressor RNA constructs (each 0.5× molar) as follows: L7Ae repressor RNA without miR target sites (R_miRless); L7Ae repressor; L7Ae repressor RNA containing miR150ts (R_3×miR150ts (AU, mm)); L7Ae repressor RNA containing miR150ts (R_3×miR150 (structure prediction)); and L7Ae repressor RNA containing miR142ts (R_3×miR142). R_3×miR150ts (AU, mm) and R_3×miR150 (structure prediction) represent L7Ae repressor RNA constructs with further modifications—AU=70% AU rich UTR; mm=mismatch 18-21. mOX40L reporter mRNAs were formulated in lipid nanoparticles containing Compound II. mOX40L expression was analyzed 24 h post transfection. As shown in FIG. 17, the expression of mOX40L was turned on selectively in monocytes (FIG. 17 left panel) but not in HSCs (FIG. 17 right panel) in an miR142 ON system.

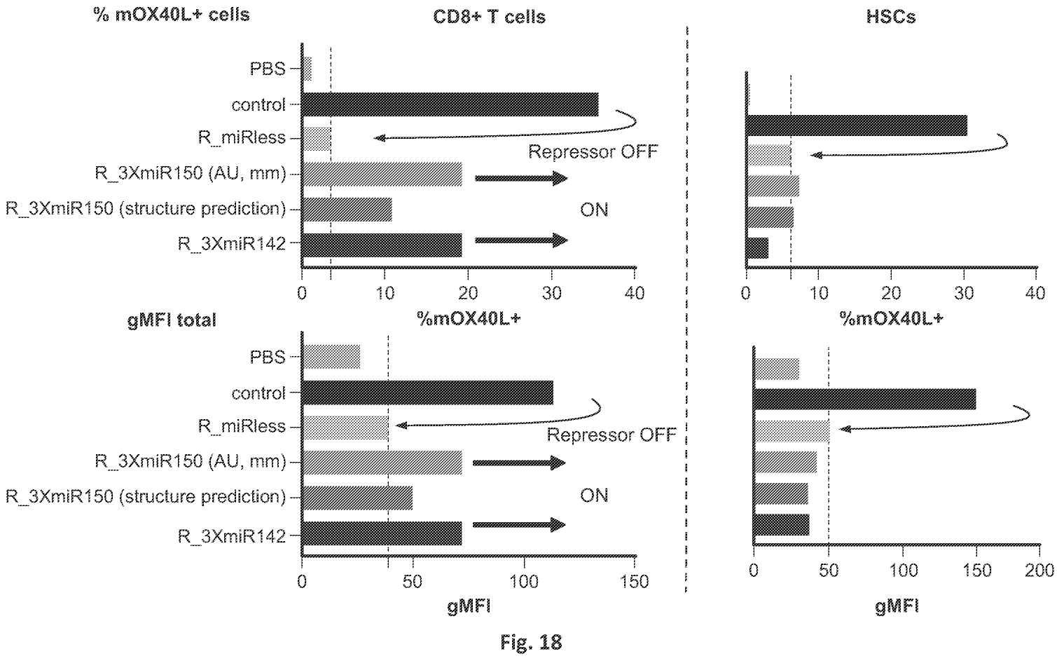

In the second study, human bone marrow mononuclear cells and PBMCs were transfected with 1 μg or 500 ng of the mOX40L_D99K target RNA with non-relevant filler mRNA (control) or with various L7Ae repressor RNA constructs (each 0.5× molar) as follows: L7Ae repressor RNA without miR target sites (R_miRless); L7Ae repressor RNA containing miR150ts (R_3×miR150ts (AU, mm)); L7Ae repressor RNA containing miR150ts (R_3×miR150 (structure prediction)); and L7Ae repressor RNA containing miR142ts (R_3×miR142). R_3×miR150ts (AU, mm) and R_3×miR150 (structure prediction) represent L7Ae repressor RNA constructs with further modifications AU=70% AU rich UTR; mm=mismatch 18-21. mOX40L reporter mRNAs were formulated in lipid nanoparticles containing Compound II. mOX40L expression was analyzed 24 h post transfection. The expression of mOX40L was turned on selectively in CD8+ T cells (FIG. 18A left panel) and monocytes (FIG. 18B) but not in HSCs (FIG. 18A right panel) in an miR142 ON system. Similar trends to those seen in CD8+ T cells were observed in T cells, hPBMCs, and BM cells.

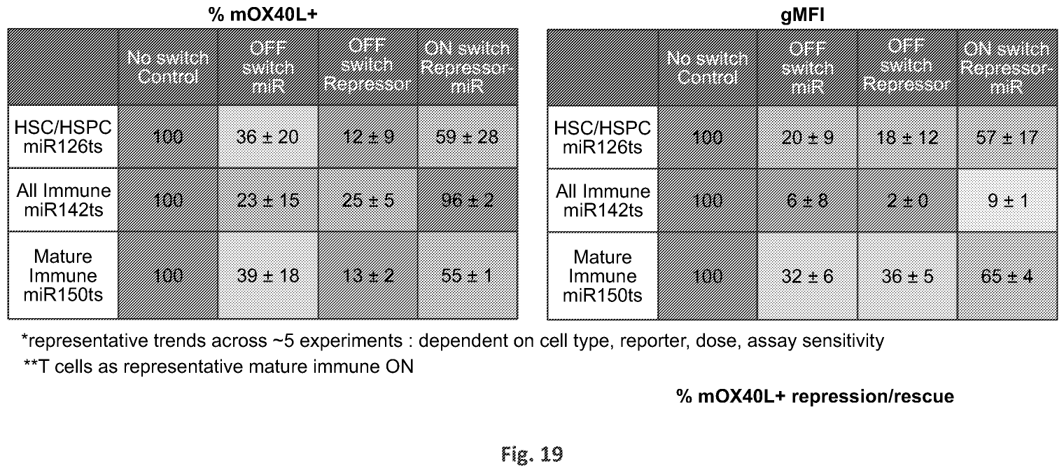

FIG. 19 is a summary consolidating the % suppression of mOX40L expression for the various ON/OFF systems exemplified in this disclosure.

Collectively, this data suggests that miR142-ts can turn ON expression in mature immune cells, and that miR150-ts can turn ON expression in CD4+ and CD8+ T cells.

### Example 9: MiRts Design Improvements can Enhance miR150ts, miR142ts, and miR122ts Efficacy

This example shows RNA design modifications that were tested in an OFF and ON system using HUH7, RAW264.7, human PBMCs and HeLa or Hep3B cells transfected with relevant miR mimic.

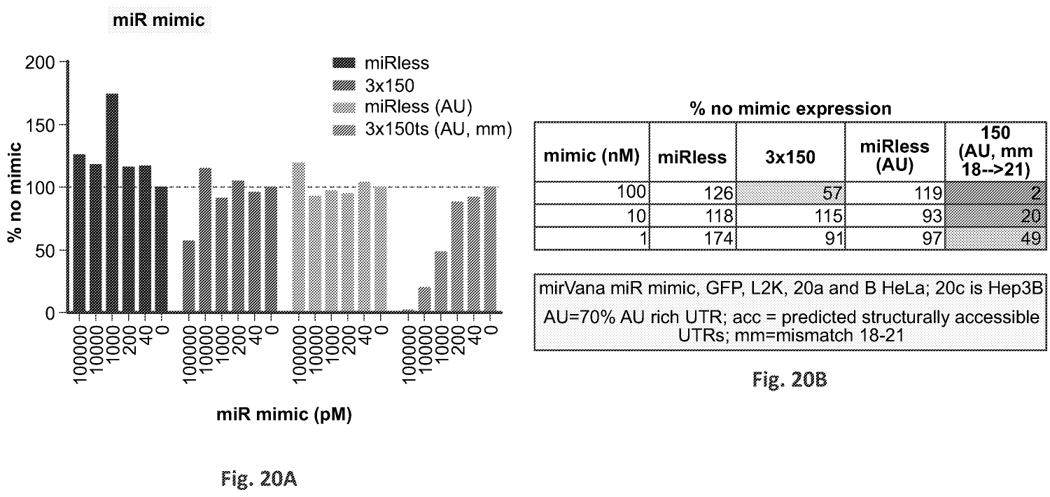

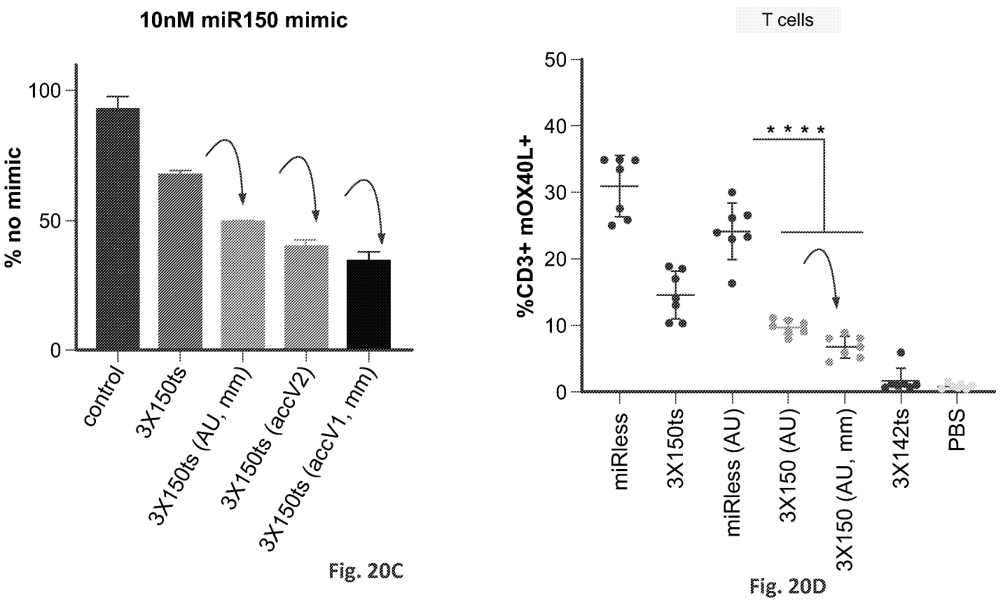

In one study, HeLa cells were transfected with mirVana miR150 mimic using lipofectamine 2000 across a dose range, along with eGFP reporter mRNAs containing 3×miR150ts, 3×miR150ts (AU, mm), or no miRts (miRless and miRless-AU). eGFP reporter expression was analyzed over 48 h using Incucyte live cell imaging. The signal was normalized to a non-relevant mimic. FIG. 20A depicts the AUC as a percentage of non-relevant mimic. miR150ts mediated suppression was increased by incorporating miR150ts in an AU rich UTR, along with adding mismatches at the 5′ target site end. The findings of FIG. 20A are depicted in tabular form in FIG. 20B.

In a second study, Hep3B cells were transfected with 10 nM mirVana miR150 mimic using lipofectamine 2000, along with eGFP reporter mRNAs containing 3×miR150ts, 3×miR150ts (AU, mm), 3×miR150ts (accV2), 3×miR150ts (accV1, mm) or no miRts (miRless). eGFP reporter expression was analyzed over 48 h using Incucyte live cell imaging. The signal was normalized to a non-relevant mimic. FIG. 20C depicts the AUC as a percentage of non-relevant mimic. miR150ts mediated suppression was increased by incorporating miR150ts in an AU rich UTR, along with adding mismatches at the 5′ target site end. miR150ts mediated suppression was even further increased by incorporating miR150ts in a structurally accessible target site design, and by adding mismatches at the 5′target site end.

In a third study, human peripheral blood mononuclear cells were transfected at 100 ng with Compound II-containing lipid nanoparticles containing mOX40L reporter mRNAs with the indicated target sites 3×miR150ts, 3×miR150t (AU), 3×miR150ts (AU, mm), 3×miR142ts, or mOX40L reporter with no target sites (miRless) or with no target sites in the AU rich UTR (miRless_AU). mOX40L expression was analyzed 24 h post transfection by flow cytometry. FIG. 20D depicts mOX40L expression in CD3+ T cells. miR150ts mediated suppression was increased by incorporating miR150ts in an AU rich UTR, and further increased by adding mismatches at the 5′ target site end.

In another study, HeLa cells were transfected with 20 ng eGFP reporter mRNAs containing indicated target sites 1×miR122ts, 3×miR122ts, 1×miR122ts (AU, mm, bridge), 3×miR122ts (AU, mm, bridge), or eGFP reporter with no target sites (miRless), along with 10 nM miR122 mimic using lipofectamine 2000. eGFP expression was analyzed over 48 h using Incucyte live cell imaging. FIG. 21A depicts AUC of total green fluorescence. miR122ts mediated suppression was improved by increasing number of target sites in the 3′UTR from 1× to 3×miR122 target sites. Suppression was further increased by addition of miR122 target sites in the 5′UTR, incorporating miR122 target sites in an AU rich 3′UTR, and adding mismatches to the 5′end of the miR122 target site.

In a subsequent study, HUH7 cells, that contain endogenous miR122, were transfected with 10 ng eGFP target RNA with non-relevant filler RNA (control), or with various L7Ae repressor RNA constructs (each 0.0625× molar) as follows: L7Ae repressor RNA without miR target sites (R_miRless); L7Ae repressor RNA containing 3×miR122ts; L7Ae repressor RNA containing 3×miR122ts (mm); L7Ae repressor RNA containing 3×miR122ts (AU, mm); L7Ae repressor RNA containing 6×miR122ts (AU, mm, bridge). eGFP expression was analyzed over 48 h using Incucyte live cell imaging. FIG. 21B depicts AUC of total green fluorescence. Expression was turned ON to a greater extent by increasing miR122ts in the 3′UTR, adding miR122ts in a 70% AU rich 3′UTR, and mismatches to the 5′ end of the miR122 target site, And incorporating 3×miR122ts in the 5′UTR.

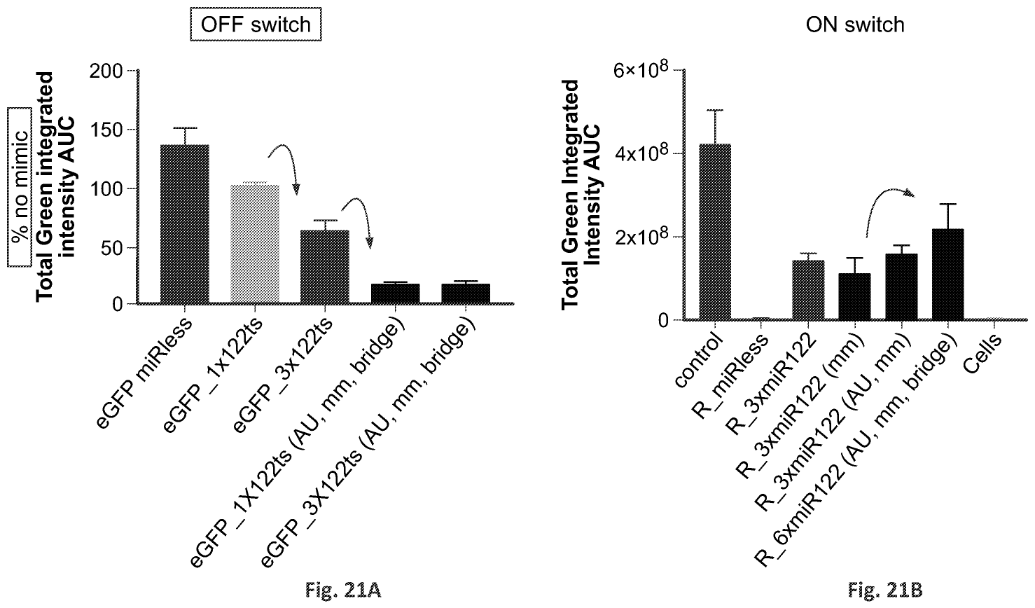

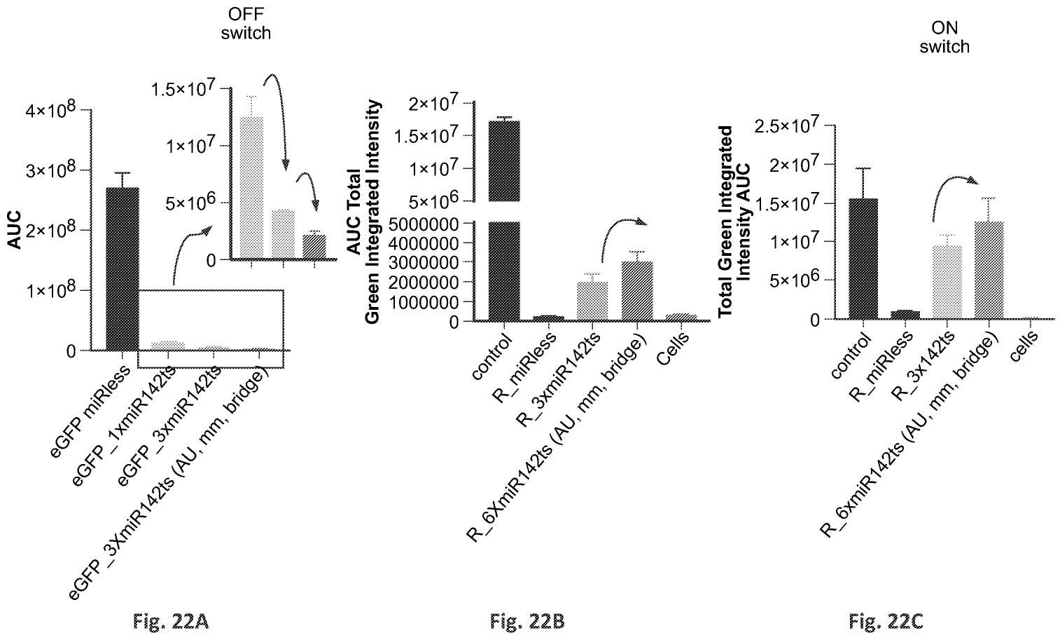

In another study, RAW264.7 cells, which contain endogenous miR142, were transfected with 10 ng eGFP reporter mRNAs containing indicated target sites 1×miR142ts, 3×miR142ts, 3×miR122ts (AU, mm, bridge) using lipofectamine 2000. eGFP expression was analyzed over 48 h using Incucyte live cell imaging. FIG. 22A depicts AUC of total green fluorescence. miR142ts mediated suppression was enhanced by increasing number of miR142ts in the 3′UTR from 1× to 3×miR142ts. Suppression was further increased by incorporating miR142ts in a 70% AU rich 3′UTR, addition of mismatches at the 5′end of the target site, and incorporating 3×miR142ts in the 5′UTR In a subsequent study, RAW264.7 cells were transfected with 10 ng eGFP target RNA with non-relevant filler RNA (control), or with various L7Ae repressor RNA constructs (each 0.0625× molar) as follows: L7Ae repressor RNA without miR target sites (R_miRless); L7Ae repressor RNA containing 3×miR142ts; 6×miR142ts (AU, mm, bridge). eGFP expression was analyzed over 48 h using Incucyte live cell imaging. FIG. 22B depicts AUC of total green fluorescence. Expression was turned ON to a greater extent by increasing number of miR142ts in the 3′UTR, addition of miR142ts in a 70% AU rich UTR, addition of mismatches at the 5′end of the target site, and incorporating 3×miR142ts in the 5′UTR.

In an additional study, RAW264.7 cells were transfected with 10 ng eGFP target RNA with non-relevant filler RNA (control), or with various Puf repressor RNA constructs (each 0.2× molar) as follows: Puf repressor RNA with no miR target sites (R_miRless); Puf repressor RNA containing 3×miR142ts (R_3×142ts); Puf repressor RNA containing 6×miR142ts (R_6×miR142ts (AU, mm, bridge)). eGFP expression was analyzed over 48 h using Incucyte live cell imaging. FIG. 22C depicts AUC of total green fluorescence. Expression was turned ON to a greater extent by increasing number of miR142ts in the 3′UTR, addition of miR142ts in a 70% AU rich UTR, addition of mismatches at the 5′end of the target site, and incorporating 3×miR142ts in the 5′UTR.

Collectively, this data suggests that suppression with miR150, miR142, and miR122 can be improved by modulating target site design.

### Example 10: Combining miR142-Ts in 3′& 5′UTR, a 3′AU-Rich UTR, and 3′End Target Site Mismatches Leads to Increased miR142-Ts Efficacy in Mice

This example shows RNA design modifications that were tested in an ON system in mice. CD-1 mice, at 5 mice/group, were administered lipid nanoparticles (containing Compound II and Compound I) containing 1 mg/kg NPI-Luciferase target mRNA with non-relevant filler mRNA or with various L7Ae repressor RNA constructs (each 0.5× molar) as follows: L7Ae repressor RNA without miR target sites (R_miRless); L7Ae repressor RNA containing 3×miR142ts, (R_3×miR142ts); L7Ae repressor RNA containing 3×miR122ts, (R_3×miR122ts), L7Ae repressor RNA containing 6×miR142ts, (R_6×miR142ts (AU, mm, bridge); and L7Ae repressor RNA containing 6×miR122ts, (R_6×miR122ts (AU, mm, bridge). Spleen and liver were collected at 6 h post dose for IHC analysis. % Positive V5 cells in the spleen, which contain endogenous miR142, are depicted in FIG. 23A. Incorporating miR142ts in a 70% AU rich UTR, increasing number of miR142ts, addition of mismatches at the 5′end of the target site, along with incorporating 3×miR142ts in the 5′UTR increased miR142 mediated suppression compared to a design which only incorporates 3×miR142ts in the 3′UTR. FIG. 23B depicts % V5 positive Kupffer cells in liver, which also contain endogenous miR142, determined by brightfield colocalization. Incorporating miR142ts in a 70% AU rich UTR, increasing number of miR142ts, addition of mismatches at the 5′end of the target site, along with incorporating 3×miR142ts in the 5′UTR increased miR142 mediated suppression compared to a design which only incorporates 3×miR142ts in the 3′UTR.

Collectively, this data suggests that suppression with miR142 in mouse splenic and Kupffer cells can be improved by modulating target site design.

### Example 11: Presence of miR142 Target Sites in 3′ and 5′ UTRs

Different RNA designs were tested to assess how the placement and number of mir142 target sites affected expression of FVIII and FVIII inhibitor formation. The following four constructs were tested:

TABLE 8

| Name | ORF | 5′UTR | 3′UTR |
|---|---|---|---|
| FVIII | FVIII_cN6_F309_mod | V1.1 5′UTR A-start | V1.1 3′UTR |
| FVIII - 3Xmir | FVIII_cN6_F309_mod | V1.1 5′UTR A-start | 3′UTR v1.1_3xmiR142-3p |
| FVIII - 6Xmir | FVIII_cN6_F309_mod | V1.1 5′UTR A-start | 3′cc_6xmiR142_mm |
| FVIII - 9Xmir | FVIII_cN6_F309_mod | V1.1_3xmiR142 | 3′cc_6xmiR142_mm |

### Sequences for the Constructs Listed in Table 8 Above:

5′ UTR; V1.1 5′UTR A-start:
(SEQ ID NO: 121)
AGGAAAUAAGAGAGAAAAGAAGAGUAAGAAGAAAUAUAAGACCCCGGCGCCGCCACC
5′ UTR; V1.1_3xmiR142 (miR142-3p are in bold):
(SEQ ID NO: 203)
AGGAAAUAAGAGAGUCCAUAAAGUAGGAAACACUACAAAAAGAAGAGUAAUCCAUAAAGUAGGAAACACUACA
AAGAAAUAUAAUCCAUAAAGUAGGAAACACUACAGACCCCGGCGCCGCCACC
3′ UTR; V1.1 3′UTR:
(SEQ ID NO: 4)
UGAUAAUAGGCUGGAGCCUCGGUGGCCUAGCUUCUUGCCCCUUGGGCCUCCCCCCAGCCCCUCCUCCCCUUCCUGC
CCCGUACCCCCGUGGUCUUUGAAUAAAGUCUGAGUGGGCGGC
3′ UTR; 3′UTR v1.1_3xmiR142-3p (miR142-3p are in bold):
(SEQ ID NO: 105)
UGAUAAUAGUCCAUAAAGUAGGAAACACUACAGCUGGAGCCUCGGUGGCCUAGCUUCUUGCCCCUUGGGCCUCCA
UAAAGUAGGAAACACUACAUCCCCCCAGCCCCUCCUCCCCUUCCUGCACCCGUACCCCCUCCAUAAAGUAGGAAAC
ACUACAGUGGUCUUUGAAUAAAGUCUGAGUGGGCGGC
3′ UTR; 3′cc_6xmiR142 mm (mismatched miR142-3p_mm are in bold):
(SEQ ID NO: 155)
UGAUAAUAGGCUGGAGCCUCAUUAAUUACAAAAGUAGGAAACACUACAUAUAAAGUAAAAUUUUACAAAAGUAG
GAAACACUACACACCAUUUUAAUUAUUACAAAAGUAGGAAACACUACAUAAUAAAAAUAAAGUUACAAAAGUAG
GAAACACUACAUAUAUAAUUCAUAGUUACAAAAGUAGGAAACACUACAUACCCCCGUGGUCUUUACAAAAGUAG
GAAACACUACAUUAAAUAAAGUCUAAGUGGGCGGC
ORF (which is the same for each of the four constructs noted above):
(SEQ ID NO: 204)
AUGCAAAUAGAGCUCUCCACCUGCUUCUUUCUGUGCCUUUUGCGAUUCUGCUUUAGUGCCACCAGAAGAUACUA [symbol]
UGGGUGCAGUGGAACUGUCCUGGGACUAUAUGCAAAGUGAUCUCGGUGAGCUGCCUGUGGACGCAAGAUUUCCU [symbol]
UAGAGUGCCCAAGUCUUUUCCAUUCAACACCUCAGUCGUGUACAAGAAGACUCUGUUUGUAGAAUUCACGGAUCA
CUUUUCAACAUCGCUAAGCCAAGGCCACCCUGGAUGGGUCUGCUAGGUCCUACCAUCCAGGCUGAGGUUUACGAU
CAGUGGUCAUUACACUUAAGAACAUGGCUUCCCAUCCUGUCAGUCUUCACGCUGUUGGUGUAUCCUACUGGAAA [symbol]
UUCUGAGGGAGCUGAAUACGACGAUCAGACCAGUCAAAGGGAGAAAGAAGACGAUAAAGUCUUCCCUGGUGGAA [symbol]
CCAUACAUACGUCUGGCAGGUCCUGAAAGAGAACGGUCCAAUGGCCUCUGACCCACUGUGCCUUACCUACUCAUA
CUUUCUCACGUGGACCUGGUCAAGGACUUGAAUUCAGGCCUCAUUGGAGCCCUACUAGUGUGUAGAGAAGGGAGU
UGGCCAAGGAGAAGACACAGACCUUGCACAAAUUUAUACUACUUUUCGCUGUAUUUGACGAAGGGAAGAGUUGG [symbol]
ACUCAGAAACAAAGAACUCCUUGAUGCAGGAUAGGGACGCUGCAUCUGCUCGGGCCUGGCCUAAGAUGCACACA [symbol]
CAACGGUUACGUAAACAGGUCCCUGCCAGGUCUGAUUGGCUGCCACAGGAAAUCAGUCUAUUGGCACGUGAUUG [symbol]
AUGGGCACCACUCCUGAAGUGCACUCAAUAUUCCUCGAAGGUCACACAUUUCUUGUGAGGAACCAUCGCCAGGCGU
CCUUGGAAAUCUCGCCAAUAACUUUCCUUACUGCUCAAACACUCUUGAUGGACCUUGGACAGUUUCUACUGUCUUG
UCAUAUCUCUUCCCACCAACACGACGGCAUGGAAGCUUACGUCAAAGUAGACAGCUGUCCAGAGGAACCCCAACUA
CGAAUGAAGAAUAACGAAGAAGCGGAAGACUACGACGACGAUCUUACUGAUUCUGAAAUGGACGUGGUCAGGUUU
GACGACGACAACUCUCCUUCCUUUAUCCAAAUUCGCUCAGUUGCCAAGAAGCAUCCUAAGACUUGGGUACAUUACA
UUGCUGCUGAAGAGGAGGACUGGGACUACGCUCCCUUAGUCCUCGCCCCUGACGACAGAAGUUAUAAGAGUCAAUA
UUUGAACAACGGCCCUCAGCGGAUUGGUAGGAAGUACAAGAAAGUCCGAUUUAUGGCAUACACAGACGAAACCUU
UAAGACUCGUGAAGCUAUUCAGCACGAAUCAGGAAUCUUGGGACCUUUACUUUACGGGGAAGUUGGAGACACACU
GUUGAUUAUAUUUAAGAAUCAAGCAAGCAGACCAUAUAACAUCUACCCUCACGGAAUCACUGACGUCCGUCCUUUG
UAUUCAAGGAGAUUACCCAAGGGUGUCAAGCAUUUGAAGGAUUUUCCAAUUCUGCCAGGAGAAAUAUUCAAAUAU
AAGUGGACAGUGACUGUAGAAGACGGGCCAACUAAAUCAGAUCCUCGGUGCCUGACCCGCUAUUACUCUAGUUUCG
UUAAUAUGGAGAGAGAUCUAGCUUCAGGACUCAUUGGCCCUCUCCUCAUCUGCUACAAAGAAUCUGUAGAUCAAAG
AGGAAACCAGAUAAUGUCAGACAAGAGGAACGUCAUCCUGUUCAGCGUCUUCGACGAGAACAGGAGCUGGUACCUC
ACAGAGAAUAUACAACGCUUUCUCCCCAAUCCAGCUGGAGUGCAGCUUGAGGAUCCAGAGUUCCAAGCCUCCAACA
UCAUGCACAGCAUCAACGGCUACGUGUUCGAUAGUUUGCAGUUGUCAGUUUGUUUGCACGAGGUGGCAUACUGGU
ACAUUCUAAGCAUUGGAGCACAGACUGACUUCCUGAGCGUGUUCUUCAGCGGCUACACCUUCAAACACAAGAUG [symbol]
CUACGAAGACACACUCACCCUAUUCCCAUUCUCAGGAGAAACUGUCUUCAUGUCGAUGGAGAACCCAGGUCUGUG
AUUCUGGGGUGCCACAACUCAGACUUUCGGAACAGAGGCAUGACCGCCUUACUGAAGGUUUCUAGUUGUGACAA [symbol]
ACACUGGUGAUUAUUACGAGGACAGUUACGAAGAUAUUUCAGCAUACUUGCUGAGUAAGAACAACGCCAUUGAA [symbol]
CAAGAAGCUUCUCCCAGAAUUCAAGACACCCUAGCACUAGGCAGAAGCAAUUUAACGCCACCACAAUUCCAGAGA
CGACAUAGAGAAGACUGACCCUUGGUUUGCACACAGAACACCUAUGCCUAAGAUACAGAACGUCUCCUCUAGUGA
UUGUUGAUGCUCUUGCGACAGAGUCCUACUCCACACGGGCUAUCCUUAUCUGAUCUCCAAGAAGCCAAAUACGAG
CAUUCUCUGACGAUCCAUCACCUGGAGCAAUAGACAGUAAUAACAGCCUGUCUGAAAUGACACACUUCAGGCCAC
GCUCCAUCACAGUGGGGACAUGGUAUUUACCCCUGAGUCAGGCCUCCAAUUAAGAUUAAACGAGAAACUGGGGA [symbol]
ACUGCAGCAACAGAGUUGAAGAAACUUGAUUUCAAAGUUUCUAGUACAUCAAAUAACCUGAUCUCCACCAUCCC [symbol]
GCGACAAUUUGGCAGCAGGUACUGAUAAUACAAGUUCCUUAGGACCACCAAGUAUGCCAGUUCAUUACGAUAGU [symbol]
AUUAGAUACCACUCUAUUUGGCAAGAAGUCAUCUCCCCUUACUGAGUCUGGUGGACCUCUGAGCUUGAGUGAAGA
AACAACGACUCCAAACUGCUGGAAUCCGGCUUAAUGAAUAGCCAAGAAAGUAGCUGGGGCAAGAACGUAUCGUCA
AACGGGAAAUAACUCGUACUACUCUUCAGUCAGAUCAAGAGGAAAUUGACUACGACGAUACCAUAUCAGUUGAAA
UGAAGAAGGAAGAUUUUGACAUUUACGACGAGGACGAGAAUCAGUCACCCCGCAGCUUUCAGAAGAAGACACGAC
ACUAUUUCAUCGCCGCCGUGGAGAGGCUGUGGGAUUACGGGAUGAGUAGCUCCCCACACGUUCUAAGAAACAGGGC
UCAGAGUGGCAGUGUCCCUCAGUUCAAGAAAGUUGUUUUCCAGGAAUUUACUGACGGCUCCUUUACUCAGCCCUUA
UACCGUGGAGAACUAAACGAACAUUUGGGACUCCUGGGGCCAUAUAUAAGAGCAGAAGUUGAAGAUAAUAUCAUG
GUAACUUUCAGAAAUCAGGCCUCUCGUCCCUAUUCCUUCUAUUCUAGCCUGAUCAGCUACGAGGAGGACCAGCGCC
AGGGAGCAGAACCUAGAAAGAACUUUGUCAAGCCUAACGAAACCAAGACUUACUUUUGGAAAGUGCAACAUCAUA
UGGCACCCACUAAAGACGAGUUUGACUGCAAAGCCUGGGCUUAUUUCUCUGACGUUGACCUGGAGAAAGACGUGCA
CUCAGGCCUGAUUGGACCCCUUCUGGUCUGCCACACUAACACACUGAACCCUGCUCACGGGAGACAAGUGACAGUA
CAGGAAUUUGCUCUGUUCUUCACCAUCUUUGACGAAACCAAGUCCUGGUACUUCACCGAGAAUAUGGAAAGAAACU
GCAGGGCUCCCUGCAAUAUCCAGAUGGAAGAUCCCACUUUUAAAGAGAAUUAUCGCUUCCACGCAAUCAACGGCUA
CAUAAUGGAUACACUACCUGGCUUAGUAAUGGCUCAGGAUCAAAGGAUUAGGUGGUAUCUGCUCAGCAUGGGCAG
CAACGAGAACAUCCAUUCUAUUCAUUUCAGUGGACACGUGUUCACUGUACGGAAGAAAGAGGAGUAUAAGAUGGC
ACUGUACAAUCUCUAUCCAGGUGUGUUCGAGACAGUGGAAAUGUUACCAUCCAAAGCUGGAAUUUGGCGGGUGGA
GUGCCUUAUUGGCGAGCAUCUACACGCUGGGAUGAGCACACUGUUCCUGGUGUACAGCAAUAAGUGUCAGACUCCC
CUGGGAAUGGCUUCUGGACACAUUAGAGAUUUUCAGAUUACAGCUUCAGGACAAUACGGACAGUGGGCCCCAAAGC
UGGCCAGACUUCAUUAUUCCGGAUCAAUCAACGCCUGGUCCACCAAGGAGCCCUUCAGCUGGAUCAAGGUGGAUC
GUUGGCACCAAUGAUUAUUCACGGCAUCAAGACCCAGGGUGCCCGUCAGAAGUUCUCCAGCCUCUACAUCUCUCA
UUUAUCAUCAUGUAUAGUCUUGACGGGAAGAAGUGGCAGACUUAUCGAGGAAAUUCCACUGGAACCUUAAUGGU [symbol]
UUCUUUGGCAACGUGGAUUCAUCUGGGAUCAAGCACAAUAUCUUCAACCCUCCAAUUAUUGCUCGAUACAUCCGU
UGCACCCAACUCAUUAUAGCAUUCGCAGCACUCUUCGCAUGGAGUUGAUGGGCUGUGAUUUAAAUAGUUGCAGCA
GCCAUUGGGAAUGGAGAGUAAAGCAAUAUCAGACGCACAGAUUACUGCUUCAUCCUACUUUACCAAUAUGUUUG [symbol]
ACCUGGUCCCCUUCCAAGGCUCGACUUCACCUCCAAGGGAGGAGUAACGCCUGGAGGCCUCAGGUGAAUAAUCCG
AGGAGUGGCUGCAAGUGGACUUCCAGAAGACAAUGAAAGUCACAGGAGUAACUACUCAGGGAGUCAAGUCUCUG [symbol]
UUACCAGCAUGUACGUGAAGGAGUUCCUCAUCUCCAGCAGUCAAGACGGCCAUCAGUGGACUCUCUUCUUUCAGA
CGGCAAAGUAAAGGUGUUUCAGGGAAAUCAAGACUCCUUCACACCUGUGGUGAACAGCCUGGACCCACCGUUACU
ACUCGCUACCUUCGAAUUCAUCCGCAGUCCUGGGUGCACCAGAUCGCCCUCAGGAUGGAGGUUCUGGGCUGCGAG
CACAGGACCUCUAC

[symbol]

indicates data missing or illegible when filed

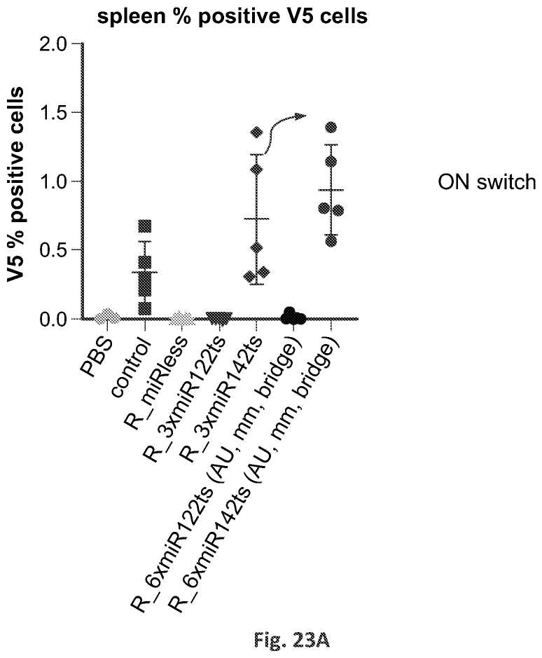

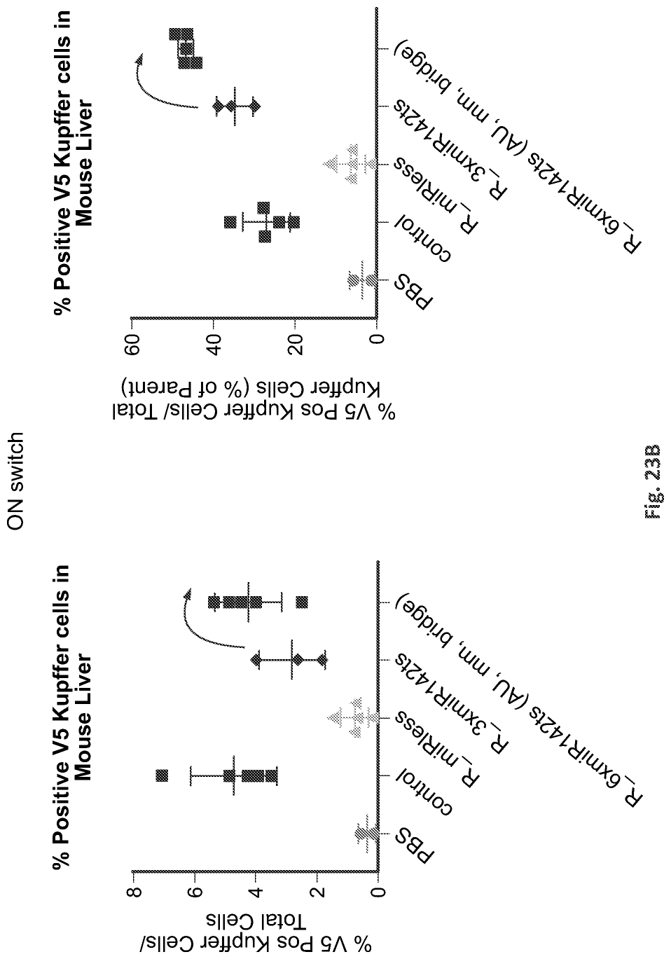

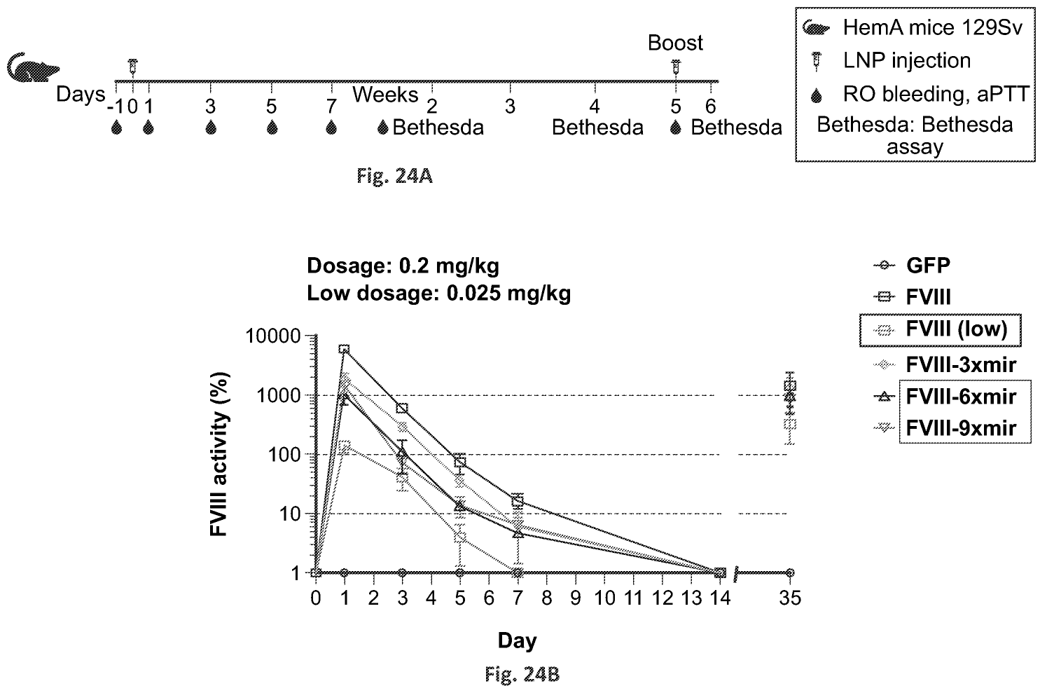

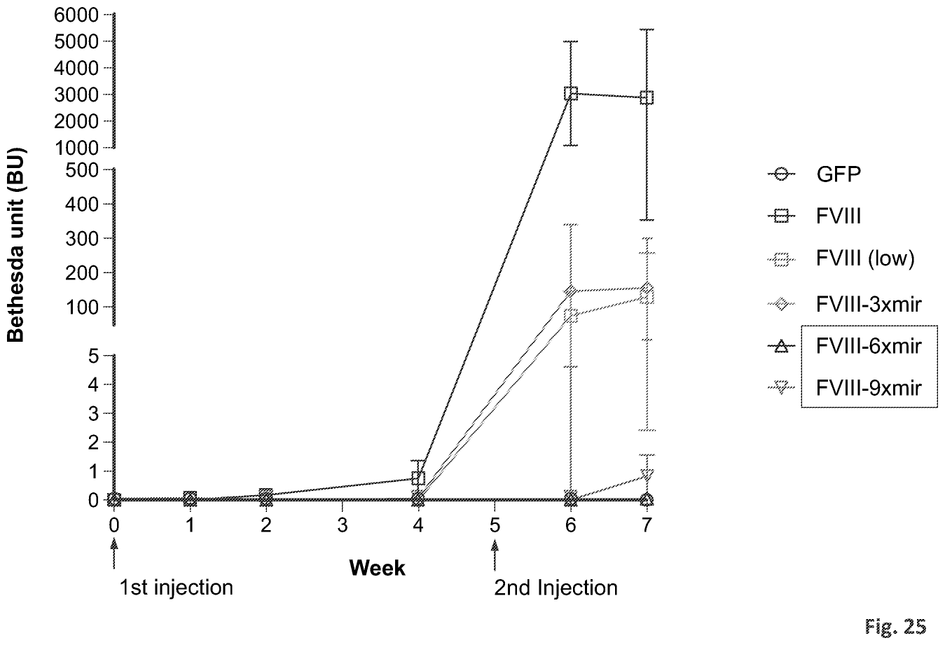

Each of the constructs were prepared and administered in lipid nanoparticles (containing Compound II and PEG-DMG). The experimental design of the first set of experiments is outlined in FIG. 24A (see also, Chen C Y, Tran D M, Cavedon A, Cai X, Rajendran R, Lyle M J, Martini P G V, Miao C H. Treatment of Hemophilia A Using Factor VIII Messenger RNA Lipid Nanoparticles. Mol Ther Nucleic Acids. 2020 Jun. 5; 20:534-544). Briefly, six cohorts of HemA 129Sv mice, at 3 mice/cohort, were administered 0.2 mg/kg mRNA containing lipid nanoparticles (a cohort for GFP, a cohort for each of the constructs noted above, and a sixth cohort that was administered a low 0.025 mg/kg dose for the FVIII mRNA construct). Retro-orbital bleeding was done 1 day prior to administration and then again at 1 day, 3 days, 5 days, 7 days, 2 weeks, and 5 weeks. A Bethesda assay was done at 2 weeks to assess the presence of FVIII inhibitors. The mice received a second dose of the 0.2 mg/kg mRNA containing lipid nanoparticles at 5 weeks. A further Bethesda assay was done at 6 weeks. As shown in FIG. 24B, mRNAs with mir142 targeting sequences showed lower FVIII expression. FIG. 25, as demonstrated by the Bethesda assays, show that mRNAs with mir142 targeting sequences showed little to no FVIII inhibitor formation, with mRNAs containing 6×mir142 or 9×mir142 target sites showing the most substantial reduction.

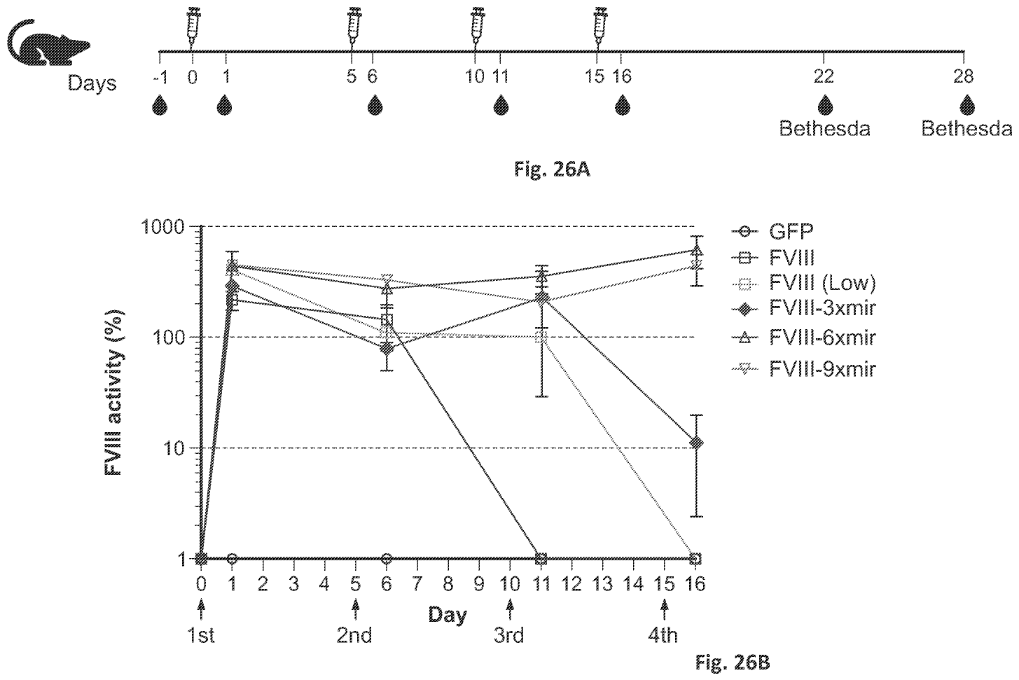

In the second set of experiments, which is outlined in FIG. 26A, six cohorts of HemA 129Sv mice, at 3 mice/cohort, were administered lipid nanoparticles containing mRNA according to the following table:

TABLE 9

| | | FVIII | FVIII | FVIII | FVIII | FVIII |
|---|---|---|---|---|---|---|
| ID | GFP | A | B | 3xmir | 6xmir | 9xmir |
| Dosage | 0.1 | 0.02 | 0.015 | 0.05 | 0.1 | 0.1 |
| (mg/kg) | | | | | | |

* FVIII A and FVIII B are the same construct (i.e., no mir142 targeting sequence), just used at different doses.

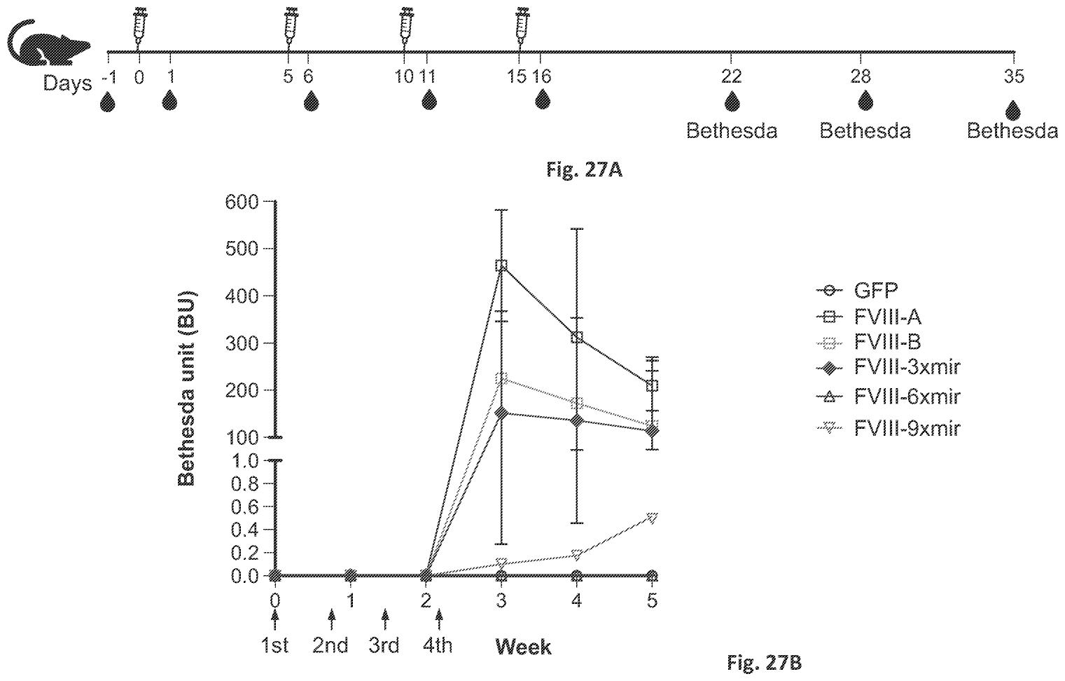

Retro-orbital bleeding was done 1 day prior to administration and then again at 1 day, 6 days, 11 days, 16 days, 22 days, and 28 days. Mice received subsequent doses at 5 days, 10 days, and 15 days. Bethesda assays were done at 22 days and 28 days. As shown in FIG. 26B, FVIII expression was maintained in HemA mice after repeated dosing of hFVIII mRNA containing 6×mir or 9×mir. FIG. 27, as demonstrated by Bethesda assays, show that incorporation of 6×mir in 3′UTR inhibited FVIII inhibitor formation after repeated hFVIII mRNA injections.

Collectively, this data suggests that FVIII expression can be improved and FVIII inhibitor formation reduced by modulating mRNA design using miR142 target sites.

### Example 12: Suppression of Expression by miR223

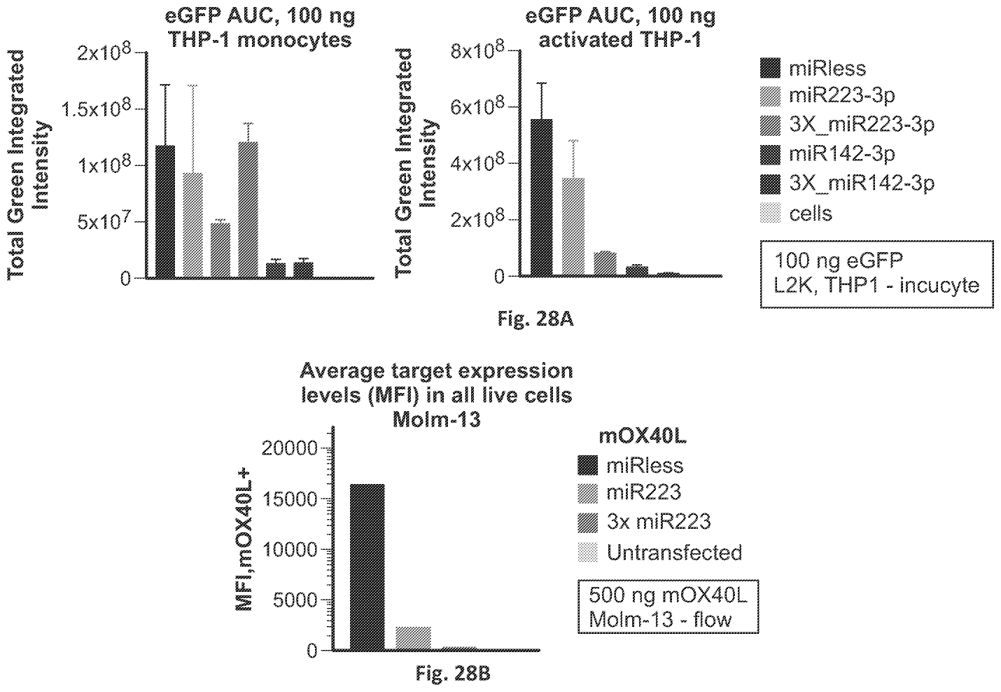

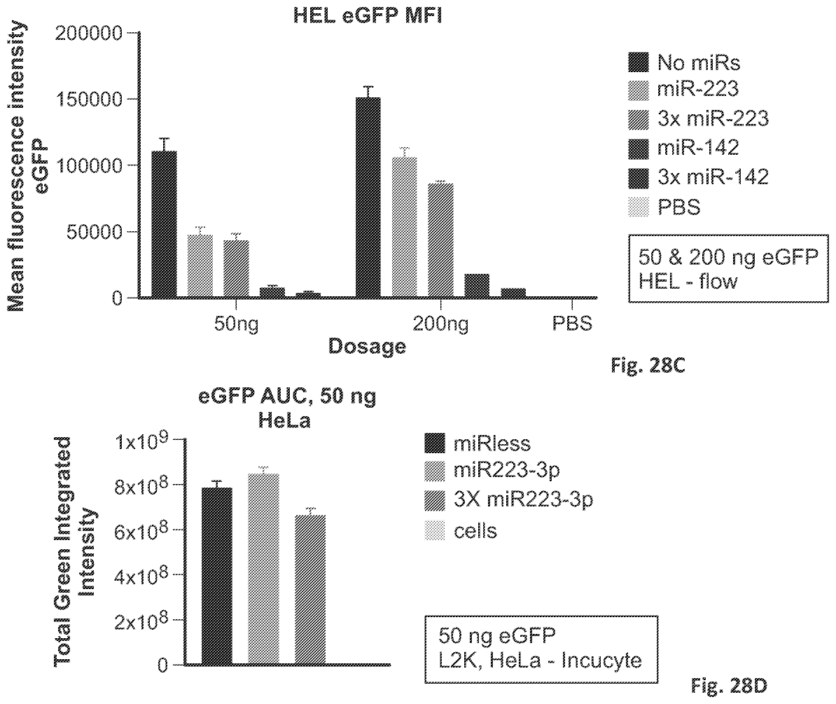

First, THP-1 non-activated and activated monocytes were transfected using lipofectamine 2000 with 100 ng eGFP reporter mRNAs containing no miR target sites (i.e., miRless), one copy of miR223-3p (“miR223-3p”), three copies of miR223-3p (“3×miR223-3p”), one copy of miR142-3p (“miR142-3p”), three copies of miR142-3p (“3×miR142-3p”), along with a non-transfected control (“cells”). eGFP reporter expression was analyzed over 48 h using Incucyte live cell imaging. FIG. 28A depicts the total green fluorescent intensity.

Then, OX40L expression in Molm-13 cells in the presence of mOX40L reporter mRNA with no miR target site (“miRless”), one copy of miR223-3p (“miR223-3p”), or three copies of miR223-3p (“3×miR223-3p”) was assessed. 500 ng mOX40L reporter mRNAs were formulated in lipid nanoparticles containing Compound II. mOX40L expression was analyzed 16 h post transfection. FIG. 28B shows the mean fluorescent intensity (MFI) of mOX40L+ cells.

Additionally, HEL cells were transfected with 50 ng or 200 ng eGFP reporter mRNAs containing no miR target sites (“miRless”), one copy of miR223-3p (“miR223-3p”), three copies of miR223-3p (“3×miR223-3p”), one copy of miR142-3p (“miR142-3p”), three copies of miR142-3p (“3×miR142-3p”), along with a non-transfected control (“PBS”). Reporter mRNAs were formulated in lipid nanoparticles containing Compound II. eGFP reporter expression was analyzed 16 h post transfection. FIG. 28C shows the MFI of eGFP+ cells.

In FIG. 28D, HeLa cells were transfected using lipofectamine 2000 with 50 ng eGFP reporter mRNAs containing no miR target sites (“miRless”), one copy of miR223-3p (“miR223-3p”), three copies of miR223-3p (“3×miR223-3p”), along with a non-transfected control (“cells”). eGFP reporter expression was analyzed over 48 h using Incucyte live cell imaging. FIG. 28D depicts the total green fluorescent intensity.

Incorporation of either miR223-3p or miR142-3p mediated suppression. The degree of suppression was increased when 3 copies of the target site was included.

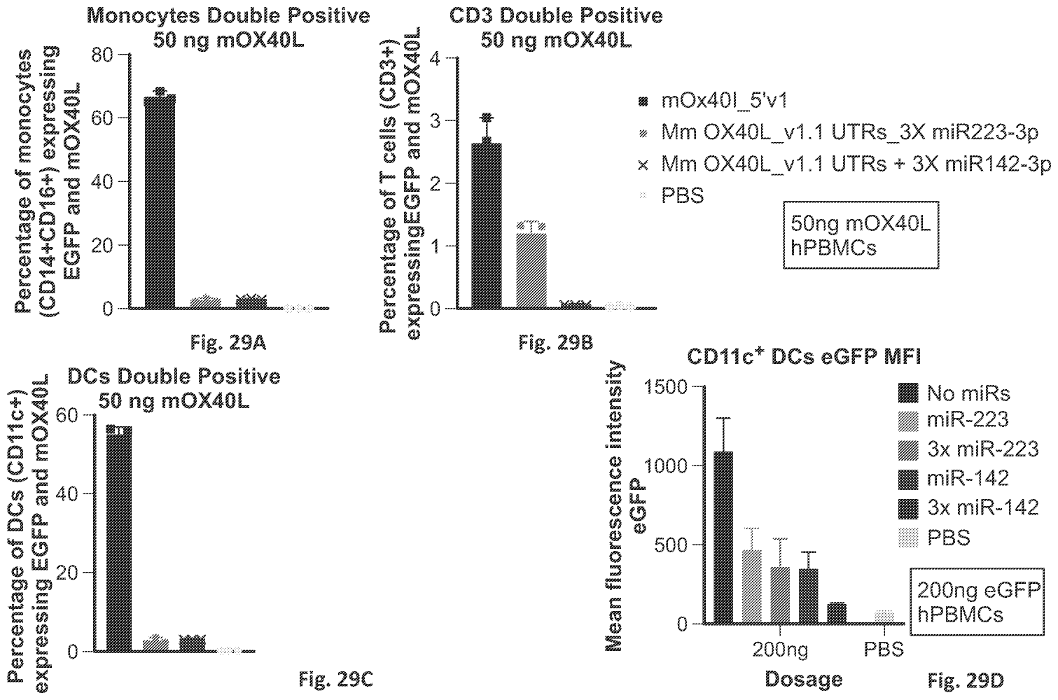

Second, OX40L and GFP expression in human PBMCs in the presence of an eGFP reporter mRNA co-dosed with mOX40L reporter mRNA with no miR target site (“miRless”), three copies of miR223-3p (“3×miR223-3p”), or three copies of miR142-3p (“3×miR142-3p”) was assessed. 50 ng mOX40L reporter mRNAs were formulated in lipid nanoparticles containing Compound II. mOX40L expression was analyzed [16 h post transfection. FIGS. 29A-C shows the percentage of the indicated cell types that were positive for both eGFP and mOX40L. An additional experiment, as shown in FIG. 29D, was done in which LNPs comprising 200 ng eGFP reporter mRNAs containing no miR target sites (“miRless”), one copy of miR223-3p (“miR223-3p”), three copies of miR223-3p (“3×miR223-3p”), one copy of miR142-3p (“miR142-3p”), three copies of miR142-3p (“3×miR142-3p”) along with a non-transfected control (“PBS”) were delivered to hPBMCs. Subsequently, CD11c+DC cells were gated by flow analysis. FIG. 29D depicts the mean green fluorescent intensity.

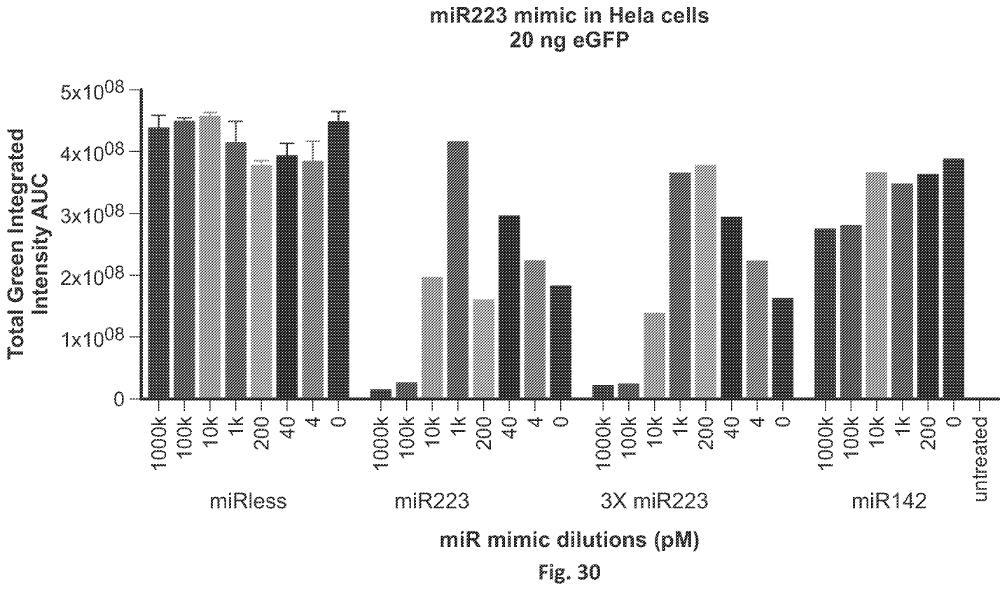

A mimic experiment was done in which HeLa cells were transfected with a mirVana miR223 mimic using lipofectamine 2000 across a dose range, along with eGFP reporter mRNAs containing miRless, miR223, 3×miR223, and miR142. eGFP reporter expression was analyzed over 48 h using Incucyte live cell imaging. FIG. 30 depicts the AUC as total green intensity.

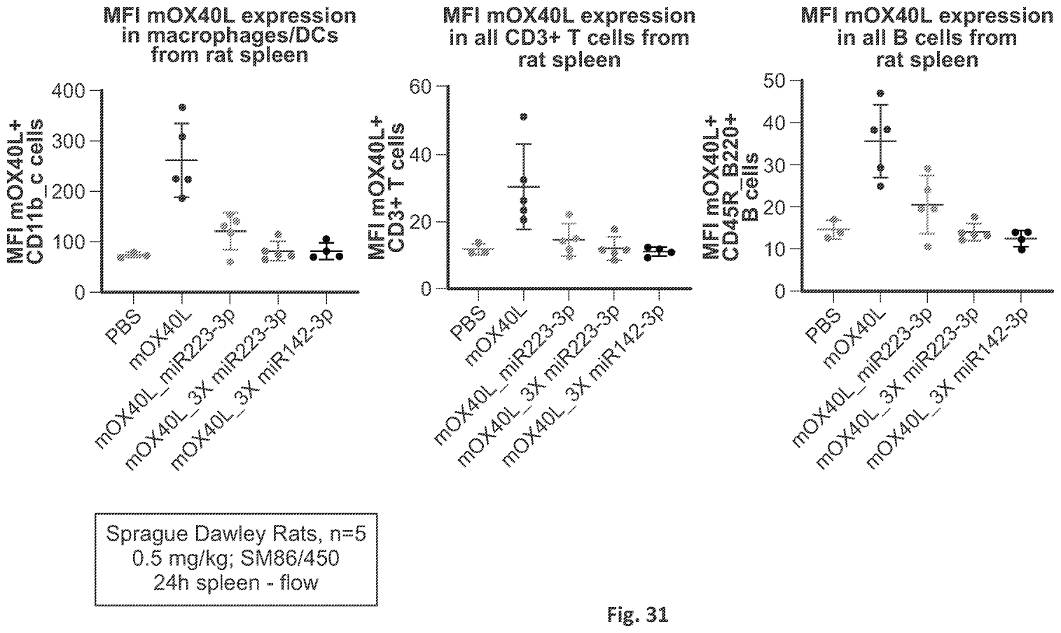

Next, ability of an mRNA containing a miR223 target site to suppress expression was addressed in rats. Sprague Dawley rats were injected IV with 0.5 mg/kg of mOX40L reporter mRNAs containing the indicated miR-target-sites (miR223-3p, 3×miR223-3p, or 3×miR142-3p) or mOX40L reporter mRNA with no miR target sites (mOX40L) or PBS (control). mOX40L reporter mRNAs were formulated in lipid nanoparticles containing Compound II. mOX40L expression in spleens was analyzed 24 h post transfection. Each graph in FIG. 31 shows the geometric MFI of mOX40L+ cells. The data shows that the expression of mOX40L (geometric MFI) was suppressed selectively in macrophages, T cells, and B cells that were exposed to miR223-3p or miR142-3p target sites containing mOX40L.

### Example 13: MiRts Design Improvements can Enhance Efficacy

Various designs of miR target sites were tested. Both the number (3 copies and 6 copies) in combination with location (3′UTR alone or in combination with 5′UTR), predicted favorable secondary structure in the 3′UTR, and AU rich context of the 3′UTR was tested.

HeLa cells were transfected using with 10 nM, 1 nM, or 0.2 nM mirVana miR126 mimic or miR150 mimic using lipofectamine 2000, along with 10 ng mGreenLantern reporter mRNAs containing the following: 5′ & 3′ v2.0 gLantern, 5′v2_mGreenLantern_3′v1.1_3×miR126ts, 5′v2_mGreenLantern_3′cc 6×miR126 mm, 5′v2 mGreenLantern 3′accV3 AU 6×miR126 mm, 5′v1.1_3×miR126ts_mGreenLantern_3′cc_6×miR126mmts, or miRless.

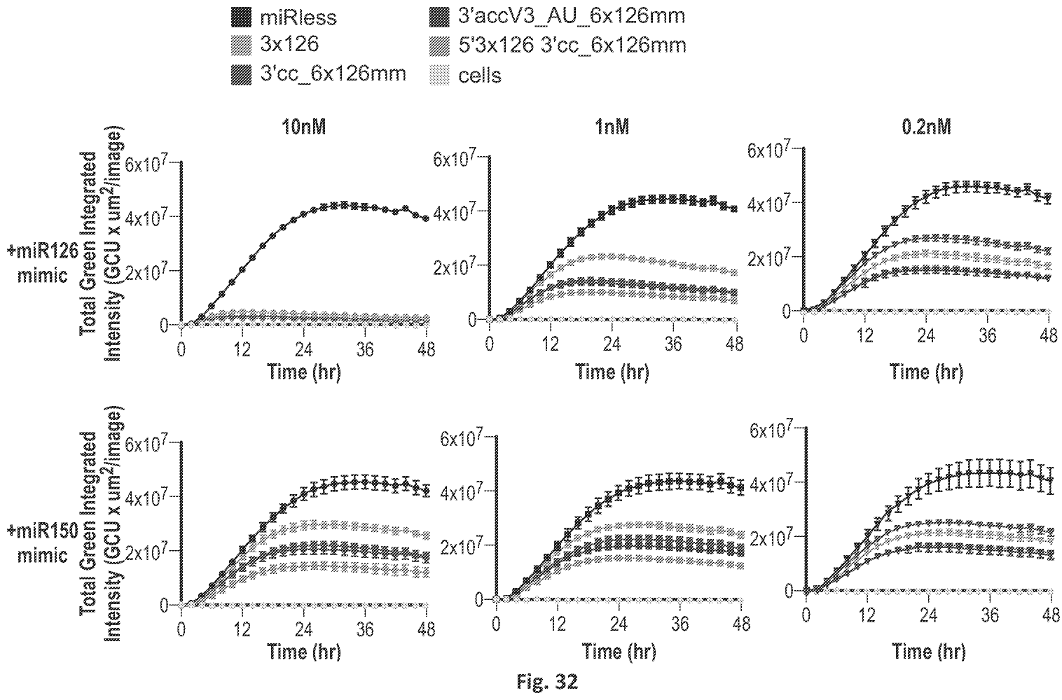

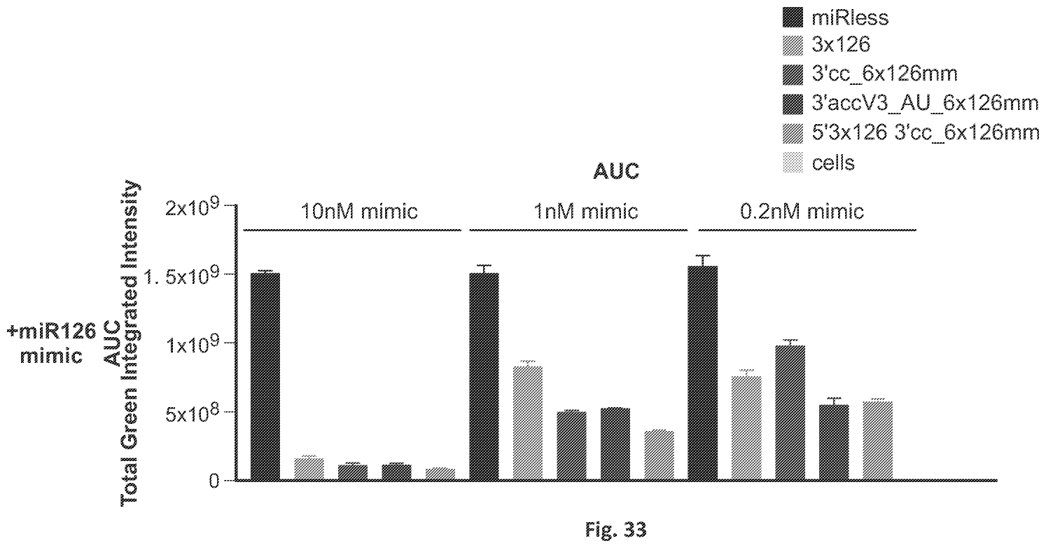

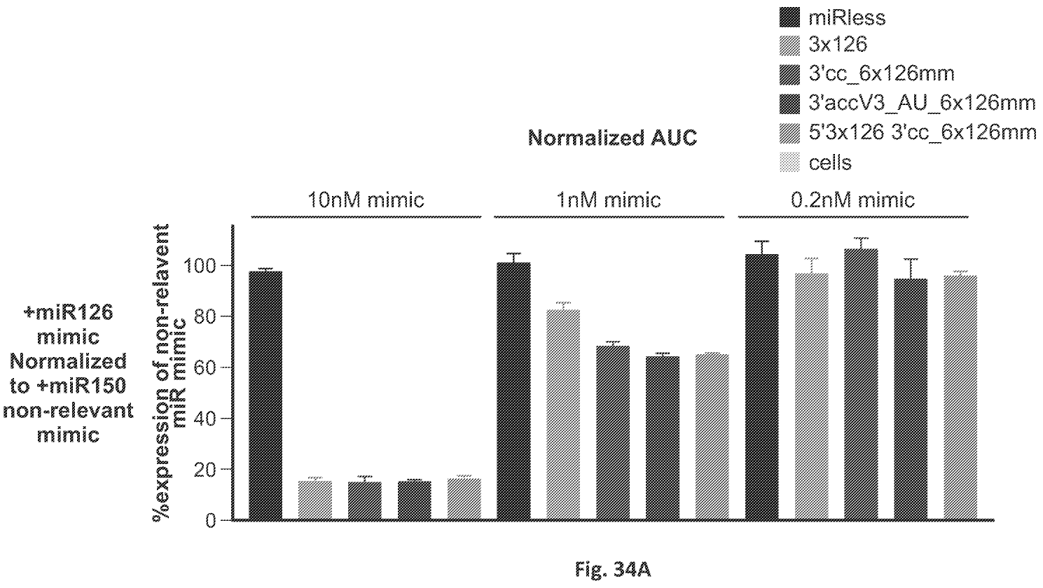

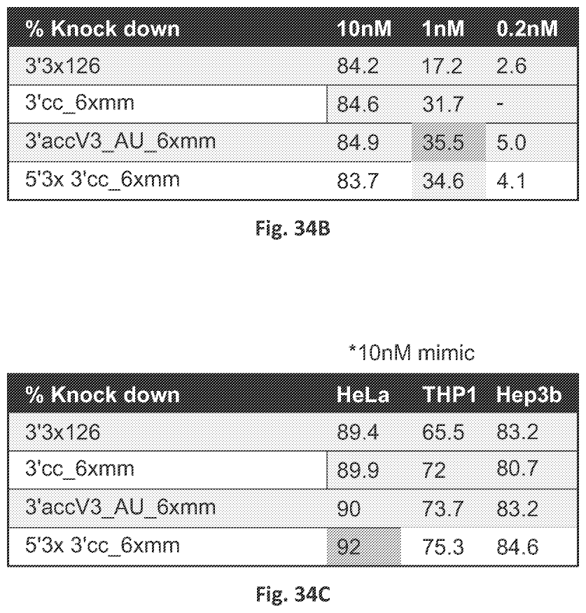

Reporter expression was analyzed over 48 h using Incucyte live cell imaging. FIG. 32 are graphs showing the total green intensity for each of the mRNA constructs. FIG. 33 is raw data showing the total green intensity for each of the mRNA constructs in the presence of miR126 mimic. FIG. 32 and FIG. 33 show that dilutions of the miR mimic better depict the degree of repression obtained for each mRNA construct. FIG. 34A are graphs showing 6×mm and ‘bridge’ designs perform comparably. Further, in FIG. 34A the miR126 mimic data is normalized to miR150 non-relevant mimic. The findings of FIG. 34A are depicted in tabular form in FIG. 34B. Similar studies were done in THP1 and Hep3b cells. Degree of knockdown achieved for the noted constructs are shown in FIG. 34C.

Collectively, this data shows that an mRNA having the 6× design with AU rich context and predicted favorable secondary structure (3′accV3_AU_6×126 mm) and ‘bridge’ design (5′3×126 3′cc_6×126 mm) perform similarly in terms of % knockdown/repression. Further, when diluting the amount of miR mimic used, the repression from each design can be visualized and compared. Specifically, at 1 and 0.2n M repression is weakened as compared to at 10 nM mimic, however the 6× favorable context and bridge design remain as top viable constructs for miR126 through each dilution.

TABLE 10
Miscellaneous sequences in this disclosure:

| SEQ | | |
|---|---|---|
| ID | Sequence | |
| NO | information | Sequence |
| 23 | mOX40L_D99K | MEGEGVQPLDENLENGSRPRFKWKKTLRLVVSGIKGAGMLLCFIY |
| | | VCLQLSSSPAKDPPIQRLRGAVTRCEDGQLFISSYKNEYQTMEVQN |
| | | NSVVIKCKGLYIIYLKGSFFQEVKIDLHFREDHNPISIPMLNDGRRIVF |
| | | TVVASLAFKDKVYLTVNAPDTLCEHLQINDGELIVVQLTPGYCAPE |
| | | GSYHSTVNQVPL |
| 24 | eGFP | MVSKGEELFTGVVPILVELDGDVNGHKFSVSGEGEGDATYGKLTL |
| | | KFICTTGKLPVPWPTLVTTLTYGVQCFSRYPDHMKQHDFFKSAMPE |
| | | GYVQERTIFFKDDGNYKTRAEVKFEGDTLVNRIELKGIDFKEDGNIL |
| | | GHKLEYNYNSHNVYIMADKQKNGIKVNFKIRHNIEDGSVQLADHY |
| | | QQNTPIGDGPVLLPDNHYLSTQSALSKDPNEKRDHMVLLEFVTAAG |
| | | ITLGMDELYK |
| 200 | NPI-Luciferase | MGKPIPNPLLGLDSTQGQEQKLISEEDLAGKPIPNPLLGLDSTQGQE |
| | | QKLISEEDLAGKPIPNPLLGLDSTQGQEQKLISEEDLAGKPIPNPLLG |
| | | LDSTQGQEQKLISEEDLAGKPIPNPLLGLDSTQGQEQKLISEEDLAPA |
| | | AKRVKLDEDAKNIKKGPAPFYPLEDGTAGEQLHKAMKRYALVPGT |
| | | IAFTDAHIEVDITYAEYFEMSVRLAEAMKRYGLNTNHRIVVCSENSL |
| | | QFFMPVLGALFIGVAVAPANDIYNERELLNSMGISQPTVVFVSKKGL |
| | | QKILNVQKKLPIIQKIIIMDSKTDYQGFQSMYTFVTSHLPPGFNEYDF |
| | | VPESFDRDKTIALIMNSSGSTGLPKGVALPHRTACVRFSHARDPIFG |
| | | NQIIPDTAILSVVPFHHGFGMFTTLGYLICGFRVVLMYRFEEELFLRS |
| | | LQDYKIQSALLVPTLFSFFAKSTLIDKYDLSNLHEIASGGAPLSKEVG |
| | | EAVAKRFHLPGIRQGYGLTETTSAILITPEGDDKPGAVGKVVPFFEA |
| | | KVVDLDTGKTLGVNQRGELCVRGPMIMSGYVNNPEATNALIDKDG |
| | | WLHSGDIAYWDEDEHFFIVDRLKSLIKYKGYQVAPAELESILLQHPN |
| | | IFDAGVAGLPDDDAGELPAAVVVLEHGKTMTEKEIVDYVASQVTT |
| | | AKKLRGGVVFVDEVPKGLTGKLDARKIREILIKAKKGGKIAV |
| 201 | AU rich element | UAUUUAUUUAUUUAUUUGUUUGUUUGUUUUAUU |
| 202 | AU rich 3′UTR | UAAUAGUAAGCUGGAGCCUCAUUAAUAUAAAGUAAAAUUCAC |
| | | CAUUUUAAUUAUAAUAAAAAUAAAGUAUAUAAUUCAAUGUAC |
| | | CCCCGUGGUCUUUAAAUAAAGUCUAAGUGGGCGGC |

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A composition comprising a messenger RNA (mRNA) comprising (i) an open reading frame encoding a polypeptide, and (ii) one or more microRNA target sites present in hematopoietic stem and progenitor cells (HSPC miRts).

2. The composition of claim 1, wherein the one or more HSPC miRts comprise miR-126-3p, miR-130a-3p, miR-10a-5p, miR-29a-3p, miR125a-5p, miR125b-5p, or miR196b-5p.

3. The composition of claim 1, wherein the polypeptide is a gene editor, a cytokine, an apoptotic protein, a transcription factor, a DNA-binding protein, a receptor, an enzyme, or a chimeric antigen receptor.

4. The composition of any one of claims 1 to 3, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

5. The composition of any one of claims 1 to 3, wherein the composition comprises a lipid nanoparticle.

6. The composition of claim 5, wherein the lipid nanoparticle comprises an ionizable amino lipid of Formula (I): 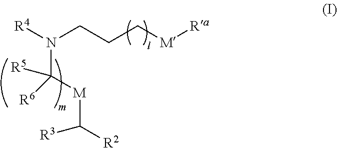
or a salt thereof,
wherein R′^{a} is R′^{branched}; wherein
R′^{branched} is: 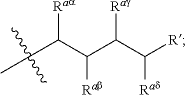
wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and 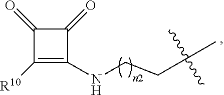
wherein [symbol] denotes a point of attachment; wherein
R¹⁰ is N(R)₂; each R is independently selected from the group consisting of C₁₋₆ alkyl, C₂-3 alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

7. The composition of claim 6, wherein the ionizable amino lipid has the formula: 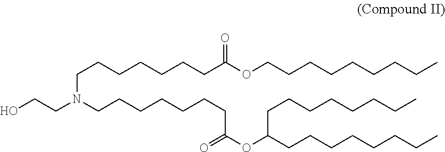 [SMILES: CCCCCCCCCOC(=O)CCCCCCCN(CCO)CCCCCCCC(=O)OC(CCCCCCCC)CCCCCCCC]
or a salt thereof.

8. The composition of any one of claims 5 to 7, wherein the lipid nanoparticle further comprises a PEG-lipid.

9. The composition of claim 8, wherein the PEG-lipid has the formula:

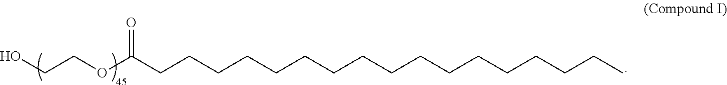

10. The composition of any one of claims 1 to 9, wherein the one or more HSPC miRts comprise at least one microRNA target site specific for miR126.

11. The composition of any one of claims 1 to 10, wherein the one or more HSPC miRts comprise at least two microRNA target sites specific for miR126.

12. The composition of any one of claims 1 to 9, wherein the one or more HSPC miRts comprise at least one microRNA target site specific for miR130a.

13. The composition of any one of claims 1 to 12, wherein the one or more HSPC miRts comprise at least two microRNA target sites specific for miR130a.

14. The composition of any one of claims 1 to 9, wherein the one or more HSPC miRts comprise at least two microRNA target sites specific for miR126 and at least two microRNA target sites specific for miR130a.

15. The composition of any one of claims 1 to 14, wherein the one or more HSPC miRts are in a non-coding region of the mRNA.

16. The composition of claim 15, wherein the 3′ untranslated region (UTR) of the mRNA comprises at least one HSPC miRts.

17. The composition of claim 15, wherein the 3′ UTR of the mRNA comprises at least two repeats of one HSPC miRts.

18. The composition of claim 15, wherein the 3′ UTR of the mRNA comprises six repeats of one HSPC miRts.

19. The composition of claim 15, wherein the 5′ UTR of the mRNA comprises at least one HSPC miRts.

20. The composition of claim 15, wherein the 5′ UTR of the mRNA comprises at least two repeats of one HSPC miRts.

21. The composition of claim 15, wherein the 5′ UTR of the mRNA comprises three repeats of one HSPC miRts.

22. The composition of claim 1, wherein the mRNA has one or more HSPC miRts in the 3′ UTR and the 5′ UTR.

23. The composition of claim 1, wherein the mRNA has one or more of the following features: (1) an AU-rich element; (2) the one or more HSPC miRts comprise at least one mismatch to the microRNA that binds the one or more HSPC miRts; (3) structurally accessible UTRs; (4) a short polyA tail; and (5) the ability to form microRNA bridges when a microRNA binds to the one or more HSPC miRts.

24. The composition of any one of claims 16-21, wherein the 5′ UTR and/or the 3′ UTR comprises an AU-rich element.

25. The composition of claim 24, wherein the 3′ UTR is 60%-90% AU-rich.

26. The composition of claim 24, wherein the 3′ UTR is about 70% AU-rich.

27. The composition of claim 23, wherein the one or more HSPC miRts comprise one to three mismatches to the microRNA that binds the one or more HSPC miRts.

28. The composition of claim 23, wherein the polyA tail is 20-100 nucleotides in length.

29. The composition of claim 23, wherein the microRNA bridge is formed by one or more miRts in the 5′ UTR and the 3′ UTR of the mRNA.

30. A method of preferentially expressing a polypeptide in hematopoietic cell types other than hematopoietic stem and progenitor cells (HSPCs), the method comprising contacting a population of hematopoietic cells with the composition of any one of claims 1 to 29, wherein the population of hematopoietic cells comprises HSPCs and hematopoietic cell types other than HSPCs.

31. The method of claim 30, wherein the contacting of the population of hematopoietic cells occurs ex vivo.

32. The method of claim 30, wherein the contacting of the population of hematopoietic cells occurs in vivo.

33. A composition comprising:
(a) a first polynucleotide comprising (i) a repressor binding element, (ii) an open reading frame encoding a polypeptide, and (iii) optionally one or more microRNA target sites present in non-hematopoietic stem and progenitor cells (non-HSPC miRts); and
(b) a second polynucleotide comprising (i) a sequence encoding a repressor that binds to the repressor binding element and (ii) one or more microRNA target sites present in hematopoietic stem and progenitor cells (HSPC miRts),
wherein binding of the repressor to the repressor binding element reduces translation of the polypeptide from the first polynucleotide.

34. The composition of claim 33, wherein the one or more HSPC miRts comprise miR-126-3p, miR-130a-3p, miR-10a-5p, miR-29a-3p, miR125a-5p, miR125b-5p, or miR196b-5p.

35. The composition of claim 33 or 34, wherein the one or more non-HSPC miRts comprise miR142-3p, miR150-5p, miR223-3p, or miR122-5p.

36. The composition of any one of claims 33 to 35, wherein the polypeptide is toxic to HSPCs.

37. The composition of any one of claims 33 to 35, wherein the polypeptide is a gene editor, a cytokine, an apoptotic protein, a transcription factor, a DNA-binding protein, a receptor, an enzyme, or a chimeric antigen receptor.

38. The composition of any one of claims 33 to 37, wherein the one or more HSPC miRts comprise at least one microRNA target site specific for miR126.

39. The composition of any one of claims 33 to 37, wherein the one or more HSPC miRts comprise at least two microRNA target sites specific for miR126.

40. The composition of any one of claims 33 to 39, wherein the one or more HSPC miRts comprise at least one microRNA target site specific for miR130a.

41. The composition of any one of claims 33 to 39, wherein the one or more HSPC miRts comprise at least two microRNA target sites specific for miR130a.

42. The composition of any one of claims 33 to 37, wherein the one or more HSPC miRts comprise at least two microRNA target sites specific for miR126 and at least two microRNA target sites specific for miR130a.

43. The composition of any one of claims 33 to 42, wherein the one or more microRNA target sites are in the non-coding region of each of the first and second polynucleotides, wherein each of the first and second polynucleotides is an mRNA.

44. The composition of claim 43, wherein the 3′ UTR of the first and second polynucleotides each comprises one microRNA target site.

45. The composition of claim 43, wherein the 3′ UTR of the first and second polynucleotides each comprises at least two repeats of one microRNA target site.

46. The composition of claim 43, wherein the 3′ UTR of the first and second polynucleotides each comprises six repeats of one microRNA target site.

47. The composition of claim 43, wherein the 5′ UTR of the first and second polynucleotides each comprises one microRNA target site.

48. The composition of claim 43, wherein the 5′ UTR of the first and second polynucleotides each comprises at least two repeats of one microRNA target site.

49. The composition of claim 43, wherein the 5′ UTR of the first and second polynucleotides each comprises three repeats of one microRNA target site.

50. The composition of claim 43, wherein the mRNA has one or more HSPC miRts in the 3′ UTR and the 5′ UTR.

51. The composition of claim 43, wherein the mRNA has one or more of the following features: (1) an AU-rich element; (2) the one or more HSPC miRts comprise at least one mismatch to the microRNA that binds the one or more HSPC miRts; (3) structurally accessible UTRs; (4) a short polyA tail; and (5) the ability to form microRNA bridges when a microRNA binds to the one or more HSPC miRts.

52. The composition of any one of claims 44-49, wherein the 5′ UTR and/or the 3′ UTR comprises an AU-rich element.

53. The composition of claim 52, wherein the 3′ UTR is 60%-90% AU-rich.

54. The composition of claim 52, wherein the 3′ UTR is about 70% AU-rich.

55. The composition of claim 51, wherein the one or more HSPC miRts comprise one to eight mismatches to the microRNA that binds the one or more HSPC miRts.

56. The composition of claim 51, wherein the polyA tail is 40-100 nucleotides in length.

57. The composition of claim 51, wherein the microRNA bridge is formed by one or more miRts in the 5′ UTR and the 3′ UTR of the mRNA.

58. The composition of any one of claims 33 to 42, wherein the first and the second polynucleotide each is an mRNA and comprises a polyA tail or is a DNA.

59. The composition of any one of claims 33 to 42 and 58, wherein the one or more HSPC miRts in the second polynucleotide are in the non-coding portion of the second polynucleotide.

60. The composition of any one of claims 33 to 42 and 58, wherein the one or more HSPC miRts in the second polynucleotide are (a) positioned between the sequence encoding the repressor and a polyA tail; or (b) positioned between a 5′ cap and a start codon, wherein the second polynucleotide is an mRNA.

61. The composition of any one of claims 33 to 60, wherein the repressor binding element comprises a kink-turn forming sequence.

62. The composition of claim 61, wherein the repressor binding element is selected from the group consisting of PRE, PRE2, MS2, PP7, BoxB, U1A hairpin, and 7SK.

63. The composition of any one of claims 33 to 62, wherein the repressor is selected from the group consisting of Snu13, 50S ribosomal L7Ae protein, Pumilio and FBF (PUF) protein, PUF2 protein, MBP-LacZ, MBP, PCP, Lambda N, U1A, 15.5kd, LARP7, L30e, and other RNA-binding proteins.

64. The composition of any one of claims 33 to 63, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

65. The composition of any one of claims 33 to 63, wherein the composition comprises a lipid nanoparticle.

66. The composition of claim 65, wherein the lipid nanoparticle comprises an ionizable amino lipid of Formula (I): 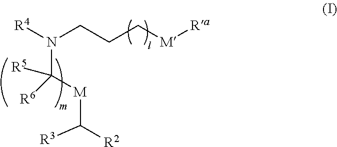
or a salt thereof,
wherein R′^{a} is R′^{branched}; wherein
R′^{branched} is: 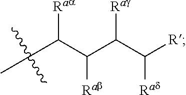
wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and 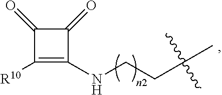
wherein [symbol] denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

67. The composition of claim 66, wherein the ionizable amino lipid has the formula: 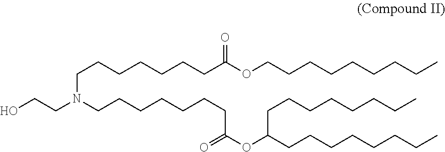 [SMILES: CCCCCCCCCOC(=O)CCCCCCCN(CCO)CCCCCCCC(=O)OC(CCCCCCCC)CCCCCCCC]
or a salt thereof.

68. The composition of any one of claims 65 to 67, wherein the lipid nanoparticle further comprises a PEG-lipid.

69. The composition of claim 68, wherein the PEG-lipid has the formula:

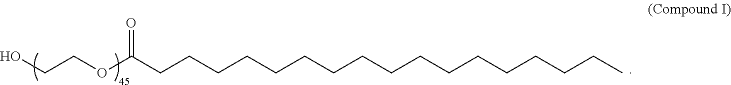

70. A method of preferentially expressing a polypeptide in hematopoietic stem and progenitor cells (HSPCs), the method comprising contacting a population of hematopoietic cells with the composition of any one of claims 33 to 69, wherein the population of hematopoietic cells comprises HSPCs.

71. The method of claim 70, wherein the contacting of the population of hematopoietic cells occurs ex vivo.

72. The method of claim 70, wherein the contacting of the population of hematopoietic cells occurs in vivo.

73. A method of expressing a polypeptide in a hematopoietic stem and progenitor cell (HSPC), the method comprising contacting the cell with:
(a) a first polynucleotide comprising (i) a repressor binding element, (ii) an open reading frame encoding a polypeptide, and (iii) optionally one or more non-hematopoietic stem cell-microRNA target sites present in non-hematopoietic stem and progenitor cells (non-HSPC miRts), wherein modification of the one or more non-HSPC miRts reduces translation of the polypeptide from the first polynucleotide; and
(b) a second polynucleotide comprising (i) a sequence encoding a repressor that binds to the repressor binding element and (ii) one or more microRNA target sites present in hematopoietic stem and progenitors (HSPC miRts),
wherein the HSPC expresses one or more microRNAs that bind to the one or more HSPC miRts and reduces translation of the repressor from the second polynucleotide.

74. A method of expressing a polypeptide in a hematopoietic stem and progenitor cell in a subject, the method comprising administering to the subject:
(a) a first polynucleotide comprising (i) a repressor binding element, (ii) an open reading frame encoding a polypeptide, and (iii) optionally one or more microRNA target sites present in non-hematopoietic stem and progenitor cells (non-HSPC miRts), wherein modification of the one or more non-HSPC miRts reduces translation of the polypeptide from the first polynucleotide; and
(b) a second polynucleotide comprising (i) a sequence encoding a repressor that binds to the repressor binding element and (ii) one or more microRNA target sites present in hematopoietic stem and progenitor cells (HSPC miRts),
wherein the HSPC expresses one or more microRNAs that bind to the one or more HSPC miRts and reduces translation of the repressor from the second polynucleotide.

75. A composition comprising:
(a) a first polynucleotide comprising (i) a repressor binding element, (ii) an open reading frame encoding a polypeptide, and (iii) optionally one or more microRNA target sites present in hematopoietic stem and progenitor cells (HSPC miRts); and
(b) a second polynucleotide comprising (i) a sequence encoding a repressor that binds to the repressor binding element and (ii) one or more microRNA target sites present in non-hematopoietic stem and progenitor cells (non-HSPC miRts),
wherein binding of the repressor to the repressor binding element reduces translation of the polypeptide from the first polynucleotide.

76. The composition of claim 75, wherein the one or more HSPC miRts comprise miR-126-3p, miR-130a-3p, miR-10a-5p, miR-29a-3p, miR125a-5p, miR125b-5p, or miR196b-5p.

77. The composition of claim 75 or 76, wherein the one or more non-HSPC miRts comprise a microRNA target site present in an immune cell or in a hepatocyte.

78. The composition of claim 77, wherein the microRNA target site present in an immune cell is miR142-3p, miR150-5p, or miR223-3p.

79. The composition of claim 77, wherein the microRNA target site present in a hepatocyte is miR-122-5p.

80. The composition of any one of claims 75 to 79, wherein the polypeptide is a gene editor, a cytokine, an apoptotic protein, a transcription factor, a DNA-binding protein, a receptor, an enzyme, or a chimeric antigen receptor.

81. The composition of any one of claims 75 to 80, wherein the one or more microRNA target sites are in the non-coding region of each of the first and second polynucleotides, wherein each of the first and second polynucleotides is an mRNA.

82. The composition of claim 81, wherein the 3′ UTR of the first and second polynucleotides each comprises one microRNA target site.

83. The composition of claim 81, wherein the 3′ UTR of the first and second polynucleotides each comprises at least two repeats of one microRNA target site.

84. The composition of claim 81, wherein the 3′ UTR of the first and second polynucleotides each comprises six repeats of one microRNA target site.

85. The composition of claim 81, wherein the 5′ UTR of the first and second polynucleotides each comprises one microRNA target site.

86. The composition of claim 81, wherein the 5′ UTR of the first and second polynucleotides each comprises at least two repeats of one microRNA target site.

87. The composition of claim 81, wherein the 5′ UTR of the first and second polynucleotides each comprises three repeats of one microRNA target site.

88. The composition of claim 81, wherein the mRNA has one or more HSPC miRts in the 3′ UTR and the 5′ UTR.

89. The composition of claim 81, wherein the mRNA has one or more of the following features: (1) an AU-rich element; (2) the one or more HSPC miRts comprise at least one mismatch to the microRNA that binds the one or more HSPC miRts; (3) structurally accessible UTRs; (4) a short polyA tail; and (5) the ability to form microRNA bridges when a microRNA binds to the one or more HSPC miRts.

90. The composition of any one of claims 82-87, wherein the 3′ UTR comprises an AU-rich element.

91. The composition of claim 90, wherein the 3′ UTR is 60%-90% AU-rich.

92. The composition of claim 90, wherein the 3′ UTR is about 70% AU-rich.

93. The composition of claim 89, wherein the one or more HSPC miRts comprise one to three mismatches to the microRNA that binds the one or more HSPC miRts.

94. The composition of claim 89, wherein the microRNA bridge is formed by one or more miRts in the 5′ UTR and the 3′ UTR of the mRNA.

95. The composition of any one of claims 75 to 80, wherein the first and second polynucleotide each is an mRNA and comprises a polyA tail or is a DNA.

96. The composition of any one of claims 75 to 80 and 95, wherein the one or more microRNA target sites in the second polynucleotide are (a) positioned between the sequence encoding the repressor and a polyA tail; or (b) positioned between a 5′ cap and a start codon, wherein the second polynucleotide is an mRNA.

97. The composition of any one of claims 75 to 96, wherein the repressor binding element comprises a kink-turn forming sequence.

98. The composition of claim 97, wherein the repressor binding element is selected from the group consisting of PRE, PRE2, MS2, PP7, BoxB, U1A hairpin, and 7SK.

99. The composition of any one of claims 75 to 98, wherein the repressor is selected from the group consisting of Snu13, 50S ribosomal L7Ae protein, Pumilio and FBF (PUF) protein, PUF2 protein, MBP-LacZ, MBP, PCP, Lambda N, U1A, 15.5kd, LARP7, L30e, and other RNA-binding proteins.

100. The composition of any one of claims 75 to 99, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

101. The composition of any one of claims 75 to 99, wherein the composition comprises a lipid nanoparticle.

102. The composition of claim 101, wherein the lipid nanoparticle comprises an ionizable amino lipid of Formula (I): 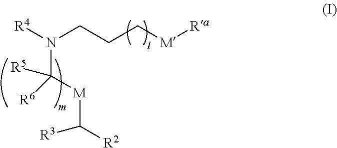
or a salt thereof,
wherein R′^{a} is R′^{branched}; wherein
R′^{branched} is: 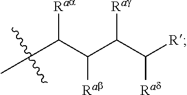
wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₄₄ alkenyl;
R⁴ is selected from the group consisting of —(CH₂)ₙOH, wherein n is selected from the group consisting of 1, 2, 3, 4, and 5, and 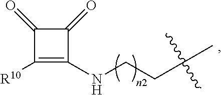
wherein [symbol] denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

103. The composition of claim 102, wherein the ionizable amino lipid has the formula: 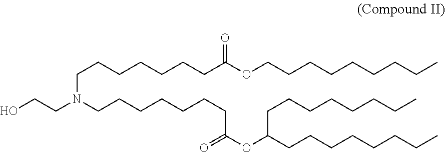 [SMILES: CCCCCCCCCOC(=O)CCCCCCCN(CCO)CCCCCCCC(=O)OC(CCCCCCCC)CCCCCCCC]
or a salt thereof.

104. The composition of any one of claims 101 to 103, wherein the lipid nanoparticle further comprises a PEG-lipid.

105. The composition of claim 104, wherein the PEG-lipid has the formula:

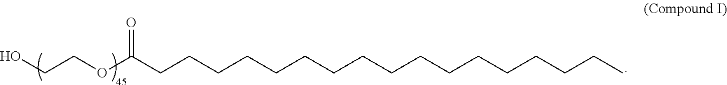

106. A method of preferentially expressing a polypeptide in hematopoietic cell types other than hematopoietic stem and progenitor cells (HSPCs), the method comprising contacting a population of hematopoietic cells with the composition of any one of claims 75 to 105, wherein the population of hematopoietic cells comprises HSPCs and hematopoietic cell types other than HSPCs.

107. The method of claim 106, wherein the contacting of the population of hematopoietic cells occurs ex vivo.

108. The method of claim 106, wherein the contacting of the population of hematopoietic cells occurs in vivo.

109. A method of expressing a polypeptide in a hematopoietic cell other than a hematopoietic stem and progenitor cell (HSPC), the method comprising contacting the cell with
(a) a first polynucleotide comprising (i) a repressor binding element, (ii) an open reading frame encoding a polypeptide, and (iii) optionally one or more microRNA target sites present in hematopoietic stem and progenitor cells (HSPC miRts), wherein modification of the one or more HSPC miRts reduces translation of the polypeptide from the polynucleotide; and
(b) an second polynucleotide comprising (i) a sequence encoding a repressor that binds to the repressor binding element and (ii) one or more non-HSPC miRts,
wherein the hematopoietic cell expresses one or more microRNAs that bind to the one or more non-HSPC miRts and reduces translation of the repressor from the second polynucleotide.

110. A method of expressing a polypeptide in a hematopoietic cell other than a hematopoietic stem and progenitor cell (HSPC) in a subject, the method comprising administering to the subject:
(a) a first polynucleotide comprising (i) a repressor binding element, (ii) an open reading frame encoding a polypeptide, and (iii) optionally one or more microRNA target sites present in hematopoietic stem and progenitor cell (HSPC miRts), wherein modification of the one or more HSPC miRts reduces translation of the polypeptide from the first polynucleotide; and
(b) a second polynucleotide comprising (i) a sequence encoding a repressor that binds to the repressor binding element and (ii) one or more non-HSPC miRts,
wherein the hematopoietic cell expresses one or more microRNAs that bind to the one or more non-HSPC miRts and reduces translation of the repressor from the second polynucleotide.

111. A composition comprising a first messenger RNA (mRNA) comprising (i) a first open reading frame encoding a first polypeptide, and (ii) at least six miR142 target sites.

112. The composition of claim 111, wherein the first polypeptide is a secreted protein.

113. The composition of claim 111 or 112, wherein the at least six miR142 target sites are in the 3′ UTR of the first mRNA.

114. The composition of any one of claims 111 to 113, wherein the at least six miR142 target sites each comprise the sequence UCCAUAAAGUAGGAAACACUACA (SEQ ID NO:191).

115. The composition of any one of claims 111 to 113, wherein the at least six miR142 target sites each comprise the sequence UUACAAAAGUAGGAAACACUACA (SEQ ID NO:197).

116. The composition of any one of claims 111 to 115, further comprising a second mRNA comprising (i) a second open reading frame encoding a second polypeptide, and (ii) at least one miR target site.

117. The composition of claim 116, wherein the second mRNA comprises at least two miR target sites.

118. The composition of claim 116, wherein the second mRNA comprises at least three miR target sites.

119. The composition of claim 116, wherein the second mRNA comprises at least four miR target sites.

120. The composition of claim 116, wherein the second mRNA comprises at least five miR target sites.

121. The composition of claim 116, wherein the second mRNA comprises at least six miR target sites.

122. The composition of any one of claims 116-121, wherein the at least one, at least two, at least three, at least four, at least five, or at least six miR target sites of the second mRNA are miR142 target sites.

123. The composition of claim 122, wherein the at least one, at least two, at least three, at least four, at least five, or at least six miR target sites of the second mRNA each comprise the sequence UCCAUAAAGUAGGAAACACUACA (SEQ ID NO:191).

124. The composition of claim 122, wherein the at least one, at least two, at least three, at least four, at least five, or at least six miR target sites of the second mRNA each comprise the sequence UUACAAAAGUAGGAAACACUACA (SEQ ID NO:197).

125. The composition of any one of claims 116-121, wherein the at least one, at least two, at least three, at least four, at least five, or at least six miR target sites of the second mRNA are selected from the group consisting of miR-126-3p, miR-130a-3p, miR-10a-5p, miR-29a-3p, miR125a-5p, miR125b-5p, miR196b-5p, miR150-5p, miR223-3p, and miR-122-5p target sites.

126. The composition of claim 125, wherein the second mRNA does not comprise a miR142 target site.

127. The composition of any one of claims 111-126, wherein the first and/or second mRNA comprise a 3′ UTR comprising the sequence
(SEQ ID NO: 154)
UGAUAAUAGGCUGGAGCCUCAUUAAUCCAUAAAGUAGGAAACACUACAUA
UAAAGUAAAAUUUCCAUAAAGUAGGAAACACUACACACCAUUUUAAUUAU
CCAUAAAGUAGGAAACACUACAUAAUAAAAAUAAAGUCCAUAAAGUAGGA
AACACUACAUAUAUAAUUCAUAGUCCAUAAAGUAGGAAACACUACAUACC
CCCGUGGUCUUCCAUAAAGUAGGAAACACUACAUUAAAUAAAGUCUAAGU
GGGCGGC.

128. The composition of any one of claims 111-126, wherein the first and/or second mRNA comprise a 3′ UTR comprising the sequence
(SEQ ID NO: 155)
UGAUAAUAGGCUGGAGCCUCAUUAAUUACAAAAGUAGGAAACACUACAUA
UAAAGUAAAAUUUUACAAAAGUAGGAAACACUACACACCAUUUUAAUUAU
UACAAAAGUAGGAAACACUACAUAAUAAAAAUAAAGUUACAAAAGUAGGA
AACACUACAUAUAUAAUUCAUAGUUACAAAAGUAGGAAACACUACAUACC
CCCGUGGUCUUUACAAAAGUAGGAAACACUACAUUAAAUAAAGUCUAAGU
GGGCGGC.

129. The composition of any one of claims 111-126, wherein the first and/or second mRNA comprise a 3′ UTR comprising the sequence
(SEQ ID NO: 170)
UAAAGCUCCCCGGGGGCUGGAGCCUCAUUAAUUACAAAAGUAGGAAACAC
UACAUAUAAAGUAAAAUUUUACAAAAGUAGGAAACACUACACACCAUUUU
AAUUAUUACAAAAGUAGGAAACACUACAUAAUAAAAAUAAAGUUACAAAA
GUAGGAAACACUACAUAUAUAAUUCAUAGUUACAAAAGUAGGAAACACUA
CAUACCCCCGUGGUCUUUACAAAAGUAGGAAACACUACAUUAAAUAAAGU
CUAAGUGGGCGGC.

130. The composition of any one of claims 111-126, wherein the first and/or second mRNA comprise a 3′ UTR comprising the sequence
(SEQ ID NO: 171)
UAAAGCUCCCCGGGGGCCUCAUUAAUUACAAAAGUAGGAAACACUACAUA
UAAAGUAAAAUUUUACAAAAGUAGGAAACACUACACACCAUUUUAAUUAU
UACAAAAGUAGGAAACACUACAUAAUAAAAAUAAAGUUACAAAAGUAGGA
AACACUACAUAUAUAAUUCAUAGUUACAAAAGUAGGAAACACUACAUACC
CCCGUGGUCUUUACAAAAGUAGGAAACACUACAUUAAAUAAAGUCUAAGU
GGGCGGC.

131. The composition of any one of claims 111 to 130, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

132. The composition of any one of claims 111 to 130, wherein the composition comprises a lipid nanoparticle.

133. A method of expressing a polypeptide in a subject, the method comprising administering to the subject the composition of any one of claims 111 to 132.

134. The method of claim 133, wherein the method comprises multiple administrations of the composition to the subject.

135. The method of claim 134, wherein the method comprises at least two, at least three, at least four, at least five, at least six, at least seven, at least eight, at least nine, or at least ten administrations of the composition to the subject.
